(19) United States
(12) Patent Application Publication
(10) Pub. No.: US 2024/0336609 A1
(43) Pub. Date: Oct. 10, 2024

(54) PYRIDAZINONE COMPOUNDS AND USES THEREOF

(71) Applicant: Edgewise Therapeutics, Inc., Boulder, CO (US)

(72) Inventors: Kevin Koch, Niwot, CO (US); Natalie Anne Hawryluk, Nederland, CO (US); Stephen Thomas Schlachter, Boulder, CO (US); Alan James Russell, Boulder, CO (US)

(21) Appl. No.: 18/666,699

(22) Filed: May 16, 2024

Related U.S. Application Data
(63) Continuation of application No. PCT/US2022/050313, filed on Nov. 17, 2022.
(60) Provisional application No. 63/280,457, filed on Nov. 17, 2021.

(51) Int. Cl. C07D 417/14; A61K 31/4439; A61K 31/444; A61K 31/501; A61K 31/5377; C07D 417/04

(52) U.S. Cl. CPC C07D 417/14; A61K 31/4439; A61K 31/444; A61K 31/501; A61K 31/5377; C07D 417/04

(57) ABSTRACT

Substituted pyridazinone compounds, conjugates, and pharmaceutical compositions for use in the treatment of neuromuscular diseases, such as Duchenne Muscular Dystrophy (DMD), are disclosed herein. The disclosed compounds are useful, among other things, in the treating of DMD and modulating inflammatory inhibitors IL-1, IL-6 or TNF-α.

### CROSS-REFERENCE

This application is the continuation application of International Patent Application No. PCT/US2022/050313, filed Nov. 17, 2022, which application claims the benefit of U.S. Provisional Application No. 63/280,457, filed Nov. 17, 2021, each of which is incorporated by reference herein in its entirety.

### BACKGROUND OF THE INVENTION

Skeletal muscle is the largest organ system in the human body, serving two primary purposes. The first is force production to enable muscle contraction, locomotion, and postural maintenance; the second is glucose, fatty acid and amino acid metabolism. The contraction of skeletal muscle during every-day activity and exercise is naturally connected to muscle stress, breakdown and remodeling which is important for muscle adaptation. In individuals with neuromuscular conditions, such as Duchenne Muscular Dystrophy (DMD), muscle contractions lead to continued rounds of amplified muscle breakdown that the body struggles to repair. Eventually, as patients age, a pathophysiological process emerges that leads to excess inflammation, fibrosis, and fatty deposit accumulation in the muscle, portending a steep decline in physical function and contribution to mortality.

DMD is a genetic disorder affecting skeletal muscle and is characterized by progressive muscle degeneration and weakness. There remains a need for treatments that reduce muscle breakdown in patients with neuromuscular conditions such as DMD.

### SUMMARY OF THE INVENTION

Disclosed herein is a compound represented by Formula (Ia) or (Ib):

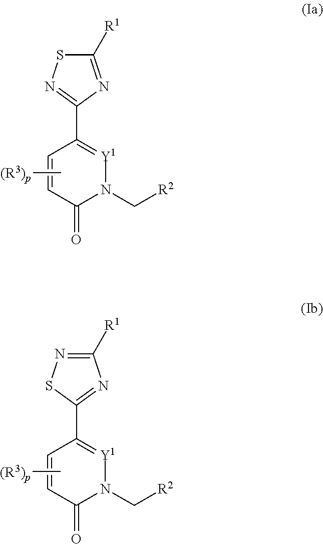

or a salt thereof, wherein:
Y¹ is N or CR⁴;
R¹ is selected from:
   hydrogen;
   halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, and —CN;
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵; and
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R² is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
   —C(O)NR⁷R⁸;
each R³ is independently selected from halogen, —OR⁶, —SR⁶, —N(R⁶)₂, —NO₂, —CN, and C₁. 6 alkyl optionally substituted with one or more substituents independently selected from halogen, —OR⁶, —SR⁶, —N(R⁶)₂, —NO₂, and —CN;
R⁴ is selected from hydrogen, halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
each R⁵ is independently selected from:
   halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN; and
   C₁₋₃ alkyl, C₂₋₃ alkenyl, C₂₋₃ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN;
each R⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁷ is selected from hydrogen and C₁₋₆ alkyl optionally substituted with one or more substituents independently selected from halogen, —OR⁶, —SR⁶, —N(R⁶)₂, —NO₂, and —CN;
R⁸ is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from: halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
p is 0, 1, or 2.

Disclosed herein is a compound represented by Formula (II):

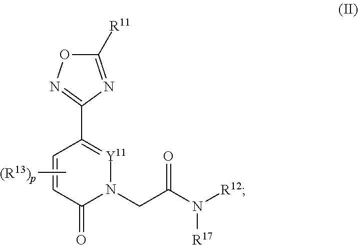

or a salt thereof, wherein:
Y¹¹ is N or CR¹⁴;
R¹¹ is selected from:
   hydrogen;
   halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵;
R¹² is selected from:
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R¹³ is independently selected from halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
R¹⁴ is independently selected from hydrogen, halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
each R¹⁵ is independently selected from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN; and C₁₋₃ alkyl, C₂₋₃ alkenyl, C₂₋₃ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R¹⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R¹⁷ is selected from hydrogen and C₁₋₆ alkyl optionally substituted with one or more substituents independently selected from halogen, —OR¹⁶, —SR¹⁶, —N(R¹⁶)₂, —NO₂, and —CN;
p is 0, 1, or 2.

Disclosed herein is a compound represented by Formula (III):

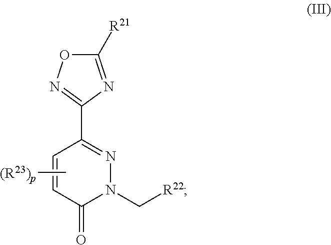

or a salt thereof, wherein:
R²¹ is selected from:
   hydrogen;
   halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, and —CN;
   C₂₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, ═O, ═S, ═N(R²⁶), —CN, C₃₋₁₀ carbocycle and 3- to 10-membered heterocycle, wherein the C₃₋₁₀ carbocycle and 3- to 10-membered heterocycle are each optionally substituted with one or more R²⁵; and
   C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵;
R²² is selected from:
   C₁₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R²⁵; and
   C₃₋₁₀ carbocycle and 3- to 10-membered heterocycle, each of which is optionally substituted with one or more substituents independently selected from: halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵;
each R²³ is independently selected from halogen, —OR²⁶, —SR²⁶, —N(R²⁶)₂, —NO₂, —CN, and C₁₋₆ alkyl optionally substituted with one or more substituents independently selected from halogen, —OR²⁶, —SR²⁶, —N(R²⁶)₂, —NO₂, and —CN;
each R²⁵ is independently selected from:
   halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN; and
   C₁₋₃ alkyl, C₂₋₃ alkenyl, C₂₋₃ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN;
each R²⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

Disclosed herein is a compound represented by Formula (IV):

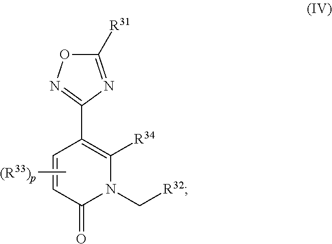

or a salt thereof, wherein:
R³¹ is selected from:
   hydrogen;
   halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂ and —CN;
   C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂, ═O, ═S, ═N(R³⁶), —CN, C₃₋₁₀ carbocycle and 3- to 10-membered heterocycle, wherein the C₃₋₁₀ carbocycle and 3- to 10-membered heterocycle are each optionally substituted with one or more R³⁵; and
   C₃₋₁₀ carbocycle and 3- to 10-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵;
R³² is selected from:
   C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵; and
   3- to 10-membered heterocycle optionally substituted with one or more substituents independently selected from: halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵;
each R³³ is independently selected from halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
R³⁴ is independently selected from hydrogen, halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
each R³⁵ is independently selected from:
   halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN; and
   C₁₋₃ alkyl, C₂₋₃ alkenyl, C₂₋₃ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN;
each R³⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
p is 0, 1, or 2.

Disclosed herein is a compound represented by Formula (V):

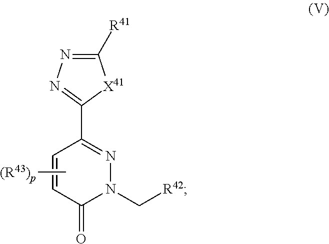

or a salt thereof, wherein:
X⁴¹ is O or S;
R⁴¹ is selected from:
   hydrogen;
   halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂ and —CN;
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
   C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴⁵;
R⁴² is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴;
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
   —C(O)NR⁴⁷R⁴⁸;
each R⁴³ is independently selected from halogen, —OR⁴⁶, —SR⁴⁶, —N(R⁴⁶)₂, —NO₂, —CN, and C₁₋₆ alkyl optionally substituted with one or more substituents independently selected from halogen, —OR⁴⁶, —SR⁴⁶, —N(R⁴⁶)₂, —NO₂, and —CN;
each R⁴¹ is independently selected from:
   halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —NO₂, ═O, ═S, ═N(R⁴⁶), and —CN; and
   C₁₋₃ alkyl, C₂₋₃ alkenyl, C₂₋₃ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN;
each R⁴⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁴⁷ is selected from hydrogen and C₁₋₆ alkyl optionally substituted with one or more substituents independently selected from halogen, —OR⁴⁶, —SR⁴⁶, —N(R⁴⁶)₂, —NO₂, and —CN;
R⁴⁸ is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
p is 0, 1, or 2.

Disclosed herein is a compound represented by Formula (VI):

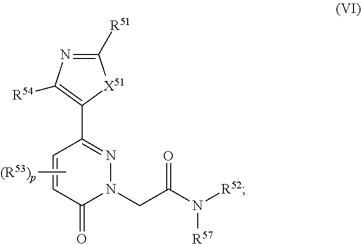

or a salt thereof, wherein:
X⁵¹ is selected from O and S;
R⁵¹ is selected from:
   hydrogen;
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂ and —CN;
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R¹⁶)₂, —N(R⁵⁶)C(O)R⁵⁶, —C(O)OR⁵⁶, —OC(O)R¹⁶, —N(R⁵⁶)C(O)N(R⁵⁶)₂, —OC(O)N(R¹⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵;
R⁵² is selected from:
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from: halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵;
each R⁵³ is independently selected from halogen, —OR⁵⁶, —SR⁵⁶, —N(R⁵⁶)₂, —NO₂, —CN, C₁₋₆ alkyl optionally substituted with one or more substituents independently selected from halogen, —OR⁵⁶, —SR⁵⁶, —N(R⁵⁶)₂, —NO₂, and —CN;
R⁵⁴ is independently selected from hydrogen, halogen, —OR⁵⁶, —SR⁵⁶, —N(R⁵⁶)₂, —NO₂, —CN, C₁₋₆ alkyl, C₃₋₁₀ carbocycle and 3- to 10-membered heterocycle, wherein C₁₋₆ alkyl, C₃₋₁₀ carbocycle and 3- to 10-membered heterocycle are each optionally substituted with one or more substituents independently selected from halogen, —OR⁵⁶, —SR⁵⁶, —N(R⁵⁶)₂, —NO₂, and —CN;
each R⁵⁵ is independently selected from:
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
   C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN;
each R⁵⁶ is independently selected from:
   hydrogen; C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2.

Disclosed herein is a compound represented by Formula (VII):

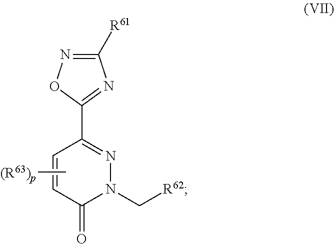

or a salt thereof, wherein:
R⁶¹ is selected from:
   hydrogen;
   halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, and —CN;
   C₁₋₆ alkyl, C₂₋₆ alkenyl, C₂₋₆ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, ═O, ═S, ═N(R⁶⁶), —CN, C₃₋₁₀ carbocycle and 3- to 10-membered heterocycle, wherein the C₃₋₁₀ carbocycle and 3- to 10-membered heterocycle are each optionally substituted with one or more R⁶⁵; and
   C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵.
R⁶² is selected from:
   5-membered heterocycle optionally substituted with one or more substituents independently selected from: halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵.
each R⁶³ is independently selected from halogen, —OR⁶⁶, —SR⁶⁶, —N(R⁶⁶)₂, —NO₂, —CN, and C₁₋₆ alkyl optionally substituted with one or more substituents independently selected from halogen, —OR⁶⁶, —SR⁶⁶, —N(R⁶⁶)₂, —NO₂, and —CN;
each R⁶⁵ is independently selected from:
   halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN; and
   C₁₋₃ alkyl, C₂₋₃ alkenyl, C₂₋₃ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN;
each R⁶⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

Disclosed herein is a compound represented by Formula (VIII):

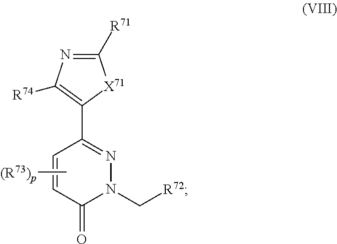

or a salt thereof, wherein:
X⁷¹ is selected from S and O;
R⁷¹ is selected from:
   hydrogen;
   halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, and —CN;
   C₁₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, ═O, ═S, ═N(R⁷⁶), —CN, C₃₋₁₀ carbocycle and 3- to 10-membered heterocycle, wherein the C₃₋₁₀ carbocycle and 3- to 10-membered heterocycle are each optionally substituted with one or more R⁷⁵; and
   C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷⁵;
R⁷² is selected from:
   3- to 10-membered heterocycle optionally substituted with one or more substituents independently selected from: halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷¹;
each R⁷³ is independently selected from halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
R⁷⁴ is independently selected from hydrogen, halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
each R⁷⁵ is independently selected from:
   halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN; and
   C₁₋₃ alkyl, C₂₋₃ alkenyl, C₂₋₃ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN;
each R⁷⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

Disclosed herein is a pharmaceutical composition comprising a compound or salt provided herein.

Disclosed herein is a method of treating a neuromuscular condition or movement disorder, comprising administering to a subject in need thereof a compound or salt provided herein. In some embodiments, the neuromuscular condition is selected from Duchenne Muscular Dystrophy, Becker muscular dystrophy, myotonic dystrophy 1, myotonic dystrophy 2, facioscapulohumeral muscular dystrophy, oculopharyngeal muscular dystrophy, limb girdle muscular dystrophy, tendinitis, carpal tunnel syndrome. In some embodiments, the neuromuscular condition is Duchenne Muscular Dystrophy. In some embodiments, the movement disorder comprises muscle spasticity. In some embodiments, the muscle spasticity is selected from spasticity associated with multiple sclerosis, Parkinson's disease, Alzheimer's disease, or cerebral palsy, or injury, or a traumatic event such as stroke, traumatic brain injury, spinal cord injury, hypoxia, meningitis, encephalitis, phenylketonuria, or amyotrophic lateral sclerosis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In certain aspects, the disclosure provides methods for treating neuromuscular conditions through selective inhibition of fast-fiber skeletal muscle myosin. In particular, methods of the disclosure may be used in the treatment of DMD and other neuromuscular conditions.

Skeletal muscle is mainly composed of two types of fibers, slow-twitch muscle fiber (i.e., type I) and fast-twitch muscle fiber (i.e., type II). In each muscle, the two types of fibers are configured in a mosaic-like arrangement, with differences in fiber type composition in different muscles and at different points in growth and development. Slow-twitch muscle fibers have excellent aerobic energy production ability. Contraction rate of the slow-twitch muscle fiber is low but tolerance to fatigue is high. Slow-twitch muscle fibers typically have a higher concentration of mitochondria and myoglobin than do fast-twitch fibers and are surrounded by more capillaries than are fast-twitch fibers. Slow-twitch fibers contract at a slower rate due to lower myosin ATPase activity and produce less power compared to fast-twitch fibers, but they are able to maintain contractile function over longer-terms, such as in stabilization, postural control, and endurance exercises.

Fast twitch muscle fibers in humans are further divided into two main fiber types depending on the specific fast skeletal myosin they express (Type IIA, IIx/d). A third type of fast fiber (Type IIB) exists in other mammals but is rarely identified in human muscle. Fast-twitch muscle fibers have excellent anaerobic energy production ability and are able to generate high amounts of tension over a short period of time. Typically, fast-twitch muscle fibers have lower concentrations of mitochondria, myoglobin, and capillaries compared to slow-twitch fibers, and thus can fatigue more quickly. Fast-twitch muscles produce quicker force required for power and resistance activities.

The proportion of the type I and type II can vary in different individuals. For example, non-athletic individuals can have close to 50% of each muscle fiber types. Power athletes can have a higher ratio of fast-twitch fibers, e.g., 70-75% type II in sprinters. Endurance athletes can have a higher ratio of slow-twitch fibers, e.g., 70-80% in distance runners. The proportion of the type I and type II fibers can also vary depending on the age of an individual. The proportion of type II fibers, especially the type Ix, can decline as an individual ages, resulting in a loss in lean muscle mass.

The contractile action of skeletal muscle leads to muscle damage in subjects with neuromuscular disease, e.g., DMD, and this damage appears to be more prevalent in fast fibers.

It has been observed that acute force drop after lengthening injury is greater in predominantly fast type II fiber muscles compared to predominantly slow type I fiber muscles in dystrophy mouse models. It has also been demonstrated that the degree of acute force drop and histological damage in dystrophy mouse models is proportional to peak force development during lengthening injury. Excessive contraction-induced injuries, which precede the inflammation and irreversible fibrosis that characterizes late-stage DMD pathology. Contraction-induced muscle damage in these patients may be reduced by limiting peak force generation in type II fibers and possibly increasing reliance on healthier type I fibers. N-benzyl-p-tolyl-sulfonamide (BTS), an inhibitor of fast-fiber skeletal muscle myosin, has been shown to protect muscles from pathological muscle derangement in embryos from zebrafish model of DMD.

Inhibitors of skeletal muscle myosin that are not selective for the type II fibers may lead to excessive inhibition of skeletal muscle contraction including respiratory function and unwanted inhibition of cardiac activity as the heart shares several structural components (such as type I myosin) with type I skeletal muscle fibers. While not wishing to be bound by a particular mechanistic theory, this disclosure provides selective inhibitors of fast-fiber skeletal muscle myosin as a treatment option for Becker muscular dystrophy (BMD), Duchenne muscular dystrophy (DMD), Limb-girdle muscular dystrophies (LGMD), McArdle disease, and other neuromuscular conditions. The targeted inhibition of type II skeletal muscle myosin may reduce skeletal muscle contractions while minimizing the impact on a subject's daily activities.

When healthy muscle is subjected to excessive, unaccustomed exercise, it develops soreness and sustained reductions in strength and range of motion. Proteins also leak from injured muscle fibers into circulation, including creatine kinase (CK), lactate dehydrogenase and myoglobin. These biomarkers are not unique to either fast or slow fibers and so do not provide detail regarding differences in fiber responses to injury. Troponin I (TNNI) is a component of the troponin complex that controls initiation of contraction of muscle by calcium. It is distinct in that there is a different isoform for each type of striated muscle: TNNI1 in slow skeletal muscle, TNN12 in fast skeletal muscle and TNNI3 in cardiac muscle. Selective enzyme-linked immunosorbent assays (ELISAs) have been used to demonstrate that TNNI2 but not TNNI1 is elevated in circulation after injurious exercise, even under extreme conditions.

DMD and BMD are caused by an absence (DMD) or truncation (BMD) of the dystrophin protein₅. Dystrophin provides a structural link between the actin cytoskeleton and the basement membrane through the dystrophin-glycoprotein complex. When dystrophin is absent or truncated, contraction of muscle leads to heightened muscle stress and injury with normal use. While the sensitivity to injury is much higher in DMD muscle than in BMD or healthy muscle, fast fibers still appear to be more susceptible than slow fibers, with young DMD patients exhibiting histological evidence of disruption in fast fibers₇ and early loss of type Ix fibers. Example 10 shows the relative susceptibility of these fibers to leak muscle contents, such as troponin, creatine kinase, or myoglobin. In some embodiments, this disclosure provides selective inhibitors of fast-fiber skeletal muscle myosin as a treatment option for DMD, BMD, McArdle's disease, or Limb-girdle muscular dystrophies.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise. The term “C_{x-y}” or “Cₓ-C_{y}” (e.g., when used in conjunction with a chemical moiety, such as alkyl, alkenyl, or alkynyl) is meant to include groups that comprise a number of carbon atoms greater than or equal to x carbon atoms and less than or equal to y carbon atoms in the chemical moiety. The term “C_{x-y}” or “Cₓ-C_{y}” is not meant to limit the number of carbon atoms which may be attached to the chemical moiety when the chemical moiety is substituted with a second chemical moiety. For example, the term “C₁₋₆ alkyl” or “C₁ to C₆ alkyl” refers to saturated, substituted or unsubstituted, hydrocarbon groups, including straight-chain alkyl groups (e.g., linear alkyl groups) and branched alkyl groups that contain 1, 2, 3, 4, 5, or 6 carbon atoms, plus however many carbon atoms may be present in any substituents of the C₁₋₆ alkyl. For example, if a C₁₋₆ alkyl is optionally substituted with a second chemical moiety comprising two carbon atoms, then it will be understood that the C₁₋₆ alkyl can include between 1 and 8 carbon atoms.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

“Amino” refers to the —NH₂ moiety.

“Cyano” refers to the —CN moiety.

“Nitro” refers to the —NO₂ moiety.

“Oxa” refers to the —O— moiety.

“Oxo” refers to the ═O moiety.

“Thioxo” refers to the ═S moiety.

“Imino” refers to the ═N—H moiety.

“Oximo” refers to the ═N—OH moiety.

“Hydrazino” refers to the ═N—NH₂ moiety.

“Alkyl” refers to a straight or branched hydrocarbon moiety consisting solely of carbon and hydrogen atoms, fully saturated. In certain embodiments, “alkyl” comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In certain embodiments, an alkyl comprises one to six carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl, e.g., methyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (2-propyl, iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond.

“Aminoalkyl” refers to a moiety boded through a nitrogen atom of the form —N(H)(alkyl) or N(alkyl)(alkyl), wherein when the moiety is N(alkyl)(alkyl), the two alkyl groups bonded to nitrogen can be the same alkyl groups or different alkyl groups.

“Alkoxy” refers to a moiety bonded through an oxygen atom of the formula —O-alkyl, where alkyl is an alkyl chain as defined above.

“Alkenyl” refers to a straight or branched hydrocarbon moiety consisting solely of carbon and hydrogen atoms, containing at least one carbon-carbon double bond. In certain embodiments, an alkenyl comprises two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon moiety consisting solely of carbon and hydrogen atoms, containing at least one carbon-carbon triple bond, having from two to twelve carbon atoms, and optionally further comprising at least one carbon-carbon double bond.

In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like.

“Alkylene” or “alkylene chain” refers to a linear (e.g., straight), or branched, divalent, hydrocarbon moiety. An “alkylene” or “alkylene chain” can link a portion of the molecule to a second moiety. An “alkylene” or “alkylene chain” consists solely of carbon and hydrogen atoms (substitution of an alkylene with one or more substituents comprising atoms other than hydrogen, such as N, O, and S, may be specified). An “alkylene” or “alkylene chain” can contain no unsaturation (notwithstanding the points of attachment of an alkylenne to the rest of the molecule). In certain embodiments, the “alkylene” or “alkylene chain” and comprises one to twelve carbon atoms, for example, methylene, ethylene, propylene, n-butylene, and the like. The alkylene chain can be attached to the portion of the molecule through a single bond and to the second moiety through a single bond. The points of attachment of an alkylene chain to the rest of the molecule and to the second moiety can be through one carbon in the alkylene chain or through any two carbons within the alkylene. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene).

“Alkenylene” or “alkenylene chain” refers to a linear (e.g., straight), or branched, divalent, hydrocarbon moiety. An “alkenylene” or “alkenylene chain” can link a portion of the molecule to a second moiety. An “alkenylene” or “alkenylene chain” consists solely of carbon and hydrogen atoms (substitution of an alkenylene with one or more substituents comprising atoms other than hydrogen, such as N, O, and S, may be specified). An “alkenylene” or “alkenylene chain” comprises at least one carbon-carbon double bond. In certain embodiments, an “alkenylene” or “alkenylene chain” comprises from two to twelve carbon atoms. The alkenylene chain can be attached to the portion of the molecule through a single bond and to the second moiety through a single bond. The points of attachment of an alkenylene chain to the rest of the molecule and to the second moiety can be through one carbon in the alkenylene chain or through any two carbons within the alkenylene chain. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene).

“Alkynylene” or “alkynylene chain” refers to a linear (e.g., straight), or branched, divalent, hydrocarbon moiety. An “alkynylene” or “alkynylene chain” can link a portion of the molecule to a second moiety. An “alkynylene” or “alkynylene chain” consists solely of carbon and hydrogen (substitution of an alkynylene with one or more substituents comprising atoms other than hydrogen, such as N, O, and S, may be specified). An “alkynylene” or “alkynylene chain” comprises at least one carbon-carbon triple bond. In certain embodiments, an “alkynylene” or “alkynylene chain” comprises from two to twelve carbon atoms. An alkynylene chain can be attached to the portion of the molecule through a single bond and to the second moiety through a single bond. The points of attachment of an alkynylene chain to the rest of the molecule and to the second moiety can be through one carbon in the alkynylene chain or through any two carbons within the alkynylene chain. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene).

The term “carbocycle” as used herein refers to a saturated or unsaturated (e.g., aromatic or nonaromatic unsaturated) ring or ring system in which each atom of the ring is carbon. For example, the term “carbocycle” includes 3- to 12-membered monocyclic rings (e.g., 3- to 10-membered monocyclic rings) and 4- to 20-membered polycyclic ring systems (e.g., 5- to 15-membered spiro polycyclic ring systems, 5- to 15-membered bridged polycyclic ring systems, or 4- to 15-membered fused polycyclic ring systems). For example, carbocycle includes 4- to 15-membered bicyclic rings (e.g., 5- to 15-membered spiro bicycles, 5- to 15-membered bridged bicyclic ring systems, or 4- to 15-membered fused bicyclic ring systems). For example, carbocycle includes tricyclic ring systems, which may be bridged, fused, spiro, or a combination thereof. For example, carbocycle includes tetracyclic ring systems, which may be bridged, fused, spiro, or a combination thereof. For example, carbocycle includes ring systems that are both fused and bridged; ring systems that are both fused and spiro; ring systems that are both bridged and spiro; and ring systems that are both fused and bridged and are also spiro. Each ring of a polycyclic carbocycle may be selected from saturated and unsaturated (e.g., aromatic or nonaromatic unsaturated) rings. In an exemplary embodiment, an aromatic ring, e.g., phenyl, of a polycyclic carbocycle may be fused to a saturated or unsaturated ring (e.g., cyclohexane, cyclopentane, cyclohexene, or phenyl). A polycyclic carbocycle includes any combination of saturated and unsaturated (e.g., aromatic or nonaromatic unsaturated) rings, as valence permits. For example, polycyclic carbocycles further include spiro bicyclic rings, such as spiropentane. For example, a polycyclic carbocycle includes any combination of ring sizes such as 2-2 spiro ring systems (e.g., spiro[2.2]pentane), 3-3 spiro ring systems, 4-4 spiro ring systems, 4-5 fused ring systems (e.g., bicyclo[4.5.0] fused ring systems), 5-5 fused ring systems, 5-6 fused ring systems, 6-6 fused ring systems (e.g., naphthalene), 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, trans-bicyclo[4.4.0]decane, cis-bicyclo[4.4.0]decane, spiro[3.4]octane, fluoranthene, and bicyclo[1.1.1]pentanyl.

The term “aryl” refers to an aromatic monocyclic or aromatic polycyclic hydrocarbon ring system comprising at least one cyclic, delocalized (4n+2) π-electronic system in accordance with Hückel theory. In some embodiments, the aromatic monocyclic or aromatic polycyclic hydrocarbon ring system comprises only hydrogen atoms and carbon atoms. In some embodiments, the aromatic monocyclic or polycyclic system contains from three to twenty carbon atoms. In some embodiments, at least one of the rings in the polycyclic aromatic ring system is aromatic. In some embodiments, the aromatic monocyclic or aromatic polycyclic hydrocarbon ring system comprises a cyclic, delocalized (4n+2) π-electronic system in accordance with Hückel theory. The ring system from which aryl groups are derived include, but are not limited to, groups such as benzene, fluorene, indane, indene, anthracene, tetralin, and naphthalene. In some embodiments, the aryl substituent is positively or negatively charged. In some embodiments, the aryl substituent is neutral. In some embodiments, the aryl substituent is zwitterionic; alternatively, or in addition, in some embodiments, the aryl substituent is not charged. In some embodiments, the aryl substituent bears no charges. In some embodiments, the aryl substituent bears no net charge. In some embodiments, the aryl substituent bears no net charge and is not zwitterionic.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises three to seven carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Examples of polycyclic cycloalkyls include, but are not limited to, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, spiropentane, and the like.

The term “cycloalkenyl” refers to a saturated ring in which each atom of the ring is carbon and there is at least one double bond between two ring carbons. Cycloalkenyl may include monocyclic and polycyclic rings, such as 3- to 10-membered monocyclic rings and 4- to 12-membered bicyclic rings (e.g., 5- to 12-membered bridged bicyclic rings, fused 4- to 12-membered bicyclic rings, and spiro 5- to 12-membered bicyclic rings). In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl, as defined above, that is substituted by one or more halogens, for example, trifluoromethyl, dichloromethyl, bromomethyl, 2,2,2-trifluoroethyl, 1-chloromethyl-2-fluoroethyl, and the like. In some embodiments, the alkyl part of the haloalkyl is optionally further substituted as described herein.

The term “heterocycle” as used herein refers to a saturated or unsaturated (e.g., aromatic or nonaromatic unsaturated) ring or ring system in which one or more heteroatom(s) is(are) member(s) of the ring or ring system. Exemplary heteroatoms include N, O, Si, P, B, and S atoms. For example, heterocycles include 3- to 12-membered monocyclic rings (e.g., 3- to 10-membered monocyclic rings) and 4- to 20-membered polycyclic ring systems (e.g., 4- to 15-membered fused poly ring systems, 5- to 15-membered spiro polycyclic ring systems, and 5- to 15-membered bridged polycyclic ring systems). For example, heterocycles include 4- to 20-membered bicyclic ring systems (e.g., 4- to 15-membered fused bicyclic ring systems, 5- to 15-membered spiro bicyclic ring systems, and 5- to 15-membered bridged bicyclic ring systems). For example, heterocycle includes tricyclic ring systems, which may be bridged, fused, spiro, or a combination thereof. For example, heterocycle includes tetracyclic ring systems, which may be bridged, fused, spiro, or a combination thereof. For example, heterocycle includes ring systems that are both fused and bridged; ring systems that are both fused and spiro; ring systems that are both bridged and spiro; and ring systems that are both fused and bridged and are also spiro. Each ring of a polycyclic heterocycle may be selected from saturated and unsaturated (e.g., aromatic or nonaromatic unsaturated) rings. A polycyclic heterocycle includes any combination of saturated, and unsaturated (e.g., aromatic or nonaromatic unsaturated) rings, as valence permits. In an exemplary embodiment, an aromatic ring, e.g., pyridyl or phenyl, may be fused to a saturated or unsaturated ring, e.g., cyclohexane, cyclopentane, morpholine, piperidine or cyclohexene, in a heterocycle, as long as at least one atom in the resulting fused ring system is a heteroatom. A polycyclic heterocycle includes any combination of ring sizes such as 3-3 spiro, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane. In some embodiments, a heterocycle comprises multiple heteroatoms. In some embodiments, a heterocycle comprises one or more atoms selected from nitrogen, oxygen, and sulfur. In some embodiments, a heterocycle comprises multiple atoms selected from nitrogen, oxygen, and sulfur. Nonlimiting examples of heterocycles include pyridine, pyrrole, indole, carbazole, piperidine, oxazole, morpholine, thiophene, benzothiophene, furan, tetrahydrofuran, and pyran. Nonlimiting examples of heterocycles include azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

One or more nitrogen atoms, if present, are optionally quaternized. In some embodiments, the heterocycle substituent is positively or negatively charged. In some embodiments, the heterocycle substituent is neutral. In some embodiments, the heterocycle substituent is zwitterionic; alternatively, or in addition, in some embodiments, the heterocycle substituent is not charged. In some embodiments, the heterocycle substituent bears no charges. In some embodiments, the heterocycle substituent bears no net charge. In some embodiments, the heterocycle substituent bears no net charge and is not zwitterionic.

The term “heteroaryl” refers to a moiety derived from an aromatic monocyclic or aromatic polycyclic ring system, in which one or more heteroatom(s) is(are) member(s) of the ring system, and the ring system comprises at least least one cyclic, delocalized (4n+2) π-electronic system in accordance with Hückel theory. Exemplary heteroatoms include N, O, Si, P, B, and S atoms. In some embodiments, a heteroaryl includes one or more heteroatoms selected from nitrogen, oxygen, and sulfur. In some embodiments, a heteroaryl includes multiple heteroatoms selected from nitrogen, oxygen, and sulfur. In certain embodiments, “heteroaryl” includes rings and ring systems comprising 3 to 20 atoms. In some embodiments, “heteroaryl” includes rings and ring systems that comprise two to seventeen carbon atoms and from one to six heteroatoms selected from nitrogen, oxygen and sulfur. As used herein, the heteroaryl moiety is a monocyclic or polycyclic (e.g., bicyclic, tricyclic or tetracyclic) ring system, wherein at least one of the rings in the ring system is aromatic, i.e., it contains a cyclic, delocalized (4n+2) π-electron system in accordance with the Hückel theory. Heteroaryl includes fused, bridged, and spiro ring systems. The heteroatom(s) in the heteroaryl moiety is(are) optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). In some embodiments, the heteroaryl substituent is positively or negatively charged. In some embodiments, the heteroaryl substituent is neutral. In some embodiments, the heteroaryl substituent is zwitterionic; alternatively, or in addition, in some embodiments, the heteroaryl substituent is not charged. In some embodiments, the heteroaryl substituent bears no charges. In some embodiments, the heteroaryl substituent bears no net charge. In some embodiments, the heteroaryl substituent bears no net charge and is not zwitterionic.

“Heterocycle” comprises “heteroaryl” and “heterocycloalkyl”. “Carbocycle” comprises “aryl” and “cycloalkyl.”

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl. In some embodiments, a heterocycloalkyl comprises one heteroatom. In some embodiments, a heterocycloalkyl comprises one heteroatom selected from N, O, and S. In some embodiments, a heterocycloalkyl comprises multiple heteroatoms. In some embodiments, a heterocycloalkyl comprises multiple heteroatoms selected from N, O, and S.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms.

The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent and further includes the proviso that the substitution results in a stable compound, e.g., a compound which does not rapidly undergo rearrangement, cyclization, elimination, etc. In certain embodiments, substituted refers to moieties having substituents replacing two hydrogen atoms on the same carbon atom, such as substituting the two hydrogen atoms on a single carbon with an oxo, imino, oxime, hydrazone, or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)OR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and/or organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and/or organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is selected from ammonium, potassium, sodium, calcium, and magnesium salts.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, all structures described herein are intended to disclose, implicitly or explicitly, all Z-, E-, and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibria include but are not limited to:

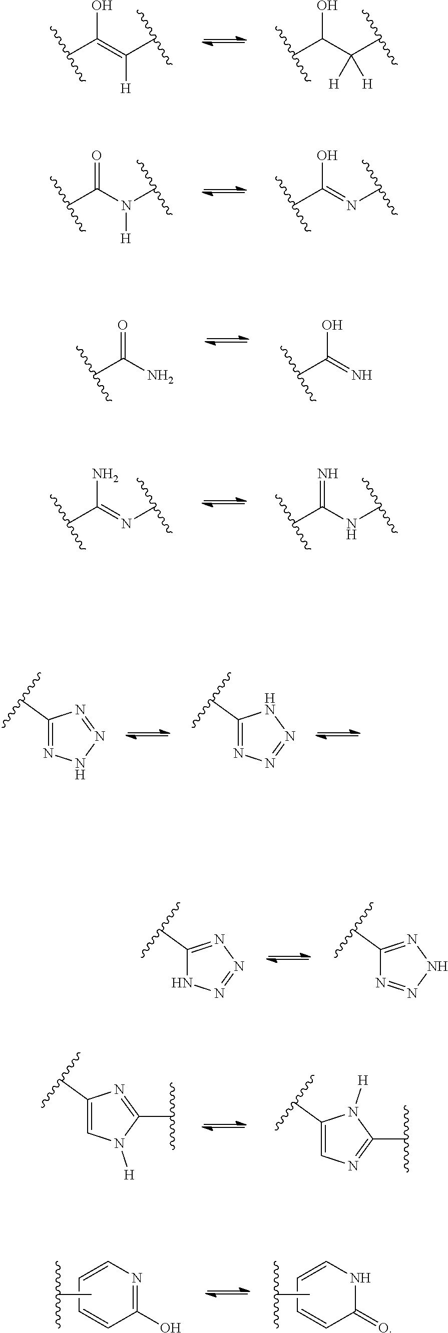

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy of drugs, thus increasing the duration of action of drugs.

Unless otherwise stated, compounds described herein are intended to include compounds which differ only in the presence of one or more isotopically enriched atoms. For example, compounds having the present structures except for the replacement of one or more proton(s) by one or more deuterium(deuteria) or tritium(tritia), or combinations thereof, or except for the replacement of one or more ¹²C atom(s) in the structure by one or more ¹³C atom(s), one or more ¹⁴C atom(s), or combinations thereof, in the structure are within the scope of the present disclosure.

The compounds of the present disclosure optionally comprise unnatural proportions of atomic isotopes at one or more atom(s) that constitute such compounds. For example, the compounds may be labeled with one or more isotope(s), such as for example, deuterium (²H), tritium (H), iodine-125 (¹²⁵I) or carbon-14 (¹⁴C). Isotopic substitution with ²H, ¹¹C, ¹³C ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I, are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium-substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as MilliporeSigma.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that comprise one or more sufficiently acidic functional group(s), one or more sufficiently basic functional group(s), or both one or more sufficiently acidic functional group(s) and one or more sufficiently basic functional group(s) to form a salt (particularly a pharmaceutically acceptable salt), can react with any of a number of inorganic organic bases or inorganic or organic acids, to form a salt. combinations thereof); or combinations thereof. Alternatively, compounds that are inherently charged, such as those with a quaternary nitrogen, can form a salt with an appropriate counterion.

The compounds and salts described herein may in some cases exist as diastereomers, enantiomers, or other stereoisomeric forms. Unless otherwise specified, the structures disclosed herein are intended to include, explicitly or implicitly, disclosure of all diastereomeric (e.g., epimeric) and enantiomeric forms as well as mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Compounds

The following is a discussion of compounds and salts thereof that may be used in the methods of the disclosure.

### Compounds

The following is a discussion of compounds and salts thereof that may be used in the methods of the disclosure. In certain embodiments, the compounds and salts are described in Formulas (Ia), (Ib), (II), (III), (IV), (V), (VI), (VI), (VII), or (VIII).

In one aspect, disclosed herein is compound represented by Formula (Ia) or (Ib):

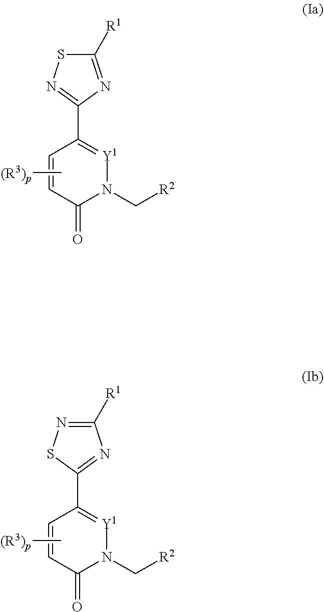

or a salt thereof, wherein:
Y¹ is N or CR⁴;
R¹ is selected from:
   hydrogen;
   halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, and —CN;
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵; and
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R² is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
   —C(O)NR⁷R⁸;
each R³ is independently selected from halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
R⁴ is selected from hydrogen, halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
each R⁵ is independently selected from:
   halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN; and
   C₁₋₃ alkyl, C₂₋₃ alkenyl, C₂₋₃ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN;
each R⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁷ is selected from hydrogen and C₁₋₆ alkyl optionally substituted with one or more substituents independently selected from halogen, —OR⁶, —SR⁶, —N(R⁶)₂, —NO₂, and —CN;
R⁸ is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from: halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
p is 0, 1, or 2.

In certain embodiments, for a compound or salt of Formula (Ia) is selected from:

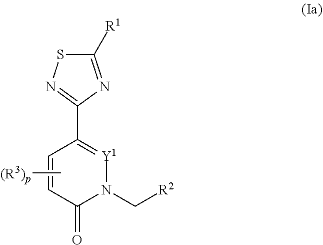

or a salt thereof.

In certain embodiments, for a compound or salt of Formula (Ib) is selected from:

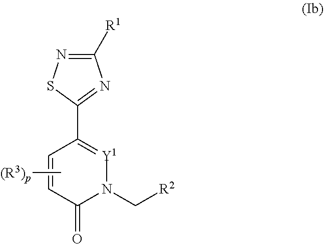

or a salt thereof.

In certain embodiments, for a compound or salt of Formula (Ia) or (Ib), Y is N.

In certain embodiments, for a compound or salt of Formula (Ia) or (Ib), Y is CR⁴.

In certain embodiments, for a compound or salt of Formula (Ia) or (Ib), R¹ is selected from:
halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, and —CN;
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵; and
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵.

In certain embodiments, for a compound or salt of Formula (Ia) or (Ib), R¹ is selected from:
halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, and —CN;
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵; and
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵.

In certain embodiments, for a compound or salt of Formula (Ia) or (Ib), R¹ is selected from:
halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (Ia) or (Ib), R¹ is selected from C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵. In certain embodiments, for a compound or salt of Formula (Ia) or (Ib), R¹ is selected from C₅₋₆ carbocycle and 5- to 6-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, —N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵. In certain embodiments, for a compound or salt of Formula (Ia) or (Ib), R¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (Ia) or (Ib), R¹ is selected from phenyl, pyridinyl, and morpholinyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In some embodiments, R¹ is selected from phenyl, pyridinyl, and morpholinyl, each of which is optionally substituted with one or more substituents selected from methyl, ethyl, —CF₃, —CHF₂, —CH₂F, —CH₂CHF₂, —CH₂CF₃, and —CH₂CH₂F. In certain embodiments, R¹ is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R¹ is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R¹ is selected from phenyl and pyridyl, each of which is optionally substituted with one or more halogen.

In certain embodiments, for a compound or salt of Formula (Ia) or (Ib), R¹ is selected from R¹ is selected from

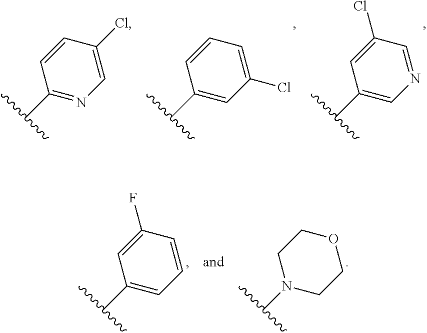 [SMILES: *c1ccc(Cl)cn1 ; *c1cccc(Cl)c1 ; *c1cncc(Cl)c1 ; *c1cccc(F)c1 ; *N1CCOCC1]

In some embodiments, R¹ is selected from

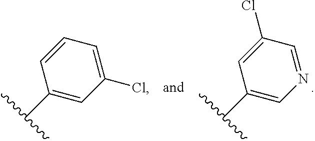 [SMILES: *c1cccc(Cl)c1 ; *c1cncc(Cl)c1]

In certain embodiments, for a compound or salt of Formula (Ia) or (Ib), R² is selected from:
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
—C(O)NR⁷R⁸.

In certain embodiments, for a compound or salt of Formula (Ia) or (Ib), R² is selected from:
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
—C(O)NR⁷R⁸.

In certain embodiments, for a compound or salt of Formula (Ia) or (Ib), R² is selected from
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
—C(O)NR⁷R⁸.

In certain embodiments, for a compound or salt of Formula (Ia) or (Ib), R² is selected from C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵. In certain embodiments, for a compound or salt of Formula (Ia) or (Ib), R² is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (Ia) or (Ib), R² is selected from pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (Ia) or (Ib), R² is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (Ia) or (Ib), R² is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R² is selected from phenyl and pyridyl, each of which is optionally substituted with one or more halogen.

In certain embodiments, for a compound or salt of Formula (Ia) or (Ib), R² is selected from:

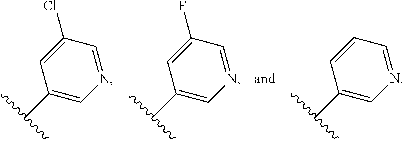 [SMILES: *c1cncc(Cl)c1 ; *c1cncc(F)c1 ; *c1cccnc1]

In certain embodiments, for a compound or salt of Formula (Ia) or (Ib), R² is C(O)NR⁷R⁸.

In certain embodiments, for a compound or salt of Formula (Ia) or (Ib), R⁷ is selected from hydrogen and C₁₋₆ alkyl optionally substituted with one or more substituents independently selected from halogen, —OH, —SH, —NH₂, —NO₂, —CN, —O—C₁₋₆ alkyl, —S—C₁₋₆ alkyl, —N(C₁₋₆ alkyl)₂, and —NH(C₁₋₆ alkyl). In certain embodiments, R⁷ is selected from hydrogen and C₁₋₆ alkyl optionally substituted with one or more substituents independently selected from halogen, —OH, —SH, —NH₂, —NO₂, —CN, and —OMe. In certain embodiments, R⁷ is selected from hydrogen and C₁₋₆ alkyl. In certain embodiments, R⁷ is hydrogen. In some embodiments, R⁷ is selected from methyl, ethyl, n-propyl and isopropyl.

In certain embodiments, for a compound or salt of Formula (Ia) or (Ib), R⁸ is selected from:
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵; and
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵.

In certain embodiments, for a compound or salt of Formula (Ia) or (Ib), R⁸ is selected from:
C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (Ia) or (Ib), R⁸ is selected from C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl. In certain embodiments, R⁸ is C₁₋₆ alkyl. In certain embodiments, R⁸ is selected from methyl, ethyl, propyl, butyl, cyclopropyl, and cyclobutyl. In certain embodiments, R⁸ is selected from methyl, ethyl, propyl, butyl, cyclopropyl, and cyclobutyl, and R⁷ is hydrogen. In some embodiments, R⁸ is selected from ethyl. In some embodiments, R⁸ is selected from ethyl, and R⁷ is hydrogen.

In certain embodiments, for a compound or salt of Formula (Ia) or (Ib), each R³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R³ is independently selected from halogen, —CN, —OH, —SH —NO₂, and —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In some embodiments, each R³ is independently selected from halogen, —CN, —OH, —SH —NO₂, —NH₂, and —OMe. In certain embodiments, each R³ is C₁₋₆ alkyl optionally substituted with one or more substituents independently selected from halogen, —CN, —OH, —SH —NO₂, and —NH₂. In some embodiments, each R³ is C₁₋₆ alkyl. In certain embodiments, R³ is selected from methyl, ethyl, propyl, butyl, cyclopropyl, and cyclobutyl.

In certain embodiments, for a compound or salt of Formula (Ia) or (Ib), p is 0. In certain embodiments, p is 1. In certain embodiments, p is 2.

In certain embodiments, for a compound or salt of Formula (Ia) or (Ib), R⁴ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, for a compound or salt of Formula (Ia) or (Ib), R⁴ is selected from hydrogen and C₁₋₆ alkyl optionally substituted with one or more substituents independently selected from halogen, —CN, —OH, —SH —NO₂, and —NH₂. In certain embodiments, for a compound or salt of Formula (Ia) or (Ib), R⁴ is hydrogen. In certain embodiments, R⁴ is selected from methyl, ethyl, propyl, butyl, cyclopropyl, and cyclobutyl.

In certain embodiments, for a compound or salt of Formula (Ia) or (Ib), each R⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R⁵ is selected from methyl, ethyl, and propyl.

In certain embodiments, each R⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R⁶ is selected from methyl, ethyl, propyl, butyl, cyclopropyl, and cyclobutyl. In certain embodiments, R⁶ is hydrogen.

In one aspect, disclosed herein is compound represented by Formula (Ia) or (Ib):

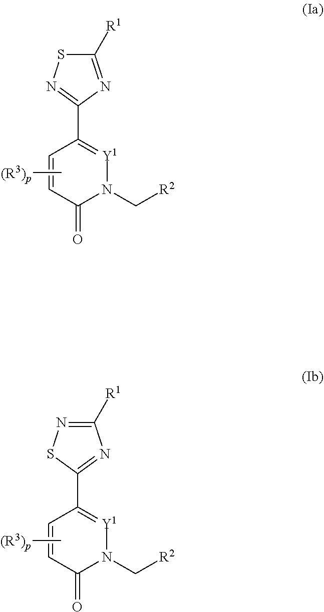

or a salt thereof, wherein:
Y¹ is N or CR⁴;
R¹ is selected from:
   C₁₋₆ alkyl optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵; and
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R² is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —OC(O)N(R⁶)₂, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
   —C(O)NR⁷R⁸;
each R³ is independently selected from halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
R⁴ is selected from hydrogen, halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
each R⁵ is independently selected from:
   halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN; and
   C₁₋₃ alkyl, C₂₋₃ alkenyl, C₂₋₃ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN;
each R⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁷ is selected from hydrogen and C₁₋₆ alkyl optionally substituted with one or more substituents independently selected from halogen, —OR⁶, —SR⁶, —N(R⁶)₂, —NO₂, and —CN;
R⁸ is selected from:
   C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —OC(O)N(R⁶)₂, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from: halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
p is 0, 1, or 2.

In one aspect, disclosed herein is compound represented by Formula (Ia) or (Ib):

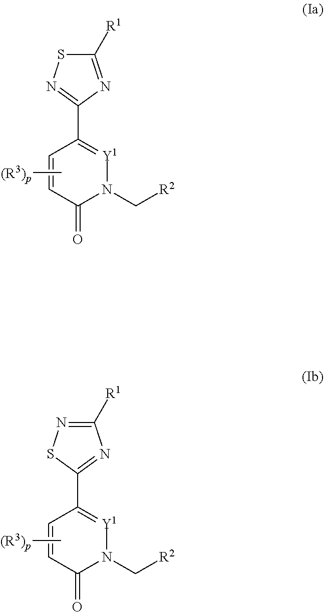

or a salt thereof, wherein:
Y¹ is N;
R¹ is selected from:
   C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
   C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R² is selected from:
   C₃₋₁₀ carbocycle and 3- to 10-membered heterocycle, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
R⁴ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
each R⁵ is independently selected from:
   halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
   and
   C₁₋₃ alkyl, C₂₋₃ alkenyl, C₂₋₃ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
each R⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
p is 0.

In one aspect, disclosed herein is compound represented by Formula (Ia) or (Ib):

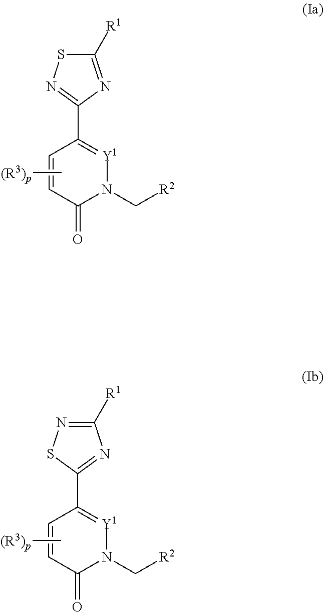

or a salt thereof, wherein:
Y¹ is N;
R¹ is selected from:
   C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
   C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R² is —C(O)NR⁷R⁸;
R⁴ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
each R⁵ is independently selected from:
   halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
   and
   C₁₋₃ alkyl, C₂₋₃ alkenyl, C₂₋₃ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂;
each R⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
R⁷ is selected from hydrogen and C₁₋₆ alkyl optionally substituted with one or more substituents independently selected from halogen, —OH, —SH, —NH₂, —NO₂, and —CN;
R⁸ is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more R⁵;
   C₃₋₁₀ carbocycle and 3- to 10-membered heterocycle, each of which is optionally substituted with one or more substituents independently selected from: halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
p is 0.

In certain embodiments, a compound or salt of Formula (Ia) or (Ib) is selected from

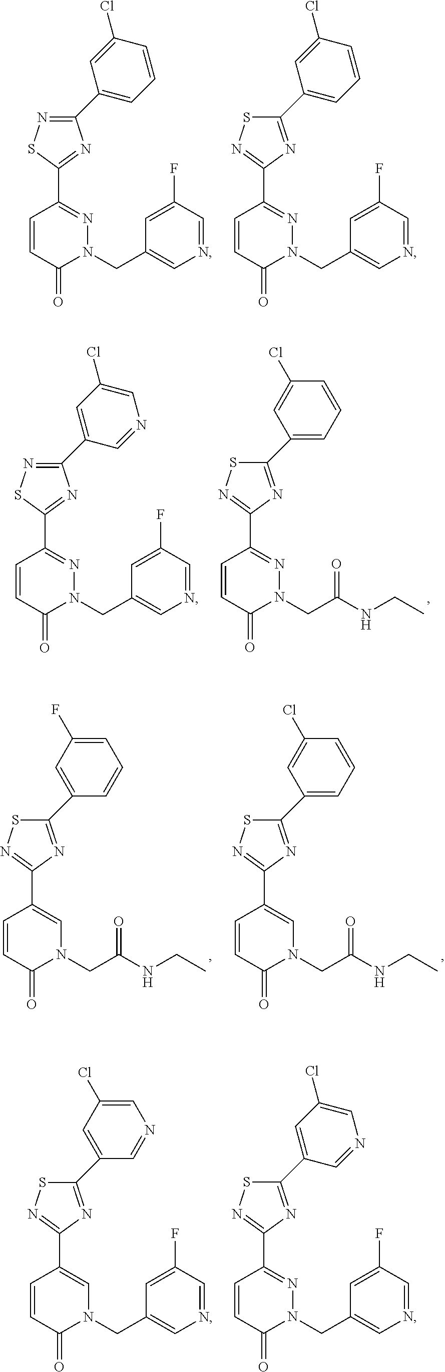

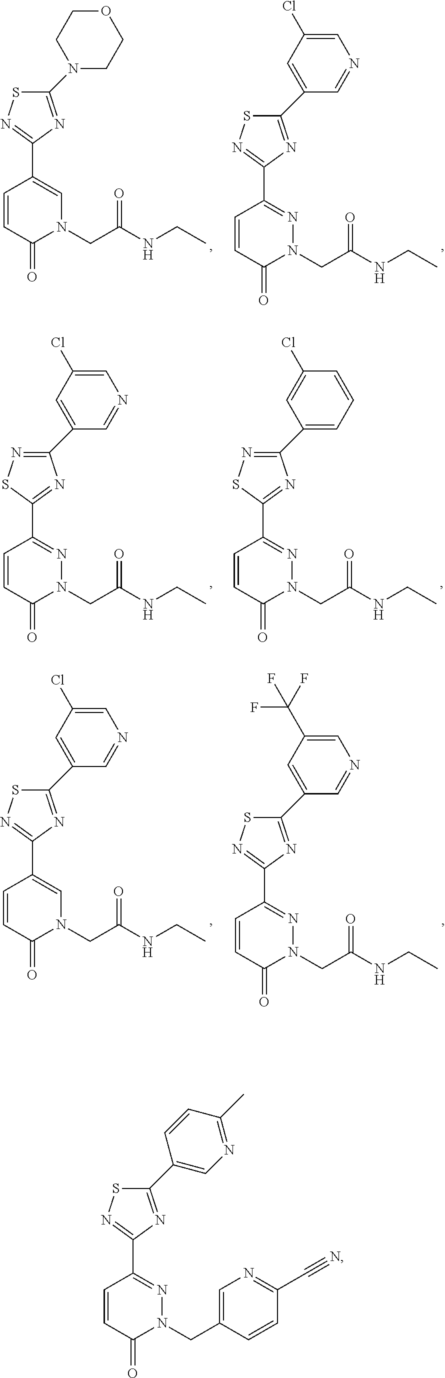

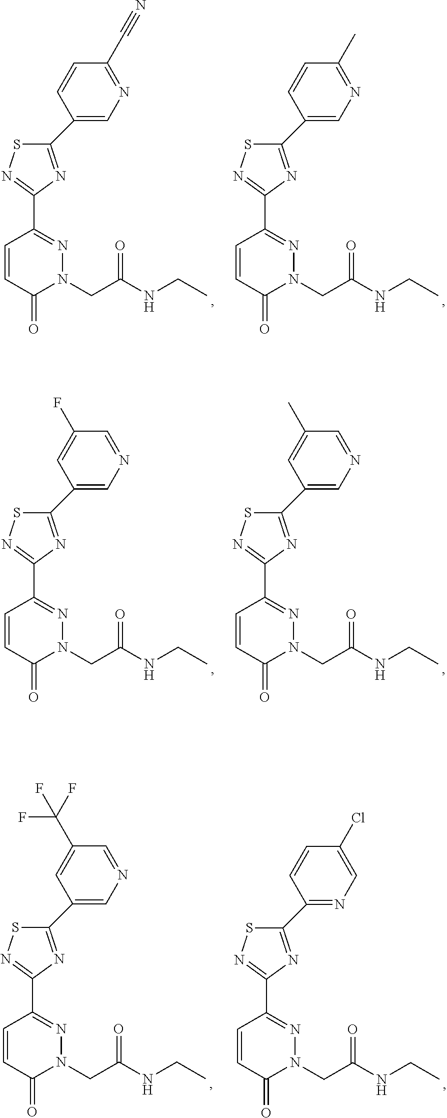

and a salt of any one thereof.

In certain embodiments, a compound or salt of Formula (Ia) or (Ib) is selected from

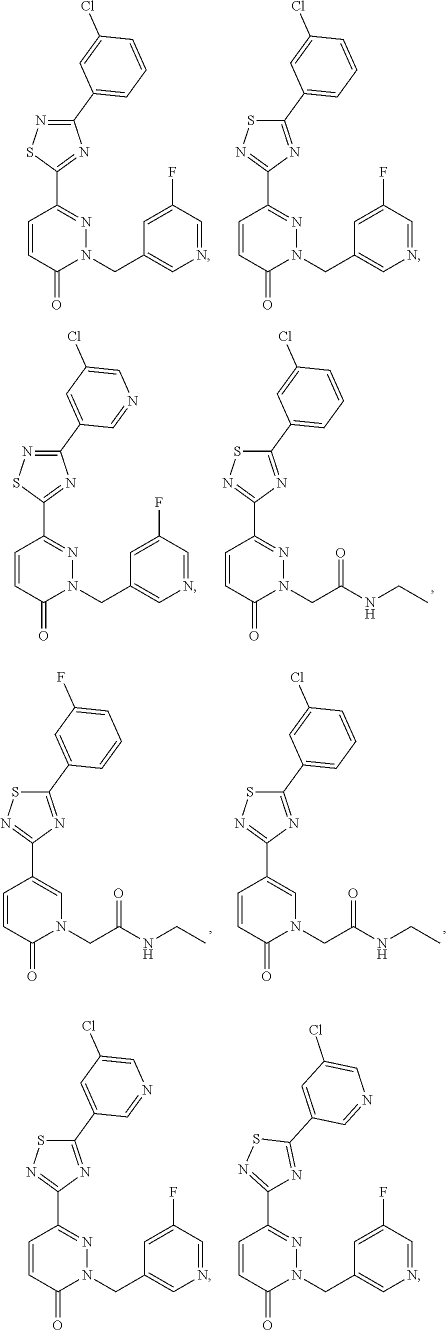

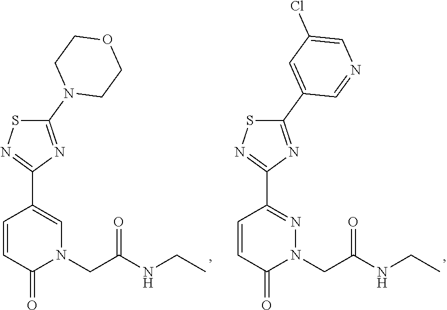

and a salt of any one thereof.

In certain embodiments, a compound or salt of Formula (Ia) or (Ib) is selected from

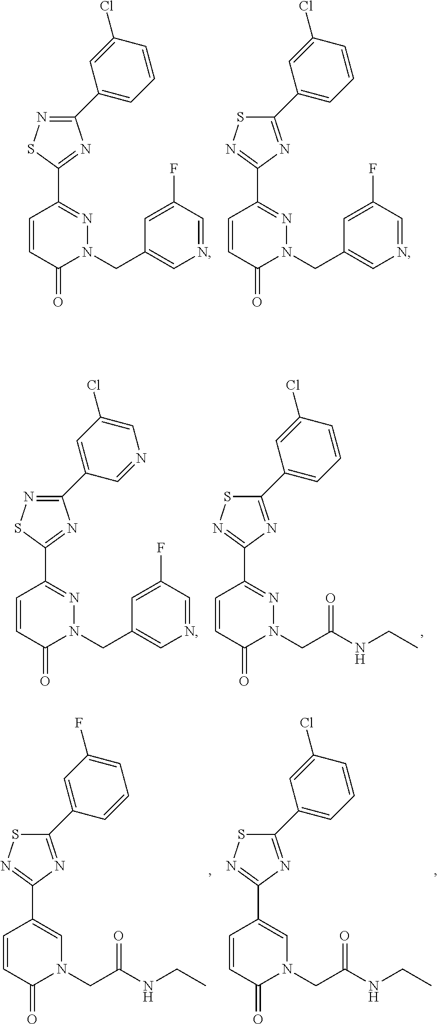

a salt of any one thereof.

In certain embodiments, a compound or salt of Formula (Ia) or (Ib) is selected from

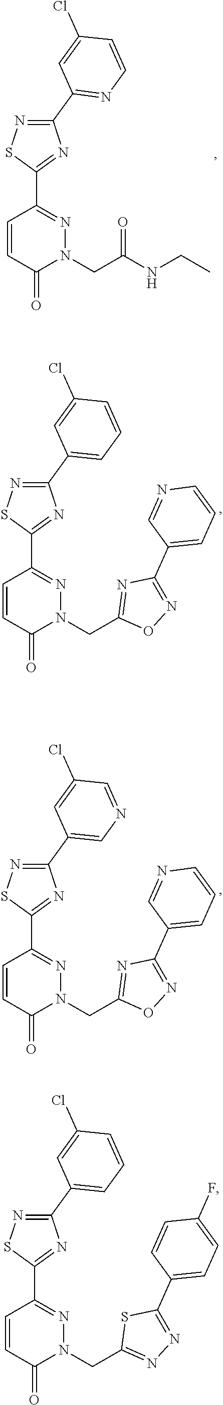 [SMILES: CCNC(=O)Cn1nc(-c2nc(-c3cc(Cl)ccn3)ns2)ccc1=O ; O=c1ccc(-c2nc(-c3cccc(Cl)c3)ns2)nn1Cc1nc(-c2cccnc2)no1 ; O=c1ccc(-c2nc(-c3cncc(Cl)c3)ns2)nn1Cc1nc(-c2cccnc2)no1 ; O=c1ccc(-c2nc(-c3cccc(Cl)c3)ns2)nn1Cc1nnc(-c2ccc(F)cc2)s1]

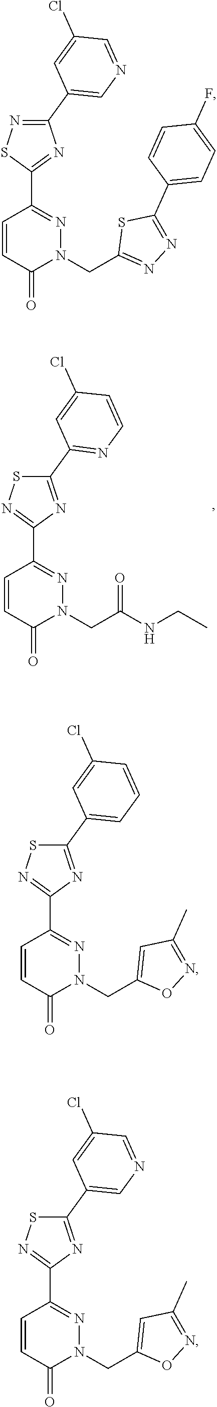 [SMILES: O=c1ccc(-c2nc(-c3cncc(Cl)c3)ns2)nn1Cc1nnc(-c2ccc(F)cc2)s1 ; CCNC(=O)Cn1nc(-c2nsc(-c3cc(Cl)ccn3)n2)ccc1=O ; Cc1cc(Cn2nc(-c3nsc(-c4cccc(Cl)c4)n3)ccc2=O)on1 ; Cc1cc(Cn2nc(-c3nsc(-c4cncc(Cl)c4)n3)ccc2=O)on1]

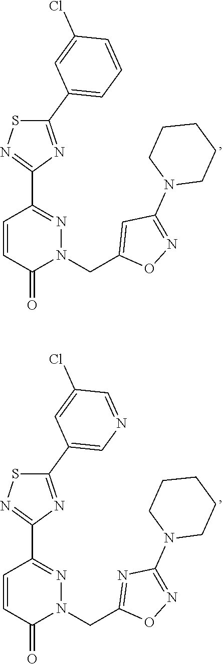 [SMILES: O=c1ccc(-c2nsc(-c3cccc(Cl)c3)n2)nn1Cc1cc(N2CCCCC2)no1 ; O=c1ccc(-c2nsc(-c3cncc(Cl)c3)n2)nn1Cc1nc(N2CCCCC2)no1]

and a salt of any one thereof.

In one aspect, disclosed herein is a compound represented by Formula (II):

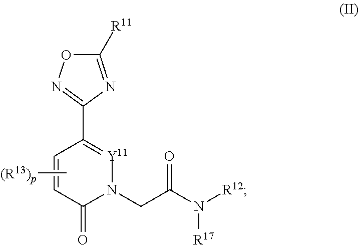

or a salt thereof, wherein:
Y¹¹ is N or CR¹⁴;
R¹¹ is selected from:
   hydrogen;
   halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R¹² is selected from:
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R¹³ is independently selected from halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
R¹⁴ is independently selected from hydrogen, halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
each R¹⁵ is independently selected from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN; and
   C₁₋₃ alkyl, C₂₋₃ alkenyl, C₂₋₃ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R¹⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R¹⁷ is selected from hydrogen and C₁₋₆ alkyl optionally substituted with one or more substituents independently selected from halogen, —OR¹⁶, —SR¹⁶, —N(R¹⁶)₂, —NO₂, and —CN;
p is 0, 1, or 2.

In certain embodiments, for a compound or salt of Formula (II), is not

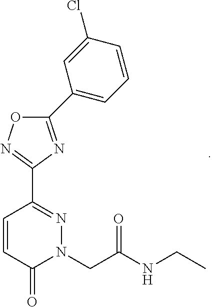 [SMILES: CCNC(=O)Cn1nc(-c2noc(-c3cccc(Cl)c3)n2)ccc1=O]

In certain embodiments, for a compound or salt of Formula (II), Y is N.

In certain embodiments, for a compound or salt of Formula (II), Y is CR¹⁴.

In certain embodiments, for a compound or salt of Formula (II), R¹¹ is selected from:
halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵.

In certain embodiments, for a compound or salt of Formula (II), R¹¹ is selected from:
halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, and —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R¹.

In certain embodiments, for a compound or salt of Formula (II), R¹¹ is selected from:
halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (II), R¹¹ is selected from C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹.

In certain embodiments, for a compound or salt of Formula (II), R¹¹ is selected from C₅₋₆ carbocycle and 5- to 6-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵.

In certain embodiments, for a compound or salt of Formula (II), R¹¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (II), R¹¹ is selected from phenyl and pyridinyl each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R¹¹ is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In some embodiments, R¹² is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R¹² is selected from phenyl and pyridyl, each of which is optionally substituted with one or more halogen.

In certain embodiments, for a compound or salt of Formula (II), R¹¹ is selected from

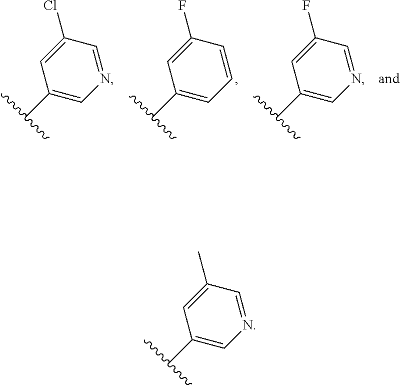

In certain embodiments, for a compound or salt of Formula (II), R¹¹ is selected from 3- to 10-membered heterocycle optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵. In certain embodiments, for a compound or salt of Formula (II), R¹¹ is selected from 5- to 6-membered heterocycle optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵. In certain embodiments, for a compound or salt of Formula (II), R¹¹ is selected from 5- to 6-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (II), R¹¹ is selected from: pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (II), R¹¹ is selected from

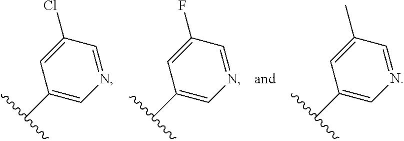

In certain embodiments, for a compound or salt of Formula (II), R¹² is selected from:
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵.

In certain embodiments, for a compound or salt of Formula (II), R¹² is selected from:
C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (II), R¹² is selected from C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl. In certain embodiments, R¹² is selected from C₁₋₆ alkyl. In certain embodiments, R¹² is selected from methyl, ethyl, and propyl. In some embodiments, R¹² is selected from methyl, ethyl, propyl, butyl, cyclopropyl, and cyclobutyl.

In certain embodiments, for a compound or salt of Formula (II), each R¹³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R¹³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R¹³ is C₁₋₆ alkyl optionally substituted with one or more substituents independently selected from halogen, —CN, —OH, —SH —NO₂, and —NH₂. In some embodiments, each R¹³ is C₁₋₆ alkyl. In certain embodiments, R¹³ is selected from methyl, ethyl, propyl, butyl, cyclopropyl, and cyclobutyl.

In certain embodiments, for a compound or salt of Formula (II), p is 0. In certain embodiments, p is 1. In certain embodiments, p is 2.

In certain embodiments, for a compound or salt of Formula (II), R¹⁴ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, for a compound or salt of Formula (II), R¹⁴ is selected from hydrogen and C₁₋₆ alkyl optionally substituted with one or more substituents independently selected from halogen, —CN, —OH, —SH —NO₂, and —NH₂. In certain embodiments, R¹⁴ is hydrogen. In certain embodiments, R¹⁴ is selected from methyl, ethyl, propyl, butyl, cyclopropyl, and cyclobutyl.

In certain embodiments, for a compound or salt of Formula (II), each R¹⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R¹⁵ is selected from methyl, ethyl, propyl, butyl, cyclopropyl, and cyclobutyl.

In certain embodiments, for a compound or salt of Formula (II), each R¹⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R¹⁶ is selected from methyl, ethyl, propyl, butyl, cyclopropyl, and cyclobutyl. In certain embodiments, R¹⁶ is hydrogen.

In one aspect, disclosed herein is a compound represented by Formula (II):

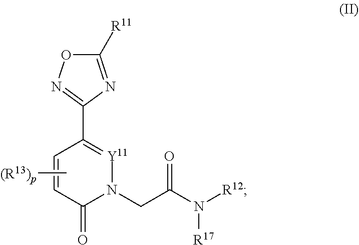

or a salt thereof, wherein:
Y¹¹ is N or CR¹⁴;
R¹¹ is selected from:
   hydrogen;
   halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R¹² is selected from:
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R¹³ is independently selected from halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
R¹⁴ is independently selected from hydrogen, halogen, —OR⁶, —SR¹⁶ —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
each R¹⁵ is independently selected from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN; and
   C₁₋₃ alkyl, C₂₋₃ alkenyl, C₂₋₃ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R¹⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R¹⁷ is selected from hydrogen and C₁₋₆ alkyl optionally substituted with one or more substituents independently selected from halogen, —OR¹⁶, —SR¹⁶, —N(R¹⁶)₂, —NO₂, and —CN;
p is 0, 1, or 2; and
   wherein the compound is not

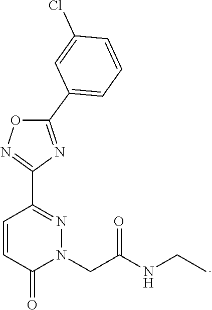 [SMILES: CCNC(=O)Cn1nc(-c2noc(-c3cccc(Cl)c3)n2)ccc1=O]

In one aspect, disclosed herein is a compound represented by Formula (II):

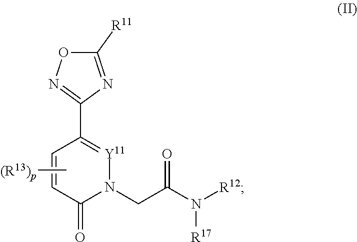

or a salt thereof, wherein:
Y¹¹ is N or CR¹⁴;
R¹¹ is selected from:
   hydrogen;
   halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   3- to 10-membered heterocycle optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵;
R¹² is selected from:
   C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
   C₃₋₁₀ carbocycle and 3- to 10-membered heterocycle, each of which is optionally substituted with one or more substituents independently selected from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R¹³ is independently selected from halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
R¹⁴ is independently selected from hydrogen, halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
each R¹⁵ is independently selected from:
   halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN; and
   C₁₋₃ alkyl, C₂₋₃ alkenyl, C₂₋₃ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R¹⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R¹⁷ is selected from hydrogen and C₁₋₆ alkyl optionally substituted with one or more substituents independently selected from halogen, —OR¹⁶, —SR¹⁶, —N(R¹⁶)₂, —NO₂, and —CN;
p is 0, 1, or 2.

In one aspect, disclosed herein is a compound represented by Formula (II):

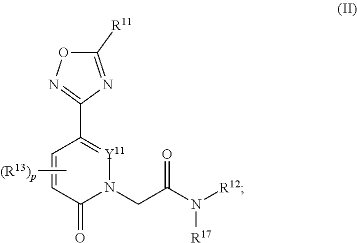

or a salt thereof, wherein:
Y¹¹ is N;
R¹¹ is selected from:
   C₁₋₆ alkyl optionally substituted with one or more substituents independently selected halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl); and
   3- to 10-membered heterocycle optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl);
R¹² is selected from:
   C₁₋₆ alkyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl); and
   C₃₋₁₀ carbocycle and 3- to 10-membered heterocycle, each of which is optionally substituted with one or more substituents independently selected from: halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl);
R¹⁷ is selected from hydrogen and C₁₋₆ alkyl optionally substituted with one or more substituents independently selected from halogen, —OH, —SH⁶, —NH₂, —NO₂, and —CN;
p is 0.

In certain embodiments, a compound or salt of Formula (II) is selected from

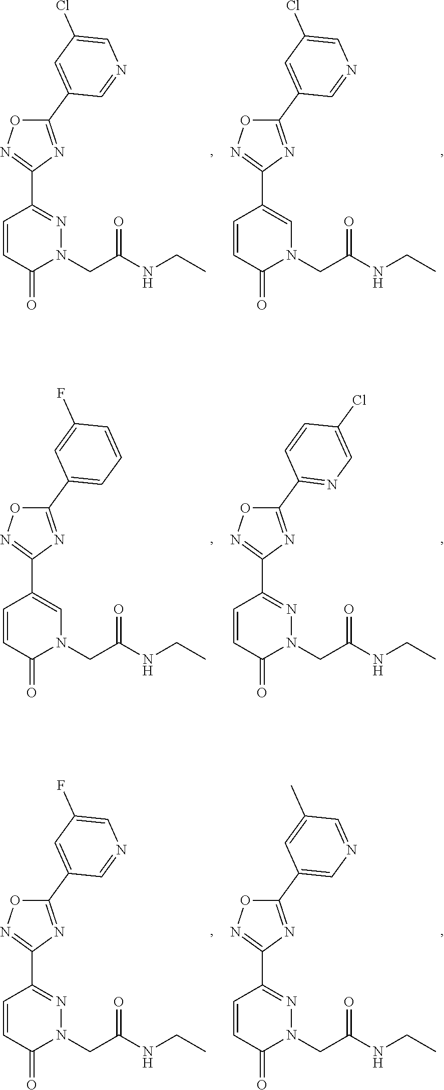

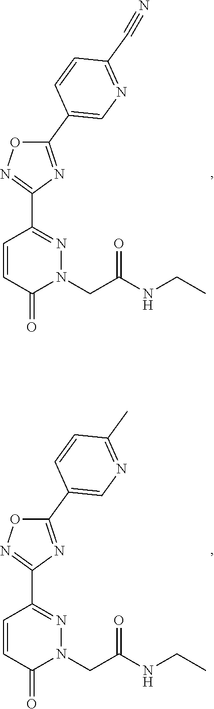 [SMILES: CCNC(=O)Cn1nc(-c2noc(-c3ccc(C#N)nc3)n2)ccc1=O ; CCNC(=O)Cn1nc(-c2noc(-c3ccc(C)nc3)n2)ccc1=O]

and a salt of ay one thereof.

In certain embodiments, a compound or salt of Formula (II) is selected from

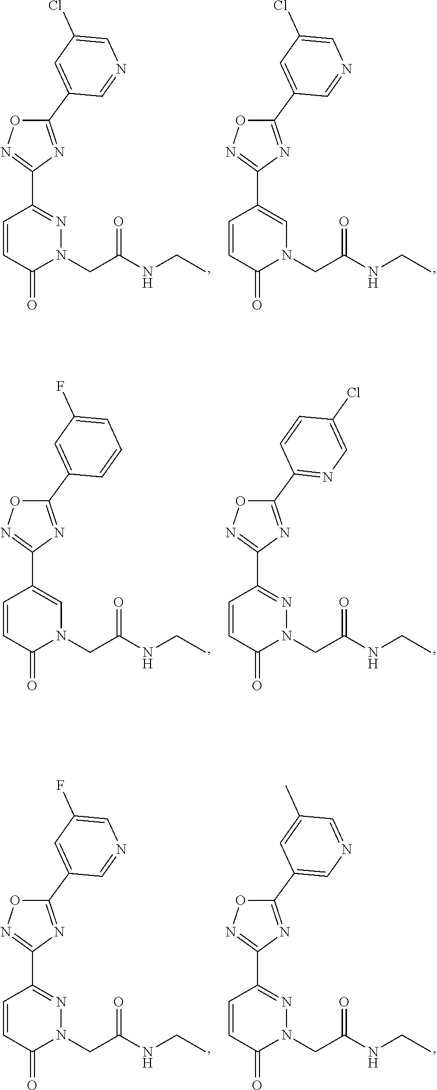

and a salt of any one thereof.

In certain embodiments, a compound or salt of Formula (II) is selected from

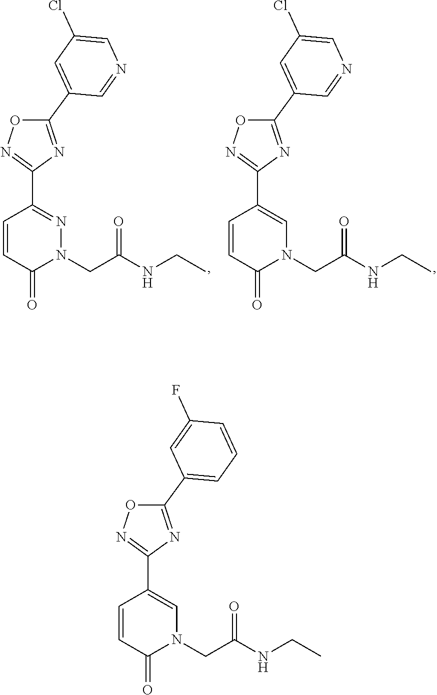

and a salt of any one thereof.

In one aspect, disclosed herein is a compound represented by Formula (III):

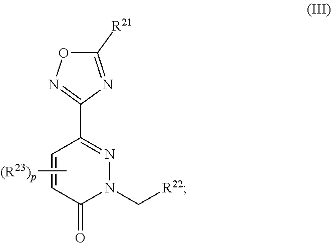

or a salt thereof, wherein:
R²¹ is selected from:
   hydrogen;
   halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, and —CN;
   C₂₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, ═O, ═S, ═N(R²⁶), —CN, C₃₋₁₀ carbocycle and 3- to 10-membered heterocycle, wherein the C₃₋₁₀ carbocycle and 3- to 10-membered heterocycle are each optionally substituted with one or more R²⁵; and
   C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵;
R²² is selected from:
   C₁₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R²⁵; and
   C₃₋₁₀ carbocycle and 3- to 10-membered heterocycle, each of which is optionally substituted with one or more substituents independently selected from: halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵;
each R²³ is independently selected from halogen, —OR²⁶, —SR²⁶, —N(R²⁶)₂, —NO₂, —CN, and C₁₋₆ alkyl optionally substituted with one or more substituents independently selected from halogen, —OR²⁶, —SR²⁶, —N(R²⁶)₂, —NO₂, and —CN;
each R²⁵ is independently selected from:
   halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN; and
   C₁₋₃ alkyl, C₂₋₃ alkenyl, C₂₋₃ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN;
each R²⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

In certain embodiments, for a compound or salt of Formula (III), R²¹ is selected from:
halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, and —CN;
C₂₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, ═O, ═S, ═N(R²⁶), —CN, C₃₋₁₀ carbocycle and 3- to 10-membered heterocycle, wherein the C₃₋₁₀ carbocycle and 3- to 10-membered heterocycle are each optionally substituted with one or more R²⁵; and
C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵.

In certain embodiments, for a compound or salt of Formula (III), R²¹ is selected from:
halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, and —CN;
C₂₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R²⁵; and
C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵;

In certain embodiments, for a compound or salt of Formula (III), R²¹ is selected from:
halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, and —CN;
C₂₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (III), R²¹ is selected from C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵. In certain embodiments, R²¹ is selected from C₅₋₆ carbocycle and 5- to 6-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵. In certain embodiments, R²¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²¹ is selected from phenyl and pyridinyl each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²¹ is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²¹ is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²¹ is selected from phenyl and pyridyl, each of which is optionally substituted with one or more halogen.

In certain embodiments, for a compound or salt of Formula (III), R²¹ is selected from

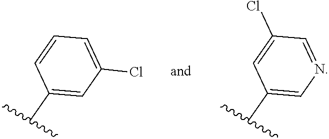 [SMILES: *c1cccc(Cl)c1 ; *c1cncc(Cl)c1]

In certain embodiments, for a compound or salt of Formula (III), R²² is selected from:
C₁₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R²⁵; and
C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵.

In certain embodiments, for a compound or salt of Formula (III), R²² is selected from C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵. In certain embodiments, R²² is selected from C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵. In certain embodiments, R²² is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²² is selected from pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R²² is selected from phenyl and pyridyl, each of which is optionally substituted with one or more halogen.

In certain embodiments, for a compound or salt of Formula (III), R²² is selected from 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, 3- to 10-membered heterocycle, wherein C₁₋₆ alkyl, C₃₋₁₀ carbocycle, 3- to 10-membered heterocycle, are optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl). In certain embodiments, R²² is selected from 5- to 9-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, 3- to 10-membered heterocycle, wherein C₁₋₆ alkyl, C₃₋₁₀ carbocycle, 3- to 10-membered heterocycle, are optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl). In certain embodiments, R²² is selected from 5-membered heterocycle optionally substituted with one or more substituents independently selected from halogen, C₁₋₆ alkyl, phenyl, and pyridyl, wherein C₁₋₆ alkyl, phenyl, and pyridyl are optionally substituted with one or more substituents independently selected from halogen, —CN, —OH, —SH, —NO₂, —NH₂, —O—C₁₋₆ alkyl, —S—C₁₋₆ alkyl, —N(C₁₋₆ alkyl)₂, —NH(C₁₋₆ alkyl). In certain embodiments, R²² is selected from 5-membered heterocycle optionally substituted with one or more substituents independently selected from halogen, C₁₋₆ alkyl, phenyl, and pyridyl, wherein C₁₋₆ alkyl, phenyl, and pyridyl are optionally substituted with one or more substituents independently selected from halogen.

In certain embodiments, for a compound or salt of Formula (III), R²² is selected from

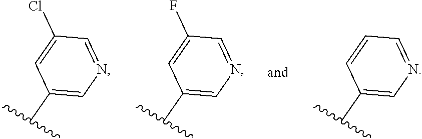 [SMILES: *c1cncc(Cl)c1 ; *c1cncc(F)c1 ; *c1cccnc1]

In certain embodiments, for a compound or salt of Formula (III), each R²³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments each R²³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R²³ is C₁₋₆ alkyl optionally substituted with one or more substituents independently selected from halogen, —CN, —OH, —SH —NO₂, and —NH₂. In some embodiments, each R²³ is C₁₋₆ alkyl. In certain embodiments, R²³ is selected from methyl, ethyl, propyl, butyl, cyclopropyl, and cyclobutyl.

In certain embodiments, for a compound or salt of Formula (III), p is 0. In certain embodiments, p is 1. In certain embodiments, p is 2.

In certain embodiments, for a compound or salt of Formula (III), each R²⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R²⁵ is selected from methyl, ethyl, propyl, butyl, cyclopropyl, and cyclobutyl. In certain embodiments, for a compound or salt of Formula (II), each R²⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, each R²⁶ is selected from hydrogen, halogen, and C₁₋₃ alkyl optionally substituted with one or more substituents independently selected from halogen, —CN, —OH, —SH —NO₂, and —NH₂. In certain embodiments, R²⁶ is selected from methyl, ethyl, propyl, butyl, cyclopropyl, and cyclobutyl. In certain embodiments, R²⁶ is hydrogen.

In certain embodiments, for a compound or salt of Formula (III), the compound is selected from

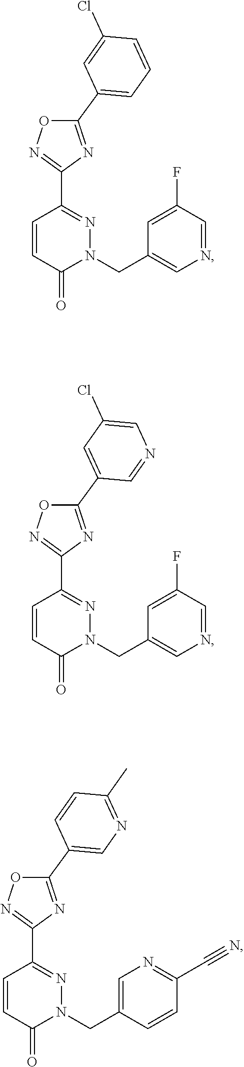 [SMILES: O=c1ccc(-c2noc(-c3cccc(Cl)c3)n2)nn1Cc1cncc(F)c1 ; O=c1ccc(-c2noc(-c3cncc(Cl)c3)n2)nn1Cc1cncc(F)c1 ; Cc1ccc(-c2nc(-c3ccc(=O)n(Cc4ccc(C#N)nc4)n3)no2)cn1]

and a salt of any one thereof.

In certain embodiments, for a compound or salt of Formula (III), the compound is selected from

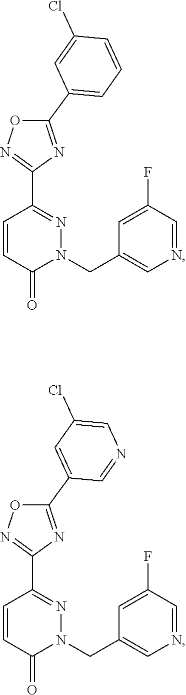 [SMILES: O=c1ccc(-c2noc(-c3cccc(Cl)c3)n2)nn1Cc1cncc(F)c1 ; O=c1ccc(-c2noc(-c3cncc(Cl)c3)n2)nn1Cc1cncc(F)c1]

and a salt of any one thereof.

In certain embodiments, for a compound or salt of Formula (III), the compound is selected from

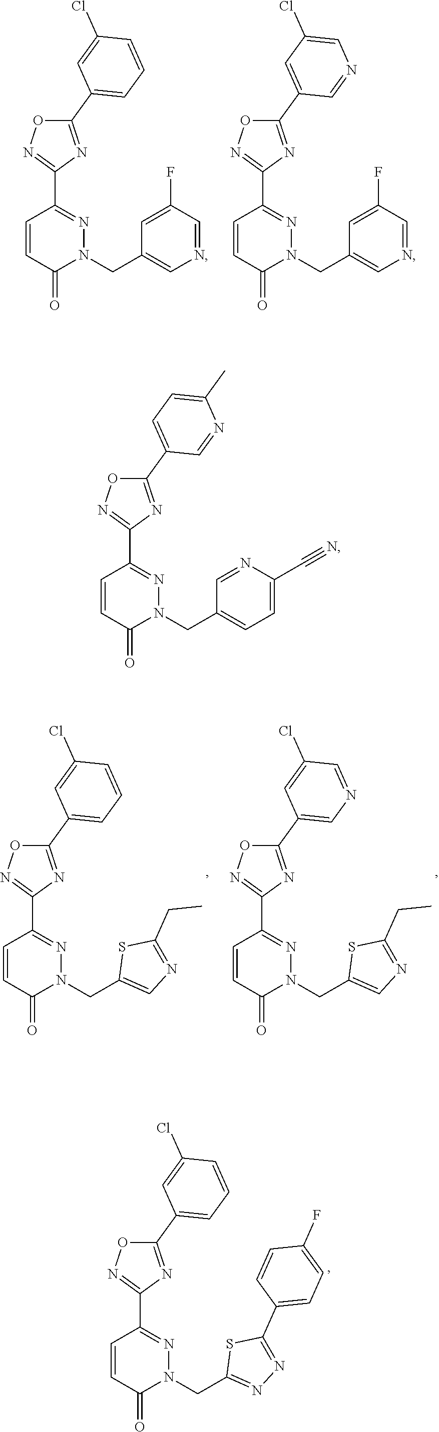

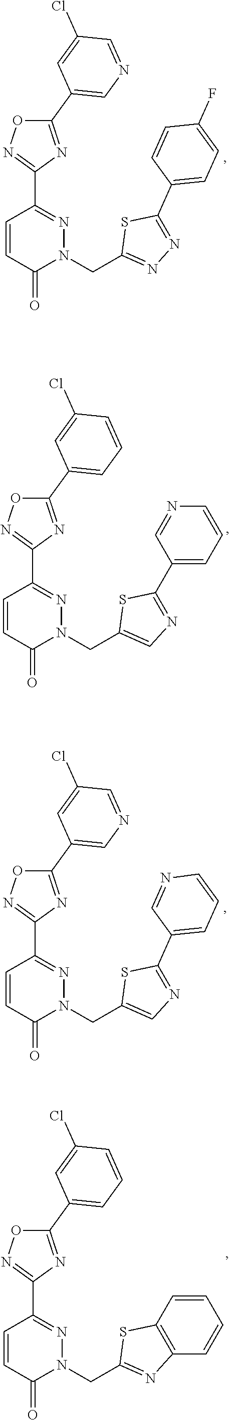 [SMILES: O=c1ccc(-c2noc(-c3cncc(Cl)c3)n2)nn1Cc1nnc(-c2ccc(F)cc2)s1 ; O=c1ccc(-c2noc(-c3cccc(Cl)c3)n2)nn1Cc1cnc(-c2cccnc2)s1 ; O=c1ccc(-c2noc(-c3cncc(Cl)c3)n2)nn1Cc1cnc(-c2cccnc2)s1 ; O=c1ccc(-c2noc(-c3cccc(Cl)c3)n2)nn1Cc1nc2ccccc2s1]

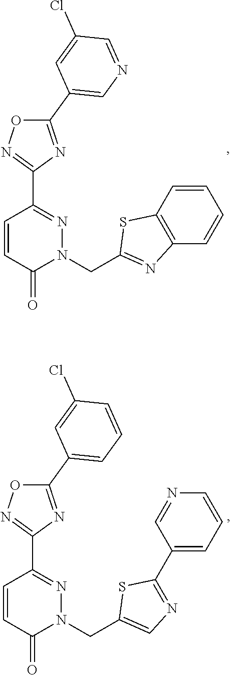 [SMILES: O=c1ccc(-c2noc(-c3cncc(Cl)c3)n2)nn1Cc1nc2ccccc2s1 ; O=c1ccc(-c2noc(-c3cccc(Cl)c3)n2)nn1Cc1cnc(-c2cccnc2)s1]

and a salt of any one thereof.

In one aspect, disclosed herein is a compound represented by Formula (IV):

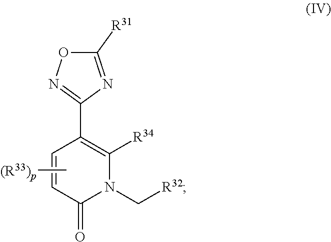

or a salt thereof, wherein:
R³¹ is selected from:
   hydrogen;
   halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂ and —CN;
   C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂, ═O, ═S, ═N(R³⁶), —CN, C₃₋₁₀ carbocycle and 3- to 10-membered heterocycle, wherein the C₃₋₁₀ carbocycle and 3- to 10-membered heterocycle are each optionally substituted with one or more R³⁵; and
   C₃₋₁₀ carbocycle and 3- to 10-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵;
R³² is selected from:
   C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R³⁵;
   and
   3- to 10-membered heterocycle optionally substituted with one or more substituents independently selected from: halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵;
each R³³ is independently selected from halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
R³⁴ is independently selected from hydrogen, halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
each R³⁵ is independently selected from:
   halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN; and
   C₁₋₃ alkyl, C₂₋₃ alkenyl, C₂₋₃ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN;
each R³⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
p is 0, 1, or 2.

In certain embodiments, for a compound or salt of Formula (IV), R³¹ is selected from:
halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂, and —CN;
C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂, ═O, ═S, ═N(R³⁶), —CN, C₃₋₁₀ carbocycle and 3- to 10-membered heterocycle, wherein the C₃₋₁₀ carbocycle and 3- to 10-membered heterocycle are each optionally substituted with one or more R³⁵; and
3- to 10-membered heterocycle, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵.

In certain embodiments, for a compound or salt of Formula (IV), R³¹ is selected from:
halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —NO₂, and —CN;
C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —C(O)OR³⁶, —OC(O)R³⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R³⁵; and
C₃₋₁₀ carbocycle and 3- to 10-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵.

In certain embodiments, for a compound or salt of Formula (IV), R³¹ is selected from:
halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (IV), R³¹ is selected from C₃₋₁₀ carbocycle and 3- to 10-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵. In certain embodiments, for a compound or salt of Formula (IV), R³¹ is selected from C₅₋₆ carbocycle and 5- to 6-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, —N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵. In certain embodiments, for a compound or salt of Formula (IV), R³¹ is selected from 5- to 6-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (IV), R³¹ is selected from pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R³¹ is selected from phenyl optionally substituted with one or more substituents independently selected from C₁₋₆ alkyl, C₂₋₆ alkenyl, C₂₋₆ alkynyl, C₃₋₁₀ carbocycle, 3- to 10-membered heterocycle, and haloalkyl. In certain embodiments, R³¹ is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R³¹ is selected from phenyl and pyridyl, each of which is optionally substituted with one or more halogen.

In certain embodiments, for a compound or salt of Formula (IV), R³¹ is selected from

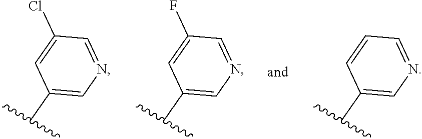 [SMILES: *c1cncc(Cl)c1 ; *c1cncc(F)c1 ; *c1cccnc1]

In certain embodiments, for a compound or salt of Formula (IV), R³² is selected from 3- to 10-membered heterocycle optionally substituted with one or more substituents independently selected from halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵. In certain embodiments, for a compound or salt of Formula (IV), R³² is selected from 5- to 6-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (IV), R³² is selected from pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R³² is pyridyl optionally substituted with one or more halogen.

In certain embodiments, for a compound or salt of Formula (IV), R³² is selected from

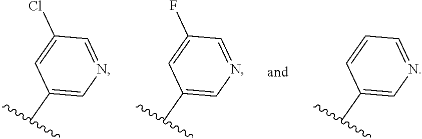 [SMILES: *c1cncc(Cl)c1 ; *c1cncc(F)c1 ; *c1cccnc1]

In certain embodiments, for a compound or salt of Formula (IV), each R³³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, for a compound or salt of Formula (IV), each R³³ is C₁₋₆ alkyl optionally substituted with one or more substituents independently selected from halogen, —CN, —OH, —SH —NO₂, and —NH₂. In some embodiments, each R³³ is C₁₋₆ alkyl. In certain embodiments, R³³ is selected from methyl, ethyl, propyl, butyl, cyclopropyl, and cyclobutyl.

In certain embodiments, for a compound or salt of Formula (IV), p is 0. In certain embodiments, p is 1. In certain embodiments, p is 2.

In certain embodiments, for a compound or salt of Formula (IV), wherein R³⁴ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In some embodiments, R³⁴ is selected from hydrogen and C₁₋₆ alkyl optionally substituted with one or more substituents independently selected from halogen, —CN, —OH, —SH —NO₂, and —NH₂. In some embodiments, R³⁴ is hydrogen. In certain embodiments, R³⁴ is selected from methyl, ethyl, propyl, butyl, cyclopropyl, and cyclobutyl.

In certain embodiments, for a compound or salt of Formula (IV), each R³⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R³⁵ is selected from methyl, ethyl, propyl, butyl, cyclopropyl, and cyclobutyl.

In certain embodiments, for a compound or salt of Formula (IV), each R³⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R³⁶ is selected from methyl, ethyl, propyl, butyl, cyclopropyl, and cyclobutyl. In certain embodiments, R³⁶ is hydrogen.

In some embodiments, each R³⁶ is selected from hydrogen, halogen, and C₁₋₃ alkyl optionally substituted with one or more substituents independently selected from halogen, —CN, —OH, —SH —NO₂, and —NH₂.

In one aspect, disclosed herein is a compound represented by Formula (IV):

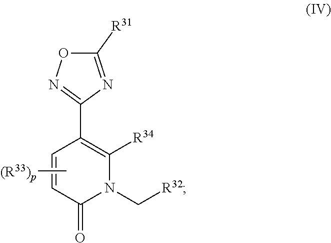

or a salt thereof, wherein:
R³¹ is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more R³⁵; and
   C₃₋₁₀ carbocycle and 3- to 10-membered heterocycle, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵;
R³² is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more R³⁵; and
   3- to 10-membered heterocycle optionally substituted with one or more substituents independently selected from: halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵;
each R³³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, and —NH(C₁₋₆ alkyl);
R³⁴ is independently selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, and —NH(C₁₋₆ alkyl);
each R³⁵ is independently selected from:
   halogen, —CN, —OH, —SH —NO₂, —NH₂, —O—C₁₋₆ alkyl, —S—C₁₋₆ alkyl, —N(C₁₋₆ alkyl)₂, and —NH(C₁₋₆ alkyl); and
   C₁₋₃ alkyl, C₂₋₃ alkenyl, C₂₋₃ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, and —NH(C₁₋₆ alkyl);
each R³⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
p is 0, 1, or 2.

In one aspect, disclosed herein is a compound represented by Formula (IV):

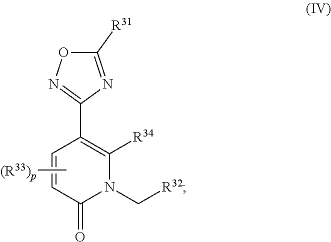

or a salt thereof, wherein:
R³¹ is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle; and
   C₃₋₁₀ carbocycle and 3- to 10-membered heterocycle, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, and 3- to 10-membered heterocycle;
R³² is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle; and
   3- to 10-membered heterocycle optionally substituted with one or more substituents independently selected from: halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle;
R³⁴ is hydrogen;
p is 0.

In certain embodiments, for a compound or salt of Formula (IV), the compound is selected from

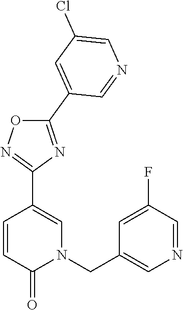 [SMILES: O=c1ccc(-c2noc(-c3cncc(Cl)c3)n2)cn1Cc1cncc(F)c1]

and a salt thereof.

In one aspect, disclosed herein is a compound represented by Formula (V):

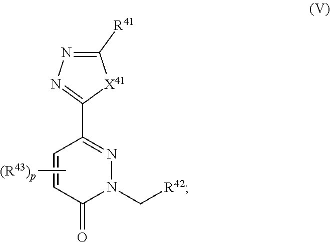

or a salt thereof, wherein:
X⁴¹ is O or S;
R⁴¹ is selected from:
   hydrogen;
   halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂ and —CN;
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
   C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴¹;
R⁴² is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
   —C(O)NR⁴⁷R⁴⁸;
each R⁴³ is independently selected from halogen, —OR⁴⁶, —SR⁴⁶, —N(R⁴⁶)₂, —NO₂, —CN, and C₁₋₆ alkyl optionally substituted with one or more substituents independently selected from halogen, —OR⁴⁶, —SR⁴⁶, —N(R⁴⁶)₂, —NO₂, and —CN;
each R⁴⁵ is independently selected from:
   halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —NO₂, ═O, ═S, ═N(R⁴⁶), and —CN; and
   C₁₋₃ alkyl, C₂₋₃ alkenyl, C₂₋₃ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN;
each R⁴⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁴⁷ is selected from hydrogen and C₁₋₆ alkyl optionally substituted with one or more substituents independently selected from halogen, —OR⁴⁶, —SR⁴⁶, —N(R⁴⁶)₂, —NO₂, and —CN;
R⁴⁸ is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
p is 0, 1, or 2.

In certain embodiments, for a compound or salt of Formula (V), the compound is not the following:

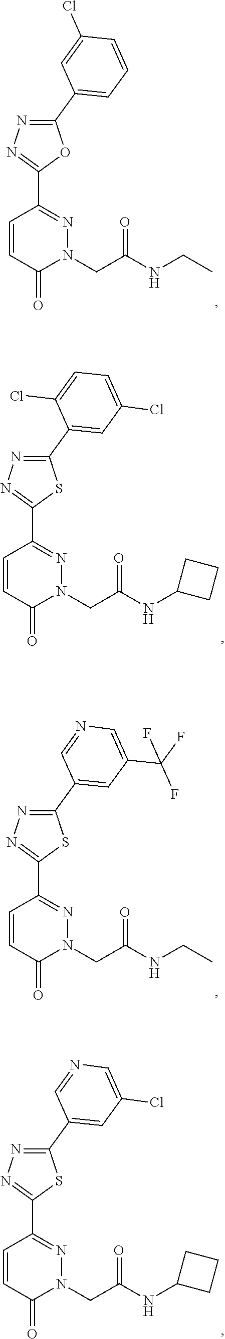 [SMILES: CCNC(=O)Cn1nc(-c2nnc(-c3cccc(Cl)c3)o2)ccc1=O ; O=C(Cn1nc(-c2nnc(-c3cc(Cl)ccc3Cl)s2)ccc1=O)NC1CCC1 ; CCNC(=O)Cn1nc(-c2nnc(-c3cncc(C(F)(F)F)c3)s2)ccc1=O ; O=C(Cn1nc(-c2nnc(-c3cncc(Cl)c3)s2)ccc1=O)NC1CCC1]

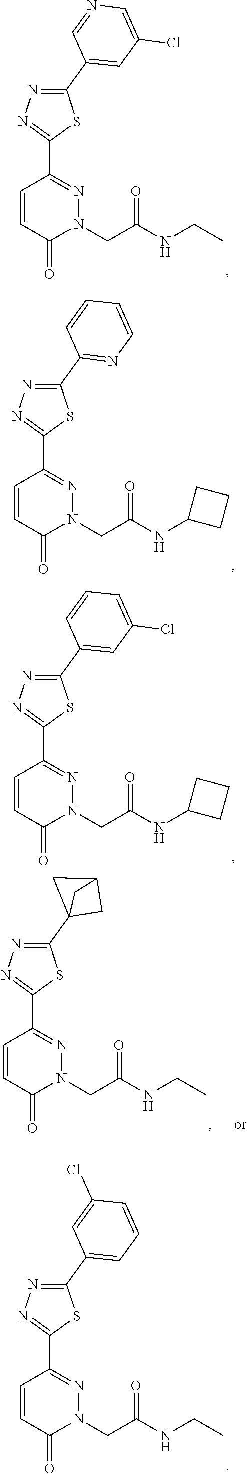

In certain embodiments, for a compound or salt of Formula (V), X⁴¹ is O. In certain embodiments, X⁴¹ is S.

In certain embodiments, for a compound or salt of Formula (V), R⁴¹ is selected from:
halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, and —CN;
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴⁵.

In certain embodiments, for a compound or salt of Formula (V), R⁴¹ is selected from:
halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —NO₂, and —CN;
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵.

In certain embodiments, for a compound or salt of Formula (V), R⁴¹ is selected from:
halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (V), R⁴¹ is selected from C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴⁵. In certain embodiments, for a compound or salt of Formula (V), R⁴¹ is selected from C₅₋₆ carbocycle and 5- to 6-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, —N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴⁵. In certain embodiments, for a compound or salt of Formula (V), R⁴¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (V), R⁴¹ is selected from phenyl and pyridinyl each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁴¹ is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁴¹ is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁴¹ is selected from phenyl and pyridyl, each of which is optionally substituted with one or more halogen. In certain embodiments, for a compound or salt of Formula (V), R⁴¹ is selected from

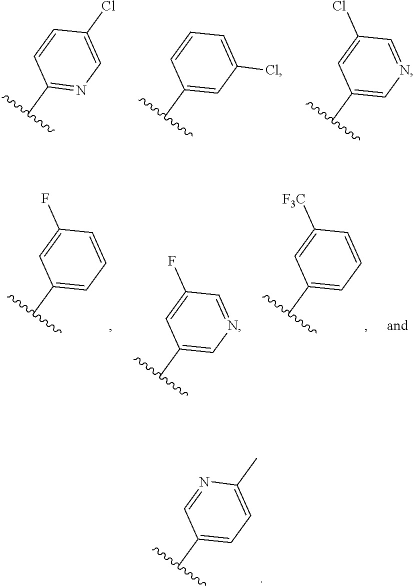

In certain embodiments, for a compound or salt of Formula (V), R⁴¹ is selected from

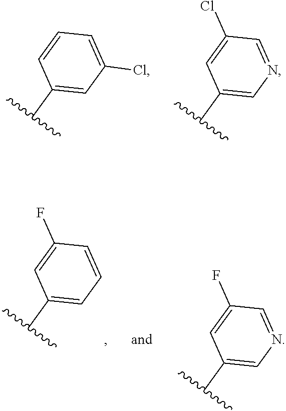 [SMILES: *c1cccc(Cl)c1 ; *c1cncc(Cl)c1 ; *c1cccc(F)c1 ; *c1cncc(F)c1]

In some embodiments, R⁴¹ is selected from

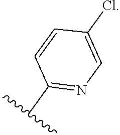 [SMILES: *c1ccc(Cl)cn1]

In certain embodiments, for a compound or salt of Formula (V), R⁴² is selected from:
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
—C(O)NR⁴⁷R⁴⁸.

In certain embodiments, for a compound or salt of Formula (V), R⁴² is selected from:
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
—C(O)NR⁴⁷R⁴⁸.

In certain embodiments, for a compound or salt of Formula (V), R⁴² is selected from:
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵.

In certain embodiments, for a compound or salt of Formula (V), R⁴² is selected from C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵. In certain embodiments, for a compound or salt of Formula (V), R⁴² is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (V), R⁴² is selected from pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁴² is selected from phenyl and pyridyl, each of which is optionally substituted with one or more halogen.

In certain embodiments, for a compound or salt of Formula (V), R⁴² is selected from

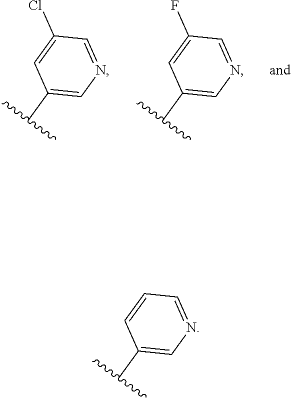 [SMILES: *c1cncc(Cl)c1 ; *c1cncc(F)c1 ; *c1cccnc1]

In certain embodiments, for a compound or salt of Formula (V), R⁴² is —C(O)NR⁴⁷R⁴⁸.

In certain embodiments, for a compound or salt of Formula (V), R⁴⁷ is selected from hydrogen and C₁₋₆ alkyl. In certain embodiments, R⁴⁷ is selected from hydrogen.

In certain embodiments, for a compound or salt of Formula (V), R⁴⁸ is selected from:
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵.

In certain embodiments, for a compound or salt of Formula (V), R⁴⁸ is selected from:
C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (V), R⁴⁸ is selected from C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl. In certain embodiments, R⁴⁸ is selected from C₁₋₆ alkyl. In certain embodiments, R⁴⁸ is selected from C₁₋₆ alkyl, and R⁴⁷ is hydrogen. In certain embodiments, R⁴⁸ is selected from methyl, ethyl, and propyl. In certain embodiments, R⁴⁸ is selected from methyl, ethyl, propyl, butyl, cyclopropyl, and cyclobutyl. In certain embodiments, R⁴⁸ is selected from ethyl. In certain embodiments, R⁴⁸ is selected from ethyl, and R⁴⁷ is hydrogen.

In certain embodiments, for a compound or salt of Formula (V), each R⁴³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, for a compound or salt of Formula (V), each R⁴³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, for a compound or salt of Formula (V), each R⁴³ is C₁₋₆ alkyl optionally substituted with one or more substituents independently selected from halogen, —CN, —OH, —SH —NO₂, and —NH₂. In some embodiments, each R⁴³ is C₁₋₆ alkyl. In certain embodiments, R⁴³ is selected from methyl, ethyl, propyl, butyl, cyclopropyl, and cyclobutyl.

In certain embodiments, for a compound or salt of Formula (V), p is 0. In certain embodiments, p is 1. In certain embodiments, p is 2.

In certain embodiments, for a compound or salt of Formula (V), each R⁴⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R⁴⁵ is selected from methyl, ethyl, propyl, butyl, cyclopropyl, and cyclobutyl.

In certain embodiments, for a compound or salt of Formula (V), each R⁴⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R⁴⁶ is selected from methyl, ethyl, and propyl.

In certain embodiments, for a compound or salt of Formula (V), each R⁴⁶ is selected from hydrogen, halogen, and C₁₋₃ alkyl optionally substituted with one or more substituents independently selected from halogen, —CN, —OH, —SH —NO₂, and —NH₂. In certain embodiments, R⁴⁶ is selected from methyl, ethyl, propyl, butyl, cyclopropyl, and cyclobutyl. In certain embodiments, R⁴⁶ is hydrogen.

In one aspect, disclosed herein is a compound represented by Formula (V):

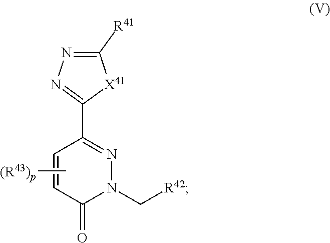

or a salt thereof, wherein:
X⁴¹ is O or S;
R⁴¹ is selected from:
   hydrogen;
   halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂ and —CN;
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
   C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴⁵;
R⁴² is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴;
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
   —C(O)NR⁴⁷R⁴⁸;
each R⁴³ is independently selected from halogen, —OR⁴⁶, —SR⁴⁶, —N(R⁴⁶)₂, —NO₂, —CN, and C₁₋₆ alkyl optionally substituted with one or more substituents independently selected from halogen, —OR⁴⁶, —SR⁴⁶, —N(R⁴⁶)₂, —NO₂, and —CN;
each R⁴¹ is independently selected from:
   halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —NO₂, ═O, ═S, ═N(R⁴⁶), and —CN; and
   C₁₋₃ alkyl, C₂₋₃ alkenyl, C₂₋₃ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN;
each R⁴⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁴⁷ is selected from hydrogen and C₁₋₆ alkyl optionally substituted with one or more substituents independently selected from halogen, —OR⁴⁶, —SR⁴⁶, —N(R⁴⁶)₂, —NO₂, and —CN;
R⁴⁸ is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
   C₃₋₁₀ carbocycle and 3- to 10-membered heterocycle, each of which is optionally substituted with one or more substituents independently selected from:
halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
p is 0, 1, or 2,
   wherein the compound is not the following:

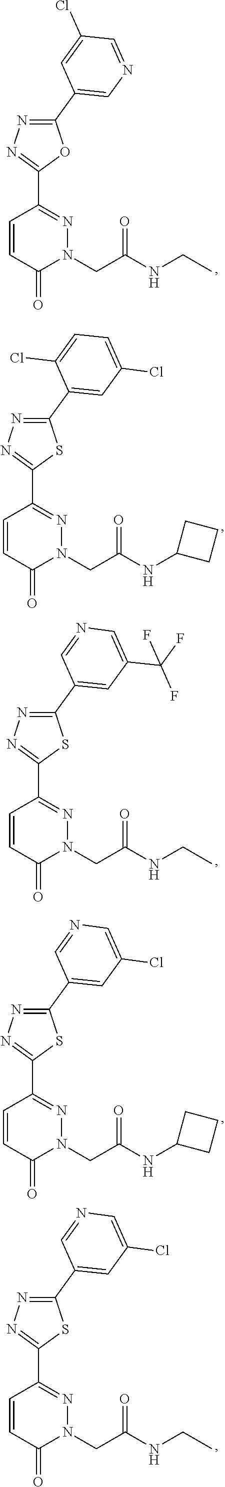

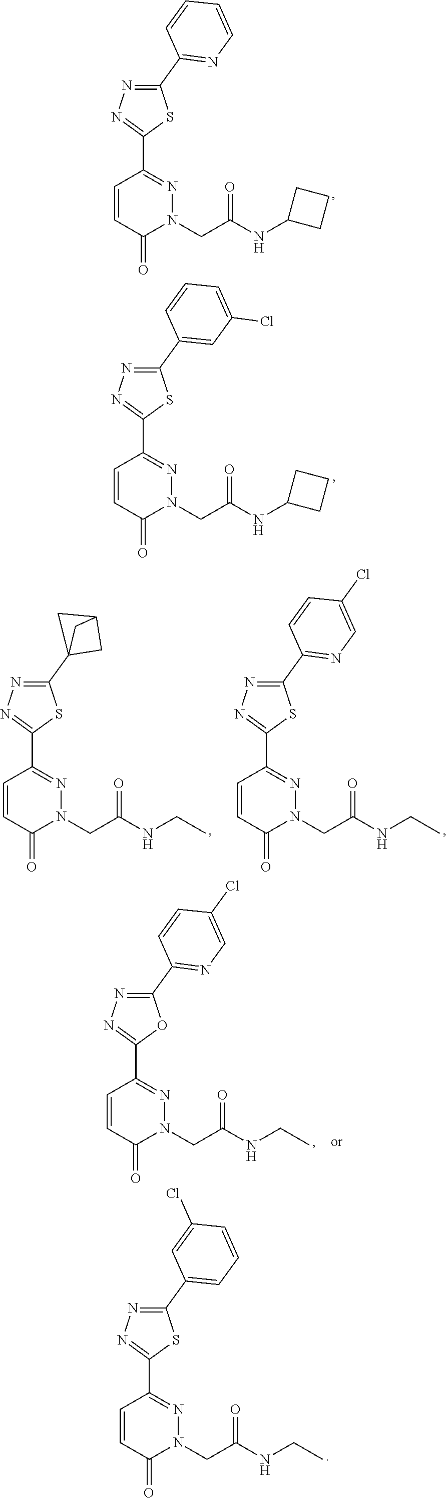

In one aspect, disclosed herein is a compound represented by Formula (V):

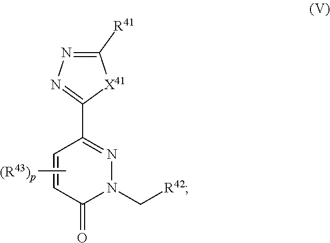

or a salt thereof, wherein:
X⁴¹ is O or S;
R⁴¹ is selected from:
   hydrogen;
   halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂ and —CN;
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
   C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴¹;
R⁴² is selected from:
   C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
   C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴¹;
each R⁴³ is independently selected from halogen, —OR⁴⁶, —SR⁴⁶, —N(R⁴⁶)₂, —NO₂, —CN, and C₁₋₆ alkyl optionally substituted with one or more substituents independently selected from halogen, —OR⁴⁶, —SR⁴⁶, —N(R⁴⁶)₂, —NO₂, and —CN;
each R⁴⁵ is independently selected from:
   halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —NO₂, ═O, ═S, ═N(R⁴⁶), and —CN; and
   C₁₋₃ alkyl, C₂₋₃ alkenyl, C₂₋₃ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN;
each R⁴⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

In one aspect, disclosed herein is a compound represented by Formula (V):

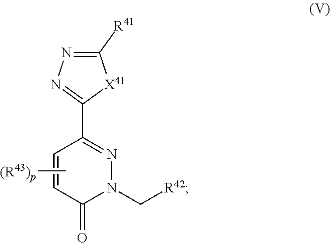

or a salt thereof, wherein:
X⁴¹ is O or S;
R⁴¹ is selected from:
   C₃₋₁₀ carbocycle and 3- to 10-membered heterocycle, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more substituents independently selected from halogen, —CN, —OH, —SH —NO₂, —NH₂, —O—C₁. 6 alkyl, —S—C₁₋₆ alkyl, —N(C₁₋₆ alkyl)₂, —NH(C₁₋₆ alkyl);
R⁴² is selected from:
   C₃₋₁₀ carbocycle and 3- to 10-membered heterocycle, each of which is optionally substituted with one or more substituents independently selected from: halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl); and
p is 0.
   In certain embodiments, for a compound or salt of Formula (V), the compound is selected from

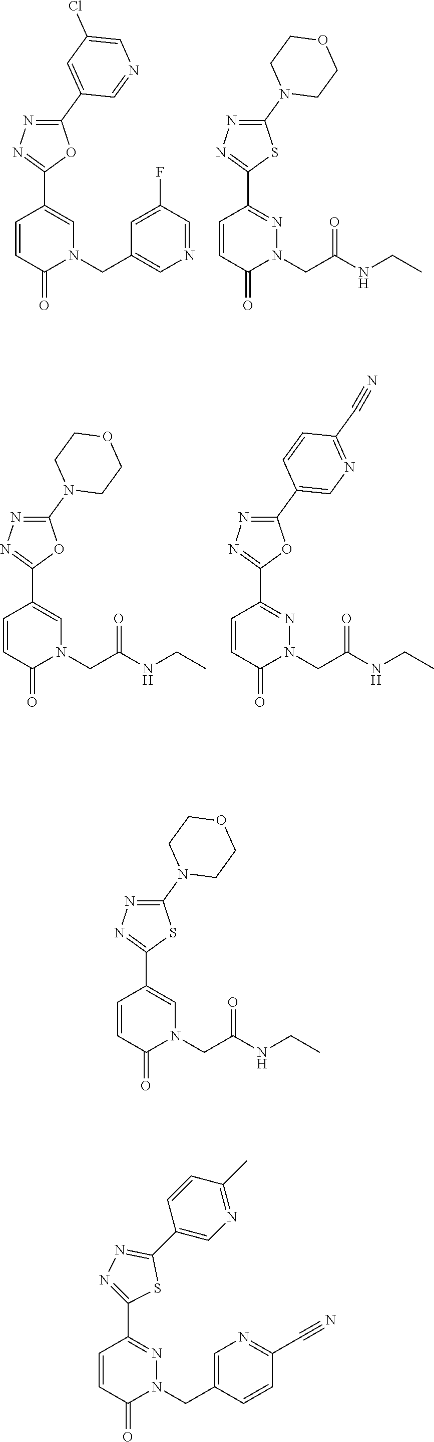

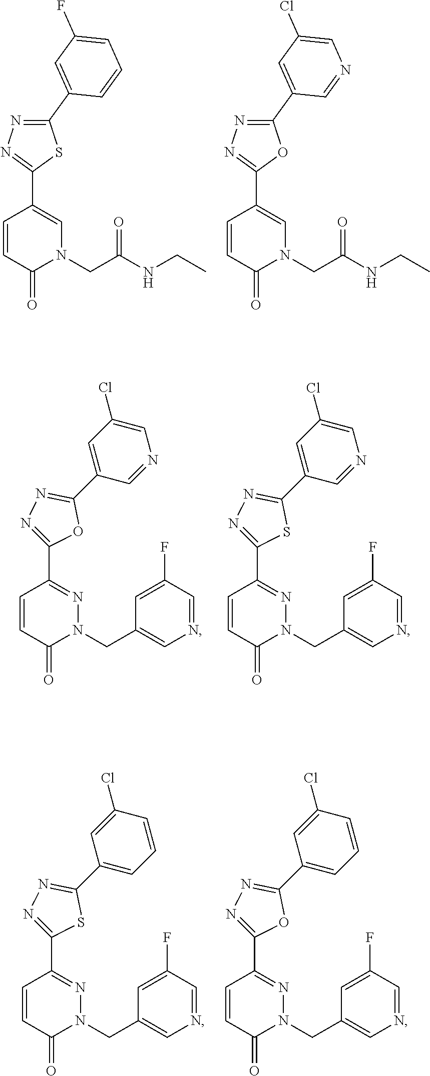

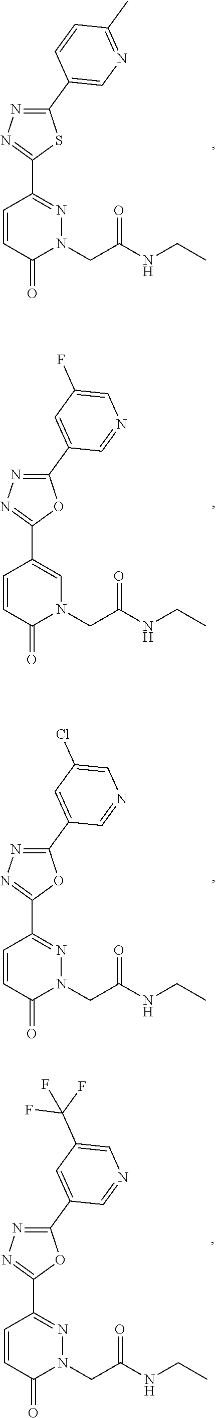 [SMILES: CCNC(=O)Cn1nc(-c2nnc(-c3ccc(C)nc3)s2)ccc1=O ; CCNC(=O)Cn1cc(-c2nnc(-c3cncc(F)c3)o2)ccc1=O ; CCNC(=O)Cn1nc(-c2nnc(-c3cncc(Cl)c3)o2)ccc1=O ; CCNC(=O)Cn1nc(-c2nnc(-c3cncc(C(F)(F)F)c3)o2)ccc1=O]

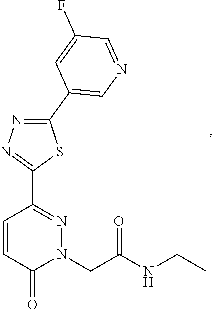 [SMILES: CCNC(=O)Cn1nc(-c2nnc(-c3cncc(F)c3)s2)ccc1=O]

and a salt of any one thereof.

In certain embodiments, for a compound or salt of Formula (V), the compound is selected from

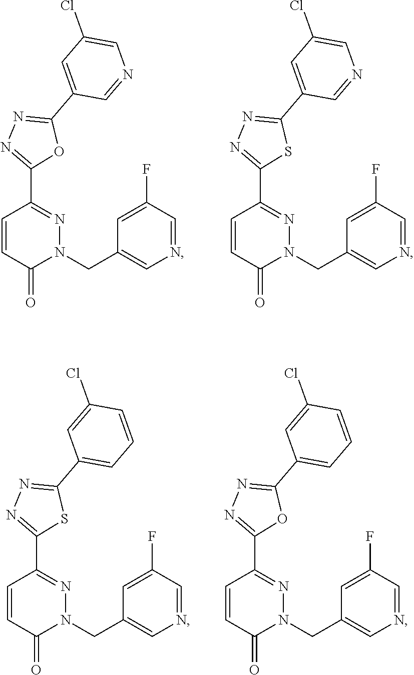

and a salt of any one thereof.

In certain embodiments, for a compound or salt of Formula (V), the compound is selected from

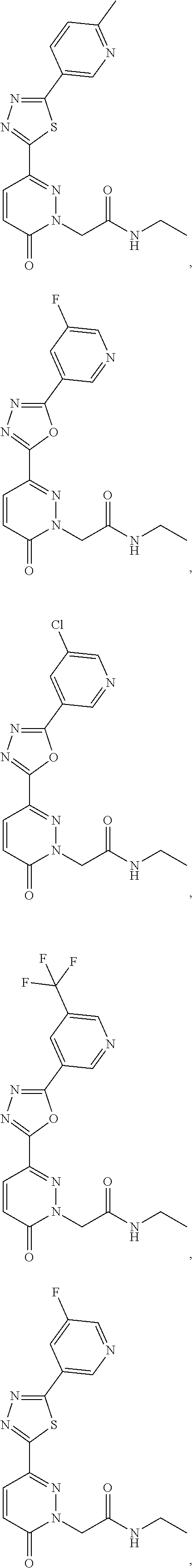 [SMILES: CCNC(=O)Cn1nc(-c2nnc(-c3ccc(C)nc3)s2)ccc1=O ; CCNC(=O)Cn1nc(-c2nnc(-c3cncc(F)c3)o2)ccc1=O ; CCNC(=O)Cn1nc(-c2nnc(-c3cncc(Cl)c3)o2)ccc1=O ; CCNC(=O)Cn1nc(-c2nnc(-c3cncc(C(F)(F)F)c3)o2)ccc1=O ; CCNC(=O)Cn1nc(-c2nnc(-c3cncc(F)c3)s2)ccc1=O]

and a salt of any one thereof.

In certain embodiments, for a compound or salt of Formula (V), the compound is selected from

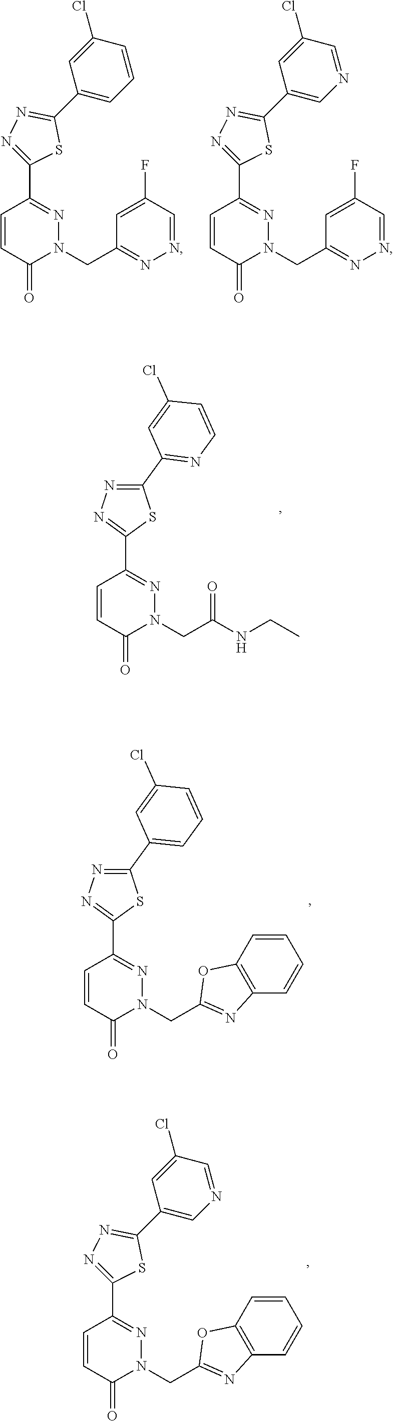

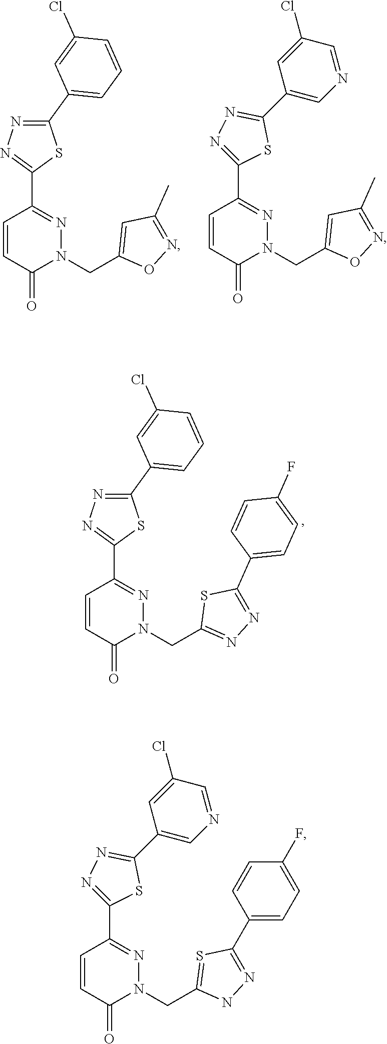

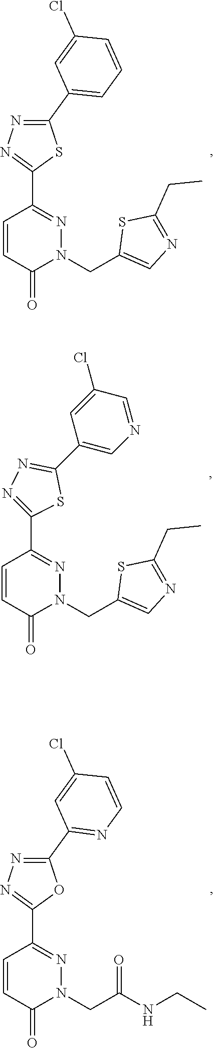 [SMILES: CCc1ncc(Cn2nc(-c3nnc(-c4cccc(Cl)c4)s3)ccc2=O)s1 ; CCc1ncc(Cn2nc(-c3nnc(-c4cncc(Cl)c4)s3)ccc2=O)s1 ; CCNC(=O)Cn1nc(-c2nnc(-c3cc(Cl)ccn3)o2)ccc1=O]

and a salt of any one thereof.

In one aspect, disclosed herein is a compound represented by Formula (VI):

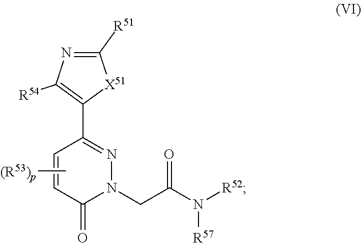

or a salt thereof, wherein:
X⁵¹ is selected from O and S;
R⁵¹ is selected from:
   hydrogen;
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂ and —CN;
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵;
R⁵² is selected from:
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from: halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R⁵³ is independently selected from halogen, —OR⁵⁶, —SR⁵⁶, —N(R¹⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
R⁵¹ is independently selected from hydrogen, halogen, —OR⁵⁶, —SR⁵⁶, —N(R⁵⁶)₂, —NO₂, —CN, C₁₋₆ alkyl, C₃₋₁₀ carbocycle and 3- to 10-membered heterocycle, wherein C₁₋₆ alkyl, C₃₋₁₀ carbocycle and 3- to 10-membered heterocycle are each optionally substituted with one or more substituents independently selected from halogen, —OR⁵⁶, —SR⁵⁶, —N(R⁵⁶)₂, —NO₂, and —CN;
each R⁵⁵ is independently selected from:
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
   C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN;
each R⁵⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2.

In certain embodiments, for a compound or salt of Formula (VI), the compound is not

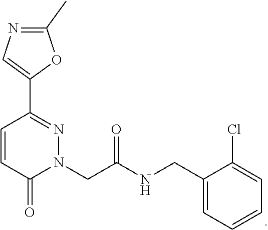 [SMILES: Cc1ncc(-c2ccc(=O)n(CC(=O)NCc3ccccc3Cl)n2)o1]

In certain embodiments, for a compound or salt of Formula (VI), wherein X⁵¹ is O. In certain embodiments, X⁵¹ is S.

In certain embodiments, for a compound or salt of Formula (VI), wherein R⁵¹ is selected from:
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂, and —CN;
C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵.

In certain embodiments, for a compound or salt of Formula (VI), wherein R⁵¹ is selected from:
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —NO₂, and —CN;
C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R¹⁶)₂, —N(R⁵⁶)C(O)R⁵⁶, —C(O)OR⁵⁶, —OC(O)R¹⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵.

In certain embodiments, for a compound or salt of Formula (VI), wherein R⁵¹ is selected from:
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VI), wherein R⁵¹ is selected from C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵. In certain embodiments, for a compound or salt of Formula (VI), wherein R⁵¹ is selected from C₅₋₆ carbocycle and 5- to 6-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R¹⁶)₂, —OC(O)N(R¹⁶)₂, —N(R⁵⁶)C(O)OR⁵⁶, —C(O)OR⁵⁶, —OC(O)R⁵⁶, —S(O)R¹⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵. In certain embodiments, for a compound or salt of Formula (VI), wherein R⁵¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (VI), wherein R⁵¹ is selected from phenyl and pyridinyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁵¹ is selected from phenyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁵¹ is selected from pyridyl, optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, R⁵¹ is selected from phenyl and pyridyl, each of which is optionally substituted with one or more halogen.

In certain embodiments, for a compound or salt of Formula (VI), wherein R⁵¹ is selected from

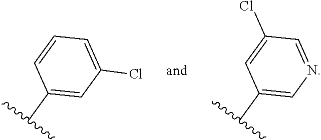 [SMILES: *c1cccc(Cl)c1 ; *c1cncc(Cl)c1]

In certain embodiments, for a compound or salt of Formula (VI), wherein R⁵⁷ is selected from hydrogen and C₁₋₆ alkyl. In certain embodiments, R⁵⁷ is selected from hydrogen. In some embodiments, R⁵⁷ is selected from hydrogen and C₁₋₆ alkyl optionally substituted with one or more substituents independently selected from halogen, —OH, —SH, —NH₂, —NO₂, —CN, —O—C₁₋₆ alkyl, —S—C₁₋₆ alkyl, —N(C₁₋₆ alkyl)₂, and —NH(C₁₋₆ alkyl). In certain embodiments, R⁵⁷ is selected from hydrogen and C₁₋₆ alkyl optionally substituted with one or more substituents independently selected from halogen, —OH, —SH, —NH₂, —NO₂, —CN, and —OMe. In certain embodiments, R⁵⁷ is selected from hydrogen and C₁₋₆ alkyl. In certain embodiments, R⁵⁷ is hydrogen. In some embodiments, R⁵⁷ is selected from methyl, ethyl, n-propyl and isopropyl.

In certain embodiments, for a compound or salt of Formula (VI), wherein R⁵² is selected from:
C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵.

In certain embodiments, for a compound or salt of Formula (VI), wherein R⁵² is selected from:
C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VI), wherein R⁵² is selected from C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl. In certain embodiments, R⁵² is selected from C₁₋₆ alkyl. In certain embodiments, R⁵² is selected from methyl, ethyl, and propyl. In certain embodiments, R⁵² is selected from methyl, ethyl, propyl, butyl, cyclopropyl, and cyclobutyl.

In certain embodiments, for a compound or salt of Formula (VI), wherein each R⁵³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁵³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁵³ is C₁₋₆ alkyl optionally substituted with one or more substituents independently selected from halogen, —CN, —OH, —SH —NO₂, and —NH₂. In some embodiments, each R⁵³ is C₁₋₆ alkyl. In certain embodiments, R⁵³ is selected from methyl, ethyl, propyl, butyl, cyclopropyl, and cyclobutyl.

In certain embodiments, for a compound or salt of Formula (VI), wherein p is 0. In certain embodiments, p is 1. In certain embodiments, p is 2.

In certain embodiments, for a compound or salt of Formula (VI), wherein R⁵⁴ is independently selected from halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN. In certain embodiments, R⁵⁴ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, R⁵⁴ is C₁₋₆ alkyl optionally substituted with one or more substituents independently selected from halogen, —CN, —OH, —SH —NO₂, and —NH₂. In certain embodiments, R⁵⁴ is C₁₋₆ alkyl. In certain embodiments, R⁵⁴ is selected from methyl, ethyl, n-propyl, and isopropyl. In certain embodiments, R⁵⁴ is selected from methyl, ethyl, propyl, butyl, cyclopropyl, and cyclobutyl. In certain embodiments, R⁵⁴ is selected from methyl and ethyl. In certain embodiments, R⁵⁴ is selected from methyl. In certain embodiments, R⁵⁴ is selected from C₁₋₃ haloalkyl. In certain embodiments, R⁵⁴ is selected from methyl, —CHF₂, —CH₂F and —CF₃. In some embodiments, R⁵⁴ is independently selected from halogen, —OR⁵⁶, —SR⁵⁶, —N(R⁵⁶)₂, —NO₂, —CN, C₁₋₆ alkyl, C₃₋₆ carbocycle and 3- to 6-membered heterocycle, wherein C₁₋₆ alkyl, C₃₋₆ carbocycle and 3- to 6-membered heterocycle are each optionally substituted with one or more substituents independently selected from halogen, —OR⁵⁶, —SR⁵⁶, —N(R⁵⁶)₂, —NO₂, and —CN. In some embodiments, R⁵⁴ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, and C₃₋₆ carbocycle, wherein C₁₋₆ alkyl, and C₃₋₆ carbocycle are each optionally substituted with one or more substituents independently selected from halogen, —CN, —OH, —SH —NO₂, and —NH₂. In some embodiments, R⁵⁴ is C₁₋₆ alkyl optionally substituted with one or more substituents independently selected from halogen, —CN, —OH, —SH —NO₂, and —NH₂. In some embodiments, R⁵⁴ is C₃₋₆ carbocycle optionally substituted with one or more substituents independently selected from halogen, —CN, —OH, —SH —NO₂, and —NH₂. In some embodiments, R⁵⁴ is selected from methyl, ethyl, n-propyl, isopropyl, cyclopropyl, cyclobutyl, t-butyl, sec-butyl and n-butyl.

In certain embodiments, for a compound or salt of Formula (VI), wherein each R⁵⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, R⁵⁵ is selected from methyl, ethyl, and propyl.

In certain embodiments, for a compound or salt of Formula (VI), wherein each R⁵⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, for a compound or salt of Formula (VI), wherein each R⁵⁶ is selected from hydrogen, halogen, and C₁₋₃ alkyl optionally substituted with one or more substituents independently selected from halogen, —CN, —OH, —SH —NO₂, and —NH₂. In certain embodiments, R⁵⁶ is selected from methyl, ethyl, propyl, butyl, cyclopropyl, and cyclobutyl. In certain embodiments, R⁵⁶ is hydrogen.

In one aspect, disclosed herein is a compound represented by Formula (VI):

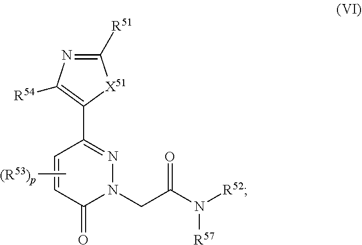

or a salt thereof, wherein:
X⁵¹ is selected from O and S;
R⁵¹ is selected from:
   hydrogen;
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂ and —CN;
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R¹⁶)₂, —N(R⁵⁶)C(O)R⁵⁶, —C(O)OR⁵⁶, —OC(O)R¹⁶, —N(R⁵⁶)C(O)N(R⁵⁶)₂, —OC(O)N(R¹⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵;
R⁵² is selected from:
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from: halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵;
each R⁵³ is independently selected from halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
R⁵⁴ is independently selected from hydrogen, halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
each R⁵⁵ is independently selected from:
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
   C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN;
each R⁵⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2,
   wherein the compound is not

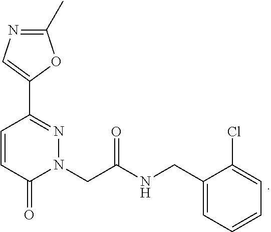 [SMILES: Cc1ncc(-c2ccc(=O)n(CC(=O)NCc3ccccc3Cl)n2)o1]

In one aspect, disclosed herein is a compound represented by Formula (VI):

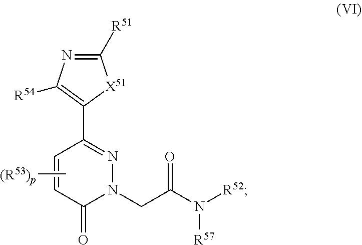

or a salt thereof, wherein:
X⁵¹ is selected from O and S;
R⁵¹ is selected from:
   hydrogen;
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂ and —CN;
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵;
R⁵² is selected from:
   C₂₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from: halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵;
each R⁵³ is independently selected from halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
R⁵¹ is independently selected from hydrogen, halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
each R⁵⁵ is independently selected from:
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
   C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN;
each R⁵⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2.

In one aspect, disclosed herein is a compound represented by Formula (VI):

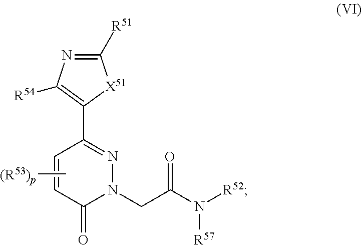

or a salt thereof, wherein:
X⁵¹ is selected from O and S;
R⁵¹ is selected from:
   C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵⁵;
R⁵² is selected from:
   C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
   C₃₋₁₀ carbocycle and 3- to 10-membered heterocycle, each of which is optionally substituted with one or more substituents independently selected from:
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵⁵;
each R⁵³ is independently selected from halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
R⁵⁴ is independently selected from hydrogen, halogen, —OR⁵⁶, —SR⁵⁶, —N(R⁵⁶)₂, —NO₂, —CN, and C₁₋₆ alkyl optionally substituted with one or more substituents independently selected from halogen, —OR⁵⁶, —SR⁵⁶, —N(R⁵⁶)₂, —NO₂, and —CN;
each R⁵⁵ is independently selected from:
   halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
   C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN;
each R⁵⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2.

In one aspect, disclosed herein is a compound represented by Formula (VI):

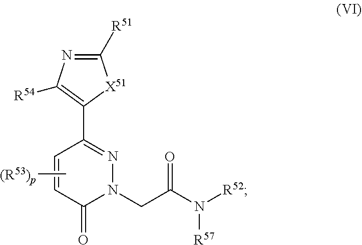

or a salt thereof, wherein:
X⁵¹ is selected from O and S;
R⁵¹ is selected from:
   C₃₋₁₀ carbocycle and 3- to 10-membered heterocycle, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more substituents independently selected from halogen, —CN, —OH, —SH —NO₂, —NH₂, —O—C₁. 6 alkyl, —S—C₁₋₆ alkyl, —N(C₁₋₆ alkyl)₂, and —NH(C₁₋₆ alkyl);
R⁵² is selected from:
   C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₃₋₁₀ carbocycle and 3- to 10-membered heterocycle, wherein the C₃₋₁₀ carbocycle and 3- to 10-membered heterocycle are each optionally substituted with one or more substituents independently selected from halogen, —CN, —OH, —SH —NO₂, —NH₂, —O—C₁. 6 alkyl, —S—C₁₋₆ alkyl, —N(C₁₋₆ alkyl)₂, and —NH(C₁₋₆ alkyl); and
R⁵⁴ is independently selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), and C₁₋₆ alkyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, and —NH(C₁₋₆ alkyl);
R⁵⁷ is hydrogen; and
p is 0.

In certain embodiments, for a compound or salt of Formula (VI), wherein the compound is selected from

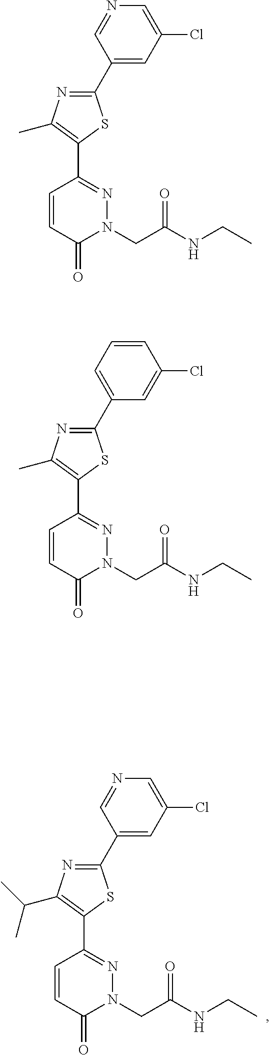

and a salt of any one thereof.

In certain embodiments, for a compound or salt of Formula (VI), wherein the compound is selected from

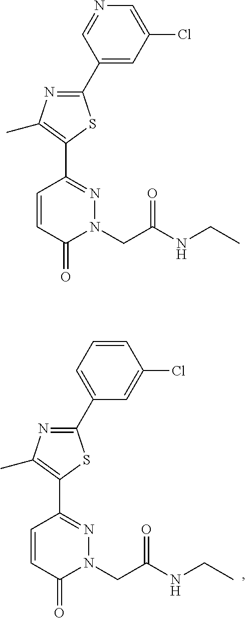

and a salt of any one thereof.

In certain embodiments, for a compound or salt of Formula (VI), wherein the compound is selected from

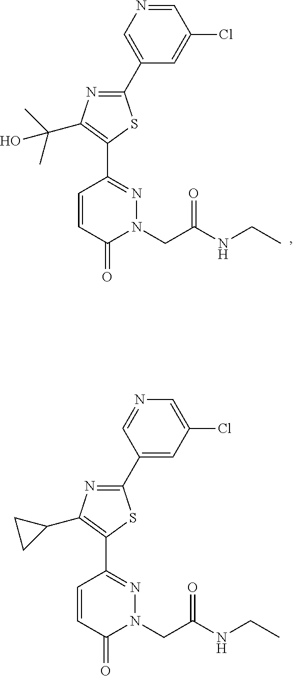 [SMILES: CCNC(=O)Cn1nc(-c2sc(-c3cncc(Cl)c3)nc2C(C)(C)O)ccc1=O ; CCNC(=O)Cn1nc(-c2sc(-c3cncc(Cl)c3)nc2C2CC2)ccc1=O]

and a salt of any one thereof.

In one aspect, disclosed herein is compound represented by Formula (VII):

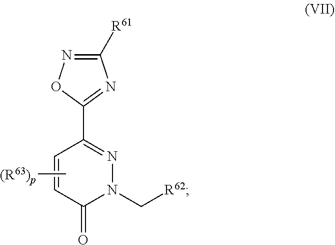

or a salt thereof, wherein:
R⁶¹ is selected from:
   hydrogen;
   halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, and —CN;
   C₁₋₆ alkyl, C₂₋₆ alkenyl, C₂₋₆ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, ═O, ═S, ═N(R⁶⁶), —CN, C₃₋₁₀ carbocycle and 3- to 10-membered heterocycle, wherein the C₃₋₁₀ carbocycle and 3- to 10-membered heterocycle are each optionally substituted with one or more R⁶⁵; and
   C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵.
R⁶² is selected from:
   5-membered heterocycle optionally substituted with one or more substituents independently selected from: halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵.
each R⁶³ is independently selected from halogen, —OR⁶⁶, —SR⁶⁶, —N(R⁶⁶)₂, —NO₂, —CN, and C₁₋₆ alkyl optionally substituted with one or more substituents independently selected from halogen, —OR⁶⁶, —SR⁶⁶, —N(R⁶⁶)₂, —NO₂, and —CN;
each R⁶⁵ is independently selected from:
   halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN; and
   C₁₋₃ alkyl, C₂₋₃ alkenyl, C₂₋₃ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN;
each R⁶⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

In certain embodiments, for a compound or salt of Formula (VII), wherein R⁶¹ is selected from:
halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, and —CN;
C₁₋₆ alkyl, C₂₋₆ alkenyl, C₂₋₆ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, ═O, ═S, ═N(R⁶⁶), —CN, C₃₋₁₀ carbocycle and 3- to 10-membered heterocycle, wherein the C₃₋₁₀ carbocycle and 3- to 10-membered heterocycle are each optionally substituted with one or more R⁶⁵; and
C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵.

In certain embodiments, for a compound or salt of Formula (VII), wherein R⁶¹ is selected from:
halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —NO₂, and —CN;
C₂₋₆ alkyl, C₂₋₆ alkenyl, C₂₋₆ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁶⁵; and
C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵.

In certain embodiments, for a compound or salt of Formula (VII), wherein R⁶¹ is selected from:
halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VII), wherein R⁶¹ is selected from C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵. In certain embodiments, R⁶¹ is selected from C₅₋₆ carbocycle and 5- to 6-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵. In certain embodiments, R⁶¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (VII), wherein R⁶¹ is selected from: phenyl and pyridinyl each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VII), wherein R⁶¹ is selected from

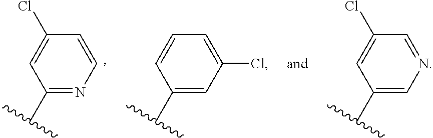 [SMILES: *c1cc(Cl)ccn1 ; "," ; *c1cccc(Cl)c1 ; "and" ; *c1cncc(Cl)c1]

In certain embodiments, for a compound or salt of Formula (VII), wherein R⁶² is selected from 5-membered heterocycle optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵. In certain embodiments, R⁶² is selected from 5-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵.

In certain embodiments, R⁶² is selected from 5-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VII), wherein R⁶² is selected from

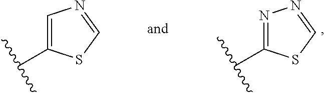 [SMILES: *c1cncs1 ; *c1nncs1]

each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VII), wherein R⁶² is selected from

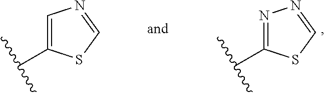 [SMILES: *c1cncs1 ; *c1nncs1]

each of which is optionally substituted with one or more substituents independently selected from fluoro, chloro, bromo, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VII), wherein R⁶² is selected from

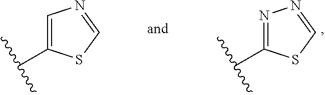 [SMILES: *c1cncs1 ; *c1nncs1]

each of which is optionally substituted with one or more substituents independently selected from fluoro, chloro, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VII), wherein R⁶² is selected from

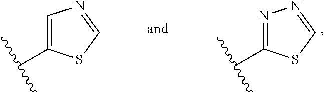 [SMILES: *c1cncs1 ; *c1nncs1]

each of which is optionally substituted with one or more substituents independently selected from fluoro, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VII), wherein R⁶² is selected from

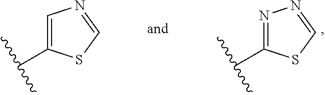 [SMILES: *c1cncs1 ; *c1nncs1]

each of which is optionally substituted with one or more substituents independently selected from fluoro, chloro, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from fluoro, chloro, bromo, —CN, —OH, —SH —NO₂, —NH₂, and C₁₋₆ alkyl.

In certain embodiments, for a compound or salt of Formula (VII), wherein R⁶² is selected from

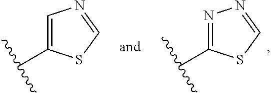 [SMILES: *c1cncs1 ; *c1nncs1]

each of which is optionally substituted with one or more substituents independently selected from fluoro, chloro, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from fluoro, chloro, —CN, —OH, —SH —NO₂, —NH₂, and C₁₋₆ alkyl.

In certain embodiments, for a compound or salt of Formula (VII), wherein R⁶² is selected from

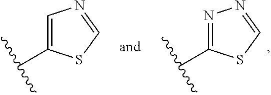 [SMILES: *c1cncs1 ; *c1nncs1]

each of which is optionally substituted with one or more substituents independently selected from fluoro, chloro, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from fluoro, —CN, —OH, —SH —NO₂, —NH₂, and C₁₋₆ alkyl.

In certain embodiments, for a compound or salt of Formula (VII), wherein R⁶² is selected from

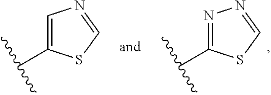 [SMILES: *c1cncs1 ; *c1nncs1]

each of which is optionally substituted with one or more substituents independently selected from pyridyl optionally substituted with one or more halogen. In certain embodiments, for a compound or salt of Formula (VII), wherein R⁶² is selected from

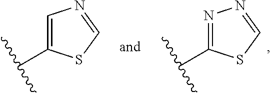 [SMILES: *c1cncs1 ; *c1nncs1]

each of which is optionally substituted with one or more substituents independently selected from pyridyl optionally substituted with one or more fluoro.

In certain embodiments, for a compound or salt of Formula (VII), wherein R⁶² is selected from

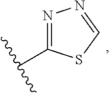 [SMILES: *c1nncs1]

each of which is optionally substituted with one or more substituents independently selected from pyridyl optionally substituted with one or more halogen.

In certain embodiments, for a compound or salt of Formula (VII), wherein R⁶² is selected from

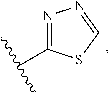 [SMILES: *c1nncs1]

each of which is optionally substituted with one or more substituents independently selected from pyridyl optionally substituted with one or more fluoro.

In certain embodiments, for a compound or salt of Formula (VII), wherein R⁶² is selected from C(O)N(R¹⁶⁷)(R¹⁶⁸). In certain embodiments, for a compound or salt of Formula (VII), R⁶² is selected from C(O)N(R⁶⁶)₂. In certain embodiments, for a compound or salt of Formula (VII), R⁶² is selected from C(O)N(H)(R⁶⁶). In certain embodiments, for a compound or salt of Formula (VII), R⁶² is selected from C(O)N(H)(R⁶⁶), and R⁶⁶ is selected from C₁₋₆ alkyl optionally substituted with one or more substituents selected from halogen, CN, NO₂, OH, SH, and NH₂. In certain embodiments, for a compound or salt of Formula (VII), R⁶² is selected from C(O)N(H)(R⁶⁶), and R⁶⁶ is selected from C₁₋₆ alkyl. In certain embodiments, for a compound or salt of Formula (VII), R⁶² is selected from C(O)N(H)(R⁶⁶), and R⁶⁶ is selected from methyl, ethyl, n-propyl, isopropyl, n-butyl, s-butyl, i-butyl, and t-butyl. In certain embodiments, for a compound or salt of Formula (VII), R⁶² is selected from C(O)N(H)(R⁶⁶), and R⁶⁶ is selected from ethyl.

In certain embodiments, for a compound or salt of Formula (VII), wherein R⁶² is selected from

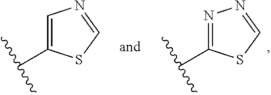 [SMILES: *c1cncs1 ; *c1nncs1]

each of which is optionally substituted with one or more substituents independently selected from C₁₋₆ alkyl, phenyl, and pyridyl wherein the C₁₋₆ alkyl, phenyl, and pyridyl are each optionally substituted with one or more substituents independently selected from halogen, —CN, —OH, —SH —NO₂, —NH₂, and C₁₋₆ alkyl.

In certain embodiments, for a compound or salt of Formula (VII), wherein each R⁶³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁶³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁶³ is C₁₋₆ alkyl optionally substituted with one or more substituents independently selected from halogen, —CN, —OH, —SH —NO₂, and —NH₂.

In certain embodiments, for a compound or salt of Formula (VII), wherein p is 0. In certain embodiments, p is 1. In certain embodiments, p is 2.

In certain embodiments, for a compound or salt of Formula (VII), wherein each R⁶⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂.

In certain embodiments, for a compound or salt of Formula (VII), wherein each R⁶⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, each R⁶⁶ is selected from hydrogen, halogen, and C₁₋₃ alkyl optionally substituted with one or more substituents independently selected from halogen, —CN, —OH, —SH —NO₂, and —NH₂. In certain embodiments, R⁶⁶ is hydrogen.

In certain embodiments, for a compound or salt of Formula (VII), wherein the compound is selected from

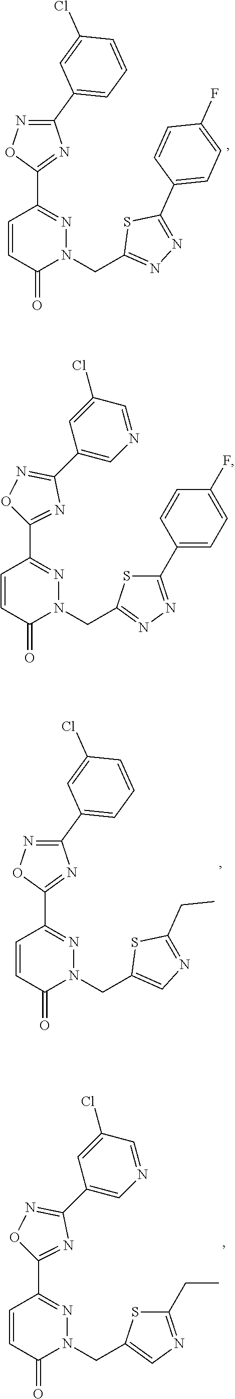 [SMILES: O=c1ccc(-c2nc(-c3cccc(Cl)c3)no2)nn1Cc1nnc(-c2ccc(F)cc2)s1 ; O=c1ccc(-c2nc(-c3cncc(Cl)c3)no2)nn1Cc1nnc(-c2ccc(F)cc2)s1 ; CCc1ncc(Cn2nc(-c3nc(-c4cccc(Cl)c4)no3)ccc2=O)s1 ; CCc1ncc(Cn2nc(-c3nc(-c4cncc(Cl)c4)no3)ccc2=O)s1]

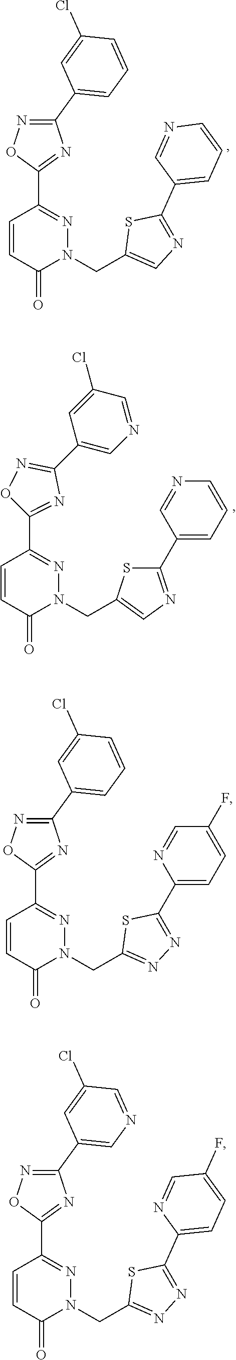 [SMILES: O=c1ccc(-c2nc(-c3cccc(Cl)c3)no2)nn1Cc1cnc(-c2cccnc2)s1 ; O=c1ccc(-c2nc(-c3cncc(Cl)c3)no2)nn1Cc1cnc(-c2cccnc2)s1 ; O=c1ccc(-c2nc(-c3cccc(Cl)c3)no2)nn1Cc1nnc(-c2ccc(F)cn2)s1 ; O=c1ccc(-c2nc(-c3cncc(Cl)c3)no2)nn1Cc1nnc(-c2ccc(F)cn2)s1]

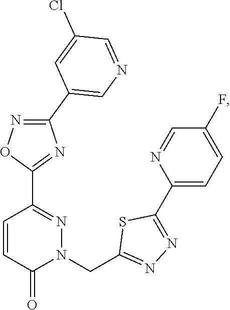 [SMILES: O=c1ccc(-c2nc(-c3cncc(Cl)c3)no2)nn1Cc1nnc(-c2ccc(F)cn2)s1]

and a salt of any one thereof.

In some embodiments, the compound or salt is selected from

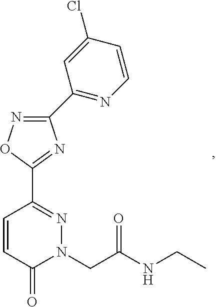 [SMILES: CCNC(=O)Cn1nc(-c2nc(-c3cc(Cl)ccn3)no2)ccc1=O]

or a salt thereof.

In one aspect, disclosed herein is compound represented by Formula (VIII):

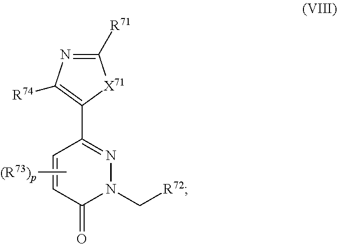

or a salt thereof, wherein:
X⁷¹ is selected from S and O;
R⁷¹ is selected from:
   hydrogen;
   halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, and —CN;
   C₁₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, ═O, ═S, ═N(R⁷⁶), —CN, C₃₋₁₀ carbocycle and 3- to 10-membered heterocycle, wherein the C₃₋₁₀ carbocycle and 3- to 10-membered heterocycle are each optionally substituted with one or more R⁷⁵; and
   C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷¹;
R⁷² is selected from:
   3- to 10-membered heterocycle optionally substituted with one or more substituents independently selected from: halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷⁵; and
   —C(O)NR⁷⁷R⁷⁸;
each R⁷³ is independently selected from halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
R⁷⁴ is independently selected from hydrogen, halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
each R⁷⁵ is independently selected from:
   halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN; and
   C₁₋₃ alkyl, C₂₋₃ alkenyl, C₂₋₃ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN;
each R⁷⁶ is independently selected from:
   hydrogen;
   C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁷⁷ is selected from hydrogen and C₁₋₆ alkyl optionally substituted with one or more substituents independently selected from halogen, —OR⁷⁶, —SR⁷⁶, —N(R⁷⁶)₂, —NO₂, and —CN;
R⁷⁸ is selected from:
   C₁₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₃₋₁₀ carbocycle and 3- to 10-membered heterocycle, wherein the C₃₋₁₀ carbocycle and 3- to 10-membered heterocycle are each optionally substituted with one or more R⁷⁵;
   C₃₋₁₀ carbocycle and 3- to 10-membered heterocycle, each of which is optionally substituted with one or more substituents independently selected from: halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁴⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷⁵; and
p is 0, 1, or 2.

In certain embodiments, for a compound or salt of Formula (VIII), X⁷¹ is O. In some embodiments, X⁷¹ is S.

In certain embodiments, for a compound or salt of Formula (VIII), R⁷¹ is selected from:
halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, and —CN;
C₁₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, ═O, ═S, ═N(R⁷⁶), —CN, C₃₋₁₀ carbocycle and 3- to 10-membered heterocycle, wherein the C₃₋₁₀ carbocycle and 3- to 10-membered heterocycle are each optionally substituted with one or more R⁷⁵; and
C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷⁵.

In certain embodiments, for a compound or salt of Formula (VIII), R⁷¹ is selected from:
halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —NO₂, and —CN;
C₂₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁷⁵; and
C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —NO₂, and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷⁵.

In certain embodiments, for a compound or salt of Formula (VIII), R⁷¹ is selected from:
halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VIII), R⁷¹ is selected from C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷⁵. In certain embodiments, for a compound or salt of Formula (VIII), R⁷¹ is selected from C₅₋₆ carbocycle and 5- to 6-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, —N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷⁵. In certain embodiments, for a compound or salt of Formula (VIII), R⁷¹ is selected from C₅₋₆ carbocycle and 5- to 6-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl. In certain embodiments, for a compound or salt of Formula (VIII), R⁷¹ is selected from phenyl and pyridinyl each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

In certain embodiments, for a compound or salt of Formula (VIII), R⁷¹ is selected from

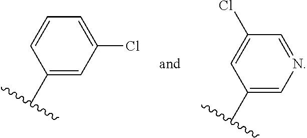 [SMILES: *c1cccc(Cl)c1 ; *c1cncc(Cl)c1]

In certain embodiments, for a compound or salt of Formula (VIII), R⁷² is selected from 5- to 6-membered heterocycle optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷⁵. In certain embodiments, for a compound or salt of Formula (VIII), R⁷² is selected from 5-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷⁵. In certain embodiments, for a compound or salt of Formula (VIII), R⁷² is selected from 5-membered heterocycle optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VIII), R⁷² is selected from

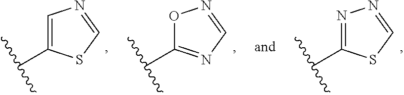 [SMILES: *c1cncs1 ; *c1ncno1 ; *c1nncs1]

each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₆₋₁₀ carbocycle, and 5- to 6-membered heterocycle, wherein C₁₋₆ alkyl, C₆₋₁₀ carbocycle, and 5- to 6-membered heterocycle are each optionally substituted with one or more substituents independently selected from halogen, —CN, —OH, —SH —NO₂, —NH₂, and C₁₋₆ alkyl.

In certain embodiments, for a compound or salt of Formula (VIII), R⁷² is selected from

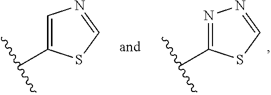 [SMILES: *c1cncs1 ; *c1nncs1]

each of which is optionally substituted with one or more substituents independently selected from C₁₋₆ alkyl, phenyl, and pyridyl wherein the C₁₋₆ alkyl, phenyl, and pyridyl are each optionally substituted with one or more substituents independently selected from halogen, —CN, —OH, —SH —NO₂, —NH₂, and C₁₋₆ alkyl.

In certain embodiments, for a compound or salt of Formula (VIII), R⁷² is selected from

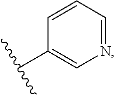 [SMILES: *c1cccnc1]

optionally substituted with one or more substituents independently selected from C₁₋₆ alkyl, phenyl, and pyridyl wherein the C₁₋₆ alkyl, phenyl, and pyridyl are each optionally substituted with one or more substituents independently selected from halogen, —CN, —OH, —SH —NO₂, —NH₂, and C₁₋₆ alkyl. In certain embodiments, for a compound or salt of Formula (VIII), R⁷² is selected from

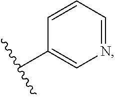 [SMILES: *c1cccnc1]

optionally substituted with one or more substituents independently selected from halogen, CN, NO₂, OH, NH₂, and C₁₋₆ alkyl. In certain embodiments, for a compound or salt of Formula (VIII), R⁷² is selected from

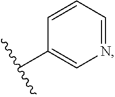 [SMILES: *c1cccnc1]

optionally substituted with one or more substituents independently selected from fluoro, chloro, bromo, CN, NO₂, OH, NH₂, and C₁₋₆ alkyl. In certain embodiments, for a compound or salt of Formula (VIII), R⁷² is selected from

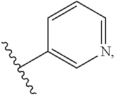 [SMILES: *c1cccnc1]

optionally substituted with one or more substituents independently selected from fluoro, chloro, CN, NO₂, OH, NH₂, and C₁₋₆ alkyl. In certain embodiments, for a compound or salt of Formula (VIII), R⁷² is selected from

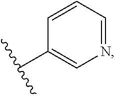 [SMILES: *c1cccnc1]

optionally substituted with one or more substituents independently selected from fluoro, chloro, CN, NO₂, OH, and NH₂. In certain embodiments, for a compound or salt of Formula (VIII), R⁷² is selected from

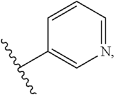 [SMILES: *c1cccnc1]

optionally substituted with one or more substituents independently selected from fluoro and chloro. In certain embodiments, for a compound or salt of Formula (VIII), R⁷² is selected from

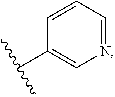 [SMILES: *c1cccnc1]

optionally substituted with one or more substituents independently selected from fluoro. In some embodiments, R⁷² is selected from

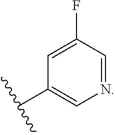 [SMILES: *c1cncc(F)c1]

In certain embodiments, for a compound or salt of Formula (VIII), each R⁷³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁷³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, each R⁷³ is C₁₋₆ alkyl optionally substituted with one or more substituents independently selected from halogen, —CN, —OH, —SH —NO₂, and —NH₂.

In certain embodiments, for a compound or salt of Formula (VIII), p is 0. In some embodiments, p is 1. In some embodiments, p is 2.

In certain embodiments, for a compound or salt of Formula (VIII), R⁷⁴ is independently selected from halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN.

In certain embodiments, for a compound or salt of Formula (VIII), R⁷⁴ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂. In certain embodiments, R⁷⁴ is C₁₋₆ alkyl optionally substituted with one or more substituents independently selected from halogen, —CN, —OH, —SH —NO₂, and —NH₂. In certain embodiments, R⁷⁴ is C₁₋₆ alkyl. In certain embodiments, R⁷⁴ is selected from methyl, ethyl, n-propyl, and isopropyl.

In certain embodiments, for a compound or salt of Formula (VIII), each R⁷⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂.

In certain embodiments, for a compound or salt of Formula (VIII), each R⁷⁶ is selected from hydrogen, halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂. In certain embodiments, each R⁷⁶ is selected from hydrogen, halogen, and C₁₋₃ alkyl optionally substituted with one or more substituents independently selected from halogen, —CN, —OH, —SH —NO₂, and —NH₂. In some embodiments, R⁷⁶ is hydrogen.

In certain embodiments, for a compound or salt of Formula (VIII), wherein the compound is selected from

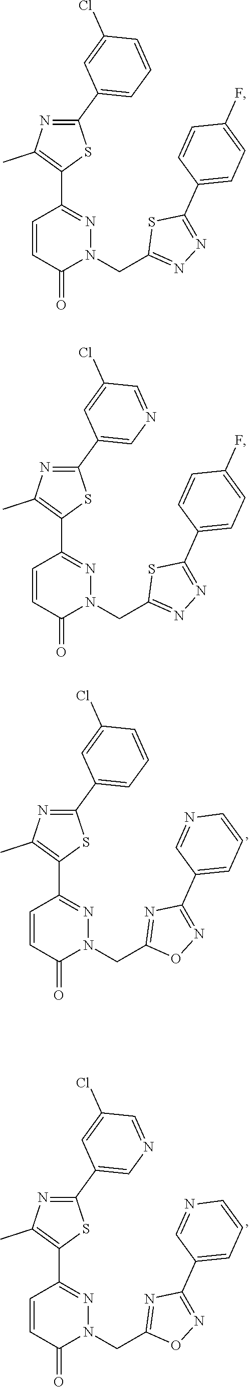 [SMILES: Cc1nc(-c2cccc(Cl)c2)sc1-c1ccc(=O)n(Cc2nnc(-c3ccc(F)cc3)s2)n1 ; Cc1nc(-c2cncc(Cl)c2)sc1-c1ccc(=O)n(Cc2nnc(-c3ccc(F)cc3)s2)n1 ; Cc1nc(-c2cccc(Cl)c2)sc1-c1ccc(=O)n(Cc2nc(-c3cccnc3)no2)n1 ; Cc1nc(-c2cncc(Cl)c2)sc1-c1ccc(=O)n(Cc2nc(-c3cccnc3)no2)n1]

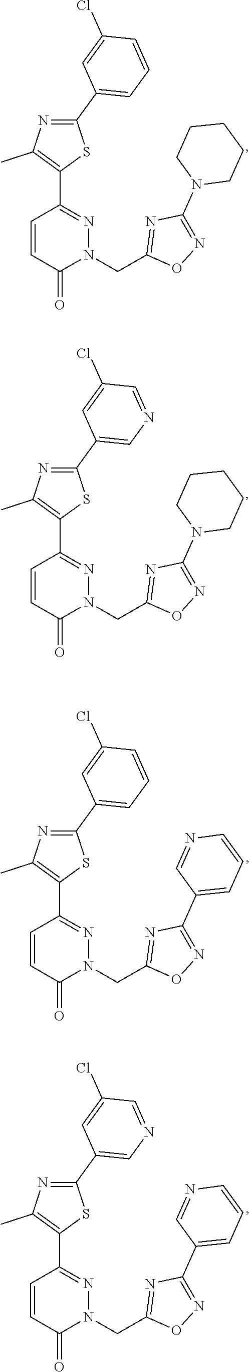 [SMILES: Cc1nc(-c2cccc(Cl)c2)sc1-c1ccc(=O)n(Cc2nc(N3CCCCC3)no2)n1 ; Cc1nc(-c2cncc(Cl)c2)sc1-c1ccc(=O)n(Cc2nc(N3CCCCC3)no2)n1 ; Cc1nc(-c2cccc(Cl)c2)sc1-c1ccc(=O)n(Cc2nc(-c3cccnc3)no2)n1 ; Cc1nc(-c2cncc(Cl)c2)sc1-c1ccc(=O)n(Cc2nc(-c3cccnc3)no2)n1]

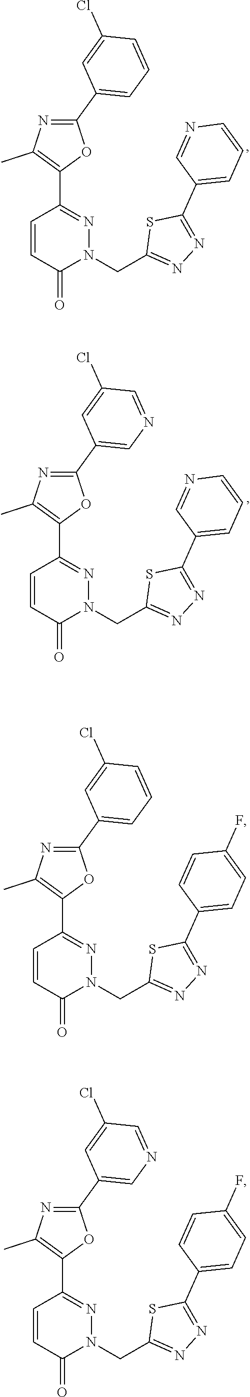 [SMILES: Cc1nc(-c2cccc(Cl)c2)oc1-c1ccc(=O)n(Cc2nnc(-c3cccnc3)s2)n1 ; Cc1nc(-c2cncc(Cl)c2)oc1-c1ccc(=O)n(Cc2nnc(-c3cccnc3)s2)n1 ; Cc1nc(-c2cccc(Cl)c2)oc1-c1ccc(=O)n(Cc2nnc(-c3ccc(F)cc3)s2)n1 ; Cc1nc(-c2cncc(Cl)c2)oc1-c1ccc(=O)n(Cc2nnc(-c3ccc(F)cc3)s2)n1]

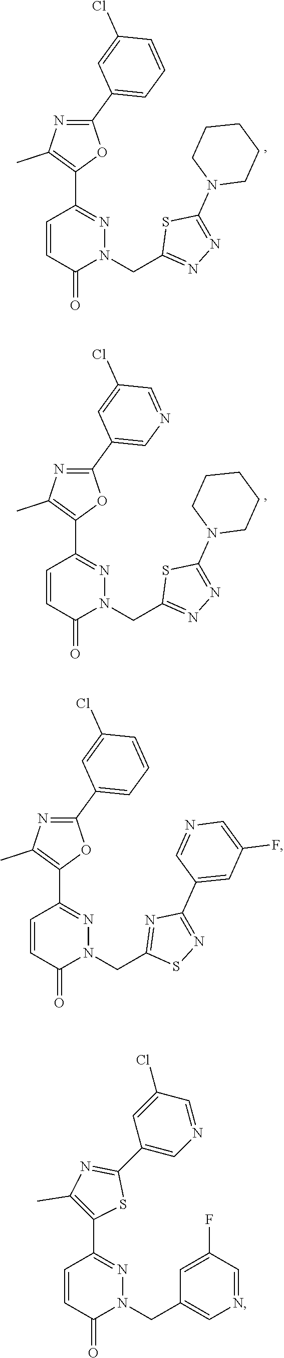 [SMILES: Cc1nc(-c2cccc(Cl)c2)oc1-c1ccc(=O)n(Cc2nnc(N3CCCCC3)s2)n1 ; Cc1nc(-c2cncc(Cl)c2)oc1-c1ccc(=O)n(Cc2nnc(N3CCCCC3)s2)n1 ; Cc1nc(-c2cccc(Cl)c2)oc1-c1ccc(=O)n(Cc2nc(-c3cncc(F)c3)ns2)n1 ; Cc1nc(-c2cncc(Cl)c2)sc1-c1ccc(=O)n(Cc2cncc(F)c2)n1]

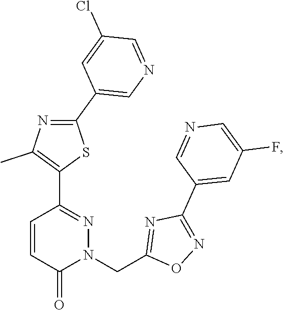 [SMILES: Cc1nc(-c2cncc(Cl)c2)sc1-c1ccc(=O)n(Cc2nc(-c3cncc(F)c3)no2)n1]

and a salt of any one thereof.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

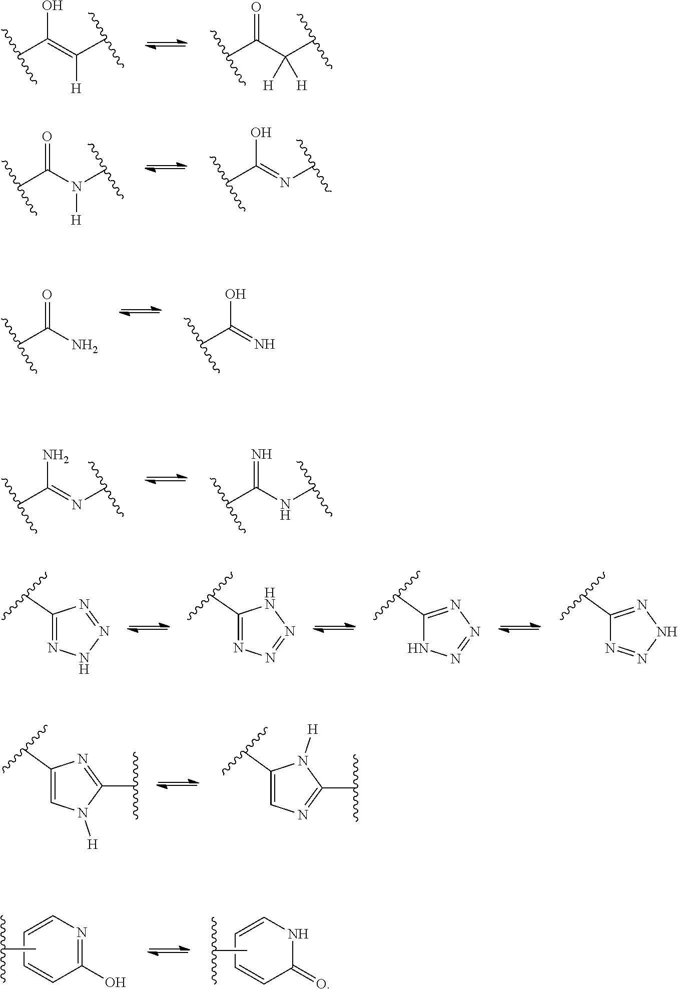

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ¹⁴C, ¹⁵C, ¹²N ¹³N ¹⁵N, ¹⁶N, ¹⁶O, ¹⁷O, ¹⁸F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Therapeutic Applications

Methods of administration of a compound or salt of Formula Formulas (Ia), (Ib), (II), (III), (IV), (V), (VI), (VI), (VII), or (VIII) discussed herein may be used for inhibiting muscle myosin II. In some embodiments, the compounds and salts thereof may be used to treat activity-induced muscle damage. In some embodiments, the compounds may be used to treat neuromuscular conditions and movement disorders (such as spasticity).

Methods of administration of a compound or salt of Formula Formulas (Ia), (Ib), (II), (III), (IV), (V), (VI), (VI), (VII), or (VIII) discussed herein may be used for the treatment of neuromuscular conditions and movement disorders. Examples of neuromuscular conditions include but are not limited to Duchenne Muscular Dystrophy, Becker muscular dystrophy, myotonic dystrophy 1, myotonic dystrophy 2, facioscapulohumeral muscular dystrophy, oculopharyngeal muscular dystrophy, limb girdle muscular dystrophies, tendinitis and carpal tunnel syndrome. Examples of movement disorders include but are not limited to muscle spasticity disorders, spasticity associated with multiple sclerosis, Parkinson's disease, Alzheimer's disease, or cerebral palsy, or injury or a traumatic event such as stroke, traumatic brain injury, spinal cord injury, hypoxia, meningitis, encephalitis, phenylketonuria, or amyotrophic lateral sclerosis. Also included are other conditions that may respond to the inhibition of skeletal myosin II, skeletal troponin C, skeletal troponin I, skeletal tropomyosin, skeletal troponin T, skeletal regulatory light chains, skeletal myosin binding protein C or skeletal actin. In some embodiments, neuromuscular conditions and movement disorders are selected from muscular dystrophies and myopathies. In some embodiments, muscular dystrophies are diseases that cause progressive weakness and loss of muscle mass where abnormal genes (mutations) interfere with the production of proteins needed to form healthy muscle. In some embodiments, muscular dystrophies are selected from Becker muscular dystrophy (BMD), Congenital muscular dystrophies (CMD), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), and Oculopharyngeal muscular dystrophy (OPMD). In some embodiments, Congenital muscular dystrophies (CMD) is selected from Bethlem CMD, Fukuyama CMD, Muscle-eye-brain diseases (MEBs), Rigid spine syndromes, Ullrich CMD, and Walker-Warburg syndromes (WWS). In some embodiments, myopathies are diseases of muscle that are not caused by nerve disorders. Myopathies cause the muscles to become weak or shrunken (atrophied). In some embodiments, myopathies are selected from congenital myopathies, distal myopathies, endocrine myopathies, inflammatory myopathies, metabolic myopathies, myofibrillar myopathies (MFM), scapuloperoneal myopathy, and cardiomyopathies. In some embodiments, congenital myopathies are selected from cap myopathies, centronuclear myopathies, congenital myopathies with fiber type disproportion, core myopathies, central core disease, multiminicore myopathies, myosin storage myopathies, myotubular myopathy, and nemaline myopathies. In some embodiments, distal myopathies are selected from, gne myopathy/Nonaka myopathy/hereditary inclusion-body myopathy (HIBM), laing distal myopathy, Markesbery-Griggs late-onset distal myopathy, Miyoshi myopathy, Udd myopathy/tibial muscular dystrophy, VCP myopathy/IBMPFD, vocal cord and pharyngeal distal myopathy, and welander distal myopathy. In some embodiments, endocrine myopathies are selected from, hyperthyroid myopathy, and hypothyroid myopathy. In some embodiments, inflammatory myopathies are selected from, dermatomyositis, inclusion-body myositis, and polymyositis. In some embodiments, metabolic myopathies are selected from, von Gierke's disease, Anderson disease, Fanconi-Bickel syndrome, aldolase A deficiency, acid maltase deficiency (Pompe disease), carnitine deficiency, carnitine palmitoyltransferase deficiency, debrancher enzyme deficiency (Cori disease, Forbes disease), lactate dehydrogenase deficiency, myoadenylate deaminase deficiency, phosphofructokinase deficiency (Tarui disease), phosphoglycerate kinase deficiency, phosphoglycerate mutase deficiency (Her's disease), and phosphorylase deficiency (McArdle disease). In some embodiments, cardiomyopathies are selected from intrinsic cardiomyopathies and extrinsic cardiomyopathies. In some embodiments, intrinsic cardiomyopathies are selected from genetic myopathies and acquired myopathies. In some embodiments, genetic myopathies are selected from Hypertrophic cardiomyopathy, arrhythmogenic right ventricular cardiomyopathy (ARVC), LV non-compaction, ion channelopathies, dilated cardiomyopathy (DCM), and restrictive cardiomyopathy (RCM). In some embodiments, acquired myopathies are selected from stress cardiomyopathy, myocarditis, eosinophilic myocarditis, and ischemic cardiomyopathy. In some embodiments, extrinsic cardiomyopathies are selected from metabolic cardiomyopathies, endomyocardial cardiomyopathies, endocrine cardiomyopathies, and cardiofacial cardiomyopathies. In some embodiments, metabolic cardiomyopathies are selected from Fabry's disease and hemochromatosis. In some embodiments, endomyocardial cardiomyopathies are selected from endomyocardial fibrosis and Hypereosinophilic syndrome. In some embodiments, endocrine cardiomyopathies are selected from diabetes mellitus, hyperthyroidism, and acromegaly. In some embodiments, the Cardiofacial cardiomyopathy is Noonan syndrome.

In some embodiments, disclosed herein are methods to treat neuromuscular and movement disorders by the administration of a compound or salt of Formula (Ia), (Ib), (II), (III), (IV), (V), (VI), (VI), (VII), or (VIII).

Presented herein are methods to treat neuromuscular and movement disorders by reduction of skeletal muscle contraction. Treatment of subjects with neuromuscular and movement disorders with a selective fast skeletal muscle (type II) myosin inhibitor of a compound or salt of Formula (Ia), (Ib), (II), (III), (IV), (V), (VI), (VI), (VII), or (VIII) may reduce muscle breakdown by preventing excessive uncoordinated muscle contractures resulting in less muscle damage. Furthermore, methods of the disclosure may reduce muscle damage while minimizing the impact on physical function in subjects. Preservation of function may occur both by limiting damaging levels of force generation in type II fibers and by increasing reliance on healthier type I fibers. Reduction of skeletal muscle contraction or uncoordinated muscle contractures can be reduced by the inhibition of skeletal myosin II. In certain embodiments, the inhibitor of skeletal myosin II is a compound or salt of Formula (Ia), (Ib), (II), (III), (IV), (V), (VI), (VI), (VII), or (VIII) as disclosed herein.

In some embodiments, disclosed herein is a method of inhibiting muscle myosin II, comprising administering a compound or salt of Formula (Ia), (Ib), (II), (III), (IV), (V), (VI), (VI), (VII), or (VIII) to a subject in need thereof. In some embodiments, the compound or salt does not appreciably inhibit cardiac muscle contraction. In some embodiments, wherein the compound or salt does not appreciably inhibit cardiac muscle contraction. In some embodiments, the compound or salt reduces cardiac muscle force by less than 10%.

In some aspects, methods of treating neuromuscular conditions or movement disorders may comprise administering a compound or salt of Formula (Ia), (Ib), (II), (III), (IV), (V), (VI), (VI), (VII), or (VIII) to inhibit skeletal muscle contraction. In some embodiments, the compound or salt of Formula (Ia), (Ib), (II), (III), (IV), (V), (VI), (VI), (VII), or (VIII) does not significantly inhibit cardiac muscle contraction. In some embodiments, cardiac muscle contraction is inhibited by 20% or less. In some embodiments, cardiac muscle contraction is inhibited by 15% or less. In some embodiments, cardiac muscle contraction is inhibited by 10% or less. In some embodiments, cardiac muscle contraction is inhibited by 9% or less. In some embodiments, cardiac muscle contraction is inhibited by 8% or less. In some embodiments, cardiac muscle contraction is inhibited by 7% or less. In some embodiments, cardiac muscle contraction is inhibited by 6% or less. In some embodiments, cardiac muscle contraction is inhibited by 5% or less. In some embodiments, cardiac muscle contraction is inhibited by 4% or less. In some embodiments, cardiac muscle contraction is inhibited by 3% or less. In some embodiments, cardiac muscle contraction is inhibited by 2% or less. In some embodiments, cardiac muscle contraction is inhibited by 1% or less.

A subject's activities of daily life (ADL) or habitual physical activity may be monitored prior to and following the treatment with a compound or salt of Formula (Ia), (Ib), (II), (III), (IV), (V), (VI), (VI), (VII), or (VIII). ADL or habitual physical activity is subject-dependent and may range from simple walking to extensive exercise depending on the subject's ability and routine. Treatment options and dosages of the skeletal muscle contraction inhibitors discussed herein may be personalized to a subject such that the ADL and habitual physical activity remains unchanged.

In some aspects, methods of treating neuromuscular conditions or movement disorders may comprise administering a compound or salt of Formula (Ia), (Ib), (II), (III), (IV), (V), (VI), (VI), (VII), or (VIII) to inhibit skeletal muscle contraction. A compound or salt of Formula (Ia), (Ib), (II), (III), (IV), (V), (VI), (VI), (VII), or (VIII) may be given in an amount relative to the amount needed to reduce skeletal muscle contraction by 50%. The compound or salt of Formula (Ia), (Ib), (II), (III), (IV), (V), (VI), (VI), (VII), or (VIII) may be administered in an amount less than the amount needed to reduce skeletal muscle contraction by 50% relative to pre-treatment skeletal muscle contraction capacity of the subject. The compound or salt of Formula (Ia), (Ib), (II), (III), (IV), (V), (VI), (VI), (VII), or (VIII) may be administered in an amount that reduces skeletal muscle contraction by 5% to 45% relative to pre-treatment skeletal muscle contraction capacity of said subject. In some cases, the compound or salt of Formula (Ia), (Ib), (II), (III), (IV), (V), (VI), (VI), (VII), or (VIII) may be administered in an amount that reduces skeletal muscle contraction by less than 10%, less than 15%, less than 20%, less than 25%, less than 30%, less than 35%, less than 40%, less than 45% or even less than 50% relative to pre-treatment skeletal muscle contraction capacity of said subject. In certain embodiments, the compound or salt of Formula (Ia), (Ib), (II), (III), (IV), (V), (VI), (VI), (VII), or (VIII) may be administered in an amount that reduces skeletal muscle contraction from 1% to 50% relative to pre-treatment skeletal muscle contraction capacity of said subject.

In some aspects, methods of treating neuromuscular conditions or movement disorders may comprise administering a compound or salt of Formula (Ia), (Ib), (II), (III), (IV), (V), (VI), (VI), (VII), or (VIII) to inhibit type I skeletal muscle contraction. The inhibitor of type I skeletal muscle contraction may be given in an amount relative to the amount needed to reduce type I skeletal muscle contraction by 20%. The inhibitor of type I skeletal muscle contraction may be administered in an amount less than the amount needed to reduce type I skeletal muscle contraction by 20% relative to pre-treatment type I skeletal muscle contraction capacity of the subject. The inhibitor of type I skeletal muscle contraction may be administered in an amount that reduces type I skeletal muscle contraction by 0.010% to 20% relative to pre-treatment type I skeletal muscle contraction capacity of said subject. In some cases, the inhibitor may be administered in an amount that reduces type I skeletal muscle contraction by less than 0.01%, less than 0.1%, less than 0.5%, less than 1%, less than 5%, less than 10%, less than 15% or less than 20% relative to pre-treatment type I skeletal muscle contraction capacity of said subject. In certain embodiments, the inhibitor may be administered in an amount that reduces type I skeletal muscle contraction from 0.010% to 20% relative to pre-treatment type I skeletal muscle contraction capacity of said subject.

In some aspects, methods of treating neuromuscular conditions or movement disorders may comprise administering a compound or salt of Formula (Ia), (Ib), (II), (III), (IV), (V), (VI), (VI), (VII), or (VIII) to inhibit type II skeletal muscle contraction. The inhibitor of type II skeletal muscle contraction may be given in an amount relative to the amount needed to reduce type II skeletal muscle contraction by 90%. The inhibitor of type II skeletal muscle contraction may be administered in an amount less than the amount needed to reduce type II skeletal muscle contraction by 90% relative to pre-treatment type II skeletal muscle contraction capacity of the subject. The inhibitor of type II skeletal muscle contraction may be administered in an amount that reduces type II skeletal muscle contraction by 5% to 75% relative to pre-treatment type II skeletal muscle contraction capacity of said subject. In some cases, the inhibitor may be administered in an amount that reduces type II skeletal muscle contraction by less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85% or even less than 90% relative to pre-treatment type II skeletal muscle contraction capacity of said subject. In certain embodiments, the inhibitor may be administered in an amount that reduces type II skeletal muscle contraction by from 1% to 50% relative to pre-treatment type II skeletal muscle contraction capacity of said subject.

In some aspects, methods of treating contraction-induced injury in skeletal muscle fiber may comprise administering a compound or salt of Formula (Ia), (Ib), (II), (III), (IV), (V), (VI), (VI), (VII), or (VIII) to inhibit skeletal muscle contraction and/or skeletal muscle myosin II. In certain embodiments, the inhibitor does not appreciably inhibit cardiac muscle contraction.

In some aspects, methods of treating metabolic myopathies, e.g. McCardle's syndrome, may comprise administering a compound or salt of Formula (Ia), (Ib), (II), (III), (IV), (V), (VI), (VI), (VII), or (VIII).

In certain embodiments, the contraction-induced injury in skeletal muscle fiber is from involuntary skeletal muscle contraction. The involuntary skeletal muscle contraction may be associated with a neuromuscular condition or spasticity-associated condition. In certain embodiments, the contraction-induced injury in skeletal muscle fiber may be from voluntary skeletal muscle contraction, e.g., physical exercise.

In certain embodiments, the administration of a compound or salt of Formula (Ia), (Ib), (II), (III), (IV), (V), (VI), (VI), (VII), or (VIII) to a subject modulates one or more biomarkers associated with muscle contraction. Examples of biomarkers include but are not limited to creatinine kinase (CK), Troponin T (TnT), Troponin C (TnC), Troponin I (TnI), pyruvate kinase (PK), lactate dehydrogenase (LDH), myoglobin, isoforms of TnI (such as cardiac, slow skeletal, fast skeletal muscles) and inflammatory markers (IL-1, IL-6, IL-4, TNF-α). Biomarkers may also include measures of muscle inflammation for example, edema. The level of biomarkers described herein may increase after the administration of the inhibitor relative to pre-treatment level of the biomarkers. Alternatively, the level of biomarkers may decrease after the administration of the inhibitor relative to pre-treatment level of the biomarkers. The modulation of one or more biomarkers with an inhibitor described herein may indicate treatment of a neuromuscular condition such as those described herein.

Levels of CK in a subject increase when the subject is active as compared to when the subject is inactive (e.g., sleeping) and therefore CK is a potential metric for evaluating skeletal muscle breakdown caused by skeletal muscle contraction. In certain embodiments, a compound or salt of Formula (Ia), (Ib), (II), (III), (IV), (V), (VI), (VI), (VII), or (VIII) may be administered to a subject prior to mild, moderate or strenuous activity to reduce or prevent skeletal muscle breakdown from the activity. Moderate to strenuous activity may be dependent on a subject's abilities and may include physical exercise that increases the heart rate by at least 20% or more, such as about 50% or more relative to the subject's resting heart rate. Examples of moderate to strenuous activity include walking, running, weight lifting, biking, swimming, hiking, etc.

In certain embodiments, a compound or salt of Formula (Ia), (Ib), (II), (III), (IV), (V), (VI), (VI), (VII), or (VIII) is administered prior to, during, or after moderate or strenuous activity to reduce or prevent skeletal muscle breakdown from the activity. The compound or salt of Formula (Ia), (Ib), (II), (III), (IV), (V), (VI), (VI), (VII), or (VIII) may reduce the subject's level of CK relative to the untreated subject performing the same activity. The level of CK may be measured in the peripheral blood of the subject during or after the activity. The administration of an inhibitor described herein may reduce the level of CK by 5% to 90% in an active subject relative to the untreated subject performing the same activity, thereby reducing or preventing skeletal muscle breakdown from the activity. The administration of an inhibitor described herein may modulate the level of CK by about 5% to about 90% relative to the untreated subject performing the same activity, thereby reducing or preventing skeletal muscle breakdown from the activity. The administration of an inhibitor described herein may reduce the level of CK by at least about 5% relative to the untreated subject performing the same activity thereby reducing or preventing skeletal muscle breakdown from the activity. The administration of an inhibitor described herein may modulate the level of CK by at most about 90% relative to the untreated subject performing the same activity. The administration of an inhibitor described herein may reduce the level of CK by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to the untreated subject performing the same activity, thereby reducing or preventing skeletal muscle breakdown from the activity. The administration of an inhibitor described herein may modulate the level of CK by about 5%, about 15%, about 25%, about 35%, about 45%, about 55%, about 65%, about 75%, about 85%, or about 90% relative to the untreated subject performing the same activity, thereby reducing or preventing skeletal muscle breakdown from the activity.

The administration of a compound or salt of Formula (Ia), (Ib), (II), (III), (IV), (V), (VI), (VI), (VII), or (VIII) to a subject may modulate the levels of inflammatory markers, e.g., reduce the level of one or more inflammatory markers relative to the untreated subject or the subject prior to treatment. The level of inflammatory markers may be measured in the peripheral blood of the subject. Examples of inflammatory markers may include but are not limited to IL-1, IL-6 and TNF-α. Inflammatory markers may also be in the form of conditions such as edema which may be measured using magnetic resonance imaging. The level of inflammatory markers in the peripheral blood may increase after the administration of the inhibitor relative to pre-treatment level of inflammatory marker for the subject. Alternatively, the level of inflammatory markers in the peripheral blood may decrease after the administration of the inhibitor relative to pre-treatment level of inflammatory marker for the subject. The administration of an inhibitor described herein may modulate the level of inflammatory markers by 5% to 90% relative to pre-treatment level of inflammatory marker for the subject. In some cases, the level of inflammatory markers may be modulated by about 5% to about 90% relative to pre-treatment level of inflammatory markers of the subject. In some cases, the level of inflammatory markers may be modulated by at least about 5% relative to pre-treatment level of inflammatory markers of the subject. In some cases, the level of inflammatory markers may be modulated by at most about 90% relative to pre-treatment level of inflammatory markers of the subject. In some cases, the level of inflammatory markers may be modulated by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to pre-treatment level of inflammatory markers of the subject. In some cases, the level of inflammatory markers may be modulated by about 5%, about 15%, about 25%, about 35%, about 45%, about 55%, about 65%, about 75%, about 85%, or about 90% relative to pre-treatment level of inflammatory markers of the subject.

The administration of a compound or salt of Formula (Ia), (Ib), (II), (III), (IV), (V), (VI), (VI), (VII), or (VIII) to a subject may modulate the levels of circulating fast skeletal muscle Troponin I (fS-TnI). The level of fS-TnI may be measured in the peripheral blood. The level of fS-TnI in the peripheral blood may increase after the administration of the inhibitor relative to pre-treatment level of fS-TnI for the subject. Alternatively, the level of fS-TnI in the peripheral blood may decrease after the administration of the inhibitor relative to pre-treatment level of fS-TnI for the subject. The administration of an inhibitor described herein may modulate the level of fS-TnI by 5% to 90% relative to pre-treatment level of fS-TnI for the subject. In some cases, the level of fS-TnI may be modulated by at least about 5% relative to pre-treatment level of fS-TnI of the subject. In some cases, the level of fS-TnI may be modulated by at most about 90% relative to pre-treatment level of fS-TnI of the subject. In some cases, the level of fS-TnI may be modulated by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to pre-treatment level of fS-TnI of the subject. In some cases, the level of fS-TnI may be modulated by about 5%, about 15%, about 25%, about 35%, about 45%, about 55%, about 65%, about 75%, about 85%, or about 90% relative to pre-treatment level of fS-TnI of the subject.

Isoforms of troponin may be measured in a subject prior to and following the administration a compound or salt of Formula (Ia), (Ib), (II), (III), (IV), (V), (VI), (VI), (VII), or (VIII). Inhibition of skeletal muscle contraction may not inhibit some isoforms of troponin, such as cardiac troponin I (cTnI) or slow skeletal troponin I (ssTnI). In some cases, the inhibition of skeletal muscle contraction may not appreciably inhibit cTnI or ssTnI. As used herein with regard to cTnI or ssTnI, the phrase not appreciably refers to the cTnI or ssTnI reduced by less than 10%, less than 8%, less than 6%, less than 4%, less than 2%, less than 1%, less than 0.5% or even less than 0.1% relative to the cTnI or ssTnI prior to the administration of the inhibitor.

The administration of a compound or salt of Formula (Ia), (Ib), (II), (III), (IV), (V), (VI), (VI), (VII), or (VIII) may reduce involuntary muscle contractions. Involuntary muscle contractions may be reduced by 20% to 90% relative to involuntary muscle contractions prior to the administration of the inhibitor. In some cases, involuntary muscle contractions may be reduced by at least about 20% relative to pre-treatment involuntary muscle contractions. In some cases, involuntary muscle contractions may be reduced by at most about 90% relative to pre-treatment involuntary muscle contractions. In some cases, involuntary muscle contractions may be reduced by about 20% to about 25%, about 20% to about 30%, about 20% to about 40%, about 20% to about 50%, about 20% to about 70%, about 20% to about 75%, about 20% to about 80%, about 20% to about 85%, about 20% to about 90%, about 25% to about 30%, about 25% to about 40%, about 25% to about 50%, about 25% to about 70%, about 25% to about 75%, about 25% to about 80%, about 25% to about 85%, about 25% to about 90%, about 30% to about 40%, about 30% to about 50%, about 30% to about 70%, about 30% to about 75%, about 30% to about 80%, about 30% to about 85%, about 30% to about 90%, about 40% to about 50%, about 40% to about 70%, about 40% to about 75%, about 40% to about 80%, about 40% to about 85%, about 40% to about 90%, about 50% to about 70%, about 50% to about 75%, about 50% to about 80%, about 50% to about 85%, about 50% to about 90%, about 70% to about 75%, about 70% to about 80%, about 70% to about 85%, about 70% to about 90%, about 75% to about 80%, about 75% to about 85%, about 75% to about 90%, about 80% to about 85%, about 80% to about 90%, or about 85% to about 90% relative to pre-treatment involuntary muscle contractions. In some cases, involuntary muscle contractions may be reduced by about 20%, about 25%, about 30%, about 40%, about 50%, about 70%, about 75%, about 80%, about 85%, or about 90% relative to pre-treatment involuntary muscle contractions.

A compound or salt of Formula (Ia), (Ib), (II), (III), (IV), (V), (VI), (VI), (VII), or (VIII) may be used to improve activities of daily living (ADL) or habitual physical activity in a subject as mature, functional undamaged muscle may be restored. Examples of ADL or habitual activities include but are not limited to stair climb, time to get up, timed chair rise, habitual walk speed, North Star Ambulatory assessment, incremental/endurance shuttle walk and 6 minute walk distance tests. ADL or habitual physical activity levels or capacity may be measured prior to and following the administration of a skeletal muscle inhibitor. Inhibition of skeletal muscle contraction may not affect ADL or habitual physical activity. In some cases, the inhibition of skeletal muscle contraction may not appreciably affect ADL or habitual physical activity. As used herein with regard to ADL or habitual physical activity, the phrase not appreciably refers to the level of ADL or habitual activity reduced by less than 20%, less than 15%, less than 10%, less than 8%, less than 6%, less than 4%, less than 2%, less than 1%, less than 0.5% or even less than 0.1% relative to the ADL or habitual activity prior to the administration of the inhibitor.

Skeletal muscle contraction or force in a subject may be measured prior to and following the administration of the compound or salt of Formula (Ia), (Ib), (II), (III), (IV), (V), (VI), (VI), (VII), or (VIII). Such measurements may be performed to generate a dose response curve for the compound or salt of Formula (Ia), (Ib), (II), (III), (IV), (V), (VI), (VI), (VII), or (VIII). Dosage of the compound or salt of Formula (Ia), (Ib), (II), (III), (IV), (V), (VI), (VI), (VII), or (VIII) may be adjusted by about 5% to 50% relative to a dose that reduces type II skeletal muscle contraction by 90%. In some cases, dosage of the skeletal muscle contraction inhibitor may be adjusted by at least about 5% relative to a dose that reduces type II skeletal muscle contraction by 90%. In some cases, dosage of the skeletal muscle contraction inhibitor may be adjusted by at most about 50% relative to a dose that reduces type II skeletal muscle contraction by 90%. In some cases, dosage of the skeletal muscle contraction inhibitor may be adjusted by about 5% to about 10%, about 5% to about 15%, about 5% to about 20%, about 5% to about 25%, about 5% to about 30%, about 5% to about 35%, about 5% to about 40%, about 5% to about 50%, about 10% to about 15%, about 10% to about 20%, about 10% to about 25%, about 10% to about 30%, about 10% to about 35%, about 10% to about 40%, about 10% to about 50%, about 15% to about 20%, about 15% to about 25%, about 15% to about 30%, about 15% to about 35%, about 15% to about 40%, about 15% to about 50%, about 20% to about 25%, about 20% to about 30%, about 20% to about 35%, about 20% to about 40%, about 20% to about 50%, about 25% to about 30%, about 25% to about 35%, about 25% to about 40%, about 25% to about 50%, about 30% to about 35%, about 30% to about 40%, about 30% to about 50%, about 35% to about 40%, about 35% to about 50%, or about 40% to about 50% relative to a dose that reduces type II skeletal muscle contraction by 90%. In some cases, dosage of the skeletal muscle contraction inhibitor may be adjusted by about 10%, about 12%, about 15%, about 18%, about 20%, about 25%, about 30%, about 35%, about 40%, about 45% or about 50% relative to a dose that reduces type II skeletal muscle contraction by 90%. Skeletal muscle contraction may be measured by a muscle force test after nerve stimulation using surface electrodes (e.g., foot plantar flexion after peroneal nerve stimulation in the leg), isolated limb assay, heart rate monitor or an activity monitor or equivalents thereof prior to and following the administration of a skeletal muscle contraction inhibitor.

Cardiac muscle force or cardiac muscle contraction of a subject may be measured prior to and following the administration of a compound or salt of Formula (Ia), (Ib), (II), (III), (IV), (V), (VI), (VI), (VII), or (VIII). Inhibition of skeletal muscle contraction may not inhibit cardiac muscle contraction or cardiac muscle force. In some embodiments, the inhibition of skeletal muscle contraction may not appreciably inhibit cardiac muscle contraction. In certain embodiments with regard to cardiac muscle contraction, the phrase not appreciably refers to cardiac muscle force reduced by less than 10%, less than 8%, less than 6%, less than 4%, less than 2%, less than 1%, less than 0.5% or even less than 0.1% relative to the cardiac muscle force prior to the administration of the inhibitor. Cardiac muscle force or cardiac muscle contraction of a subject following the administration of a compound or salt of Formula (Ia), (Ib), (II), (III), (IV), (V), (VI), (VI), (VII), or (VIII) may be within 0.1% to 10% of the cardiac muscle contraction or cardiac muscle force prior to the administration of the inhibitor. In some embodiments, administration of a compound or salt of Formula (Ia), (Ib), (II), (III), (IV), (V), (VI), (VI), (VII), or (VIII) may inhibit skeletal muscle contraction and cardiac muscle contraction or cardiac muscle force. In some embodiments, cardiac muscle force reduced by more than 0.1%, more than 0.5%, more than 1%, more than 2%, more than 4%, more than 6%, more than 8%, or more than 10%. In some embodiments, a reduction of skeletal muscle contraction and cardiac muscle contraction are described by a ratio to one another. For example, in some embodiments, the ratio of the reduction in skeletal muscle contraction to reduction in cardiac muscle contraction is from about 1:1 to about 100:1, about 2:1 to about 50:1, about 3:1 to about 40:1, about 4:1 to about 30:1, about 5:1 to about 20:1, about 7:1 to about 15:1, or about 8:1 to about 12:1. Cardiac muscle force or cardiac muscle contraction may be measured using an echocardiogram (fractional shortening) or other equivalent tests.

Tidal volume in lung in a subject may be measured prior to and following the administration of a compound or salt of Formula (Ia), (Ib), (II), (III), (IV), (V), (VI), (VI), (VII), or (VIII). Administration may not inhibit tidal volume in a lung. In some cases, administration may not appreciably inhibit tidal volume in a lung. In certain embodiments with regard to tidal lung volume in a lung, the phrase not appreciably refers to the tidal volume in a lung reduced by less than 10%, less than 8%, less than 6%, less than 4%, less than 2%, less than 1%, less than 0.5% or less than 0.1% relative to the tidal volume in a lung prior to the administration of the inhibitor. Tidal volume in a lung in a subject may be measured using forced volume in one second test (FEV1) or forced vital capacity test (FVC) or equivalent tests thereof.

Smooth muscle contraction in a subject may be measured prior to and following the administration of a skeletal muscle contraction inhibitor. Inhibition of skeletal muscle contraction may not inhibit smooth muscle contraction. In some cases, the inhibition of skeletal muscle contraction may not appreciably inhibit smooth muscle contraction. As used herein with regard to smooth muscle contraction, the phrase not appreciably refers to the smooth muscle contraction reduced by less than 10%, less than 8%, less than 6%, less than 4%, less than 2%, less than 1%, less than 0.5% or even less than 0.1% relative to the smooth muscle contraction prior to the administration of the inhibitor. Smooth muscle contraction in a subject may be evaluated by measuring a subject's blood pressure.

Neuromuscular coupling in a subject may be measured prior to and following the administration of a compound or salt of Formula (Ia), (Ib), (II), (III), (IV), (V), (VI), (VI), (VII), or (VIII). Inhibition of skeletal muscle contraction, with an inhibitor described herein, may not impair nerve conduction, neurotransmitter release or electrical depolarization of skeletal muscle in a subject. In some cases, the inhibition of skeletal muscle contraction may not appreciably impair neuromuscular coupling in a subject. As used herein with regard to neuromuscular coupling, the phrase not appreciably refers to a level of neuromuscular coupling in the subject reduced by less than 10%, less than 8%, less than 6%, less than 4%, less than 2%, less than 1%, less than 0.5% or less than 0.1% relative to the level of neuromuscular coupling in the subject prior to the administration of the inhibitor. Neuromuscular coupling in a subject may be evaluated by measuring nerve induced electrical depolarization of skeletal muscle by the recording of electrical activity produced by skeletal muscles after electrical or voluntary stimulation with electromyography (EMG) using surface or needle electrodes.

In some aspects, the method of treating a neuromuscular condition or movement disorder can comprise administering a compound or salt of Formula (Ia), (Ib), (II), (III), (IV), (V), (VI), (VI), (VII), or (VIII) wherein the compound or salt of Formula (Ia), (Ib), (II), (III), (IV), (V), (VI), (VI), (VII), or (VIII) may inhibit myosin ATPase activity, native skeletal muscle myofibril ATPase (calcium regulated) or a reconstituted Si with actin, tropomyosin and troponin. In vitro assays may be used to test the effect of the test compound or inhibitor on the myosin ATPase activity. Test compounds can be screened for assessing their inhibitory activity of muscle contraction. Inhibitory activity can be measured using an absorbance assay to determine actin-activated ATPase activity. Rabbit muscle myosin sub-fragment 1 (S1) can be mixed with polymerized actin and distributed into wells of assay plates without nucleotides. Test compounds can then be added into the wells with a pin array. The reaction can be initiated with MgATP. The amount of ATP consumption over a defined time period in the test vessel may be compared to the amount of ATP consumption in a control vessel. The defined period of time may be 5 minutes to 20 minutes. The ATP consumption can be determined by direct or indirect assays. The test compounds that reproducibly and strongly inhibited the myosin S1 ATPase activity can be evaluated further in dose response assay to determine IC50 for the compound ex vivo on dissected muscles. The assay may measure ATPase activity indirectly by coupling the myosin to pyruvate kinase and lactate dehydrogenase to provide an absorbance detection method at 340 nm based upon the conversion of NADH to NAD+ driven by ADP accumulation. In some cases, wherein ATP consumption is decreased by at least 20% in said test vessel than said control vessel, said test compound may be selected as a compound or salt of Formula (Ia), (Ib), (II), (III), (IV), (V), (VI), (VI), (VII), or (VIII). A test compound may be selected when there is at least 20% greater inhibition of NAD+ generation in a kinetic assay.

The inhibitor or test compound selected may not inhibit cardiac muscle myosin S1 ATPase in in vitro assays. In some cases, the cardiac muscle myosin S1 ATPase or cardiac myofibrils or reconstituted system may be inhibited by less than 10%, less than 8%, less than 5%, less than 3%, less than 2%, less than 1% or less than 0.5% when a test compound or compound or salt of Formula (Ia), (Ib), (II), (III), (IV), (V), (VI), (VI), (VII), or (VIII) is tested in an in-vitro assay.

Test compounds of skeletal muscle contraction may be tested on skinned fibers. Single skeletal muscle fibers, treated so as to remove membranes and allow for a direct activation of contraction after calcium administration may be used. An inhibitor compound or salt of Formula (Ia), (Ib), (II), (III), (IV), (V), (VI), (VI), (VII), or (VIII) may inhibit contraction of a single skeletal muscle fiber by about 5% to about 90% relative to pre-treatment value or an untreated control single skeletal muscle fiber. An inhibitor may inhibit contraction of a single skeletal muscle fiber by at least about 5% relative to pre-treatment value or an untreated control single skeletal muscle fiber. An inhibitor may inhibit contraction of a single skeletal muscle fiber by at most about 90% relative to pre-treatment value or an untreated control single skeletal muscle fiber. An inhibitor may inhibit contraction of a single skeletal muscle fiber by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% relative to pre-treatment capacity or an untreated control single skeletal muscle fiber. An inhibitor may inhibit contraction of a single skeletal muscle fiber by about 5%, about 10%, about 20%, about 30%, about 40%, about 50%, about 60%, about 70%, about 80%, or about 90% relative to pre-treatment capacity or an untreated control single skeletal muscle fiber.

An inhibitor compound or salt of Formula (Ia), (Ib), (II), (III), (IV), (V), (VI), (VI), (VII), or (VIII) may inhibit contraction of a single skeletal muscle by about 5% to about 90% relative to pre-treatment value or an untreated control single skeletal muscle. An inhibitor may inhibit contraction of a single skeletal muscle by at least about 5% relative to pre-treatment value or an untreated control single skeletal muscle. An inhibitor may inhibit contraction of a single skeletal muscle by at most about 90% relative to pre-treatment value or an untreated control single skeletal muscle. An inhibitor may inhibit contraction of a single skeletal muscle by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% relative to pre-treatment capacity or an untreated control single skeletal muscle. An inhibitor may inhibit contraction of a single skeletal muscle by about 5%, about 10%, about 20%, about 30%, about 40%, about 50%, about 60%, about 70%, about 80%, or about 90% relative to pre-treatment capacity or an untreated control single skeletal muscle.

The effect of a test compound on slow type I skeletal muscle fibers, cardiac muscle bundles or lung muscle fibers, may be evaluated. A test compound or inhibitor compound or salt of Formula (Ia), (Ib), (II), (III), (IV), (V), (VI), (VI), (VII), or (VIII) may be selected so as not to appreciably modulate the function of slow type I skeletal muscle fibers, cardiac muscle bundles or lung muscle fibers and be specific for type II skeletal muscles. As used herein, the term “appreciably modulate” can refer to the contraction capacity of muscles following the inhibitor administration to be reduced less than 10%, less than 8%, less than 6%, less than 4%, less than 2%, less than 1%, less than 0.5% or even less than 0.1% relative to the muscle force/contraction prior to the administration of the inhibitor.

In some aspects, a method of treating a neuromuscular condition or a movement disorder may comprise administering to a subject in need thereof a compound or salt of Formula (Ia), (Ib), (II), (III), (IV), (V), (VI), (VI), (VII), or (VIII) wherein the compound or salt of Formula (Ia), (Ib), (II), (III), (IV), (V), (VI), (VI), (VII), or (VIII) reduces skeletal muscle contraction by 5% to 90% in an ex vivo assay. The ex vivo assays used may be mouse models. The mouse models used may be dystrophy mouse models such as an mdx mouse. The mdx mouse has a point mutation in its dystrophin gene, changing the amino acid coding for a glutamine to a threonine producing a nonfunctional dystrophin protein resulting in DMD where there is increased muscle damage and weakness. Extensor digitorum longus muscles may be dissected from mdx mice and mounted on a lever arm. The muscles may be bathed in an oxygenated Krebs solution to maintain muscle function. A test compound or compound or salt of Formula (Ia), (Ib), (II), (III), (IV), (V), (VI), (VI), (VII), or (VIII) may be applied to the muscles. An isometric (fixed length) contraction step may then be performed wherein the muscles are stimulated with a series of electrical pulses. An eccentric (lengthening) contraction step may be performed wherein the muscles are stretched to 10%, 15%, 20%, 25%, or 30% greater than its rested length, while relaxed or while stimulated with an electrical pulse. In some embodiments, the eccentric contraction step is repeated from 2 to 50 times. In some embodiments, the eccentric contraction step is repeated from 2 to 40 times. In some embodiments, the eccentric contraction step is repeated from 2 to 30 times. In some embodiments, the eccentric contraction step is repeated from 2 to 20 times. In some embodiments, the eccentric contraction step is repeated from 2 to 10 times. In some embodiments, the eccentric contraction step is repeated 4, 5, 6, 7, 8, 9, 10, 11, 12, 13, 14, or 15 times to cause muscle fiber injury. In some embodiments, the electric pulses may have a frequency of about 1 Hz to about 500 Hz. In some embodiments, the electric pulses may have a frequency of about 1 Hz to about 400 Hz. In some embodiments, the electric pulses may have a frequency of about 1 Hz to about 300 Hz. In some embodiments, the electric pulses may have a frequency of about 1 Hz to about 200 Hz. In some embodiments, the electric pulses may have a frequency of about 1 Hz to about 100 Hz. The electric pulse may have a frequency of about 50, 55, 60, 65, 70, 75, 80, 85, 90, 95, 100, 105, 110, 115, 120, 125, 130, 135, 140, 145 or 150 Hz. A series of electric pulses may comprise of individual pulses of different frequencies. The time period of each pulse in the series of electric pulses may be between 0.1 second to 0.5 seconds for each pulse. The time for each pulse may be 0.1, 0.2, 0.3, 0.35, 0.4 or 0.5 seconds. Muscle membrane damage may also be measured by incubating muscles in procion orange after the isometric or eccentric contraction. Procion orange is a fluorescent dye that is taken up by muscle fibers with injured membranes. The number or proportion of dye-positive fibers may then quantified by histology. When the test force drop and/or proportion of dye-positive fibers may be at least 20% less than the control force drop and/or dye uptake, the test compound may be selected as a compound or salt of Formula (Ia), (Ib), (II), (III), (IV), (V), (VI), (VI), (VII), or (VIII).

Using an isometric or eccentric set of contractions, the force generated by the muscle may be measured. The change in force generated by the muscle before and after an isometric or eccentric set of contractions may be calculated as the test force drop. The calculations may be compared to the change in force generated by the muscle contraction from the first pulse to the last pulse in a control sample without exposure to the test compound (control force drop). Force drop can be used as a surrogate of muscle injury and a test compound or inhibitor compound or salt of Formula (Ia), (Ib), (II), (III), (IV), (V), (VI), (VI), (VII), or (VIII) may be selected when the test force drop is at least 20% less than the control force drop.

### Pharmaceutical Formulations

The compositions and methods described herein may be considered useful as pharmaceutical compositions for administration to a subject in need thereof. Pharmaceutical compositions may comprise a compound or salt of Formula (Ia), (Ib), (II), (III), (IV), (V), (VI), (VI), (VII), or (VIII) described herein and one or more pharmaceutically acceptable carriers, diluents, excipients, stabilizers, dispersing agents, suspending agents, and/or thickening agents.

Pharmaceutical compositions comprising a compound or salt of Formula (Ia), (Ib), (II), (III), (IV), (V), (VI), (VI), (VII), or (VIII) may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate.

The pharmaceutical compositions may also include the compounds, salts or conjugates in a free-base form or pharmaceutically-acceptable salt form.

Methods for formulation of a compound or salt of Formula (Ia), (Ib), (II), (III), (IV), (V), (VI), (VI), (VII), or (VIII) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions comprising a compound or salt of Formula (Ia), (Ib), (II), (III), (IV), (V), (VI), (VI), (VII), or (VIII) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration.

The compositions comprising a compound or salt of Formula (Ia), (Ib), (II), (III), (IV), (V), (VI), (VI), (VII), or (VIII) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes.

Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or salt of Formula (Ia), (Ib), (II), (III), (IV), (V), (VI), (VI), (VII), or (VIII) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

In one embodiment the invention relates to methods and compositions of Formula (Ia), (Ib), (II), (III), (IV), (V), (VI), (VI), (VII), or (VIII) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment compositions of Formula (Ia), (Ib), (II), (III), (IV), (V), (VI), (VI), (VII), or (VIII) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment compositions of Formula (Ia), (Ib), (II), (III), (IV), (V), (VI), (VI), (VII), or (VIII) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise glycerol monostearate 40-50, hydroxypropyl cellulose, pyridazine, magnesium stearate, methacrylic acid copolymer type C, polysorbate 80, sugar spheres, talc, or triethyl citrate.

In another embodiment the compositions of Formula (Ia), (Ib), (II), (III), (IV), (V), (VI), (VI), (VII), or (VIII) are enteric-coated controlled-release tablets for oral administration. The compositions can further comprise camauba wax, crospovidone, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or salt of Formula (Ia), (Ib), (II), (III), (IV), (V), (VI), (VI), (VII), or (VIII) may be also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or salt of Formula (Ia), (Ib), (II), (III), (IV), (V), (VI), (VI), (VII), or (VIII) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment the compositions of Formula (Ia), (Ib), (II), (III), (IV), (V), (VI), (VI), (VII), or (VIII) can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment compositions of Formula (Ia), (Ib), (II), (III), (IV), (V), (VI), (VI), (VII), or (VIII) are provided in effervescent dosage forms. These effervescent dosage forms can also comprise non-release controlling excipients.

In another embodiment compositions of Formula (Ia), (Ib), (II), (III), (IV), (V), (VI), (VI), (VII), or (VIII) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment compositions of Formula (Ia), (Ib), (II), (III), (IV), (V), (VI), (VI), (VII), or (VIII) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, the compositions of Formula (Ia), (Ib), (II), (III), (IV), (V), (VI), (VI), (VII), or (VIII) provided herein can be in unit-dosage forms or multiple-dosage forms.

Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form.

Examples of multiple-dosage forms include, but are not limited to, vials, bottles of tablets or capsules, or bottles of pints or gallons. In another embodiment the multiple dosage forms comprise different pharmaceutically active agents.

In some embodiments, the compositions of Formula (Ia), (Ib), (II), (III), (IV), (V), (VI), (VI), (VII), or (VIII) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually hours, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

In certain embodiments, a compound or salt of the disclosure may be administered in combination with an oral corticosteroid. In certain embodiments, a compound or salt of the disclosure is administered in combination with deflazacort. In certain embodiments, a compound or salt of the disclosure is administered in combination with prednisone. In certain embodiments, a compound or salt of the disclosure is administered in combination with a morpholino antisense oligomer. In certain embodiments, a compound or salt of the disclosure is administered in combination with and exon skipping therapy. In certain embodiments, the additional therapeutic agent is eteplirsen or ataluren.

In certain embodiments, a compound or salt of the disclosure is used in combination with a gene therapy. In certain embodiments, the compound or salt of the disclosure is used in combination with adeno-associated virus (AAV) containing genes encoding replacement proteins, e.g., dystrophin, or truncated version thereof, e.g., microdystrophin. In certain embodiments, a compound or salt of the disclosure is administered in combination with vamorolone.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1: 2-(5-(5-(3-Chlorophenyl)-1,2,4-thiadiazol-3-yl)-2-oxopyridin-1(2H)-yl)-N-ethylacetamide (Compound 10)

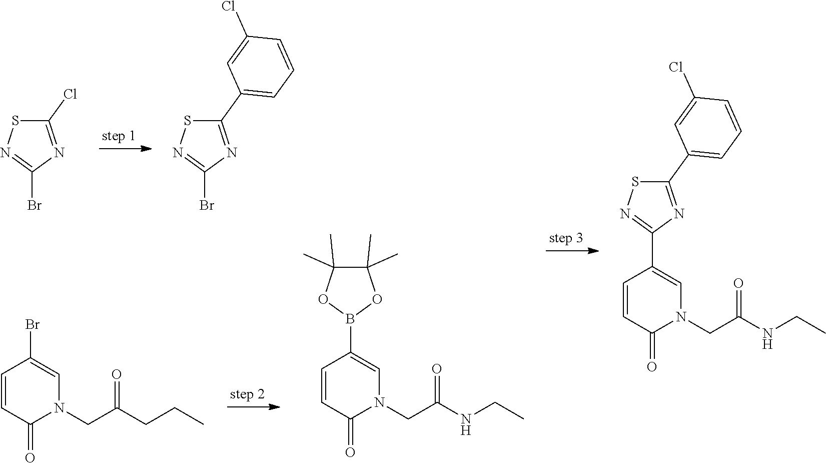 [SMILES: CCCC(=O)Cn1cc(Br)ccc1=O ; Clc1nc(Br)ns1 ; Clc1cccc(-c2nc(Br)ns2)c1 ; CCNC(=O)Cn1cc(B2OC(C)(C)C(C)(C)O2)ccc1=O ; CCNC(=O)Cn1cc(-c2nsc(-c3cccc(Cl)c3)n2)ccc1=O]

### Step 1: 3-Bromo-5-(3-chlorophenyl)-1,2,4-thiadiazole

To a stirred solution of 3-chlorophenylboronic acid (500 mg, 3.198 mmol, 1.00 equiv) in DMF (8 mL) and H₂O (1.6 mL), 3-bromo-5-chloro-1,2,4-thiadiazole (1.28 g, 6.396 mmol, 2.00 equiv), K₃PO₄ (2.04 g, 9.594 mmol, 3.00 equiv) and Pd(dppf)Cl₂·CH₂Cl₂ (1.30 g, 1.599 mmol, 0.50 equiv) were added portion wise at room temperature under argon atmosphere. The resulting mixture was stirred for 3 h at 80° C. under argon atmosphere. The resulting mixture was diluted with water and extracted with EtOAc. The combined organic layers were washed with brine, dried over anhydrous Na₂SO₄, filtered and the filtrate was concentrated under reduced pressure. The residue was purified by silica gel column chromatography to afford 3-bromo-5-(3-chlorophenyl)-1,2,4-thiadiazole (420 mg, 47.67%). LCMS (ES, m/z): 275 [M+H]⁺

### Step 2: N-Ethyl-2-(2-oxo-5-(4,4,5,5-tetramethyl-1,3,2-dioxaborolan-2-yl)pyridin-1(2H)-yl)acetamide

To a stirred solution of 2-(5-bromo-2-oxopyridin-1-yl)-N-ethylacetamide (700 mg, 2.702 mmol, 1.00 equiv) in 1,4-dioxane (8 mL), 4,4,4′,4′,5,5,5′,5′-octamethyl-2,2′-bi(1,3,2-dioxaborolane) (1.37 g, 5.404 mmol, 2.00 equiv), KOAc (795.43 mg, 8.106 mmol, 3.00 equiv) and Pd(dppf)Cl₂ (197.68 mg, 0.270 mmol, 0.10 equiv) were added portion wise at room temperature under argon atmosphere. The resulting mixture was stirred for 2 h at 80° C. under argon atmosphere resulting in N-ethyl-2-(2-oxo-5-(4,4,5,5-tetramethyl-1,3,2-dioxaborolan-2-yl)pyridin-1(2H)-yl)acetamide (700 mg, crude). The crude mixture was used directly in the next step without further purification. LCMS (ES, m/z): 307 [M+H]⁺

### Step 3: 2-(5-(5-(3-Chlorophenyl)-1,2,4-thiadiazol-3-yl)-2-oxopyridin-1(2H)-yl)-N-ethylacetamide

To a stirred solution of 3-bromo-5-(3-chlorophenyl)-1,2,4-thiadiazole (400 mg, 1.452 mmol, 1.00 equiv) in 1,4-dioxane (5 mL) and H₂O (1 mL) was added N-ethyl-2-(2-oxo-5-(4,4,5,5-tetramethyl-1,3,2-dioxaborolan-2-yl)pyridin-1(2H)-yl)acetamide (666.67 mg, 2.178 mmol, 1.50 equiv), Na₂CO₃ (384.64 mg, 3.630 mmol, 2.50 equiv), and Pd(dppf)Cl₂ (212.44 mg, 0.290 mmol, 0.20 equiv) portion wise at room temperature under argon atmosphere. The resulting mixture was stirred for 3 h at 80° C. under argon atmosphere. The resulting mixture was diluted with water (80 mL). The resulting mixture was extracted with EtOAc and the combined organic layers were washed with brine, dried over anhydrous Na₂SO₄ and filtered. The filtrate was concentrated under reduced pressure and the residue purified by silica gel column chromatography and then the crude product (300 mg) was purified by Prep-HPLC (Column: XBridge C18 OBD Prep Column, 100 Å 5 μm, 19 mm×250 mm; Mobile Phase A: Water (10 mmol/L NH₄HCO₃), Mobile Phase B: ACN; Flow rate: 25 mL/min; Gradient: 30% B to 54% B in 7 min, 54% B; Wave Length: 254/220 nm; RT1 (min): 6.83) to afford 2-(5-(5-(3-chlorophenyl)-1,2,4-thiadiazol-3-yl)-2-oxopyridin-1(2H)-yl)-N-ethylacetamide (46 mg, 8.45%) LCMS (ES, m/z): 375 [M+H]⁺

### Example 2: 6-(3-(5-Chloropyridin-3-yl)-1,2,4-thiadiazol-5-yl)-2-((5-fluoropyridin-3-yl)methyl)pyridazin-3(2H)-one (Compound 6)

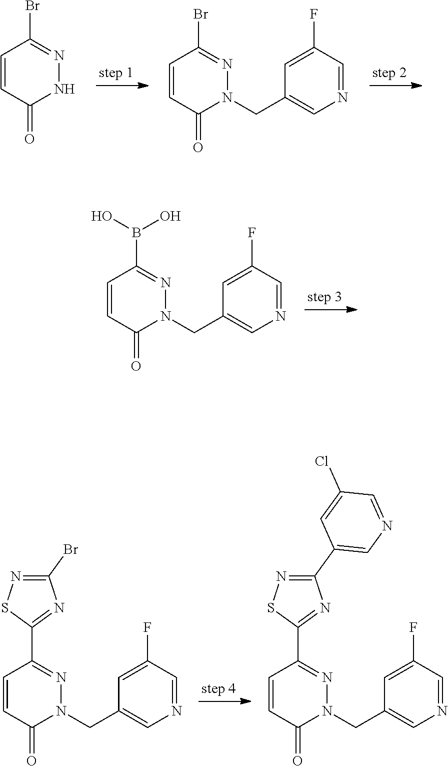 [SMILES: O=c1ccc(Br)n[nH]1 ; O=c1ccc(Br)nn1Cc1cncc(F)c1 ; O=c1ccc(B(O)O)nn1Cc1cncc(F)c1 ; O=c1ccc(-c2nc(Br)ns2)nn1Cc1cncc(F)c1 ; O=c1ccc(-c2nc(-c3cncc(Cl)c3)ns2)nn1Cc1cncc(F)c1]

### Step 1: 6-Bromo-2-((5-fluoropyridin-3-yl)methyl)pyridazin-3(2H)-one

A mixture of 6-Bromo-2H-pyridazin-3-one (1.00 g, 5.715 mmol, 1.00 equiv), 3-(chloromethyl)-5-fluoropyridine hydrochloride (0.83 g, 5.702 mmol, 1.10 equiv), and K₂CO₃ (2.37 g, 17.144 mmol, 3 equiv) in DMF (10 mL) was stirred for 1 h at room temperature. The resulting mixture was extracted with EtOAc, and the combined organic layers were dried over anhydrous Na₂SO₄ and filtered. The filtrate was concentrated under reduced pressure and the residue was purified using silica gel column chromatography which afforded 6-bromo-2-((5-fluoropyridin-3-yl)methyl)pyridazin-3(2H)-one, 800 mg (49.28%). LCMS (ES, m/z): 284 [M+H]⁺.

### Step 2: (1-((5-Fluoropyridin-3-yl)methyl)-6-oxo-1,6-dihydropyridazin-3-yl)boronic acid

A mixture of 6-Bromo-2-[(5-fluoropyridin-3-yl)methyl]pyridazin-3-one (1.0 g, 3.520 mmol, 1 equiv), 4,4,4′,4′,5,5,5′,5′-octamethyl-2,2′-bi(1,3,2-dioxaborolane) (1.79 g, 7.040 mmol, 2.00 equiv), KOAc (690.93 mg, 7.040 mmol, 2.00 equiv), and Pd(dppf)Cl₂ (257.56 mg, 0.352 mmol, 0.10 equiv) and in dioxane (10 mL) was stirred for 2 h at 80° C. under argon atmosphere. The crude product was used directly in the next step without further purification. LCMS (ES, m/z): 250 [M+H]⁺.

### Step 3: 6-(3-Bromo-1,2,4-thiadiazol-5-yl)-2-((5-fluoropyridin-3-yl)methyl)pyridazin-3(2H)-one

A mixture of 3-bromo-5-chloro-1,2,4-thiadiazole (702 mg, 3.520 mmol, 1.00 equiv), (1-((5-fluoropyridin-3-yl)methyl)-6-oxo-1,6-dihydropyridazin-3-yl)boronic acid (880 mg, 3.520 mmol, 1.00 equiv), K₂CO₃ (972.84 mg, 7.040 mmol, 2.00 equiv), and Pd(dppf)Cl₂ (257.53 mg, 0.352 mmol, 0.10 equiv) in dioxane (10 mL) was stirred for 18 h at 80° C. under argon atmosphere. The resulting mixture was concentrated under reduced pressure and the residue purified by silica gel column chromatography to afford 6-(3-bromo-1,2,4-thiadiazol-5-yl)-2-((5-fluoropyridin-3-yl)methyl)pyridazin-3(2H)-one (620 mg, 47.85%). LCMS (ES, m/z): 368 [M+H]⁺.

### Step 4: 6-(3-(5-chloropyridin-3-yl)-1,2,4-thiadiazol-5-yl)-2-((5-fluoropyridin-3-yl)methyl)pyridazin-3(2H)-one

A mixture of 6-(3-bromo-1,2,4-thiadiazol-5-yl)-2-((5-fluoropyridin-3-yl)methyl)pyridazin-3(2H)-one (310 mg, 0.842 mmol, 1.00 equiv), 5-chloropyridin-3-ylboronic acid (158.99 mg, 1.010 mmol, 1.20 equiv), K₂CO₃ (232.73 mg, 1.684 mmol, 2.00 equiv), and Pd(dppf)Cl₂ (61.61 mg, 0.084 mmol, 0.10 equiv) in dioxane (5 mL) and H₂O (1 mL) was stirred for 3 h at 80° C. under argon atmosphere. The resulting mixture was extracted with EtOAc and the combined organic layers were dried over anhydrous Na₂SO₄, filtered and the filtrate was concentrated under reduced pressure. The residue was purified by silica gel column chromatography to afford 6-(3-(5-chloropyridin-3-yl)-1,2,4-thiadiazol-5-yl)-2-((5-fluoropyridin-3-yl)methyl)pyridazin-3(2H)-one (180 mg, 53.34%). LCMS (ES, m/z): 401 [M+H]⁺.

The following compounds of formula Ia and 1b were synthesized following Examples 1 and 2:

| Compound | | | |
|---|---|---|---|
| No. | Structure | NMR | [M + H]⁺ |
| 1 | 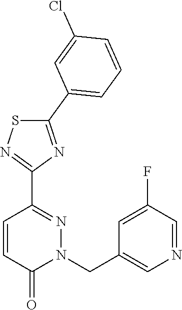 [SMILES: O=c1ccc(-c2nsc(-c3cccc(Cl)c3)n2)nn1Cc1cncc(F)c1] | 1H NMR (400 MHZ, DMSO-d6) δ 8.55-8.50 (m, 2H), 8.32 (d, J = 9.6 Hz, 1H), 8.19 (s, 1H), 8.09 (d, J = 6.8 Hz, 1H), 7.78-7.19 (m, 3H), 7.21 (d, J = 9.6 Hz, 1H), 5.50 (s, 2H). 19F NMR (376 MHz, DMSO-d6) δ-127.14 (1F). | 400.1 |
| 2 | 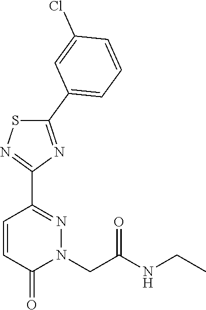 [SMILES: CCNC(=O)Cn1nc(-c2nsc(-c3cccc(Cl)c3)n2)ccc1=O] | 1H NMR (300 MHZ, DMSO-d6) δ 8.31 (d, J = 9.9 Hz, 1H), 8.20-8.19 (m, 2H), 8.18-8.09 (m, 1H), 7.74-7.73 (m, 1H), 7.66 (t, J = 7.8 Hz, 1H), 7.16 (d, J = 9.9 Hz, 1H), 4.81 (s, 2H), 3.14-3.10 (m, 2H), 1.06 (t, J = 7.2 Hz, 3H). | 376.0 |
| 3 | 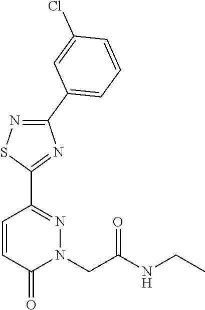 [SMILES: CCNC(=O)Cn1nc(-c2nc(-c3cccc(Cl)c3)ns2)ccc1=O] | | 376.0 |
| 4 | 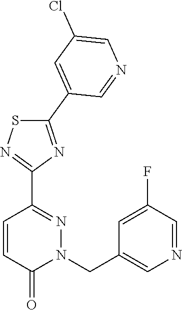 [SMILES: O=c1ccc(-c2nsc(-c3cncc(Cl)c3)n2)nn1Cc1cncc(F)c1] | 1H NMR (300 MHZ, DMSO-d6) δ 9.27 (s, 1H), 8.91 (s, 1H), 8.67 (s, 1H), 8.55 (d, J = 9.6 Hz, 2H), 8.34 (d, J = 9.6 Hz, 1H), 7.48- 7.71 (m, 1H), 7.22 (d, J = 9.9 Hz, 1H), 5.50 (s, 2H). 19F NMR (282 MHZ, DMSO-d6) δ-127.12 (1F). | 401.1 |
| 5 | 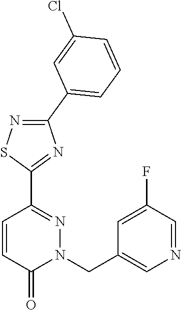 [SMILES: O=c1ccc(-c2nc(-c3cccc(Cl)c3)ns2)nn1Cc1cncc(F)c1] | 1H NMR (400 MHZ, DMSO-d6) δ 8.56-8.52 (m, 2H), 8.29-8.23 (m, 3H), 7.77 (dd, J = 9.6, 2.8 Hz, 1H), 7.74-7.60 (m, 2H), 7.25 (d, J = 9.6 Hz, 1H), 5.46 (s, 2H). 19F NMR (376 MHz, DMSO-d6) δ-127.08 (1F). | 400.0 |
| 6 | 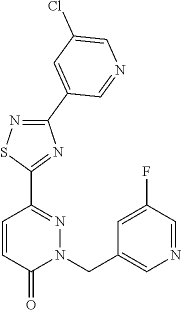 [SMILES: O=c1ccc(-c2nc(-c3cncc(Cl)c3)ns2)nn1Cc1cncc(F)c1] | 1H NMR (400 MHZ, DMSO-d6) δ 9.38 (s, 1H), 8.84 (m, 1H), 8.66 (s, 1H), 8.59-8.50 (m, 2H), 8.29 (d, J = 9.6 Hz, 1H), 7.76 (d, J = 9.6, 1H), 7.27 (d, J = 9.6 Hz, 1H), 5.46 (s, 2H). 19F NMR (376 MHz, DMSO-d6) δ- 127.08 (1F). | 401.0 |
| 7 | 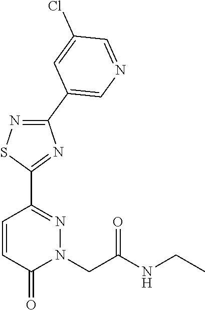 [SMILES: CCNC(=O)Cn1nc(-c2nc(-c3cncc(Cl)c3)ns2)ccc1=O] | | 377.0 |
| 8 | 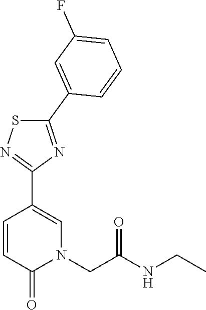 [SMILES: CCNC(=O)Cn1cc(-c2nsc(-c3cccc(F)c3)n2)ccc1=O] | 1H NMR (400 MHZ, DMSO-d6) δ 8.73 (s, 1H), 8.28-8.21 (m, 2H), 8.00- 7.95 (m, 2H), 7.70-7.66 (m, 1H), 7.54-7.51 (m, 1H), 6.55 (d, J = 9.6 Hz, 1H), 4.70 (s, 2H), 3.12-3.09 (m, 2H), 1.06 (t, J = 7.2 Hz, 3H). 19F NMR (376 MHz, DMSO-d6) δ-111.55 (1F). | 359.0 |
| 9 | 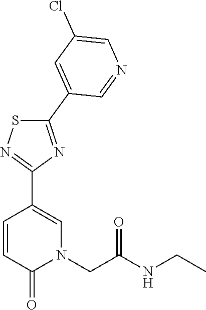 [SMILES: CCNC(=O)Cn1cc(-c2nsc(-c3cncc(Cl)c3)n2)ccc1=O] | | 376.0 |
| | 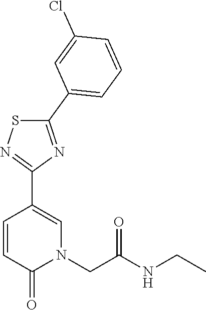 [SMILES: CCNC(=O)Cn1cc(-c2nsc(-c3cccc(Cl)c3)n2)ccc1=O] | 1H NMR (300 MHz, DMSO-d6) δ 8.74 (s, 1H), 8.27-8.21 (m, 3H), 8.07 (d, J = 7.5 Hz, 1H), 7.72-7.65 (m, 2H), 6.55 (d, J = 9.3 Hz, 1H), 4.70 (s, 2H), 3.17-3.08 (m, 2H), 1.06 (t, J = 7.2 Hz, 3H). | |
| 10 | 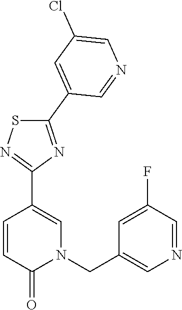 [SMILES: O=c1ccc(-c2nsc(-c3cncc(Cl)c3)n2)cn1Cc1cncc(F)c1] | | 375.1 |
| 11 | 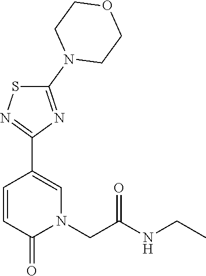 [SMILES: CCNC(=O)Cn1cc(-c2nsc(N3CCOCC3)n2)ccc1=O] | | 399.95 |
| 12 | 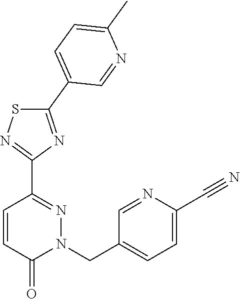 [SMILES: Cc1ccc(-c2nc(-c3ccc(=O)n(Cc4ccc(C#N)nc4)n3)ns2)cn1] | | 349.95 |
| 13 | 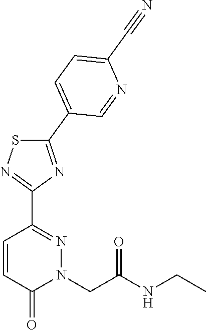 [SMILES: CCNC(=O)Cn1nc(-c2nsc(-c3ccc(C#N)nc3)n2)ccc1=O] | | 388.05 |
| 14 | 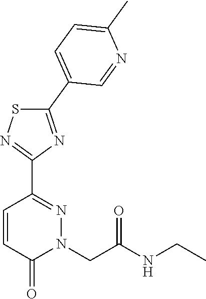 [SMILES: CCNC(=O)Cn1nc(-c2nsc(-c3ccc(C)nc3)n2)ccc1=O] | | 368.20 |
| 15 | 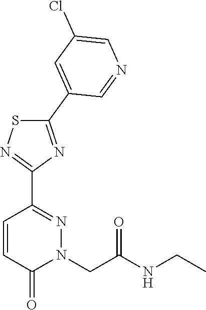 [SMILES: CCNC(=O)Cn1nc(-c2nsc(-c3cncc(Cl)c3)n2)ccc1=O] | | 357.00 |
| 16 | 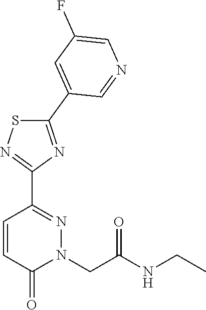 [SMILES: CCNC(=O)Cn1nc(-c2nsc(-c3cncc(F)c3)n2)ccc1=O] | ¹H NMR (400 MHZ, DMSO- d₆) δ 9.26 (s, 1H), 8.90 (s, 1H), 8.67 (s, 1H), 8.34-8.31 (m, 1H), 8.22 (s, 1H), 7.17 (dd, J = 9.6, 2.8 Hz, 1H), 4.80 (s, 2H), 3.16-3.07 (m, 2H), 1.06 (t, J = 7.2 Hz, 3H). | 377.0 |
| 17 | 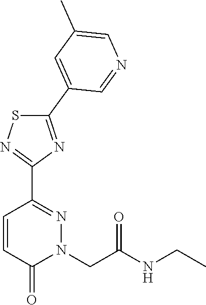 [SMILES: CCNC(=O)Cn1nc(-c2nsc(-c3cncc(C)c3)n2)ccc1=O] | | 360.95 |
| 18 | 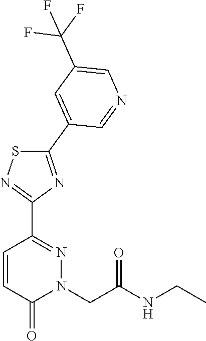 [SMILES: CCNC(=O)Cn1nc(-c2nsc(-c3cncc(C(F)(F)F)c3)n2)ccc1=O] | | 357.00 |
| 19 | 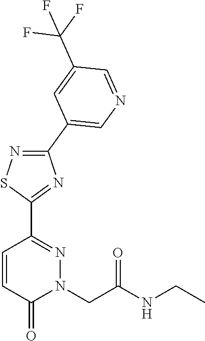 [SMILES: CCNC(=O)Cn1nc(-c2nc(-c3cncc(C(F)(F)F)c3)ns2)ccc1=O] | | 411.00 |
| 20 | 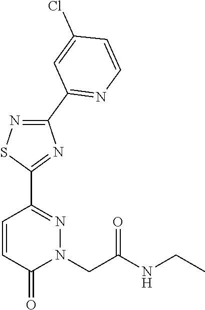 [SMILES: CCNC(=O)Cn1nc(-c2nc(-c3cc(Cl)ccn3)ns2)ccc1=O] | | 411.2 |
| 21 | 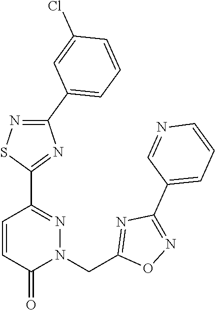 [SMILES: O=c1ccc(-c2nc(-c3cccc(Cl)c3)ns2)nn1Cc1nc(-c2cccnc2)no1] | | 450.05 |
| 22 | 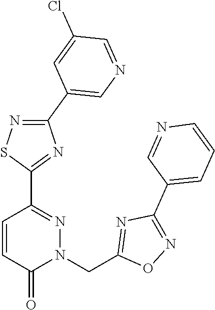 [SMILES: O=c1ccc(-c2nc(-c3cncc(Cl)c3)ns2)nn1Cc1nc(-c2cccnc2)no1] | | |
| 23 | 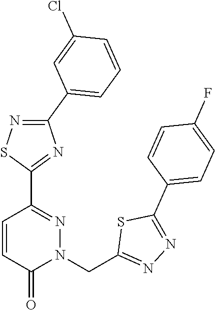 [SMILES: O=c1ccc(-c2nc(-c3cccc(Cl)c3)ns2)nn1Cc1nnc(-c2ccc(F)cc2)s1] | | 451.10 |
| 24 | 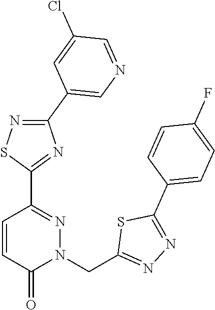 [SMILES: O=c1ccc(-c2nc(-c3cncc(Cl)c3)ns2)nn1Cc1nnc(-c2ccc(F)cc2)s1] | | 483.10 |
| 25 | 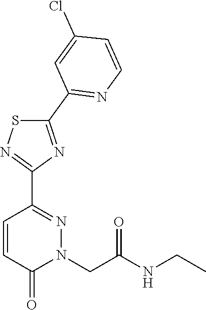 [SMILES: CCNC(=O)Cn1nc(-c2nsc(-c3cc(Cl)ccn3)n2)ccc1=O] | | 484.10 |
| 26 | 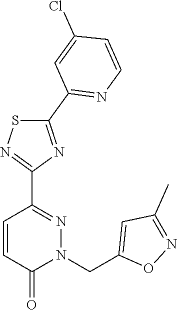 [SMILES: Cc1cc(Cn2nc(-c3nsc(-c4cc(Cl)ccn4)n3)ccc2=O)on1] | | |
| 27 | 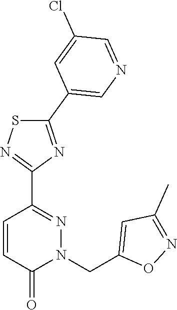 [SMILES: Cc1cc(Cn2nc(-c3nsc(-c4cncc(Cl)c4)n3)ccc2=O)on1] | | 387.10 |
| 28 | 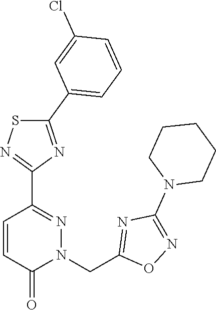 [SMILES: O=c1ccc(-c2nsc(-c3cccc(Cl)c3)n2)nn1Cc1nc(N2CCCCC2)no1] | | 386.10 |
| 29 | 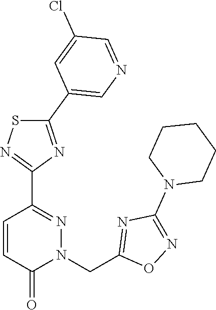 [SMILES: O=c1ccc(-c2nsc(-c3cncc(Cl)c3)n2)nn1Cc1nc(N2CCCCC2)no1] | | 456.15 |
| 30 | 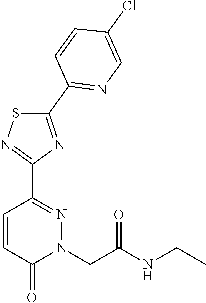 [SMILES: CCNC(=O)Cn1nc(-c2nsc(-c3ccc(Cl)cn3)n2)ccc1=O] | | 457.10 |
| 31 | 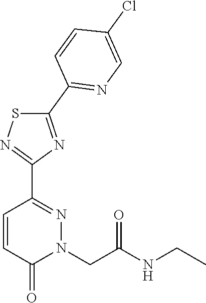 [SMILES: CCNC(=O)Cn1nc(-c2nsc(-c3ccc(Cl)cn3)n2)ccc1=O] | | 376.95 |

### Example 3: 2-(3-(5-(5-Chloropyridin-3-yl)-1,2,4-oxadiazol-3-yl)-6-oxopyridazin-1(6H)-yl)-N-ethylacetamide (Compound 52)

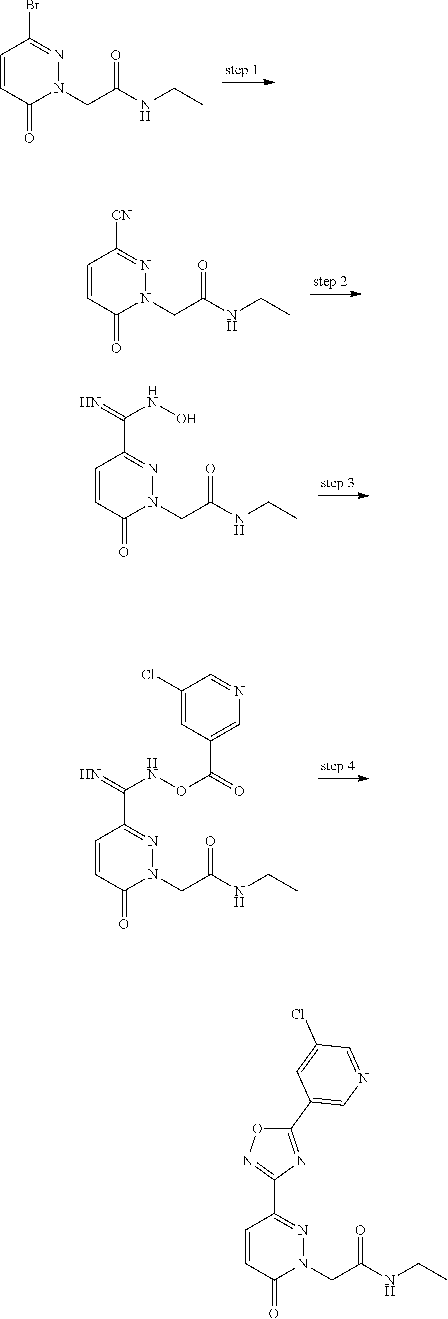 [SMILES: CCNC(=O)Cn1nc(Br)ccc1=O ; CCNC(=O)Cn1nc(C#N)ccc1=O ; CCNC(=O)Cn1nc(C(=N)NO)ccc1=O ; CCNC(=O)Cn1nc(C(=N)NOC(=O)c2cncc(Cl)c2)ccc1=O ; CCNC(=O)Cn1nc(-c2noc(-c3cncc(Cl)c3)n2)ccc1=O]

### Step 1: 2-(3-Cyano-6-oxopyridazin-1(6H)-yl)-N-ethylacetamide

A mixture of 2-(3-bromo-6-oxopyridazin-1-yl)-N-ethylacetamide (2.00 g, 7.690 mmol, 1.00 equiv), Zn(CN)₂ (0.99 g, 8.459 mmol, 1.10 equiv), DIEA (0.10 g, 0.769 mmol, 0.10 equiv) and 2nd generation XantPhos precatalyst (0.68 g, 0.769 mmol, 0.10 equiv) in DMAc (20 mL) was stirred for 18 h at 100° C. under argon atmosphere. The mixture was cooled to room temperature and the aqueous layer was extracted with EtOAc. The combined organic layers were dried over anhydrous Na₂SO₄, filtered and the filtrate was concentrated under reduced pressure. The residue was purified by silica gel column chromatography to afford 2-(3-cyano-6-oxopyridazin-1(6H)-yl)-N-ethylacetamide (1.2 g, 75.68%). LCMS (ES, m/z): 207 [M+H]⁺.

### Step 2: N-Ethyl-2-(3-(N-hydroxycarbamimidoyl)-6-oxopyridazin-1(6H)-yl)acetamide

A mixture of 2-(3-cyano-6-oxopyridazin-1(6H)-yl)-N-ethylacetamide (950 mg, 4.607 mmol, 1.00 equiv), hydroxylamine hydrochloride (384.17 mg, 5.528 mmol, 1.20 equiv), and K₂CO₃ (1.27 g, 9.214 mmol, 2.00 equiv) in MeOH (15 mL) was stirred for 1 h at 70° C. under argon atmosphere. The mixture was cooled to room temperature and the resulting mixture was concentrated under reduced pressure. The resulting residue was purified by silica gel column chromatography to afford N-ethyl-2-(3-(N-hydroxycarbamimidoyl)-6-oxopyridazin-1(6H)-yl)acetamide (450 mg, 40.83%) as a white solid. LCMS (ES, m/z): 240 [M+H]⁺.

### Step 3: 2-(3-(N-((5-Chloronicotinoyl)oxy)carbamimidoyl)-6-oxopyridazin-1(6H)-yl)-N-ethylacetamide

A mixture of N-ethyl-2-(3-(N-hydroxycarbamimidoyl)-6-oxopyridazin-1(6H)-yl)acetamide (400 mg, 1.672 mmol, 1.00 equiv), 5-chloropyridine-3-carboxylic acid (316.11 mg, 2.006 mmol, 1.20 equiv), N,N′-diisopropylcarbodiimide (DIC) (316.52 mg, 2.508 mmol, 1.50 equiv) and HOBt (338.90 mg, 2.508 mmol, 1.50 equiv) in DMF (10 mL) was stirred at room temperature for 2 h under argon atmosphere. The residue was purified by trituration with MeCN (30 mL) resulting in 2-(3-(N-((5-chloronicotinoyl)oxy)carbamimidoyl)-6-oxopyridazin-1(6H)-yl)-N-ethylacetamide (500 mg, 78.95%). LCMS (ES, m/z): 379 [M+H]⁺.

### Step 4: 2-(3-(5-(5-Chloropyridin-3-yl)-1,2,4-oxadiazol-3-yl)-6-oxopyridazin-1(6H)-yl)-N-ethylacetamide

A mixture of 2-(3-(N-((5-chloronicotinoyl)oxy)carbamimidoyl)-6-oxopyridazin-1(6H)-yl)-N-ethylacetamide (500 mg, 1.320 mmol, 1.00 equiv) in pyridine (10 mL) was stirred for 4 h at 100° C. under N₂ atmosphere. The mixture was cooled to room temperature and the resulting mixture was concentrated under reduced pressure. The residue was purified by trituration with MeCN (50 mL) affording 2-(3-(5-(5-chloropyridin-3-yl)-1,2,4-oxadiazol-3-yl)-6-oxopyridazin-1(6H)-yl)-N-ethylacetamide (260 mg, 54.62%)s. LCMS (ES, m/z): 361 [M+H]⁺.

The following compounds of formula II were synthesized following Example 3:

| Compound No. | Structure | NMR | [M + H]⁺ |
|---|---|---|---|
| 51 | 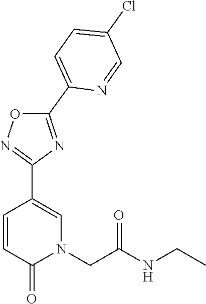 [SMILES: CCNC(=O)Cn1cc(-c2noc(-c3ccc(Cl)cn3)n2)ccc1=O] | ¹H NMR (400 MHz, DMSO- d₆) δ 8.94 (d, J = 2.0 Hz, 1H), 8.37-8.22 (m, 3H), 8.07 (d, J = 9.6 Hz, 1H), 7.19 (d, J = 10.0 Hz, 1H), 4.80 (s, 2H), 3.14- 3.11 (m, 2H), 1.05 (t, J = 7.2 Hz, 3H). | 361.0 |
| 52 | 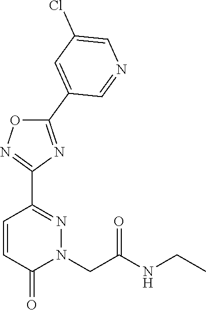 [SMILES: CCNC(=O)Cn1nc(-c2noc(-c3cncc(Cl)c3)n2)ccc1=O] | ¹H NMR (300 MHz, DMSO- d₆) δ 9.36 (s, 1H), 9.07 (s, 1H), 8.76 (d, J = 2.4 Hz, 1H), 8.31 (t, J = 5.4 Hz, 1H), 8.15 (d, J = 9.6 Hz, 1H), 7.27 (d, J = 9.6 Hz, 1H), 4.88 (s, 2H), 3.24- 3.15 (m, 2H), 1.13 (t, J = 7.2 Hz, 3H). | 361.0 |
| 53 | 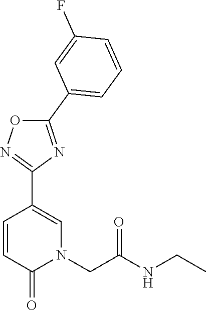 [SMILES: CCNC(=O)Cn1cc(-c2noc(-c3cccc(F)c3)n2)ccc1=O] | ¹H NMR (300 MHz, DMSO- d₆) δ 8.53 (s, 1H), 8.28 (s, 1H), 8.04-7.95 (m, 3H), 7.79- 7.71 (m, 1H), 7.64-7.60 (m, 1H), 6.58 (d, J = 9.6 Hz, 1H), 4.70 (s, 2H), 3.12 (q, J = 6.6 Hz, 2H), 1.06 (t, J = 7.2 Hz, 3H). ¹⁹F NMR (282 MHz, DMSO-d₆) δ −111.05 (1F). | 343.0 |
| 54 | 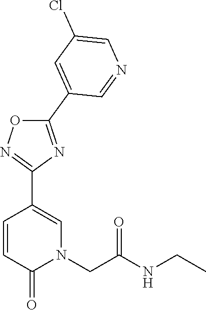 [SMILES: CCNC(=O)Cn1cc(-c2noc(-c3cncc(Cl)c3)n2)ccc1=O] | | 360.00 |
| 55 | 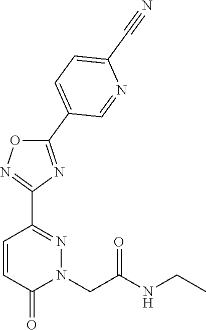 [SMILES: CCNC(=O)Cn1nc(-c2noc(-c3ccc(C#N)nc3)n2)ccc1=O] | | 352.10 |
| 56 | 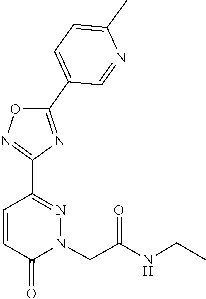 [SMILES: CCNC(=O)Cn1nc(-c2noc(-c3ccc(C)nc3)n2)ccc1=O] | | 341.10 |
| 57 | 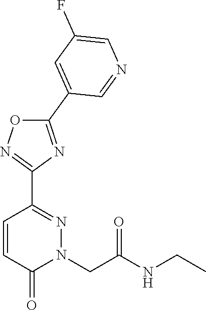 [SMILES: CCNC(=O)Cn1nc(-c2noc(-c3cncc(F)c3)n2)ccc1=O] | ¹H NMR (300 MHz, DMSO- d₆) δ 9.30 (s, 1H), 9.04 (s, 1H), 8.61 (d, J = 8.7 Hz, 1H), 8.32 (s, 1H), 8.15 (d, J = 9.6 Hz, 1H), 7.27 (d, J = 9. 9 Hz, 1H), 4.88 (s, 2H), 3.21-3.17 (m, 2H), 1.13 (t, J = 7.2 Hz, 3H). ¹⁹F NMR (282 MHz, DMSO- d₆) δ −124.90 (1F). | 345.1 |
| 58 | 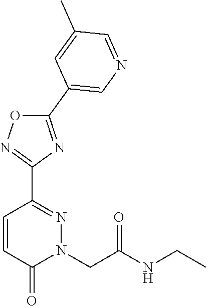 [SMILES: CCNC(=O)Cn1nc(-c2noc(-c3cncc(C)c3)n2)ccc1=O] | ¹H NMR (300 MHz, DMSO- d₆) δ 9.22 (s, 1H), 8.82 (s, 1H), 8.49 (s, 1H), 8.30 (t, J = 5.7 Hz, 1H), 8.14 (d, J = 9.9 Hz, 1H), 7.26 (d, J = 9.9 Hz, 1H), 4.87 (s, 2H), 3.24-3.15 (m, 2H), 2.57 (s, 3H), 1.13 (t, J = 7.2 Hz, 3H). | 341.1 |

### Example 4: 6-(5-(5-Chloropyridin-3-yl)-1,2,4-oxadiazol-3-yl)-2-((5-fluoropyridin-3-yl)methyl)pyridazin-3(2H)-one (Compound 71)

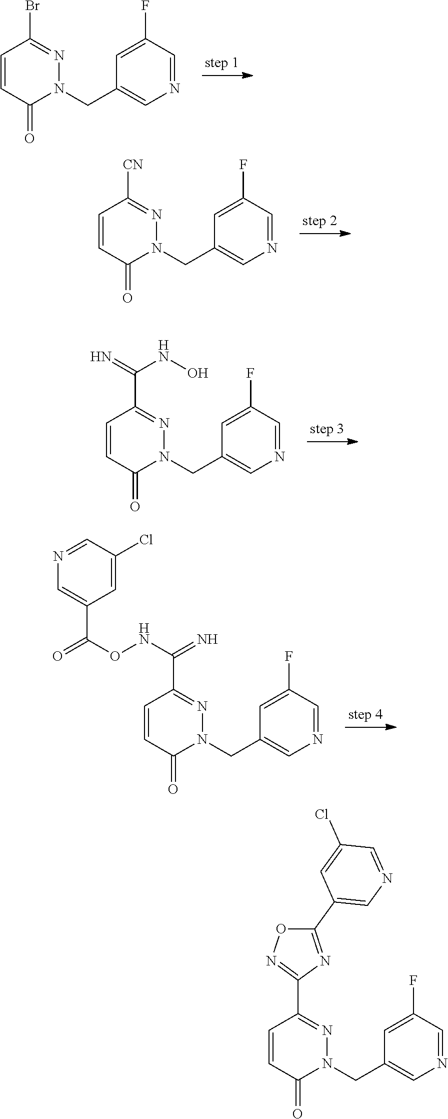

### Step 1: 1-((5-Fluoropyridin-3-yl)methyl)-6-oxo-1,6-dihydropyridazine-3-carbonitrile

To a stirred solution of 6-bromo-2-[(5-fluoropyridin-3-yl)methyl]pyridazin-3-one (1.0 g, 3.520 mmol, 1.00 equiv) and Zn(CN)₂ (1.03 g, 8.800 mmol, 2.50 equiv) in DMAc (12 mL) was added Zn powder (0.03 g, 0.528 mmol, 0.15 equiv) and Pd(dppf)Cl₂ (0.39 g, 0.528 mmol, 0.15 equiv) portion-wise. The reaction mixture was irradiated with microwave radiation for 1 h at 120° C. and then cooled to room temperature. The resulting mixture was extracted with EtOAc and the combined organic layers were washed with brine, dried over anhydrous Na₂SO₄ and filtered. The filtrate was concentrated under reduced pressure and the residue was purified by silica gel column chromatography to afford 1-[(5-fluoropyridin-3-yl)methyl]-6-oxopyridazine-3-carbonitrile (680 mg, 83.92%). LCMS (ES, m/z): 231 [M+H].

### Step 2: 1-((5-Fluoropyridin-3-yl)methyl)-N-hydroxy-6-oxo-1,6-dihydropyridazine-3-carboximidamide

To a stirred solution of 1-[(5-fluoropyridin-3-yl)methyl]-6-oxopyridazine-3-carbonitrile (660 mg, 2.867 mmol, 1.00 equiv) and NH₂OH·HCl (239.08 mg, 3.440 mmol, 1.20 equiv) in MeOH (10 mL) was added K₂CO₃ (792.48 mg, 5.734 mmol, 2.00 equiv) portion wise at 80° C. The reaction mixture was cooled to room temperature, filtered, and the filter cake was washed with MeOH. The resulting filtrate was concentrated under reduced pressure providing 1-((5-fluoropyridin-3-yl)methyl)-N-hydroxy-6-oxo-1,6-dihydropyridazine-3-carboximidamide (600 mg, 79.50%). LCMS (ES, m/z): 264 [M+H]⁺.

### Step 3: N-((5-Chloronicotinoyl)oxy)-1-((5-fluoropyridin-3-yl)methyl)-6-oxo-1,6-dihydropyridazine-3-carboximidamide

To a stirred solution of 1-((5-fluoropyridin-3-yl)methyl)-N-hydroxy-6-oxo-1,6-dihydropyridazine-3-carboximidamide (200 mg, 0.760 mmol, 1.00 equiv) and 5-chloropyridine-3-carboxylic acid (143.65 mg, 0.912 mmol, 1.20 equiv) in DMF (3 mL) was added HOBt (154.00 mg, 1.140 mmol, 1.50 equiv) and N,N′-diisopropylcarbodiimide (DIC) (143.83 mg, 1.140 mmol, 1.50 equiv) portion wise at room temperature. The resulting residue was purified by reverse flash chromatography (column, C18; mobile phase, MeCN in water, 10% to 50% gradient in 10 min; detector, UV 254 nm) to provide N-((5-chloronicotinoyl)oxy)-1-((5-fluoropyridin-3-yl)methyl)-6-oxo-1,6-dihydropyridazine-3-carboximidamide (220 mg, 71.89%). LCMS (ES, m/z): 403 [M+H]⁺.

### Step 4: 6-(5-(5-Chloropyridin-3-yl)-1,2,4-oxadiazol-3-yl)-2-((5-fluoropyridin-3-yl)methyl)pyridazin-3(2H)-one

N-((5-chloronicotinoyl)oxy)-1-((5-fluoropyridin-3-yl)methyl)-6-oxo-1,6-dihydropyridazine-3-carboximidamide (200 mg, 0.497 mmol, 1.00 equiv) in pyridine (5 mL) was stirred 18 h at 100° C. under open atmosphere. The mixture cooled to room temperature and the crude product (150 mg) was purified by Prep-HPLC (Column: Xselect CSH C18 OBD Column 30*150 mm 5 μm, n; Mobile Phase A: Water (0.1% FA), Mobile Phase B: ACN; Flow rate: 60 mL/min; Gradient: 24% B to 56% B in 9 min, 56% B; Wave Length: 254/220 nm; RT1 (min): 8.78; Number Of Runs: 0) to afford 6-(5-(5-chloropyridin-3-yl)-1,2,4-oxadiazol-3-yl)-2-((5-fluoropyridin-3-yl)methyl)pyridazin-3(2H)-one (40 mg, 20.94%). LCMS (ES, m/z): 385.1 [M+H]⁺.

The following compounds of formula III and IV were synthesized following Example 4:

| Compound No. | Structure | NMR | [M + H]⁺ |
|---|---|---|---|
| 70 | 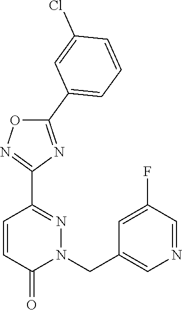 [SMILES: O=c1ccc(-c2noc(-c3cccc(Cl)c3)n2)nn1Cc1cncc(F)c1] | ¹H NMR (400 MHz, DMSO- d₆) δ 8.55 (s, 1H), 8.49 (s, 1H), 8.18-8.15 (m, 2H), 8.07 (d, J = 9.6 Hz, 1H), 7.73- 7.69 (m, 3H), 7.23 (d, J = 9.6 Hz, 1H), 5.49 (s, 2H). ¹⁹F NMR (376 MHz, DMSO-d₆) δ −127.10 (1F). | 384.1 |
| 71 | 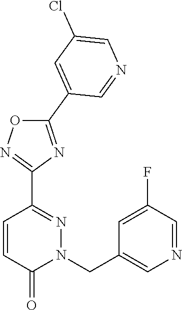 [SMILES: O=c1ccc(-c2noc(-c3cncc(Cl)c3)n2)nn1Cc1cncc(F)c1] | ¹H NMR (400 MHz, DMSO- d₆) δ 9.28 (s, 1H), 8.99 (s, 1H), 8.67 (s, 1H), 8.55 (s, 1H), 8.49 (s, 1H), 8.08 (d, J = 9.6 Hz, 1H), 7.72-7.69 (m, 1H), 7.24 (d, J = 9.6 Hz, 1H), 5.49 (s, 2H). ¹⁹F NMR (376 MHz, DMSO-d₆) δ −127.10 (1F). | 385.1 |
| 72 | 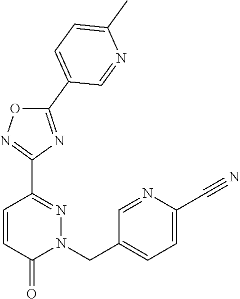 [SMILES: Cc1ccc(-c2nc(-c3ccc(=O)n(Cc4ccc(C#N)nc4)n3)no2)cn1] | | 372.1 |
| 73 | 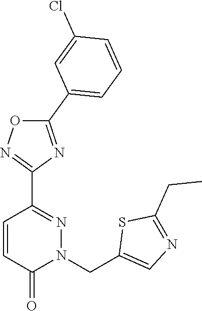 [SMILES: CCc1ncc(Cn2nc(-c3noc(-c4cccc(Cl)c4)n3)ccc2=O)s1] | | 400.05 |
| 74 | 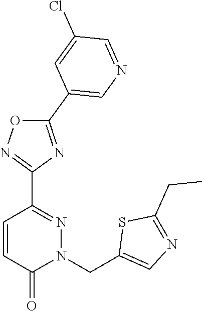 [SMILES: CCc1ncc(Cn2nc(-c3noc(-c4cncc(Cl)c4)n3)ccc2=O)s1] | | 400.95 |
| 75 | 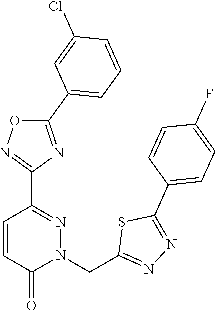 [SMILES: O=c1ccc(-c2noc(-c3cccc(Cl)c3)n2)nn1Cc1nnc(-c2ccc(F)cc2)s1] | | 467.10 |
| 76 | 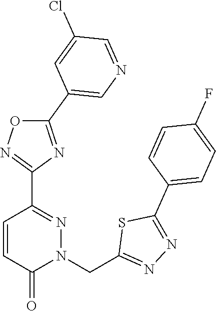 [SMILES: O=c1ccc(-c2noc(-c3cncc(Cl)c3)n2)nn1Cc1nnc(-c2ccc(F)cc2)s1] | | 467.90 |
| 77 | 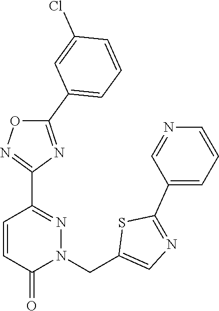 [SMILES: O=c1ccc(-c2noc(-c3cccc(Cl)c3)n2)nn1Cc1cnc(-c2cccnc2)s1] | | 449.10 |
| 78 | 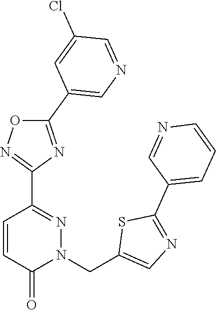 [SMILES: O=c1ccc(-c2noc(-c3cncc(Cl)c3)n2)nn1Cc1cnc(-c2cccnc2)s1] | | 449.90 |
| 79 | 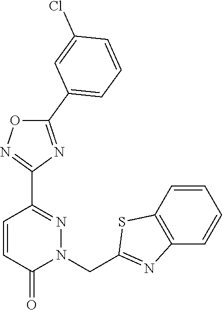 [SMILES: O=c1ccc(-c2noc(-c3cccc(Cl)c3)n2)nn1Cc1nc2ccccc2s1] | | 422.00 |
| 80 | 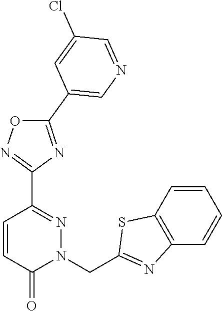 [SMILES: O=c1ccc(-c2noc(-c3cncc(Cl)c3)n2)nn1Cc1nc2ccccc2s1] | | 423.05 |
| 81 | 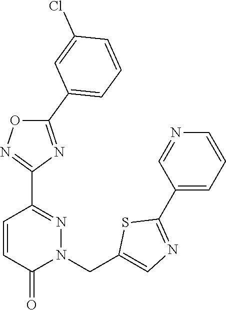 [SMILES: O=c1ccc(-c2noc(-c3cccc(Cl)c3)n2)nn1Cc1cnc(-c2cccnc2)s1] | | 449.10 |

### Example 5: 6-(5-(5-Chloropyridin-3-yl)-1,3,4-thiadiazol-2-yl)-2-((5-fluoropyridin-3-yl)methyl)pyridazin-3(2H)-one (Compound 104)

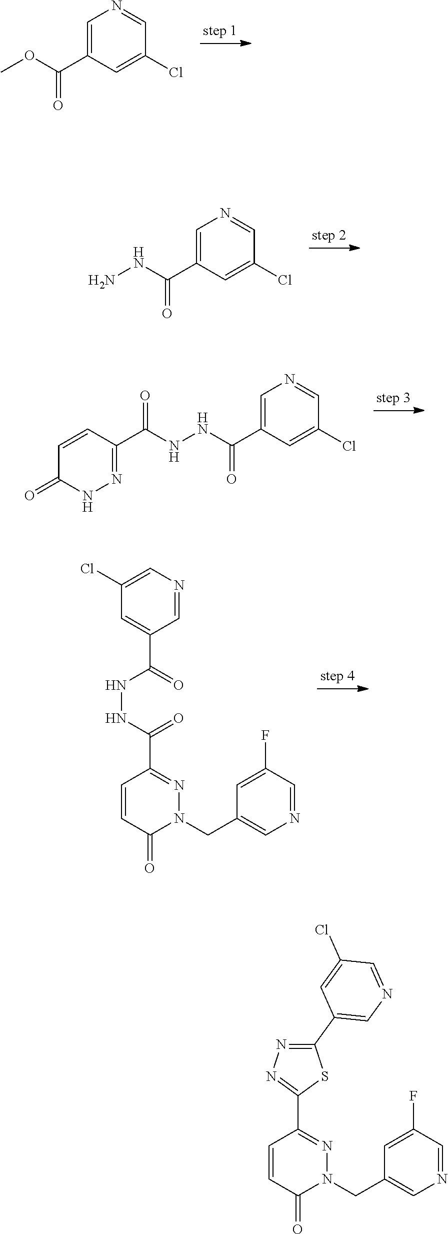 [SMILES: COC(=O)c1cncc(Cl)c1 ; NNC(=O)c1cncc(Cl)c1 ; O=C(NNC(=O)c1ccc(=O)[nH]n1)c1cncc(Cl)c1 ; O=C(NNC(=O)c1ccc(=O)n(Cc2cncc(F)c2)n1)c1cncc(Cl)c1 ; O=c1ccc(-c2nnc(-c3cncc(Cl)c3)s2)nn1Cc1cncc(F)c1]

### Step 1: 5-Chloropyridine-3-carbohydrazide

A mixture of methyl 5-chloropyridine-3-carboxylate (2.00 g, 11.656 mmol, 1.00 equiv) and hydrazine hydrate (1.81 g, 58.280 mmol, 5.00 equiv.) in MeOH (20 mL) was stirred for 2 h at 70° C. under nitrogen atmosphere. The resulting mixture was cooled to 0° C., filtered, and the filter cake was washed with MeOH. The filtrate was concentrated under reduced pressure to afford 5-chloropyridine-3-carbohydrazide (1.60 g, 80.00%). LCMS (ES, m/z): 172 [M+H]⁺

### Step 2: N′-(5-chloronicotinoyl)-6-oxo-1,6-dihydropyridazine-3-carboxydrazide

1-Ethyl-3-(3-dimethylaminopropyl)carbodiimide (EDCI) (1.20 g, 6.294 mmol, 1.20 equiv.) and HOBt (850.54 mg, 6.294 mmol, 1.20 equiv.) were added in portions at 0° C. to a stirred mixture of 5-chloropyridine-3-carboxydrazide (900 mg, 5.245 mmol, 1.00 equiv.) and 6-oxo-1H-pyridazine-3-carboxylic acid (734.87 mg, 5.245 mmol, 1.00 equiv.) in DMF (10 mL). The resulting mixture was stirred for 1 h at RT under nitrogen atmosphere. The resulting mixture was diluted with water and extracted with EtOAc. The combined organic layers were washed with brine and dried over anhydrous Na₂SO₄. After filtration, the filtrate was concentrated under reduced pressure. The residue was purified by column chromatography to afford N′-(5-chloronicotinoyl)-6-oxo-1,6-dihydropyridazine-3-carboxydrazide (600 mg, 35.83%). L CMS (ES, m/z): 294 [M+H]+

### Step 3: 5-Chloro-N′-{1-[(5-fluoropyridin-3-yl)methyl]-6-oxopyridazine-3-carbonyl}pyridine-3-carbohydrazide

To a stirred mixture of N′-(5-chloronicotinoyl)-6-oxo-1,6-dihydropyridazine-3-carbohydrazide (500 mg, 1.703 mmol, 1.00 equiv), Cs₂CO₃ (1.66 g, 5.109 mmol, 3.00 equiv), and 3-(chloromethyl)-5-fluoropyridine hydrochloride (297.40 mg, 2.044 mmol, 1.20 equiv) in DMF (10 mL). The resulting mixture was filtered, the filter cake was washed with MeOH (3×10 mL). The filtrate was concentrated under reduced pressure. This resulted in 5-chloro-N′-{1-[(5-fluoropyridin-3-yl)methyl]-6-oxopyridazine-3-carbonyl}pyridine-3-carbohydrazide as a yellow solid. LCMS (ES, m/z): 403 [M+H]⁺

### Step 4: 6-(5-(5-Chloropyridin-3-yl)-1,3,4-thiadiazol-2-yl)-2-((5-fluoropyridin-3-yl)methyl)pyridazin-3(2H)-one

A mixture of 5-chloro-N′-{1-[(5-fluoropyridin-3-yl)methyl]-6-oxopyridazine-3-carbonyl}pyridine-3-carbohydrazide (180 mg, 0.447 mmol, 1.00 equiv) and Lawesson Reagent (361.50 mg, 0.894 mmol, 2.00 equiv) in THF (5 mL) was stirred for 2 h at 70° C. under N₂ atmosphere. The mixture was cooled to room temperature and the residue was purified by reverse flash chromatography (column, silica gel; mobile phase, MeCN in water, 0% to 100% gradient in 10 min; detector, UV 254,220 nm) to afford 6-[5-(5-chloropyridin-3-yl)-1,3,4-thiadiazol-2-yl]-2-[(5-fluoropyridin-3-yl)methyl]pyridazin-3-one (80 mg, 44.66%). LCMS (ES, m/z): 401 [M+H]⁺.

The following compounds of formula IV were synthesized following Example 5:

| Compound | | |
|---|---|---|
| No. | Structure | [M + H]⁺ |
| 90 | 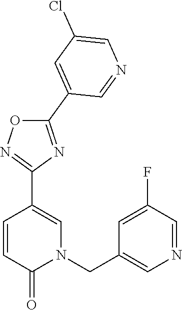 [SMILES: O=c1ccc(-c2noc(-c3cncc(Cl)c3)n2)cn1Cc1cncc(F)c1] | 384.00 |

### Example 6: 6-(5-(5-Chloropyridin-3-yl)-1,3,4-oxadiazol-2-yl)-2-((5-fluoropyridin-3-yl)methyl)pyridazin-3(2H)-one (Compound 103)

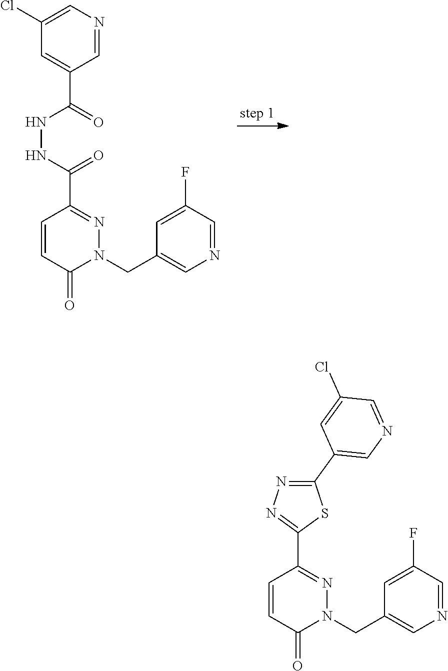 [SMILES: O=C(NNC(=O)c1ccc(=O)n(Cc2cncc(F)c2)n1)c1cncc(Cl)c1 ; O=c1ccc(-c2nnc(-c3cncc(Cl)c3)s2)nn1Cc1cncc(F)c1]

### Step 1: 6-[5-(5-Chloropyridin-3-yl)-1,3,4-oxadiazol-2-yl]-2-[(5-fluoropyridin-3-yl)methyl]pyridazin-3-one

To a stirred mixture of N′-(5-chloronicotinoyl)-1-((5-fluoropyridin-3-yl)methyl)-6-oxo-1,6-dihydropyridazine-3-carbohydrazide (120 mg, 0.298 mmol, 1.00 equiv) and 2-chloro-1,3-dimethyl-4,5-dihydro-1H-imidazol-3-ium (60.44 mg, 0.358 mmol, 1.20 equiv) in DCM (2 mL) was added TEA (90.45 mg, 0.894 mmol, 3.00 equiv) portion wise at room temperature. The resulting mixture was stirred for overnight at 40° C. under argon atmosphere. The reaction mixture was concentrated under reduced pressure and the crude product (120 mg) was purified by Prep-HPLC with the following conditions (Column: XBridge Shield RP18 OBD Column, 19*150 mm, 5 μm; Mobile Phase A: Water (0.1% FA), Mobile Phase B: ACN; Flow rate: 25 m/min; Gradient: 24% B to 43% B in 9 min, 43% B; Wave Length: 254/220 nm; RT1 (min): 7.98) to afford 6-[5-(5-chloropyridin-3-yl)-1,3,4-oxadiazol-2-yl]-2-[(5-fluoropyridin-3-yl)methyl]pyridazin-3-one (63 mg, 54.96%). LCMS (ES, m/z): 385 [M+H]⁺.

The following compounds of formula V were synthesized following Examples 5 and 6:

| Com- | | | |
|---|---|---|---|
| pound No. | Structure | NMR | [M + H]⁺ |
| 100 | 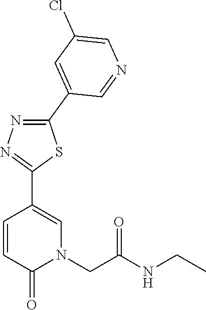 [SMILES: CCNC(=O)Cn1cc(-c2nnc(-c3cncc(Cl)c3)s2)ccc1=O] | | 376.2 |
| 101 | 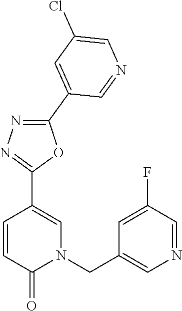 [SMILES: O=c1ccc(-c2nnc(-c3cncc(Cl)c3)o2)cn1Cc1cncc(F)c1] | | 384.1 |
| 102 | 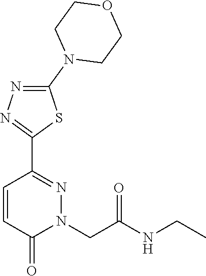 [SMILES: CCNC(=O)Cn1nc(-c2nnc(N3CCOCC3)s2)ccc1=O] | | 351.1 |
| 103 | 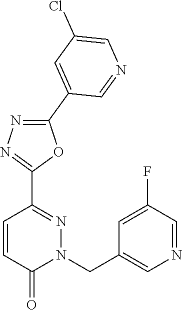 [SMILES: O=c1ccc(-c2nnc(-c3cncc(Cl)c3)o2)nn1Cc1cncc(F)c1] | ¹H NMR (400 MHz, DMSO-d₆) δ 9.21 (s, 1H), 8.93 (s, 1H), 8.57-8.53 (m, 3H), 8.20 (d, J = 9.6 Hz, 1H), 7.77-7.74 (m, 1H), 7.26 (d, J = 10.0 Hz, 1H), 5.49 (s, 2H). ¹⁹F NMR (376 MHz, DMSO- d₆) δ −127.11 (1F). | 385.1 |
| 104 | 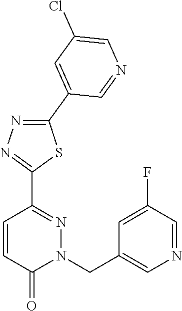 [SMILES: O=c1ccc(-c2nnc(-c3cncc(Cl)c3)s2)nn1Cc1cncc(F)c1] | ¹H NMR (400 MHz, DMSO-d₆) δ 9.21 (s, 1H), 8.86 (s, 1H), 8.61 (s, 1H), 8.56-8.51 (m, 2H), 8.25 (d, J = 9.6 Hz, 1H), 7.74- 7.72 (m, 1H), 7.25 (d, J = 9.6 Hz, 1H), 5.45 (s, 2H). ¹⁹F NMR (376 MHz, DMSO-d₆) δ −127.02 (1F). | 401.1 |
| 105 | 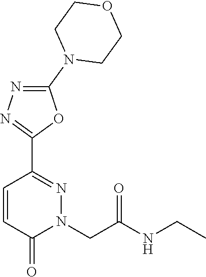 [SMILES: CCNC(=O)Cn1nc(-c2nnc(N3CCOCC3)o2)ccc1=O] | | 334.2 |
| 106 | 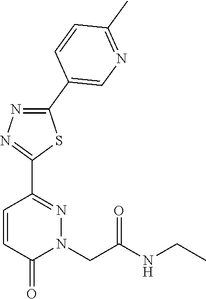 [SMILES: CCNC(=O)Cn1nc(-c2nnc(-c3ccc(C)nc3)s2)ccc1=O] | ¹H NMR (400 MHz, DMSO-d₆) δ 9.10 (s, 1H), 8.33 (d, J = 4.0 Hz, 1H), 8.25-8.20 (m, 2H), 7.49 (d, J = 8.0 Hz, 1H), 7.20 (d, J = 9.6 Hz, 1H), 4.75 (s, 2H), 3.14-3.11 (m, 2H), 2.57 (s, 3H), 1.05 (t, J = 7.2 Hz, 3H). | 357.1 |
| 107 | 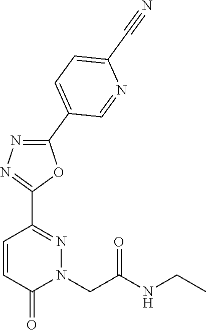 [SMILES: CCNC(=O)Cn1nc(-c2nnc(-c3ccc(C#N)nc3)o2)ccc1=O] | | 352.1 |
| 108 | 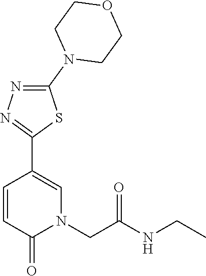 [SMILES: CCNC(=O)Cn1cc(-c2nnc(N3CCOCC3)s2)ccc1=O] | | 350.0 |
| 109 | 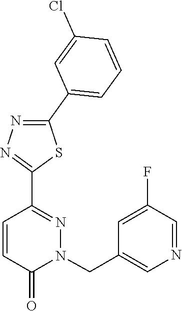 [SMILES: O=c1ccc(-c2nnc(-c3cccc(Cl)c3)s2)nn1Cc1cncc(F)c1] | ¹H NMR (400 MHz, DMSO-d₆) δ 8.56-8.52 (m, 2H), 8.23 (d, J = 9.6 Hz, 1H), 8.12 (s, 1H), 8.03 (d, J = 7.6 Hz, 1H), 7.75-7.66 (m, 2H), 7.62 (t, J = 7.8 Hz, 1H), 7.23 (d, J = 9.6 Hz, 1H), 5.44 (s, 2H). ¹⁹F NMR (376 MHz, DMSO-d₆) δ −127.03 (1F). | 400.1 |
| 110 | 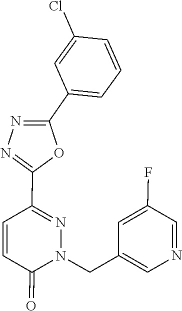 [SMILES: O=c1ccc(-c2nnc(-c3cccc(Cl)c3)o2)nn1Cc1cncc(F)c1] | ¹H NMR (300 MHz, DMSO-d₆) δ 8.56-8.52 (m, 2H), 8.19 (d, J = 9.6 Hz, 1H), 8.09-8.05 (m, 2H), 7.79-7.67 (m, 3H), 7.25 (d, J = 9.6 Hz, 1H), 5.50 (s, 2H). ¹⁹F NMR (282 MHz, DMSO-d₆) δ −127.12 (1F). | 384.1 |
| 111 | 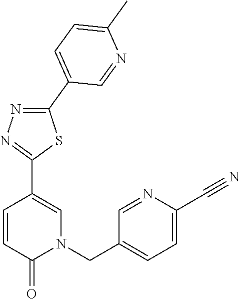 [SMILES: Cc1ccc(-c2nnc(-c3ccc(=O)n(Cc4ccc(C#N)nc4)c3)s2)cn1] | | 388.00 |
| 112 | 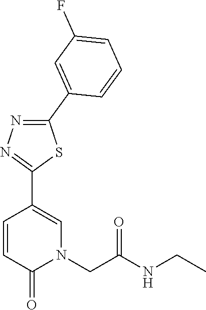 [SMILES: CCNC(=O)Cn1cc(-c2nnc(-c3cccc(F)c3)s2)ccc1=O] | | 359.10 |
| 113 | 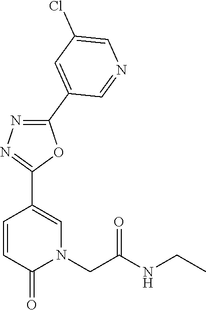 [SMILES: CCNC(=O)Cn1cc(-c2nnc(-c3cncc(Cl)c3)o2)ccc1=O] | | 360.00 |
| 114 | 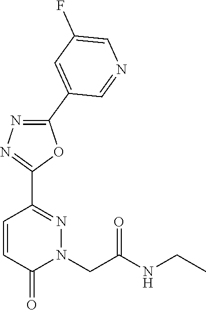 [SMILES: CCNC(=O)Cn1nc(-c2nnc(-c3cncc(F)c3)o2)ccc1=O] | ¹H NMR (300 MHz, DMSO-d₆) δ 9.14 (s, 1H), 8.90 (s, 1H), 8.43-8.39 (dd, J = 9.0, 1.8 Hz, 1H), 8.26-8.21 (m, 1H), 8.18 (d, J = 9.6 Hz, 1H), 7.23 (d, J = 9.9 Hz, 1H), 4.82 (s, 2H), 3.17-3.08 (m, 2H), 1.06 (t, J = 7.2 Hz, 3H). ¹⁹F NMR (282 MHz, DMSO-d₆) δ −125.14 (1F). | 344.8 |
| 115 | 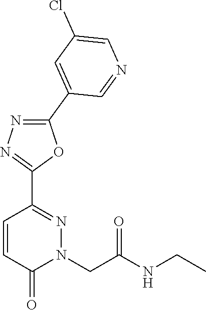 [SMILES: CCNC(=O)Cn1nc(-c2nnc(-c3cncc(Cl)c3)o2)ccc1=O] | ¹H NMR (300 MHz, DMSO-d₆) δ 9.21 (s, 1H), 8.93 (s, 1H), 8.57 (d, J = 4.2 Hz, 1H), 8.27-8.24 (m, 1H), 8.19 (s, 1H), 7.23 (d, J = 9.6 Hz, 1H), 4.82 (s, 2H), 3.31-3.08 (m, 2H), 1.06 (t, J = 7.2 Hz, 3H). | 361.0 |
| 116 | 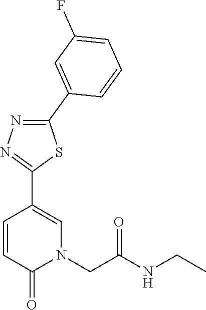 [SMILES: CCNC(=O)Cn1cc(-c2nnc(-c3cccc(F)c3)s2)ccc1=O] | ¹H NMR (300 MHz, DMSO-d₆) δ 9.55 (s, 1H), 9.29 (s, 1H), 8.77 (s, 1H), 8.26-8.22 (m, 2H), 7.24 (d, J = 9.9 Hz, 1H), 4.83 (s, 2H), 3.26-3.09 (m, 2H), 1.06 (t, J = 7.2 Hz, 3H). ¹⁹F NMR (282 MHz, DMSO-d₆) δ −61.06 (3F). | 359.0 |
| 117 | 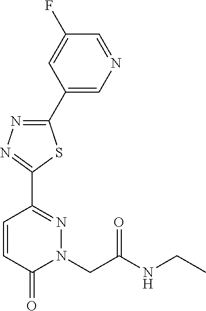 [SMILES: CCNC(=O)Cn1nc(-c2nnc(-c3cncc(F)c3)s2)ccc1=O] | ¹H NMR (300 MHz, DMSO-d₆) δ 9.15 (s, 1H), 8.84 (s, 1H), 8.47-8.42 (dd, J = 9.3, 1.8 Hz, 1H), 8.27 (s, 1H), 8.25-8.21 (m, 1H), 7.22 (d, J = 9.6 Hz, 1H), 4.76 (s, 2H), 3.17-3.08 (m, 2H), 1.06 (t, J = 7.2 Hz, 3H). ¹⁹F NMR (282 MHz, DMSO- d₆) δ −125.71 (1F). | 361.1 |
| 121 | 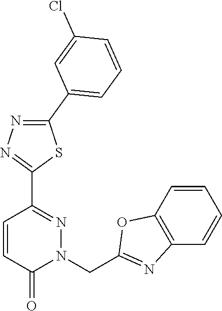 [SMILES: O=c1ccc(-c2nnc(-c3cccc(Cl)c3)s2)nn1Cc1nc2ccccc2o1] | | 421.80 |
| 122 | 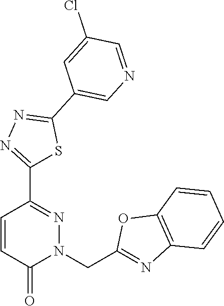 [SMILES: O=c1ccc(-c2nnc(-c3cncc(Cl)c3)s2)nn1Cc1nc2ccccc2o1] | | 423.10 |
| 123 | 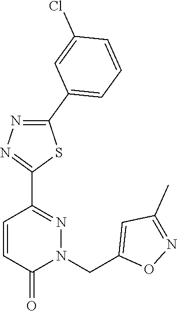 [SMILES: Cc1cc(Cn2nc(-c3nnc(-c4cccc(Cl)c4)s3)ccc2=O)on1] | | 386.00 |
| 124 | 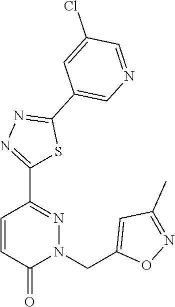 [SMILES: Cc1cc(Cn2nc(-c3nnc(-c4cncc(Cl)c4)s3)ccc2=O)on1] | | 387.10 |
| 125 | 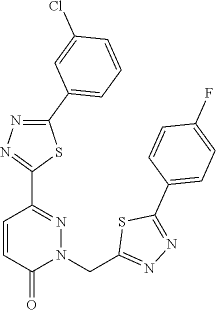 [SMILES: O=c1ccc(-c2nnc(-c3cccc(Cl)c3)s2)nn1Cc1nnc(-c2ccc(F)cc2)s1] | | 482.90 |
| 126 | 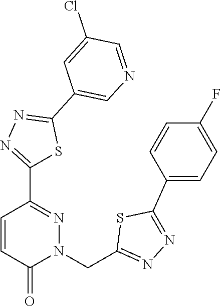 [SMILES: O=c1ccc(-c2nnc(-c3cncc(Cl)c3)s2)nn1Cc1nnc(-c2ccc(F)cc2)s1] | | 484.00 |
| 127 | 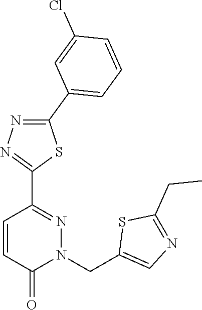 [SMILES: CCc1ncc(Cn2nc(-c3nnc(-c4cccc(Cl)c4)s3)ccc2=O)s1] | | 415.95 |
| 128 | 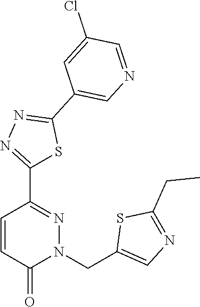 [SMILES: CCc1ncc(Cn2nc(-c3nnc(-c4cncc(Cl)c4)s3)ccc2=O)s1] | | 416.90 |
| 129 | 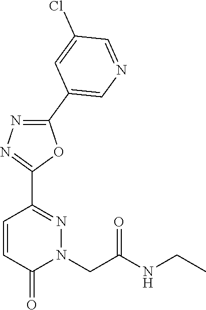 [SMILES: CCNC(=O)Cn1nc(-c2nnc(-c3cncc(Cl)c3)o2)ccc1=O] | | 361.00 |
| 130 | 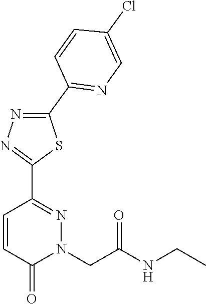 [SMILES: CCNC(=O)Cn1nc(-c2nnc(-c3ccc(Cl)cn3)s2)ccc1=O] | | 377.10 |

### Example 7: 2-(3-(2-(5-Chloropyridin-3-yl)-4-methylthiazol-5-yl)-6-oxopyridazin-1(6H)-yl)-N-ethylacetamide (Compound 151)

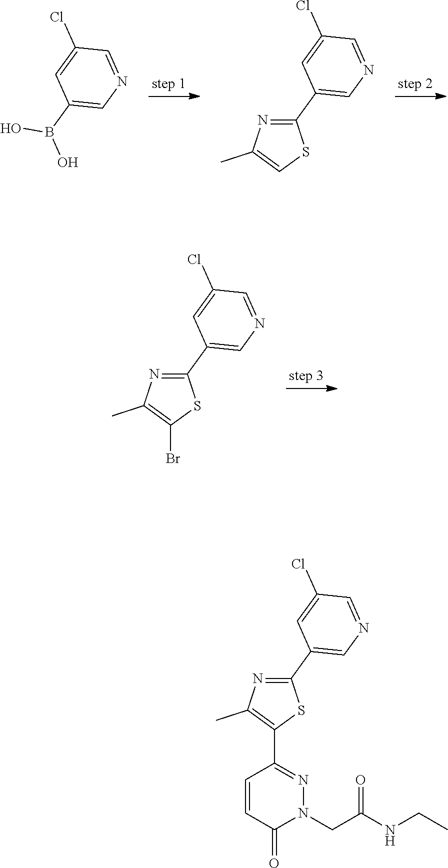 [SMILES: OB(O)c1cncc(Cl)c1 ; Cc1csc(-c2cncc(Cl)c2)n1 ; Cc1nc(-c2cncc(Cl)c2)sc1Br ; CCNC(=O)Cn1nc(-c2sc(-c3cncc(Cl)c3)nc2C)ccc1=O]

### Step 1: 2-(5-Chloropyridin-3-yl)-4-methylthiazole

To a stirred solution of 5-chloropyridin-3-ylboronic acid (500 mg, 3.177 mmol, 1.00 equiv)) in toluene (20 mL), EtOH (10 mL), and H₂O (5 mL) was added 2-bromo-4-methyl-1,3-thiazole (678.89 mg, 3.812 mmol, 1.2 equiv), K₂CO₃ (878.27 mg, 6.354 mmol, 2.00 equiv) and Pd(PPh₃)₄ (367.18 mg, 0.318 mmol, 0.10 equiv. The resulting mixture was stirred for 18 h at 100° C. under nitrogen atmosphere. The resulting mixture was concentrated under reduced pressure and the residue was purified by Prep-TLC to afford 3-chloro-5-(4-methyl-1,3-thiazol-2-yl)pyridine (470 mg, 70.21%). LCMS (ES, m/z): 211 [M+H]⁺.

### Step 2: 5-Bromo-2-(5-chloropyridin-3-yl)-4-methylthiazole

NBS (595.59 mg, 3.346 mmol, 1.50 equiv) was added to a stirred solution of 3-chloro-5-(4-methyl-1,3-thiazol-2-yl)pyridine (470 mg, 2.231 mmol, 1.00 equiv) in DMF (5 mL) and the resulting mixture was stirred for 4 h at 60° C. under nitrogen atmosphere. The reaction was quenched by the addition of water at room temperature and the residue was dissolved in DCM (10 mL) and purified by Prep-TLC (PE/EA 10:1) to afford 3-(5-bromo-4-methyl-1,3-thiazol-2-yl)-5-chloropyridine (450 mg, 69.66%). LCMS (ES, m/z): 289 [M+H]⁺.

### Step 3: 2-(3-(2-(5-Chloropyridin-3-yl)-4-methylthiazol-5-yl)-6-oxopyridazin-1(6H)-yl)-N-ethylacetamide

To a stirred solutions of 3-(5-bromo-4-methyl-1,3-thiazol-2-yl)-5-chloropyridine (200 mg, 0.691 mmol, 1.00 equiv) in dioxane (5 mL) and H₂O (1 mL) was added N-ethyl-2-[6-oxo-3-(4,4,5,5-tetramethyl-1,3,2-dioxaborolan-2-yl)pyridazin-1-yl]acetamide (424.28 mg, 1.382 mmol, 2 equiv), Na₂CO₃ (217.63 mg, 1.727 mmol, 2.50 equiv), and Pd(dppf)Cl₂ (50.54 mg, 0.069 mmol, 0.10 equiv). The resulting mixture was stirred for 4 h at 80° C. under nitrogen atmosphere. The reaction was quenched by the addition of water at room temperature and the resulting mixture was extracted with EtOAc. The combined organic layers were washed with EtOAc, dried over anhydrous Na₂SO₄ and filtered. The filtrate was concentrated under reduced pressure and the residue was purified by trituration with MeCN (10 mL) affording 2-{3-[2-(5-chloropyridin-3-yl)-4-methyl-1,3-thiazol-5-yl]-6-oxopyridazin-1-yl}-N-ethylacetamide (100 mg, 37.14%). LCMS (ES, m/z): 390 [M+H]⁺.

The following compounds of formula VI were synthesized following Example 7

| Compound | | | |
|---|---|---|---|
| No. | Structure | NMR | [M + H]⁺ |
| 150 | 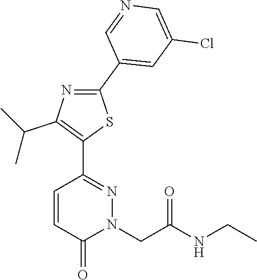 [SMILES: CCNC(=O)Cn1nc(-c2sc(-c3cncc(Cl)c3)nc2C(C)C)ccc1=O] | | 418.3 |
| 151 | 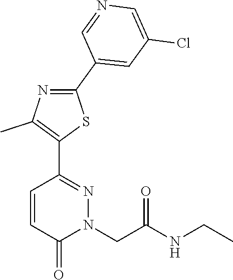 [SMILES: CCNC(=O)Cn1nc(-c2sc(-c3cncc(Cl)c3)nc2C)ccc1=O] | ¹H NMR (300 MHz, DMSO-d₆) δ 9.10 (s, 1H), 8.76 (s, 1H), 8.43 (s, 1H), 8.19 (t, J = 5.7 Hz, 1H), 7.88 (d, J = 9.9 Hz, 1H), 7.12 (d, J = 9.6 Hz, 1H), 4.70 (s, 2H), 3.16-3.08 (m, 2H), 2.64 (s, 3H), 1.05 (t, J = 7.2 Hz, 3H). | 390.1 |
| 152 | 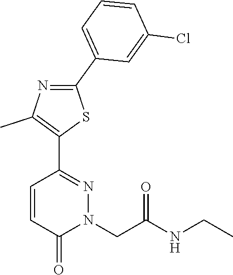 [SMILES: CCNC(=O)Cn1nc(-c2sc(-c3cccc(Cl)c3)nc2C)ccc1=O] | ¹H NMR (300 MHz, DMSO-d₆) δ 8.19 (t, J = 5.4 Hz, 1H), 7.97 (d, J = 2.0 Hz, 1H), 7.95-7.81 (m, 2H), 7.64-7.49 (m, 2H), 7.10 (d, J = 9.9 Hz, 1H), 4.70 (s, 2H), 3.13 (q, J = 5.4 Hz, 2H), 2.61 (s, 3H), 1.05 (t, J = 7.2 Hz, 3H). | 359.0 |
| 210 | 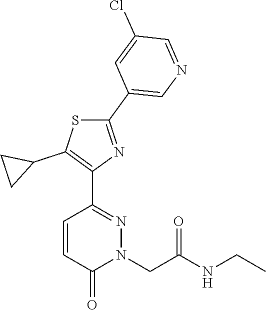 [SMILES: CCNC(=O)Cn1nc(-c2nc(-c3cncc(Cl)c3)sc2C2CC2)ccc1=O] | | 416.10 |
| 211 | 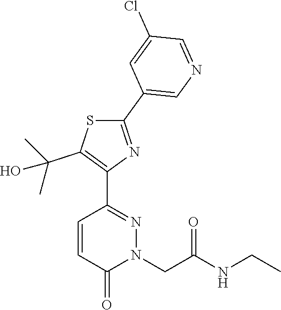 [SMILES: CCNC(=O)Cn1nc(-c2nc(-c3cncc(Cl)c3)sc2C(C)(C)O)ccc1=O] | | 434.10 |

### Example 8

### 6-(3-(3-Chlorophenyl)-1,2,4-oxadiazol-5-yl)-2-((5-(4-fluorophenyl)-1,3,4-thiadiazol-2-yl)methyl)pyridazin-3(2H)-one

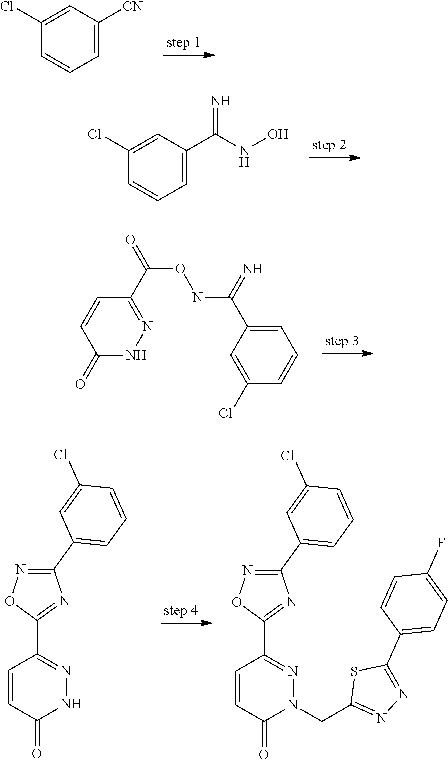

### Step 1: 3-Chloro-N-hydroxybenzimidamide

To a stirred solution of 3-chlorobenzonitrile (400 mg, 2.908 mmol, 1.00 equiv) and K₂CO₃ (803.69 mg, 5.816 mmol, 2.00 equiv) in MeOH (4 mL) were added NH₂OH·HCl (242.46 mg, 3.490 mmol, 1.20 equiv) at room temperature under nitrogen atmosphere. The reaction mixture was stirred for 2 h at 70° C. under nitrogen atmosphere and then diluted with MeOH. The reaction mixture was filtered, the filter cake was washed with MeOH and the filtrate concentrated under reduced pressure resulting in 3-chloro-N-hydroxybenzenecarboximidamide (500 mg, 90.72%). LCMS (ES, m/z): 171[M+H]⁺

### Step 2: 3-Chloro-N-((6-oxo-1,6-dihydropyridazine-3-carbonyl)oxy)benzimidamide

To a stirred solution of 3-chloro-N-hydroxybenzenecarboximidamide (500 mg, 2.931 mmol, 1.00 equiv) and 6-oxo-1H-pyridazine-3-carboxylic acid (410.60 mg, 2.931 mmol, 1.00 equiv) in DMF (5 mL) were added DIC (554.82 mg, 4.396 mmol, 1.5 equiv) and HOBt (594.05 mg, 4.396 mmol, 1.50 equiv) at room temperature under nitrogen atmosphere. The reaction mixture was stirred for 2 h at room temperature under nitrogen atmosphere. The reaction mixture was diluted with EtOAc and the precipitated solids were collected by filtration and washed with EtOAc resulting in 3-chloro-N-((6-oxo-1,6-dihydropyridazine-3-carbonyl)oxy)benzimidamide (700 mg, 73.44%). LCMS (ES, m/z): 293[M+H]⁺

### Step 3: 6-(3-(3-Chlorophenyl)-1,2,4-oxadiazol-5-yl)pyridazin-3(2H)-one

A solution of 3-chloro-N-((6-oxo-1,6-dihydropyridazine-3-carbonyl)oxy)benzimidamide (700 mg, 2.392 mmol, 1.00 equiv) in pyridine (7 mL) was stirred for 2 h at 100° C. under nitrogen atmosphere. The reaction mixture was concentrated under reduced pressure and purified by trituration with ACN (20 mL). This resulted in 6-(3-(3-chlorophenyl)-1,2,4-oxadiazol-5-yl)pyridazin-3(2H)-one (600 mg, 82.20%) as a white solid. LCMS (ES, m/z): 275[M+H]⁺.

### Step 4: 6-(3-(3-chlorophenyl)-1,2,4-oxadiazol-5-yl)-2-((5-(4-fluorophenyl)-1,3,4-thiadiazol-2-yl)methyl)pyridazin-3(2H)-one

To a stirred solution of 6-(3-(3-chlorophenyl)-1,2,4-oxadiazol-5-yl)pyridazin-3(2H)-one (300 mg, 1.092 mmol, 1.00 equiv) and K₂CO₃ (301.91 mg, 2.184 mmol, 2.00 equiv) in DMF (5 mL) was added 2-(chloromethyl)-5-(4-fluorophenyl)-1,3,4-thiadiazole (249.77 mg, 1.092 mmol, 1.00 equiv) at 0° C. under nitrogen atmosphere. The reaction mixture was stirred for overnight at room temperature under nitrogen atmosphere and then diluted with water. The reaction mixture was extracted with EtOAc and the combined organic layers were washed with brine, dried over anhydrous Na₂SO₄ and the filtrate was concentrated under reduced pressure and purified by silica gel column chromatography to afford the crude product (200 mg). Further purification by trituration with MeCN resulted in 6-(3-(3-chlorophenyl)-1,2,4-oxadiazol-5-yl)-2-((5-(4-fluorophenyl)-1,3,4-thiadiazol-2-yl)methyl)pyridazin-3(2H)-one (90 mg, 17.35%). LCMS (ES, m/z): 467[M+H]^{+ 1}H NMR (400 MHz, DMSO-d₆) δ 8.22 (d, J=9.8 Hz, 1H), 8.11-8.01 (m, 4H), 7.77-7.70 (m, 1H), 7.66 (t, J=7.9 Hz, 1H), 7.44-7.36 (m, 2H), 7.33 (d, J=9.8 Hz, 1H), 5.91 (s, 2H). ¹⁹F NMR (376 MHz, DMSO-d₆) δ −108.57.

### Example 9

### 6-(3-(5-Chloropyridin-3-yl)-1,2,4-oxadiazol-5-yl)-2-((2-ethylthiazol-5-yl)methyl)pyridazin-3(2H)-one

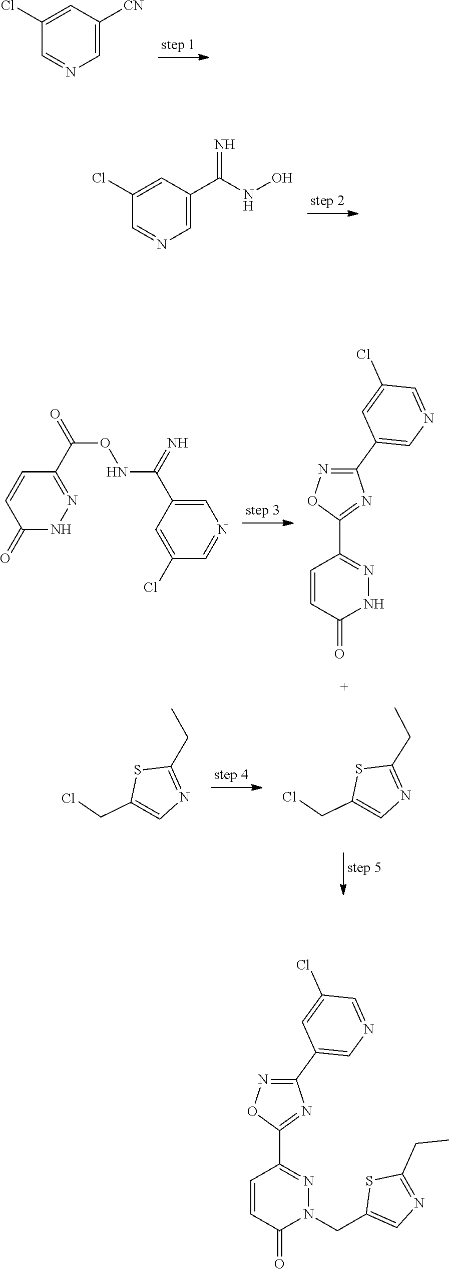

### Step 1: 5-Chloro-N-hydroxynicotinimidamide

To a stirred solution of 5-chloronicotinonitrile (4.00 g, 28.870 mmol, 1.00 equiv) and K₂CO₃ (7.98 g, 57.740 mmol, 2.00 equiv) in MeOH (40 mL) was added NH₂OH·HCl (2.41 g, 34.644 mmol, 1.20 equiv) in portions at room temperature under nitrogen atmosphere. The reaction mixture was stirred for 1 h at 70° C. under nitrogen atmosphere and then cooled room temperature. The reaction mixture was concentrated under reduced pressure and diluted with MeCN. The residue was purified by trituration with MeCN resulting in 5-chloro-N-hydroxynicotinimidamide (3.60 g, 72.67%). LCMS (ES, m/z): 172 [M+H]⁺

### Step 2: 5-Chloro-N-((6-oxo-1,6-dihydropyridazine-3-carbonyl)oxy)nicotinimidamide

To a stirred solution of 5-chloro-N-hydroxynicotinimidamide (3.00 g, 17.485 mmol, 1.00 equiv) and 6-oxo-1,6-dihydropyridazine-3-carboxylic acid (2.94 g, 20.982 mmol, 1.20 equiv) in DMF (30 mL) were added DIC (3.31 g, 26.227 mmol, 1.50 equiv) and HOBt (3.54 g, 26.227 mmol, 1.50 equiv) in portions at room temperature under nitrogen atmosphere. The reaction mixture was stirred for 2 h at room temperature under nitrogen atmosphere and diluted with water. The precipitated solids were collected by filtration and washed with MeCN. The residue was purified by trituration with MeCN to afford 5-chloro-N-((6-oxo-1,6-dihydropyridazine-3-carbonyl)oxy)nicotinimidamide (3.80 g, 74.01%) as a white solid. LCMS (ES, m/z): 294 [M+H]³⁰

### Step 3: 6-(3-(5-Chloropyridin-3-yl)-1,2,4-oxadiazol-5-yl)pyridazin-3(2H)-one

A solution of 5-chloro-N-((6-oxo-1,6-dihydropyridazine-3-carbonyl)oxy)nicotinimidamide (400 mg, 1.362 mmol, 1.00 equiv) in pyridine (8 mL) was stirred for 3 h at 100° C. under nitrogen atmosphere. The reaction mixture was concentrated under reduced pressure. The precipitated solids were collected by filtration and washed with MeCN. The residue was purified by trituration with MeCN to afford 6-(3-(5-chloropyridin-3-yl)-1,2,4-oxadiazol-5-yl)pyridazin-3(2H)-one (330 mg, 87.89%). LCMS (ES, m/z): 276 [M+H]⁺

### Step 4: 5-(Chloromethyl)-2-ethylthiazole

To a stirred solution of 5-(chloromethyl)-2-ethylthiazole (350 mg, 2.165 mmol, 1.00 equiv) in DCM (5 mL) was added SOCl₂ (1.29 g, 10.825 mmol, 5.00 equiv) at 0° C. under nitrogen atmosphere. The reaction mixture was stirred for 2 h at room temperature under nitrogen atmosphere and then concentrated under reduced pressure. The reaction mixture was washed with DCM. The reaction mixture was concentrated under reduced pressure resulting in 5-(chloromethyl)-2-ethylthiazole (300 mg, crude). The crude product was used in the next step directly without further purification. LCMS (ES, m/z): 162 [M+H]⁺

### Step 5: 6-(3-(5-chloropyridin-3-yl)-1,2,4-oxadiazol-5-yl)-2-((2-ethylthiazol-5-yl)methyl)pyridazin-3(2H)-one

To a stirred solution of 6-(3-(5-chloropyridin-3-yl)-1,2,4-oxadiazol-5-yl)pyridazin-3(2H)-one (250 mg, 0.907 mmol, 1.00 equiv) and Cs₂CO₃ (1.03 g, 3.175 mmol, 3.50 equiv) in DMF (5 mL) were added 5-(chloromethyl)-2-ethylthiazole (219.91 mg, 1.361 mmol, 1.50 equiv) in portions at room temperature under nitrogen atmosphere. The reaction mixture was stirred for 36 h at room temperature under nitrogen atmosphere and diluted with water. The reaction mixture was extracted with EtOAc and the combined organic layers were washed with brine, dried over anhydrous Na₂SO₄ and the filtrate was concentrated under reduced pressure and purified by silica gel column chromatography to afford the crude product (200 mg). Further purification by reverse flash chromatography with the following conditions: column, C18 gel; mobile phase, MeCN in Water (0.1% FA), 20% to 80% gradient in 25 min; detector, UV 254/220 nm. RT1:24 min. This resulted in 6-(3-(5-chloropyridin-3-yl)-1,2,4-oxadiazol-5-yl)-2-((2-ethylthiazol-5-yl)methyl)pyridazin-3(2H)-one (81.5 mg, 23.51%) as an off-white solid. LCMS (ES, m/z): 401 [M+H]^{+ 1}H NMR (400 MHz, DMSO-d₆) δ 9.19 (d, J=2 Hz, 1H), 8.92 (d, J=2.4 Hz, 1H), 8.51 (t, J=1.6 Hz, 1H), 8.16 (d, J=9.6 Hz, 1H), 7.77 (s, 1H), 7.27 (d, J=10 Hz, 1H), 5.58 (s, 2H), 2.94 (q, J=7.5 Hz, 2H), 1.25 (t, J=7.5 Hz, 3H).

The following compounds of formula VII were synthesized following Example 8 and Example 9.

| Compound No. | Structure | [M + H]⁺ |
|---|---|---|
| 190 | 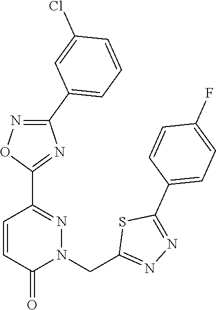 [SMILES: O=c1ccc(-c2nc(-c3cccc(Cl)c3)no2)nn1Cc1nnc(-c2ccc(F)cc2)s1] | 466.95 |
| 191 | 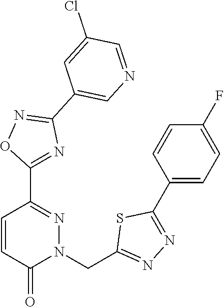 [SMILES: O=c1ccc(-c2nc(-c3cncc(Cl)c3)no2)nn1Cc1nnc(-c2ccc(F)cc2)s1] | 467.95 |
| 192 | 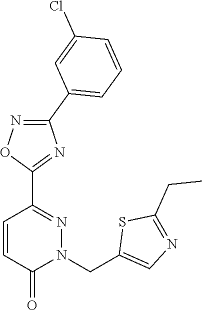 [SMILES: CCc1ncc(Cn2nc(-c3nc(-c4cccc(Cl)c4)no3)ccc2=O)s1] | 399.95 |
| 193 | 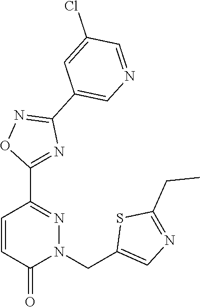 [SMILES: CCc1ncc(Cn2nc(-c3nc(-c4cncc(Cl)c4)no3)ccc2=O)s1] | 401.05 |
| 194 | 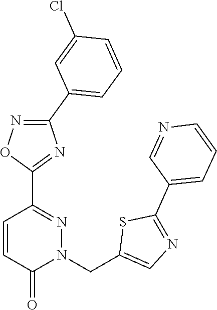 [SMILES: O=c1ccc(-c2nc(-c3cccc(Cl)c3)no2)nn1Cc1cnc(-c2cccnc2)s1] | 449.00 |
| 195 | 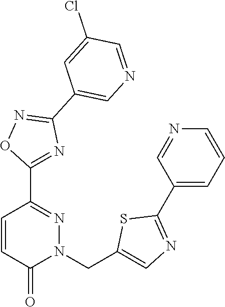 [SMILES: O=c1ccc(-c2nc(-c3cncc(Cl)c3)no2)nn1Cc1cnc(-c2cccnc2)s1] | 450.10 |
| 196 | 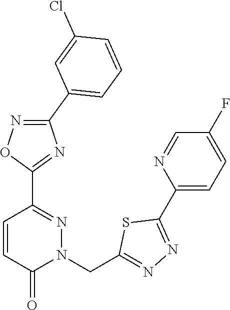 [SMILES: O=c1ccc(-c2nc(-c3cccc(Cl)c3)no2)nn1Cc1nnc(-c2ccc(F)cn2)s1] | 467.95 |
| 197 | 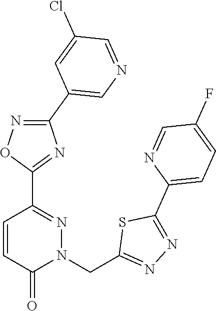 [SMILES: O=c1ccc(-c2nc(-c3cncc(Cl)c3)no2)nn1Cc1nnc(-c2ccc(F)cn2)s1] | 464.00 |
| 198 | 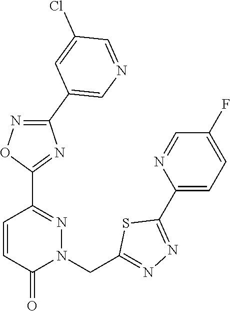 [SMILES: O=c1ccc(-c2nc(-c3cncc(Cl)c3)no2)nn1Cc1nnc(-c2ccc(F)cn2)s1] | 469.00 |
| 199 | 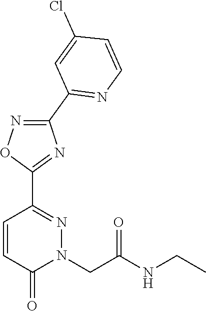 [SMILES: CCNC(=O)Cn1nc(-c2nc(-c3cc(Cl)ccn3)no2)ccc1=O] | 361.10 |

### Example 10—6-(2-(5-Chloropyridin-3-yl)-4-methylthiazol-5-yl)-2-((3-(5-fluoropyridin-3-yl)-1,2,4-oxadiazol-5-yl)methyl)pyridazin-3(2H)-one

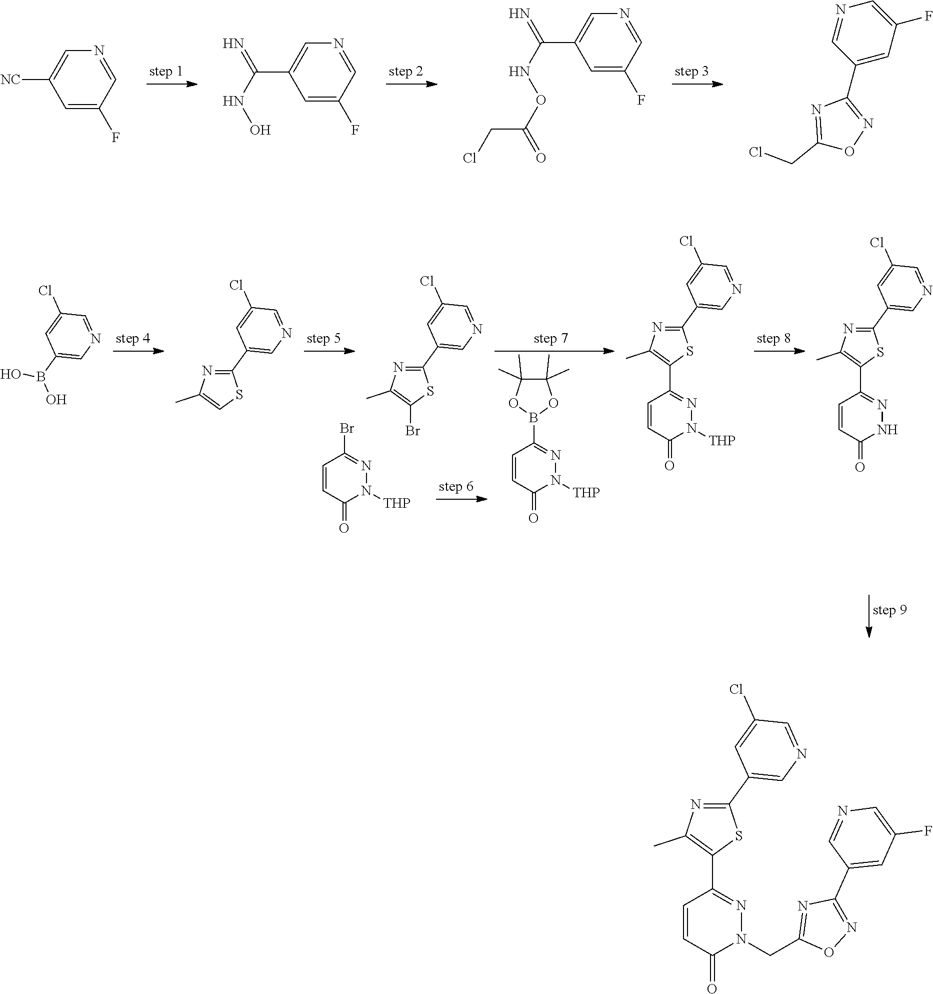

### Step 1: 5-Fluoro-N-hydroxynicotinimidamide

To a stirred solution of 5-fluoronicotinonitrile (1.00 g, 8.190 mmol, 1.00 equiv) and NaHCO₃ (0.83 g, 9.828 mmol, 1.20 equiv) in EtOH (10 mL) and H₂O (0.6 mL) was added NH₂OH·HCl (0.68 g, 9.828 mmol, 1.20 equiv) dropwise at room temperature under nitrogen atmosphere. The resulting mixture was stirred for 2 h at 80° C. under nitrogen atmosphere and then filtered, the filter cake was washed with EtOH. The filtrate was concentrated under reduced pressure resulting in 5-fluoro-N-hydroxynicotinimidamide (1.05 g, crude) as a white solid. The crude product was used in the next step directly without further purification. LCMS (ES, m/z): 156 [M+H]⁺

### Step 2: N-(2-Chloroacetoxy)-5-fluoronicotinimidamide

To a stirred solution of 5-fluoro-N-hydroxynicotinimidamide (1.00 g, 6.446 mmol, 1.00 equiv) in MeCN (10 mL) was added 2-chloroacetyl chloride (1.09 g, 9.669 mmol, 1.50 equiv) dropwise at 0° C. under nitrogen atmosphere. The resulting mixture was stirred for 3 h at room temperature under nitrogen atmosphere. The resulting mixture was filtered, the filter cake was washed with MeCN and the filtrate was concentrated under reduced pressure resulting in N-(2-chloroacetoxy)-5-fluoronicotinimidamide (500 mg, crude). The crude product was used in the next step directly without further purification. LCMS (ES, m/z): 232 [M+H]⁺

### Step 3: 5-(Chloromethyl)-3-(5-fluoropyridin-3-yl)-1,2,4-oxadiazole

N-(2-chloroacetoxy)-5-fluoronicotinimidamide (500 mg, 2.159 mmol, 1.00 equiv) was stirred in toluene (5 mL) at room temperature under nitrogen atmosphere. The reaction mixture was stirred for 5 h at 100° C. under nitrogen atmosphere and then concentrated under reduced pressure and purified by silica gel column chromatography to afford 5-(chloromethyl)-3-(5-fluoropyridin-3-yl)-1,2,4-oxadiazole (150 mg, 32.53%) as a white solid. LCMS (ES, m/z): 214 [M+H]⁺

### Step 4: 2-(5-Chloropyridin-3-yl)-4-methylthiazole

To a stirred solution of (5-chloropyridin-3-yl)boronic acid (500 mg, 3.177 mmol, 1.00 equiv) and K₂CO₃ (878.27 mg, 6.354 mmol, 2.00 equiv) in toluene (20 mL), EtOH (10 mL) and H₂O (5 mL) was added 2-bromo-4-methylthiazole (678.89 mg, 3.812 mmol, 1.20 equiv) and Pd(PPh₃)₄ (367.18 mg, 0.318 mmol, 0.10 equiv) at room temperature under nitrogen atmosphere. The reaction mixture was stirred overnight at 100° C. under nitrogen atmosphere. The reaction mixture was diluted with water and extracted with EtOAc. The combined organic layers were washed with brine, dried over anhydrous Na₂SO₄ and filtered. The filtrate was concentrated under reduced pressure and the residue was purified by Prep-TLC to afford 3-chloro-5-(4-methyl-1,3-thiazol-2-yl)pyridine (470 mg, 70.21%). LCMS (ES, m/z): 211 [M+H]⁺

### Step 5: 5-Bromo-2-(5-chloropyridin-3-yl)-4-methylthiazole

To a stirred solution of 2-(5-chloropyridin-3-yl)-4-methylthiazole (470 mg, 2.231 mmol, 1.00 equiv) in DMF (5 mL) was added NBS (595.59 mg, 3.346 mmol, 1.50 equiv) at room temperature under nitrogen atmosphere. The reaction mixture was stirred for 4 h at 60° C. under nitrogen atmosphere and then diluted with water. The resulting mixture was extracted with EtOAc and the combined organic layers were washed with brine, dried over anhydrous Na₂SO₄ and filtered. The filtrate was concentrated under reduced pressure and the residue was purified by Prep-TLC to afford 5-bromo-2-(5-chloropyridin-3-yl)-4-methylthiazole (480 mg, 74.30%). LCMS (ES, m/z): 289 [M+H]⁺

### Step 6: 2-(Tetrahydro-2H-pyran-2-yl)-6-(4,4,5,5-tetramethyl-1,3,2-dioxaborolan-2-yl)pyridazin-3(2H)-one

To a stirred solution of 6-bromo-2-(tetrahydro-2H-pyran-2-yl)pyridazin-3(2H)-one (2.50 g, 9.649 mmol, 1.00 equiv) and 4,4,4′,4′,5,5,5′,5′-octamethyl-2,2′-bi(1,3,2-dioxaborolane) (4.90 g, 19.298 mmol, 2.00 equiv) in 1,4-dioxane (20 mL) was added KOAc (1.89 g, 19.298 mmol, 2.00 equiv) and Pd(dppf)Cl₂ (0.71 g, 0.965 mmol, 0.10 equiv) at room temperature under nitrogen atmosphere. The reaction mixture was stirred for 3 h at 80° C. under argon atmosphere then concentrated under reduced pressure resulting in 2-(tetrahydro-2H-pyran-2-yl)-6-(4,4,5,5-tetramethyl-1,3,2-dioxaborolan-2-yl)pyridazin-3(2H)-one (2.50 g, crude). The crude product was used in the next step directly without further purification. LCMS (ES, m/z): 307 [M+H]⁺

### Step 7: 6-(2-(5-Chloropyridin-3-yl)-4-methylthiazol-5-yl)-2-(tetrahydro-2H-pyran-2-yl)pyridazin-3(2H)-one

To a stirred solution of 5-bromo-2-(5-chloropyridin-3-yl)-4-methylthiazole (470 mg, 1.623 mmol, 1.00 equiv) and Na₂CO₃ (430.06 mg, 4.058 mmol, 2.50 equiv) in dioxane (5 mL) and H₂O (1 mL) was added 2-(oxan-2-yl)-6-(4,4,5,5-tetramethyl-1,3,2-dioxaborolan-2-yl)pyridazin-3-one (993.85 mg, 3.246 mmol, 2.00 equiv) and Pd(dppf)Cl₂ (118.76 mg, 0.162 mmol, 0.10 equiv) at room temperature under nitrogen atmosphere. The resulting mixture was stirred for 2 h at 80° C. under argon atmosphere. The resulting mixture was diluted with water, extracted with EtOAc, and the combined organic layers were washed with brine dried over anhydrous Na₂SO₄ and filtered. The filtrate was concentrated under reduced pressure and the residue purified by silica gel column chromatography to afford 6-(2-(5-chloropyridin-3-yl)-4-methylthiazol-5-yl)-2-(tetrahydro-2H-pyran-2-yl)pyridazin-3(2H)-one (450 mg, 71.30%). LCMS (ES, m/z): 389 [M+H]⁺

### Step 8: 6-(2-(5-Chloropyridin-3-yl)-4-methylthiazol-5-yl)pyridazin-3(2H)-one

To a stirred solution of 6-(2-(5-chloropyridin-3-yl)-4-methylthiazol-5-yl)-2-(tetrahydro-2H-pyran-2-yl)pyridazin-3(2H)-one (250 mg, 0.643 mmol, 1.00 equiv) in dioxane (2 mL) was added 4M HCl(gas) in 1,4-dioxane (4 mL, 131.651 mmol, 204.78 equiv) dropwise at 0° C. under nitrogen atmosphere. The reaction mixture was stirred for 2 h at room temperature under nitrogen atmosphere and then concentrated under vacuum. The resulting mixture was washed with DCM resulting in 6-(2-(5-chloropyridin-3-yl)-4-methylthiazol-5-yl)pyridazin-3(2H)-one (250 mg, crude). The crude product was used in the next step directly without further purification. LCMS (ES, m/z): 305 [M+H]⁺

### Step 9. 6-(2-(5-Chloropyridin-3-yl)-4-methylthiazol-5-yl)-2-((3-(5-fluoropyridin-3-yl)-1,2,4-oxadiazol-5-yl)methyl)pyridazin-3(2H)-one

To a stirred solution of 6-(2-(5-chloropyridin-3-yl)-4-methylthiazol-5-yl)pyridazin-3(2H)-one (150 mg, 0.492 mmol, 1 equiv) and Cs₂CO₃ (481.11 mg, 1.476 mmol, 3.00 equiv) in DMSO (2 mL) was added 5-(chloromethyl)-3-(5-fluoropyridin-3-yl)-1,2,4-oxadiazole (105.14 mg, 0.492 mmol, 1.00 equiv) dropwise at room temperature under nitrogen atmosphere. The resulting mixture was stirred for 30 min at room temperature under nitrogen atmosphere and diluted with water. The resulting mixture was extracted with EtOAc and the combined organic layers were washed with brine, dried over anhydrous Na₂SO₄ and filtered. The filtrate was concentrated under reduced pressure and the residue was purified by silica gel column chromatography, to afford the crude product (150 mg) and the further purified by reverse flash chromatography: column, C18 gel; mobile phase, MeCN in Water (10 mmol/L NH₄HCO₃), 10% to 50% gradient in 20 min; detector, UV 254 nm resulting in 6-(2-(5-chloropyridin-3-yl)-4-methylthiazol-5-yl)-2-((3-(5-fluoropyridin-3-yl)-1,2,4-oxadiazol-5-yl)methyl)pyridazin-3(2H)-one (67.2 mg, 28.33%). LCMS (ES, m/z): 482 [M+H]^{+ 1}H NMR (300 MHz, DMSO-d₆) δ 9.08 (d, J=1.8 Hz, 1H), 9.03 (s, 1H), 8.85 (d, J=3 Hz, 1H), 8.76 (d, J=2.1 Hz, 1H), 8.42 (t, J=2.1 Hz, 1H), 8.28-8.23 (m, 1H), 7.97 (d, J=9.6 Hz, 1H), 7.24 (d, J=9.9 Hz, 1H), 5.80 (s, 2H), 2.64 (s, 3H). ¹⁹F NMR (282 MHz, DMSO-d₆) δ −125.37 (1F).

### Example 11

### 6-(2-(5-Chloropyridin-3-yl)-4-methylthiazol-5-yl)-2-((5-(4-fluorophenyl)-1,3,4-thiadiazol-2-yl)methyl)pyridazin-3(2H)-one

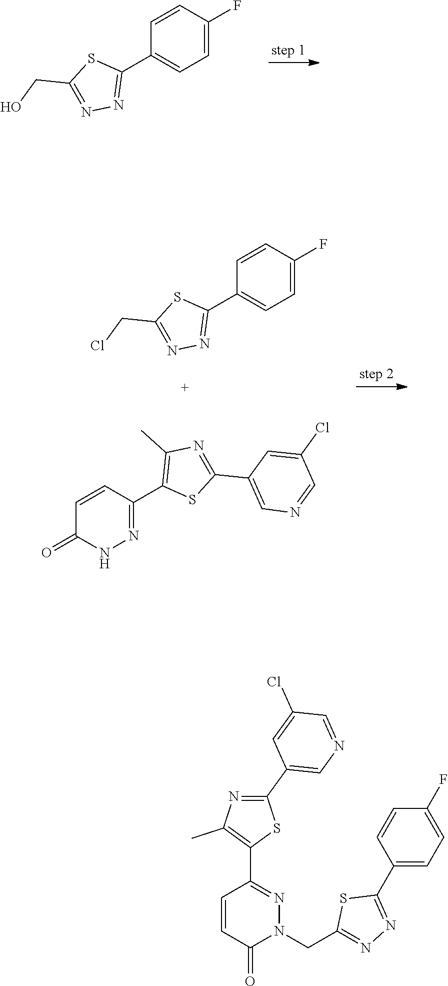 [SMILES: OCc1nnc(-c2ccc(F)cc2)s1 ; Fc1ccc(-c2nnc(CCl)s2)cc1 ; Cc1nc(-c2cncc(Cl)c2)sc1-c1ccc(=O)[nH]n1 ; Cc1nc(-c2cncc(Cl)c2)sc1-c1ccc(=O)n(Cc2nnc(-c3ccc(F)cc3)s2)n1]

### Step 1: 2-(Chloromethyl)-5-(4-fluorophenyl)-1,3,4-thiadiazole

To a stirred solution of (5-(4-fluorophenyl)-1,3,4-thiadiazol-2-yl)methanol (300 mg, 1.427 mmol, 1.00 equiv) in DCM (3 mL, 47.192 mmol) was added SOCl₂ (848.78 mg, 7.135 mmol, 5.00 equiv) at room temperature under nitrogen atmosphere. The reaction mixture was stirred for 3 h at room temperature under nitrogen atmosphere, and then concentrated under vacuum. The resulting mixture was washed with DCM resulting in 2-(chloromethyl)-5-(4-fluorophenyl)-1,3,4-thiadiazole (320 mg, crude). The crude product was used in the next step directly without further purification. LCMS (ES, m/z): 229 [M+H]⁺

### Step 2: 6-(2-(5-chloropyridin-3-yl)-4-methylthiazol-5-yl)-2-((5-(4-fluorophenyl)-1,3,4-thiadiazol-2-yl)methyl)pyridazin-3(2H)-one

To a stirred solution of 6-(2-(5-chloropyridin-3-yl)-4-methylthiazol-5-yl)pyridazin-3(2H)-one (199.91 mg, 0.656 mmol, 1.00 equiv) and Cs₂CO₃ (854.91 mg, 2.624 mmol, 4.00 equiv) in DMSO (4 mL) was added 2-(chloromethyl)-5-(4-fluorophenyl)-1,3,4-thiadiazole (150 mg, 0.656 mmol, 1.00 equiv) dropwise at room temperature under nitrogen atmosphere. The reaction mixture was stirred for 30 min at room temperature under nitrogen atmosphere and then diluted with water and extracted with EtOAc. The combined organic layers were washed with brine, dried over anhydrous Na₂SO₄ and filtered. The filtrate was concentrated under reduced pressure and the residue was purified by silica gel column chromatography to afford the crude product (150 mg). Further trituration with DMSO resulted in 6-(2-(5-chloropyridin-3-yl)-4-methylthiazol-5-yl)-2-((5-(4-fluorophenyl)-1,3,4-thiadiazol-2-yl)methyl)pyridazin-3(2H)-one (105.7 mg, 32.42%) as a yellow solid. LCMS (ES, m/z): 497 [M+H]^{+ 1}H NMR (300 MHz, DMSO-d₆) δ 9.09 (d, J=1.8 Hz, 1H), 8.75 (d, J=2.1 Hz, 1H), 8.43 (t, J=2.1 Hz, 1H), 8.11-8.01 (m, 2H), 7.93 (d, J=9.9 Hz, 1H), 7.46-7.34 (m, 2H), 7.22 (d, J=9.6 Hz, 1H), 5.79 (s, 2H), 2.65 (s, 3H). ¹⁹F NMR (282 MHz, DMSO-d₆) δ −108.61 (1F).

### Example 12

### 6-(2-(3-Chlorophenyl)-4-methyloxazol-5-yl)-2-((5-(piperidin-1-yl)-1,3,4-thiadiazol-2-yl)methyl)pyridazin-3(2H)-one

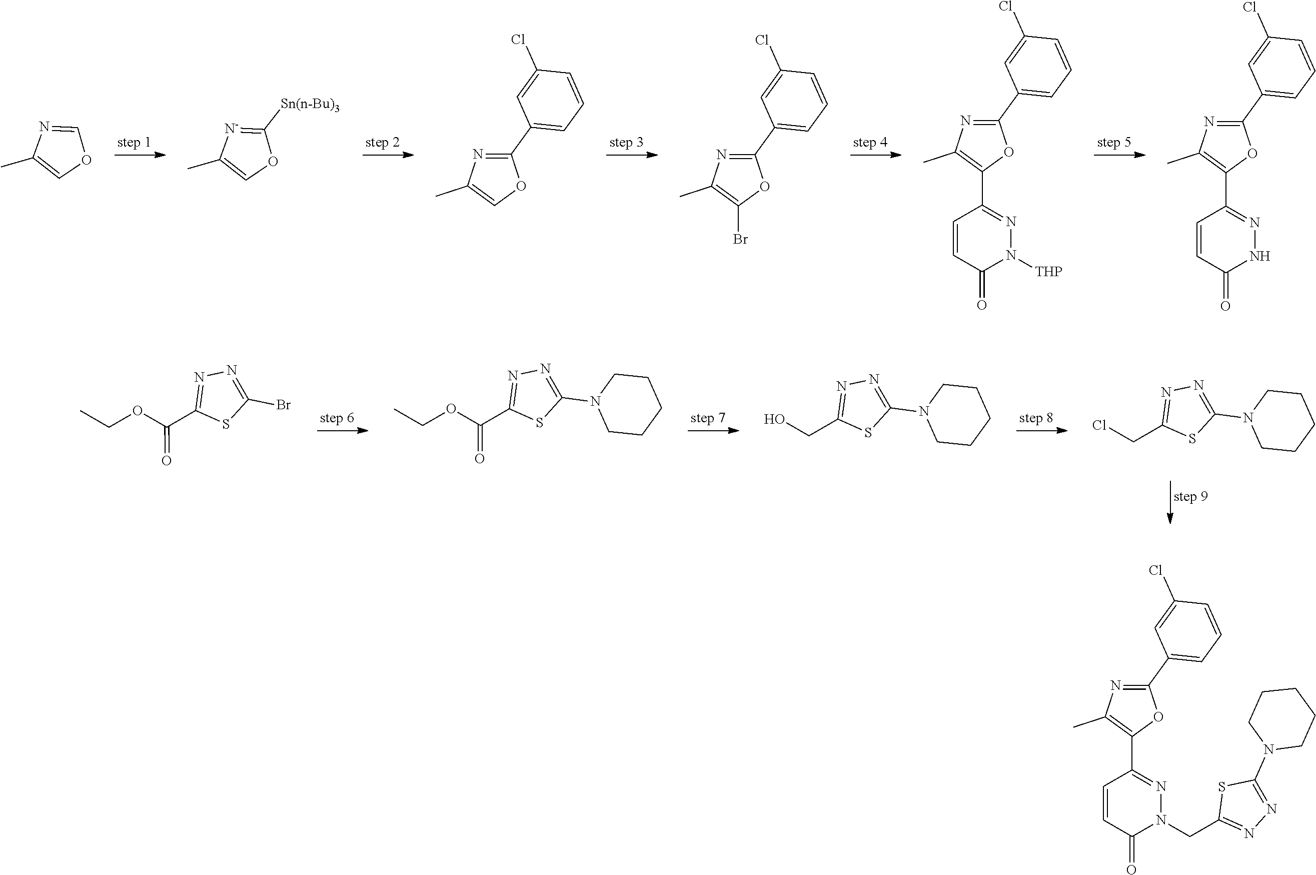

### Step 1: 4-Methyl-2-(tributylstannyl)oxazole

To a stirred solution of 4-methyloxazol (8.00 g, 96.281 mmol, 1.00 equiv) in THF (80 mL) was added 2.5M n-BuLi in hexanes (57.77 mL, 144.422 mmol, 1.50 equiv) dropwise at −78° C. under argon atmosphere. The reaction mixture was stirred for 1 h at −78° C. under argon atmosphere and Bu₃SnCl (34.47 g, 105.909 mmol, 1.10 equiv) was added at −78° C. to the mixture. The reaction mixture was stirred for additional 2 h at −78° C. and quenched with sat. NH₄Cl (aq.) at −78° C. The reaction mixture was diluted with water and extracted with EtOAc. The combined organic layers were washed with brine, dried over anhydrous Na₂SO₄ and the filtrate was concentrated under reduced pressure and purified by silica gel column chromatography to afford 4-methyl-2-(tributylstannyl)oxazole (32 g, crude). LCMS (ES, m/z): 374 [M+H]⁺

### Step 2: 2-(3-Chlorophenyl)-4-methyloxazole

To a stirred solution of 1-chloro-3-iodobenzene (5.00 g, 20.969 mmol, 1.00 equiv) and 4-methyl-2-(tributylstannyl)oxazole (31.21 g, 83.876 mmol, 4.00 equiv) in DMF (50 mL) were added Pd(PPh₃)₂Cl₂ (1.47 g, 2.097 mmol, 0.10 equiv) and CuI (3.99 g, 20.969 mmol, 1.00 equiv) at room temperature under argon atmosphere. The final reaction mixture was irradiated with microwave radiation for 1 h at 100° C. The reaction mixture was diluted with water (150 mL). The reaction mixture was extracted with EtOAc (5×100 mL). The combined organic layers were washed with brine (5×100 mL), dried over anhydrous Na₂SO₄ and the filtrate was concentrated under reduced pressure and purified by silica gel column chromatography, eluted with PE/EA (5:1) to afford 2-(3-chlorophenyl)-4-methyloxazole (2.30 g, 56.65%) as a yellow oil. LCMS (ES, m/z): 194 [M+H]⁺

### Step 3: 5-Bromo-2-(3-chlorophenyl)-4-methyloxazole

To a stirred solution of 2-(3-chlorophenyl)-4-methyloxazole (2.20 g, 11.362 mmol, 1.00 equiv) in DMF (25 mL) was added NBS (3.03 g, 17.043 mmol, 1.50 equiv) dropwise at room temperature under nitrogen atmosphere. The reaction mixture was stirred for 2 h at 60° C. under nitrogen atmosphere. The reaction mixture was diluted with water and extracted with EtOAc. The combined organic layers were washed with brine, dried over anhydrous Na₂SO₄ and the filtrate was concentrated under reduced pressure and purified by silica gel column chromatography to afford 5-bromo-2-(3-chlorophenyl)-4-methyloxazole (1.80 g, 58.13%). LCMS (ES, m/z): 272 [M+H]⁺

### Step 4: 6-(2-(3-Chlorophenyl)-4-methyloxazol-5-yl)-2-(tetrahydro-2H-pyran-2-yl)pyridazin-3(2H)-one

To a stirred solution of 5-bromo-2-(3-chlorophenyl)-4-methyloxazole (1.70 g, 6.238 mmol, 1.00 equiv) and Na₂CO₃ (1.65 g, 15.595 mmol, 2.50 equiv) in 1,4-dioxane (20 mL) and H₂O (4 mL) were added 2-(tetrahydro-2H-pyran-2-yl)-6-(4,4,5,5-tetramethyl-1,3,2-dioxaborolan-2-yl)pyridazin-3(2H)-one (2.29 g, 7.486 mmol, 1.20 equiv) and Pd(dppf)Cl₂ (912.86 mg, 1.248 mmol, 0.20 equiv) in portions at room temperature under argon atmosphere. The reaction mixture was stirred for 3 h at 80° C. under argon atmosphere and then diluted with water. The reaction mixture was extracted with and the combined organic layers were washed with brine (3×50 mL), dried over anhydrous Na₂SO₄ and the filtrate was concentrated under reduced pressure and purified by silica gel column chromatography to afford 6-(2-(3-chlorophenyl)-4-methyloxazol-5-yl)-2-(tetrahydro-2H-pyran-2-yl)pyridazin-3(2H)-one (1.30 g, 56.05%). LCMS (ES, m/z): 372 [M+H]⁺

### Step 5: 6-(2-(3-Chlorophenyl)-4-methyloxazol-5-yl)pyridazin-3(2H)-one

To a stirred solution of 6-(2-(3-chlorophenyl)-4-methyloxazol-5-yl)-2-(tetrahydro-2H-pyran-2-yl)pyridazin-3(2H)-one (1.30 g, 3.496 mmol, 1.00 equiv) in DCM (13 mL) was added 4M HCl(gas) in 1,4-dioxane (13.00 mL, 427.840 mmol, 122.38 equiv) dropwise at 0° C. under nitrogen atmosphere. The reaction mixture was stirred for 1 h at room temperature under nitrogen atmosphere. The reaction mixture was concentrated under reduced pressure resulting in 6-(2-(3-chlorophenyl)-4-methyloxazol-5-yl)pyridazin-3(2H)-one (1.30 g, crude). The crude product was used in the next step directly without further purification. LCMS (ES, m/z): 288 [M+H]⁺.

### Step 6: Ethyl 5-(piperidin-1-yl)-1,3,4-thiadiazole-2-carboxylate

To a stirred solution of ethyl 5-bromo-1,3,4-thiadiazole-2-carboxylate (3.00 g, 12.654 mmol, 1.00 equiv) and TEA (2.56 g, 25.308 mmol, 2.00 equiv) in MeCN (30 mL) was added piperidine (1.62 g, 18.981 mmol, 1.50 equiv) dropwise at 0° C. under nitrogen atmosphere. The reaction mixture was stirred for 2 h at 40° C. under nitrogen atmosphere. The reaction mixture was concentrated under reduced pressure and diluted with water. The reaction mixture was extracted with EtOAc and the combined organic layers were washed with brine, dried over anhydrous Na₂SO₄ and the filtrate was concentrated under reduced pressure and purified by silica gel column chromatography to afford ethyl 5-(piperidin-1-yl)-1,3,4-thiadiazole-2-carboxylate (2.00 g, 65.50%). LCMS (ES, m/z): 242 [M+H]⁺.

### Step 7: (5-(Piperidin-1-yl)-1,3,4-thiadiazol-2-yl)methanol

To a stirred solution of ethyl 5-(piperidin-1-yl)-1,3,4-thiadiazole-2-carboxylate (2.00 g, 8.288 mmol, 1.00 equiv) in MeOH (20 mL) was added NaBH₄ (0.94 g, 24.864 mmol, 3.00 equiv) dropwise at 0° C. under nitrogen atmosphere. The reaction mixture was stirred for 1 h at room temperature under nitrogen atmosphere. The reaction was quenched by the addition of water at 0° C. The residue was purified by silica gel column chromatography to afford (5-(piperidin-1-yl)-1,3,4-thiadiazol-2-yl)methanol (1.40 g, 84.77%). LCMS (ES, m/z): 200 [M+H]⁺.

### Step 8: 2-(Chloromethyl)-5-(piperidin-1-yl)-1,3,4-thiadiazole

To a stirred solution of (5-(piperidin-1-yl)-1,3,4-thiadiazol-2-yl)methanol (350 mg, 1.756 mmol, 1.00 equiv) in DCM (4 mL) was added SOCl₂ (417.89 mg, 3.512 mmol, 2.00 equiv) at 0° C. under nitrogen atmosphere. The reaction mixture was stirred for 1 h at room temperature under nitrogen atmosphere and then concentrated under reduced pressure resulting in 2-(chloromethyl)-5-(piperidin-1-yl)-1,3,4-thiadiazole (350 mg, crude). The crude product was used in the next step directly without further purification. LCMS (ES, m/z): 218 [M+H]⁺.

### Step 9: 6-(2-(3-Chlorophenyl)-4-methyloxazol-5-yl)-2-((5-(piperidin-1-yl)-1,3,4-thiadiazol-2-yl)methyl)pyridazin-3(2H)-one

To a stirred solution of 6-(2-(3-chlorophenyl)-4-methyloxazol-5-yl)pyridazin-3(2H)-one (400 mg, 1.390 mmol, 1.00 equiv), K₂CO₃ (576.45 mg, 4.170 mmol, 3.00 equiv) and TBAB (448.21 mg, 1.390 mmol, 1.00 equiv) in DMF (8 mL) were added 2-(chloromethyl)-5-(piperidin-1-yl)-1,3,4-thiadiazole (332.97 mg, 1.529 mmol, 1.10 equiv) dropwise at 0° C. under nitrogen atmosphere. The reaction mixture was stirred for overnight at room temperature under nitrogen atmosphere. The reaction mixture was diluted with water and extracted with EtOAc. The combined organic layers were washed with brine, dried over anhydrous Na₂SO₄ and the filtrate was concentrated under reduced pressure and purified by silica gel column chromatography to afford the crude product (200 mg). Further purification by reverse phase flash with the following conditions (Column: XBridge Prep OBD C₁₈ Column, 50*250 mm, 10 μm; Mobile Phase A: Water (10 mmol/L NH₄HCO₃), Mobile Phase B: ACN; Flow rate: 60 mL/min; Gradient: 45% B to 60% B in 15 min, 60% B; Wave Length: 254/220 nm; RT1 (min): 8.32; Number Of Runs: 0) afforded 6-(2-(3-chlorophenyl)-4-methyloxazol-5-yl)-2-((5-(piperidin-1-yl)-1,3,4-thiadiazol-2-yl)methyl)pyridazin-3(2H)-one (92 mg, 14.11%). LCMS (ES, m/z): 469 [M+H]⁺. ¹H NMR (400 MHz, DMSO-d₆) δ 8.12-8.10 (m, 2H), 8.03 (dt, J=7.6, 1.2 Hz, 1H), 7.63 (dd, J=8.4, 1.6 Hz, 2H), 7.20 (d, J=9.6 Hz, 1H), 5.51 (s, 2H), 3.42 (s, 4H), 2.45 (s, 3H), 1.57 (s, 6H).

The following compounds of formula VIII were synthesized following Examples 10, 11, and 12.

| Compound | | |
|---|---|---|
| No. | Structure | [M + H]⁺ |
| 153 | 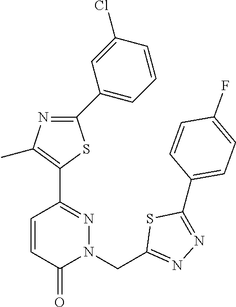 [SMILES: Cc1nc(-c2cccc(Cl)c2)sc1-c1ccc(=O)n(Cc2nnc(-c3ccc(F)cc3)s2)n1] | 495.90 |
| 154 | 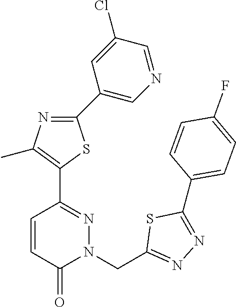 [SMILES: Cc1nc(-c2cncc(Cl)c2)sc1-c1ccc(=O)n(Cc2nnc(-c3ccc(F)cc3)s2)n1] | 560.00 |
| 155 | 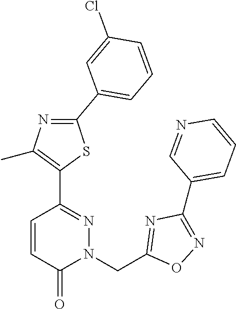 [SMILES: Cc1nc(-c2cccc(Cl)c2)sc1-c1ccc(=O)n(Cc2nc(-c3cccnc3)no2)n1] | 462.90 |
| 156 | 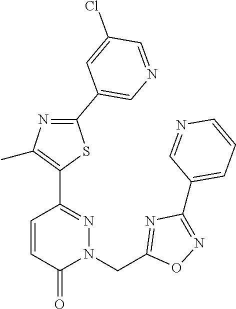 [SMILES: Cc1nc(-c2cncc(Cl)c2)sc1-c1ccc(=O)n(Cc2nc(-c3cccnc3)no2)n1] | 464.00 |
| 157 | 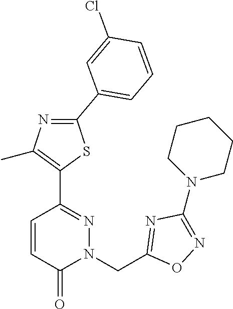 [SMILES: Cc1nc(-c2cccc(Cl)c2)sc1-c1ccc(=O)n(Cc2nc(N3CCCCC3)no2)n1] | 469.15 |
| 158 | 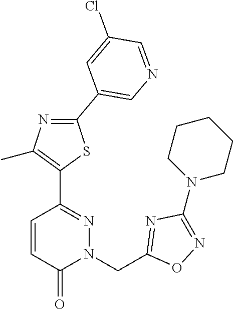 [SMILES: Cc1nc(-c2cncc(Cl)c2)sc1-c1ccc(=O)n(Cc2nc(N3CCCCC3)no2)n1] | 470.20 |
| 159 | 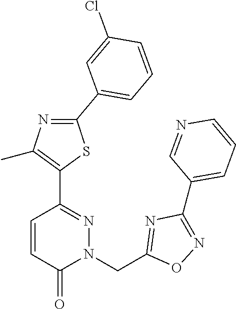 [SMILES: Cc1nc(-c2cccc(Cl)c2)sc1-c1ccc(=O)n(Cc2nc(-c3cccnc3)no2)n1] | |
| 160 | 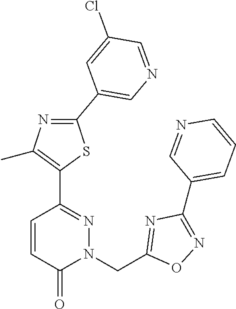 [SMILES: Cc1nc(-c2cncc(Cl)c2)sc1-c1ccc(=O)n(Cc2nc(-c3cccnc3)no2)n1] | |
| 161 | 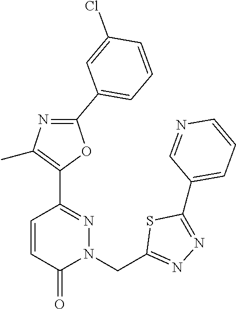 [SMILES: Cc1nc(-c2cccc(Cl)c2)oc1-c1ccc(=O)n(Cc2nnc(-c3cccnc3)s2)n1] | 462.95 |
| 162 | 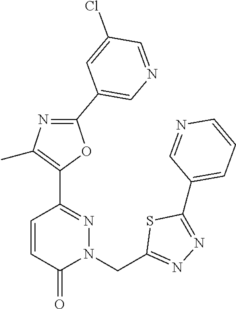 [SMILES: Cc1nc(-c2cncc(Cl)c2)oc1-c1ccc(=O)n(Cc2nnc(-c3cccnc3)s2)n1] | 463.95 |
| 163 | 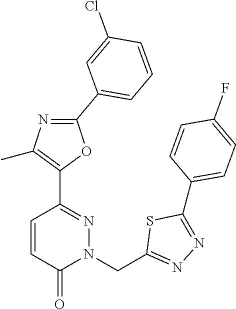 [SMILES: Cc1nc(-c2cccc(Cl)c2)oc1-c1ccc(=O)n(Cc2nnc(-c3ccc(F)cc3)s2)n1] | 480.00 |
| 164 | 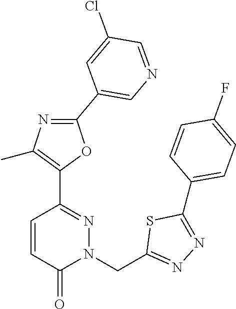 [SMILES: Cc1nc(-c2cncc(Cl)c2)oc1-c1ccc(=O)n(Cc2nnc(-c3ccc(F)cc3)s2)n1] | 481.10 |
| 165 | 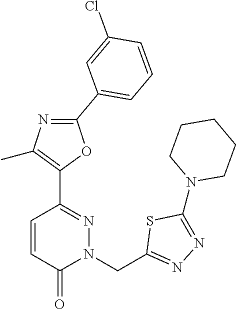 [SMILES: Cc1nc(-c2cccc(Cl)c2)oc1-c1ccc(=O)n(Cc2nnc(N3CCCCC3)s2)n1] | 469.00 |
| 166 | 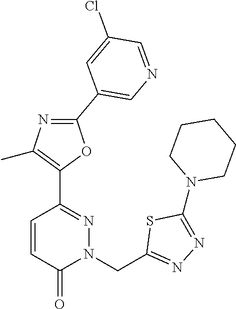 [SMILES: Cc1nc(-c2cncc(Cl)c2)oc1-c1ccc(=O)n(Cc2nnc(N3CCCCC3)s2)n1] | 470.15 |
| 167 | 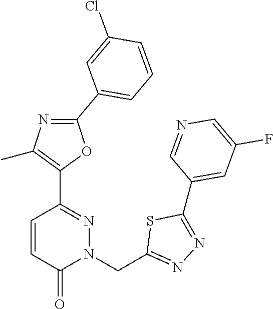 [SMILES: Cc1nc(-c2cccc(Cl)c2)oc1-c1ccc(=O)n(Cc2nnc(-c3cncc(F)c3)s2)n1] | 396.95 |
| 168 | 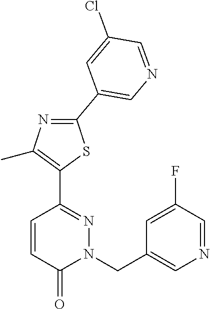 [SMILES: Cc1nc(-c2cncc(Cl)c2)sc1-c1ccc(=O)n(Cc2cncc(F)c2)n1] | 413.90 |
| 169 | 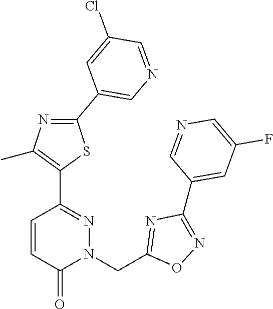 [SMILES: Cc1nc(-c2cncc(Cl)c2)sc1-c1ccc(=O)n(Cc2nc(-c3cncc(F)c3)no2)n1] | 482.00 |
| 212 | 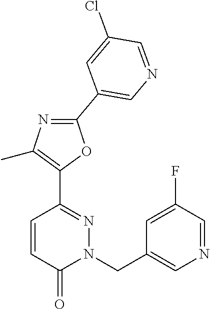 [SMILES: Cc1nc(-c2cncc(Cl)c2)oc1-c1ccc(=O)n(Cc2cncc(F)c2)n1] | 398.10 |

### Example 8. Skeletal Myofibril ATPase Assay

Overview: Myosin ATPase activity was assessed by using a coupled reaction system, in which ADP generated by the myosin ATPase function was coupled to the disappearance of NADH through the pyruvate kinase/lactate dehydrogenase (PK-LDH) system. Myosin ATPase activity produces ADP, which was used as a substrate for PK to produce pyruvate and regenerate ATP. The pyruvate was then used as a substrate by LDH to oxidize NADH to NAD+. The rate of the reaction was monitored through the time-dependent disappearance of NADH using absorbance at 340 nm. Inhibition of ATPase activity by the assayed compounds was indicated by a reduced rate of NADH loss, relative to vehicle-treated controls, over the experimental time window. To assess the selectivity of the assayed compounds for skeletal myofibrils, the compounds were counter-screened in cardiac myofibrils.

Materials: The following stock solutions and reagents were used in the Skeletal Myofibril ATPase Assay:

Stock Solutions

PIPES, 200 mM in H₂O, pH 7.0
MgCl₂ in H₂O, 200 mM
PM12 Buffer, 10X: 12 mM PIPES (from 200 mM stock), 20 mM
MgCl₂ (from 200 mM stock)
EGTA in H₂O, 500 mM
CaCl₂ in H₂O, 500 mM
DTT in H₂O, 1M
BSA in H₂O, 20 mg/mL
KCl in H₂O, 600 mM
ATP in 1X PM12, 100 mM
NADH in 1X PM12, 30 mM
PEP in 1X PM12, 100 mM, pH 7.0
Antifoam 204, 1% in H₂O

Stock Solutions of pCa buffer. Combine PIPES, CaCl₂), and EGTA solutions with 70 mL of water. Adjust pH to 7.0 and bring final volume to 100 mL.

PREPARATION OF STOCKS SOLUTIONS
FOR 100 ML OF PCA BUFFER

| | 200 mM | Approx. | | |
|---|---|---|---|---|
| | PIPES | Water | | |
| pCA | (mL) | (mL) | CaCl₂ | EGTA |
| 4.0 | 6 | 74 | 10.025 | 9.975 |
| 4.5 | 6 | 74 | 9.800 | 10.200 |
| 5.0 | 6 | 74 | 9.325 | 10.675 |
| 5.5 | 6 | 74 | 8.100 | 11.900 |
| 5.75 | 6 | 74 | 7.200 | 12.800 |
| 6.0 | 6 | 74 | 6.000 | 14.000 |
| 6.25 | 6 | 74 | 4.500 | 15.500 |
| 6.5 | 6 | 74 | 3.025 | 16.975 |
| 6.75 | 6 | 74 | 1.975 | 18.025 |
| 7.0 | 6 | 74 | 1.165 | 18.835 |
| 8.0 | 6 | 74 | 0.126 | 19.874 |
| 10.0 | 6 | 74 | 0.001 | 19.999 |

Buffer A & Buffer B. Butters were stored on ice until use.

### Buffer Preparation

| | | | | | Final | | | |
|---|---|---|---|---|---|---|---|---|
| Total Well | | | Stock | | Concentrations in | | Reaction | |
| Volume | | | Concentrations | | Specific Buffer | | Concentrations | |
| (μL) | 50 | Component | Value Unit | | | | | |
| Buffer A | 25 | PM12 Buffer | 10 | x | 1.00 | x | 1.00 | x |
| (μL) | | KCl | 600 | mM | 60.00 | mM | 60.00 | mM |
| | | BSA | 20 | mg/mL | 0.10 | mg/mL | 0.10 | mg/mL |
| | | DTT | 1000 | mM | 1.00 | mM | 1.00 | mM |
| | | PK/LDH | 80 | mM | 0.80 | mM | 0.40 | mM |
| | | Rabbit Psoas Prep 11 | 5.83 | mg/mL | 0.50 | mg/mL | 0.25 | mg/mL |
| | | Antifoam | 1.00% | | 0.01% | | 0.01% | |
| | | Water | | | | | | |
| Buffer B | 25 | PM12 Buffer | 10 | x | 1.00 | x | 1.00 | x |
| (μL) | | pCa Solution | 10 | x | 2.00 | x | 1.00 | x |
| | | KCl | 600 | mM | 60.00 | mM | 60.00 | mM |
| | | BSA | 20 | mg/mL | 0.10 | mg/mL | 0.10 | mg/mL |
| | | DTT | 1000 | mM | 1.00 | mM | 1.00 | mM |
| | | ATP | 100 | mM | 0.10 | mM | 0.05 | mM |
| | | NADH | 30 | mM | 1.00 | mM | 0.50 | mM |
| | | PEP | 100 | mM | 3.00 | mM | 1.50 | mM |

| Antifoam | 1.00% | 0.01% | 0.01% |
|---|---|---|---|
| Water | | | |

| | | | | Number of | | |
|---|---|---|---|---|---|---|
| Total Well | | | Volume | Wells | | |
| Volume | | | per well | Total | 96 | |
| (μL) | 50 | Component | (μL) | Volume (μL) | Prepare Volume (μL) | |
| Buffer A | 25 | PM12 Buffer | 2.50 | 240.00 | 312.00 | PM12 Buffer (1 x) |
| (μL) | | KCl | 2.50 | 240.00 | 312.00 | KCl (60 mM) |
| | | BSA | 0.13 | 12.00 | 15.60 | BSA (0.1 mg/mL) |
| | | DTT | 0.03 | 2.40 | 3.12 | DTT (1 mM) |
| | | PK/LDH | 0.25 | 24.00 | 31.20 | PK/LDH (0.4 mM) |
| | | Rabbit Psoas Prep 11 | 2.14 | 205.83 | 267.58 | Rabbit Psoas Prep 11 (0.25 mg/mL) |
| | | Antifoam | 0.25 | 24.00 | 31.20 | Antifoam (0.01%) |
| | | Water | 17.21 | 1651.77 | 2147.30 | Water |
| | | | 25.00 | 2400.00 | 3120.00 | |
| Buffer B | 25 | PM12 Buffer | 2.50 | 240.00 | 312.00 | PM12 Buffer (1 x) |
| (μL) | | pCa Solution | 5.00 | 480.00 | 624.00 | pCa Solution (1 x) |
| | | KCl | 2.50 | 240.00 | 312.00 | KCl (60 mM) |
| | | BSA | 0.13 | 12.00 | 15.60 | BSA (0.1 mg/mL) |
| | | DTT | 0.03 | 2.40 | 3.12 | DTT (1 mM) |
| | | ATP | 0.03 | 2.40 | 3.12 | ATP (0.05 mM) |
| | | NADH | 0.83 | 80.00 | 104.00 | NADH (0.5 mM) |
| | | PEP | 0.75 | 72.00 | 93.60 | PEP (1.5 mM) |
| | | Antifoam | 0.25 | 24.00 | 31.20 | Antifoam (0.01%) |
| | | Water | 12.99 | 1247.20 | 1621.38 | Water ( ) |
| | | | 25.00 | 2400.00 | 3120.00 | Total |

Skeletal Myofibril ATPase Assay Procedure: BSA, ATP, NADH, PEP, and DTT solutions were thawed at room temperature, then transferred to ice. Pellet-frozen myofibrils (approximately twice the required volume) were transferred into a sufficiently large tube and capped. Myofibrils were thawed by rolling in a water bath for approximately 15 min at room temperature and cooled on ice. Buffers A and B were prepared by adjusting volumes as necessary for required number of wells and stored on ice. 0.5 μL of the compounds to be assayed were added into wells of a 384-well plate. Buffers A and B were mixed by inversion immediately prior to use, then 25 μL of each was dispensed using a Multidrop dispenser (Buffer A first, then Buffer B). The absorbance within the wells was measured at 340 nm, using a kinetic protocol in which the wells are read every 1.5-2 min for 1 h. The reaction rate was qualitatively assessed by subtracting the minimum absorbance value from the maximum value for each well, using either the SoftMax Pro plate reader software or a spreadsheet program such as Excel. Using GraphPad Prism 8.0, the data was normalized, with 100% activity defined as the absorbance change in the 1% DMSO vehicle wells and 0% assigned to no change in absorbance over the course of the experiment. The normalized data were fit to a variable-slope four-parameter logistic model, constraining the bottom to be 0 or greater. Compounds of Table 1 to 3 were tested and results of the assay appear in Table 4 herein. A=IC₅₀ is less than or equal to 10 μM; B=IC₅₀ is greater than 10 μM and less than 100 μM; C=IC₅₀ is greater than 100 μM.

In some embodiments, compounds of the disclosure of Formula (Ia) and (Ib) are below in Table 1.

TABLE 1

| Compound | | |
|---|---|---|
| No. | Structure | Name |
| 1 | 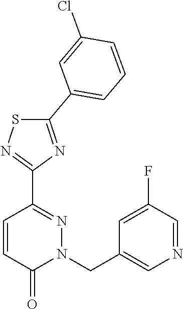 [SMILES: O=c1ccc(-c2nsc(-c3cccc(Cl)c3)n2)nn1Cc1cncc(F)c1] | 6-(5-(3-chlorophenyl)-1,2,4- thiadiazol-3-yl)-2-((5-fluoro- pyridin-3-yl)methyl)pyridazin- 3(2H)-one |
| 2 | 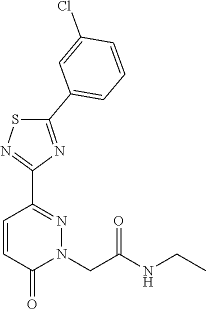 [SMILES: CCNC(=O)Cn1nc(-c2nsc(-c3cccc(Cl)c3)n2)ccc1=O] | 2-(3-(5-(3-chlorophenyl)- 1,2,4-thiadiazol-3-yl)-6-oxo- pyridazin-1(6H)-yl)-N-ethyl- acetamide |
| 3 | 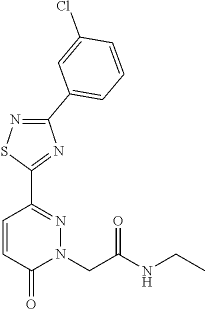 [SMILES: CCNC(=O)Cn1nc(-c2nc(-c3cccc(Cl)c3)ns2)ccc1=O] | 2-(3-(3-(3-chlorophenyl)- 1,2,4-thiadiazol-5-yl)-6-oxo- pyridazin-1(6H)-yl)-N-ethyl- acetamide |
| 4 | 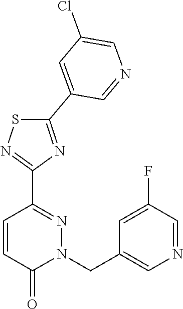 [SMILES: O=c1ccc(-c2nsc(-c3cncc(Cl)c3)n2)nn1Cc1cncc(F)c1] | 6-(5-(5-chloropyridin-3-yl)- 1,2,4-thiadiazol-3-yl)-2-((5- fluoropyridin-3-yl)methyl)- pyridazin-3(2H)-one |
| 5 | 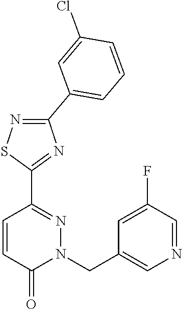 [SMILES: O=c1ccc(-c2nc(-c3cccc(Cl)c3)ns2)nn1Cc1cncc(F)c1] | 6-(3-(3-chlorophenyl)-1,2,4- thiadiazol-5-yl)-2-((5-fluoro- pyridin-3-yl)methyl)pyridazin- 3(2H)-one |
| 6 | 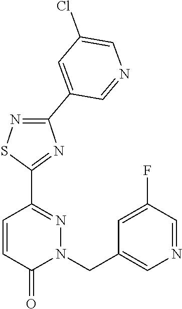 [SMILES: O=c1ccc(-c2nc(-c3cncc(Cl)c3)ns2)nn1Cc1cncc(F)c1] | 6-(3-(5-chloropyridin-3-yl)- 1,2,4-thiadiazol-5-yl)-2-((5- fluoropyridin-3-yl)methyl)- pyridazin-3(2H)-one |
| 7 | 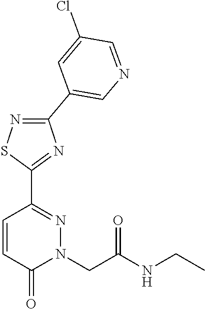 [SMILES: CCNC(=O)Cn1nc(-c2nc(-c3cncc(Cl)c3)ns2)ccc1=O] | 2-(3-(3-(5-chloropyridin-3-yl)- 1,2,4-thiadiazol-5-yl)-6-oxo- pyridazin-1(6H)-yl)-N-ethyl- acetamide |
| 8 | 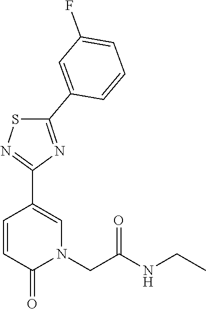 [SMILES: CCNC(=O)Cn1cc(-c2nsc(-c3cccc(F)c3)n2)ccc1=O] | N-ethyl-2-(5-(5-(3-fluorophen- yl)-1,2,4-thiadiazol-3-yl)-2- oxopyridin-1(2H)-yl)acetamide |
| 9 | 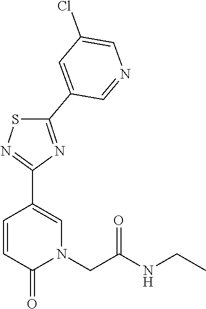 [SMILES: CCNC(=O)Cn1cc(-c2nsc(-c3cncc(Cl)c3)n2)ccc1=O] | 2-(5-(5-(5-chloropyridin-3-yl)- 1,2,4-thiadiazol-3-yl)-2-oxo- pyridin-1(2H)-yl)-N-ethyl- acetamide |
| 10 | 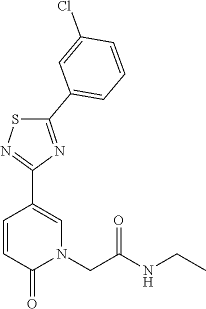 [SMILES: CCNC(=O)Cn1cc(-c2nsc(-c3cccc(Cl)c3)n2)ccc1=O] | 2-(5-(5-(3-chlorophenyl)-1,2,4- thiadiazol-3-yl)-2-oxopyridin- 1(2H)-yl)-N-ethylacetamide |
| 11 | 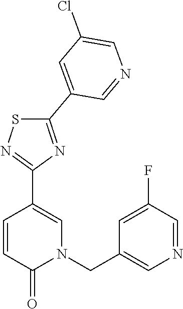 [SMILES: O=c1ccc(-c2nsc(-c3cncc(Cl)c3)n2)cn1Cc1cncc(F)c1] | N-ethyl-2-(5-(5-morpholino- 1,2,4-thiadiazol-3-yl)-2-oxo- pyridin-1(2H)-yl)acetamide |
| 12 | 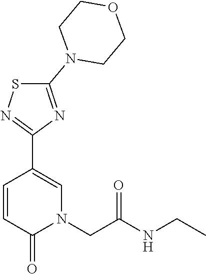 [SMILES: CCNC(=O)Cn1cc(-c2nsc(N3CCOCC3)n2)ccc1=O] | N-ethyl-2-(5-(5-morpholino- 1,2,4-thiadiazol-3-yl)-2-oxo- pyridin-1(2H)-yl)acetamide |
| 13 | 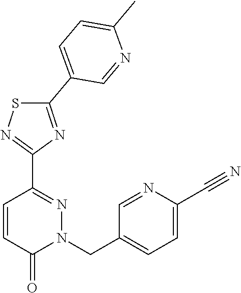 [SMILES: Cc1ccc(-c2nc(-c3ccc(=O)n(Cc4ccc(C#N)nc4)n3)ns2)cn1] | 5-((3-(5-(6-methylpyridin-3- yl)-1,2,4-thiadiazol-3-yl)-6- oxopyridazin-1(6H)-yl)meth- yl)picolinonitrile |
| 14 | 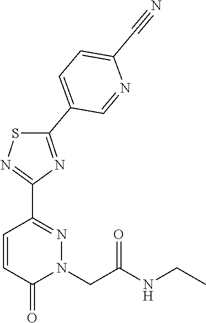 [SMILES: CCNC(=O)Cn1nc(-c2nsc(-c3ccc(C#N)nc3)n2)ccc1=O] | 2-(3-(5-(6-cyanopyridin-3-yl)- 1,2,4-thiadiazol-3-yl)-6-oxo- pyridazin-1(6H)-yl)-N-ethyl- acetamide |
| 15 | 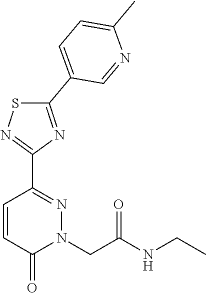 [SMILES: CCNC(=O)Cn1nc(-c2nsc(-c3ccc(C)nc3)n2)ccc1=O] | N-ethyl-2-(3-(5-(6-methyl- pyridin-3-yl)-1,2,4-thiadiazol- 3-yl)-6-oxopyridazin-1(6H)- yl)acetamide |
| 16 | 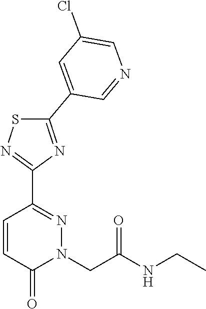 [SMILES: CCNC(=O)Cn1nc(-c2nsc(-c3cncc(Cl)c3)n2)ccc1=O] | 2-(3-(5-(5-chloropyridin-3-yl)- 1,2,4-thiadiazol-3-yl)-6-oxo- pyridazin-1(6H)-yl)-N-ethyl- acetamide |
| 17 | 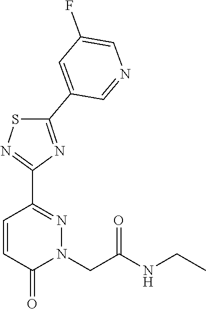 [SMILES: CCNC(=O)Cn1nc(-c2nsc(-c3cncc(F)c3)n2)ccc1=O] | N-ethyl-2-(3-(5-(5-fluoro- pyridin-3-yl)-1,2,4-thiadiazol- 3-yl)-6-oxopyridazin-1(6H)- yl)acetamide |
| 18 | 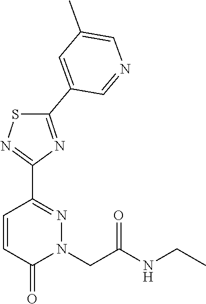 [SMILES: CCNC(=O)Cn1nc(-c2nsc(-c3cncc(C)c3)n2)ccc1=O] | N-ethyl-2-(3-(5-(5-methyl- pyridin-3-yl)-1,2,4-thiadiazol- 3-yl)-6-oxopyridazin-1(6H)- yl)acetamide |
| 19 | 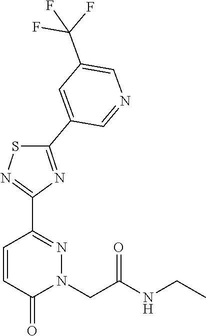 [SMILES: CCNC(=O)Cn1nc(-c2nsc(-c3cncc(C(F)(F)F)c3)n2)ccc1=O] | N-ethyl-2-(6-oxo-3-(5-(5- (trifluoromethyl)pyridin-3-yl)- 1,2,4-thiadiazol-3-yl)pyridazin- 1(6H)-yl)acetamide |
| 20 | 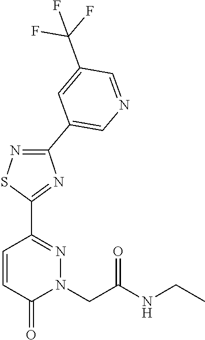 [SMILES: CCNC(=O)Cn1nc(-c2nc(-c3cncc(C(F)(F)F)c3)ns2)ccc1=O] | N-ethyl-2-(6-oxo-3-(5-(5- (trifluoromethyl)pyridin-3-yl)- 1,2,4-thiadiazol-3-yl)pyridazin- 1(6H)-yl)acetamide |
| 21 | 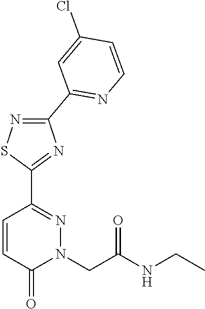 [SMILES: CCNC(=O)Cn1nc(-c2nc(-c3cc(Cl)ccn3)ns2)ccc1=O] | 2-(3-(3-(4-chloropyridin-2-yl)- 1,2,4-thiadiazol-5-yl)-6-oxo- pyridazin-1(6H)-yl)-N-ethyl- acetamide |
| 22 | 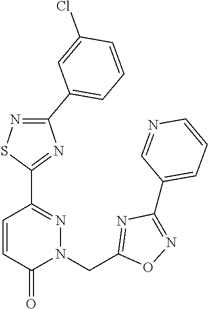 [SMILES: O=c1ccc(-c2nc(-c3cccc(Cl)c3)ns2)nn1Cc1nc(-c2cccnc2)no1] | 6-(3-(3-chlorophenyl)-1,2,4- thiadiazol-5-yl)-2-((3-(pyridin- 3-yl)-1,2,4-oxadiazol-5-yl)- methyl)pyridazin-3(2H)-one |
| 23 | 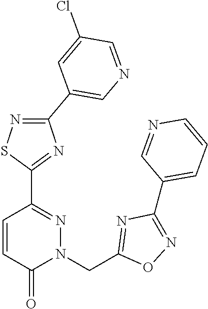 [SMILES: O=c1ccc(-c2nc(-c3cncc(Cl)c3)ns2)nn1Cc1nc(-c2cccnc2)no1] | 6-(3-(5-chloropyridin-3-yl)- 1,2,4-thiadiazol-5-yl)-2-((3- (pyridin-3-yl)-1,2,4-oxadiazol- 5-yl)methyl)pyridazin-3(2H)- one |
| 24 | 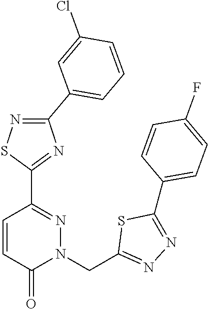 [SMILES: O=c1ccc(-c2nc(-c3cccc(Cl)c3)ns2)nn1Cc1nnc(-c2ccc(F)cc2)s1] | 6-(3-(3-chlorophenyl)-1,2,4- thiadiazol-5-yl)-2-((5-(4- fluorophenyl)-1,3,4-thiadiazol- 2-yl)methyl)pyridazin-3(2H)- one |
| 25 | 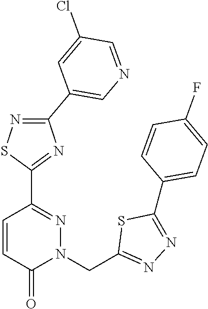 [SMILES: O=c1ccc(-c2nc(-c3cncc(Cl)c3)ns2)nn1Cc1nnc(-c2ccc(F)cc2)s1] | 6-(3-(5-chloropyridin-3-yl)- 1,2,4-thiadiazol-5-yl)-2-((5-(4- fluorophenyl)-1,3,4-thiadiazol- 2-yl)methyl)pyridazin-3(2H)- one |
| 26 | 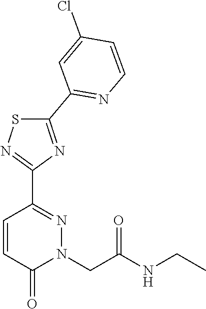 [SMILES: CCNC(=O)Cn1nc(-c2nsc(-c3cc(Cl)ccn3)n2)ccc1=O] | 2-(3-(5-(4-chloropyridin-2-yl)- 1,2,4-thiadiazol-3-yl)-6-oxo- pyridazin-1(6H)-yl)-N-ethyl- acetamide |
| 27 | 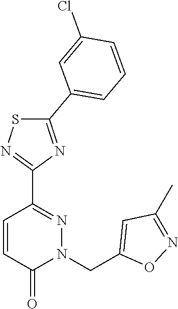 [SMILES: Cc1cc(Cn2nc(-c3nsc(-c4cccc(Cl)c4)n3)ccc2=O)on1] | 6-(5-(3-chlorophenyl)-1,2,4- thiadiazol-3-yl)-2-((3-methyl- isoxazol-5-yl)methyl)pyridazin- 3(2H)-one |
| 28 | 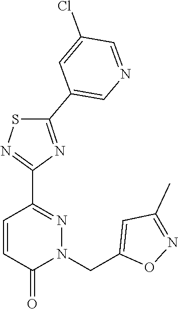 [SMILES: Cc1cc(Cn2nc(-c3nsc(-c4cncc(Cl)c4)n3)ccc2=O)on1] | 6-(5-(5-chloropyridin-3-yl)- 1,2,4-thiadiazol-3-yl)-2-((3- methylisoxazol-5-yl)methyl)- pyridazin-3(2H)-one |
| 29 | 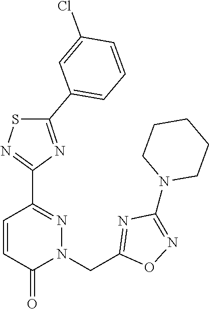 [SMILES: O=c1ccc(-c2nsc(-c3cccc(Cl)c3)n2)nn1Cc1nc(N2CCCCC2)no1] | 6-(5-(3-chlorophenyl)-1,2,4- thiadiazol-3-yl)-2-((3-(piperi- din-1-yl)-1,2,4-oxadiazol-5-yl)- methyl)pyridazin-3(2H)-one |
| 30 | 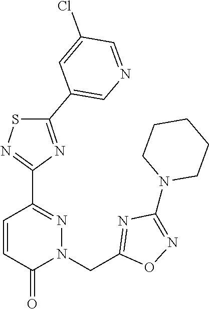 [SMILES: O=c1ccc(-c2nsc(-c3cncc(Cl)c3)n2)nn1Cc1nc(N2CCCCC2)no1] | 6-(5-(5-chloropyridin-3-yl)- 1,2,4-thiadiazol-3-yl)-2-((3- (piperidin-1-yl)-1,2,4-oxadi- azol-5-yl)methyl)pyridazin- 3(2H)-one |
| 31 | 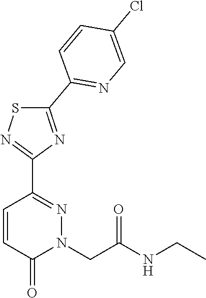 [SMILES: CCNC(=O)Cn1nc(-c2nsc(-c3ccc(Cl)cn3)n2)ccc1=O] | 2-(3-(5-(5-chloropyridin-2-yl)- 1,2,4-thiadiazol-3-yl)-6-oxo- pyridazin-1(6H)-yl)-N-ethyl- acetamide |
| 32 | 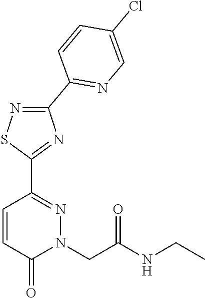 [SMILES: CCNC(=O)Cn1nc(-c2nc(-c3ccc(Cl)cn3)ns2)ccc1=O] | 2-(3-(3-(5-chloropyridin-2-yl)- 1,2,4-thiadiazol-5-yl)-6-oxo- pyridazin-1(6H)-yl)-N-ethyl- acetamide |

In some embodiments, compounds of the disclosure of Formula (II) are below in Table 2.

TABLE 2

| Compound | | |
|---|---|---|
| No. | Structure | Name |
| 51 | 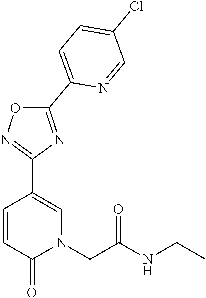 [SMILES: CCNC(=O)Cn1cc(-c2noc(-c3ccc(Cl)cn3)n2)ccc1=O] | 2-(5-(5-(4-chloropyridin-2-yl)- 1,2,4-oxadiazol-3-yl)-2-oxopyri- din-1(2H)-yl)-N-ethylacetamide |
| 52 | 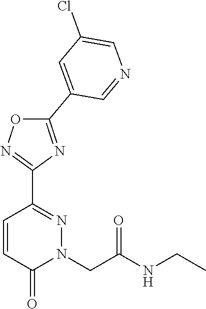 [SMILES: CCNC(=O)Cn1nc(-c2noc(-c3cncc(Cl)c3)n2)ccc1=O] | 2-(3-(5-(5-chloropyridin-3-yl)- 1,2,4-oxadiazol-3-yl)-6-oxopyrid- azin-1(6H)-yl)-N-ethylacetamide |
| 53 | 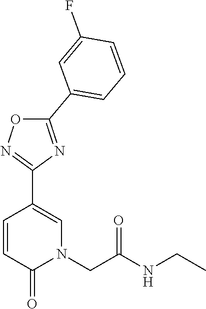 [SMILES: CCNC(=O)Cn1cc(-c2noc(-c3cccc(F)c3)n2)ccc1=O] | N-ethyl-2-(5-(5-(3-fluorophenyl)- 1,2,4-oxadiazol-3-yl)-2-oxopyri- din-1(2H)-yl)acetamide |
| 54 | 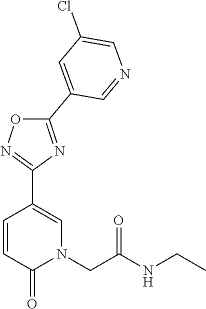 [SMILES: CCNC(=O)Cn1cc(-c2noc(-c3cncc(Cl)c3)n2)ccc1=O] | 2-(5-(5-(5-chloropyridin-3-yl)- 1,2,4-oxadiazol-3-yl)-2-oxopyri- din-1(2H)-yl)-N-ethylacetamide |
| 55 | 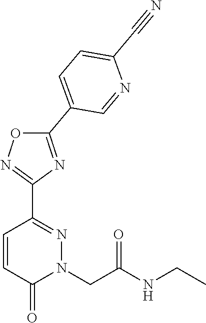 [SMILES: CCNC(=O)Cn1nc(-c2noc(-c3ccc(C#N)nc3)n2)ccc1=O] | 2-(3-(5-(6-cyanopyridin-3-yl)- 1,2,4-oxadiazol-3-yl)-6-oxopyrid- azin-1(6H)-yl)-N-ethylacetamide |
| 56 | 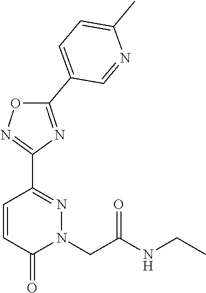 [SMILES: CCNC(=O)Cn1nc(-c2noc(-c3ccc(C)nc3)n2)ccc1=O] | N-ethyl-2-(3-(5-(6-methylpyridin- 3-yl)-1,2,4-oxadiazol-3-yl)-6-oxo- pyridazin-1(6H)-yl)acetamide |
| 57 | 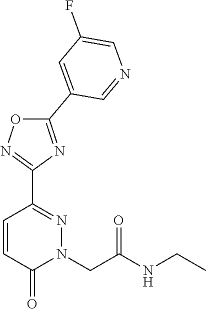 [SMILES: CCNC(=O)Cn1nc(-c2noc(-c3cncc(F)c3)n2)ccc1=O] | N-ethyl-2-(3-(5-(5-fluoropyridin- 3-yl)-1,2,4-oxadiazol-3-yl)-6-oxo- pyridazin-1(6H)-yl)acetamide |
| 58 | 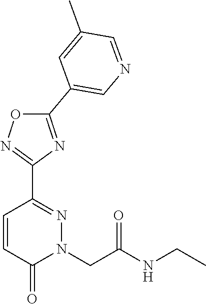 [SMILES: CCNC(=O)Cn1nc(-c2noc(-c3cncc(C)c3)n2)ccc1=O] | N-ethyl-2-(3-(5-(5-methylpyridin- 3-yl)-1,2,4-oxadiazol-3-yl)-6-oxo- pyridazin-1(6H)-yl)acetamide |

In some embodiments, compounds of the disclosure of Formula (III) are below in Table 3.

TABLE 3

| Compound | | |
|---|---|---|
| No. | Structure | Name |
| 70 | 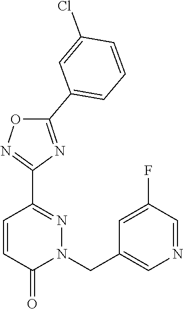 [SMILES: O=c1ccc(-c2noc(-c3cccc(Cl)c3)n2)nn1Cc1cncc(F)c1] | 6-(5-(3-chlorophenyl)-1,2,4- oxadiazol-3-yl)-2-((5-fluoro- pyridin-3-yl)methyl)pyrid- azin-3(2H)-one |
| 71 | 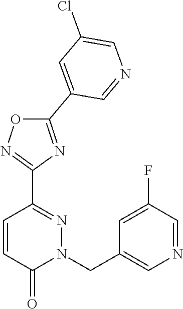 [SMILES: O=c1ccc(-c2noc(-c3cncc(Cl)c3)n2)nn1Cc1cncc(F)c1] | 6-(5-(5-chloropyridin-3-yl)- 1,2,4-oxadiazol-3-yl)-2-((5- fluoropyridin-3-yl)methyl)- pyridazin-3(2H)-one |
| 72 | 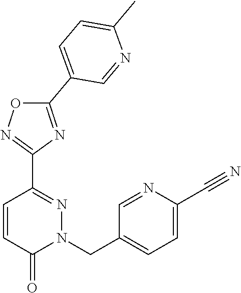 [SMILES: Cc1ccc(-c2nc(-c3ccc(=O)n(Cc4ccc(C#N)nc4)n3)no2)cn1] | 5-((3-(5-(6-methylpyridin-3- yl)-1,2,4-oxadiazol-3-yl)-6- oxopyridazin-1(6H)-yl)- methyl)picolinonitrile |
| 73 | 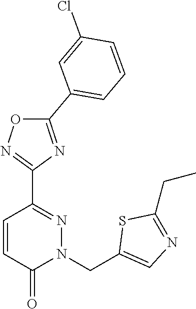 [SMILES: CCc1ncc(Cn2nc(-c3noc(-c4cccc(Cl)c4)n3)ccc2=O)s1] | 6-(5-(3-chlorophenyl)-1,2,4- oxadiazol-3-yl)-2-((2-ethyl- thiazol-5-yl)methyl)pyridazin- 3(2H)-one |
| 74 | 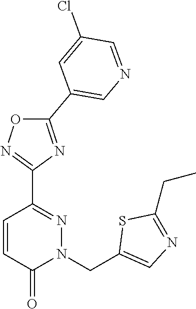 [SMILES: CCc1ncc(Cn2nc(-c3noc(-c4cncc(Cl)c4)n3)ccc2=O)s1] | 6-(5-(5-chloropyridin-3-yl)- 1,2,4-oxadiazol-3-yl)-2-((2- ethylthiazol-5-yl)methyl)- pyridazin-3(2H)-one |
| 75 | 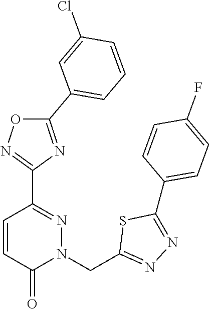 [SMILES: O=c1ccc(-c2noc(-c3cccc(Cl)c3)n2)nn1Cc1nnc(-c2ccc(F)cc2)s1] | 6-(5-(3-chlorophenyl)-1,2,4- oxadiazol-3-yl)-2-((5-(4- fluorophenyl)-1,3,4-thiadi- azol-2-yl)methyl)pyridazin- 3(2H)-one |
| 76 | 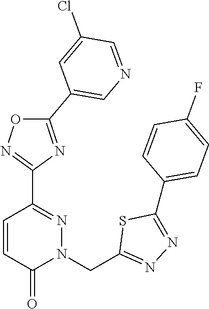 [SMILES: O=c1ccc(-c2noc(-c3cncc(Cl)c3)n2)nn1Cc1nnc(-c2ccc(F)cc2)s1] | 6-(5-(5-chloropyridin-3-yl)- 1,2,4-oxadiazol-3-yl)-2-((5- (4-fluorophenyl)-1,3,4-thiadi- azol-2-yl)methyl)pyridazin- 3(2H)-one |
| 77 | 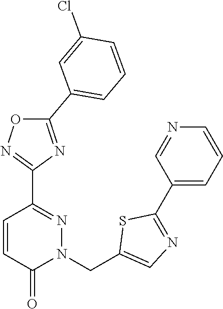 [SMILES: O=c1ccc(-c2noc(-c3cccc(Cl)c3)n2)nn1Cc1cnc(-c2cccnc2)s1] | 6-(5-(3-chlorophenyl)-1,2,4- oxadiazol-3-yl)-2-((2-(pyri- din-3-yl)thiazol-5-yl)methyl)- pyridazin-3(2H)-one |
| 78 | 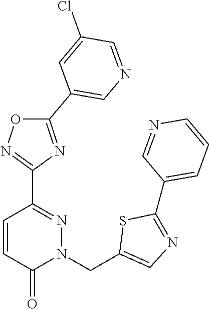 [SMILES: O=c1ccc(-c2noc(-c3cncc(Cl)c3)n2)nn1Cc1cnc(-c2cccnc2)s1] | 6-(5-(5-chloropyridin-3-yl)- 1,2,4-oxadiazol-3-yl)-2-((2- (pyridin-3-yl)thiazol-5-yl)- methyl)pyridazin-3(2H)-one |
| 79 | 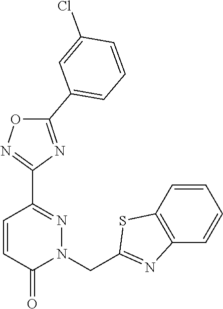 [SMILES: O=c1ccc(-c2noc(-c3cccc(Cl)c3)n2)nn1Cc1nc2ccccc2s1] | 2-(benzo[d]thiazol-2-ylmeth- yl)-6-(5-(3-chlorophenyl)- 1,2,4-oxadiazol-3-yl)pyrid- azin-3(2H)-one |
| 80 | 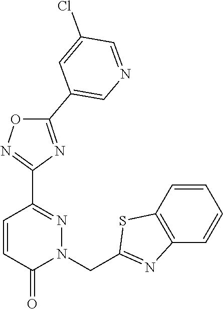 [SMILES: O=c1ccc(-c2noc(-c3cncc(Cl)c3)n2)nn1Cc1nc2ccccc2s1] | 2-(benzo[d]thiazol-2-ylmeth- yl)-6-(5-(5-chloropyridin-3- yl)-1,2,4-oxadiazol-3-yl)- pyridazin-3(2H)-one |
| 81 | 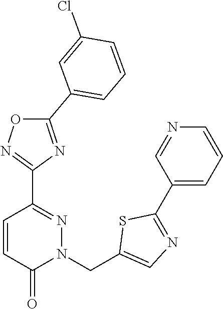 [SMILES: O=c1ccc(-c2noc(-c3cccc(Cl)c3)n2)nn1Cc1cnc(-c2cccnc2)s1] | 6-(5-(3-chlorophenyl)-1,2,4- oxadiazol-3-yl)-2-((2-(pyri- din-3-yl)thiazol-5-yl)meth- yl)pyridazin-3(2H)-one |

In some embodiments, compounds of the disclosure of Formula (IV) are below in Table 4.

TABLE 4

| Compound | | |
|---|---|---|
| No. | Structure | Name |
| 90 | 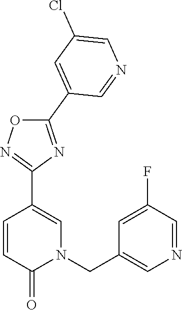 [SMILES: O=c1ccc(-c2noc(-c3cncc(Cl)c3)n2)cn1Cc1cncc(F)c1] | 5-(5-(5-chloropyridin-3-yl)-1,2,4- oxadiazol-3-yl)-1-((5- fluoropyridin-3-yl)methyl)pyridin- 2(1H)-one |

In some embodiments, compounds of the disclosure of Formula (V) are below in Table 5.

TABLE 5

| Compound | | |
|---|---|---|
| No. | Structure | Name |
| 100 | 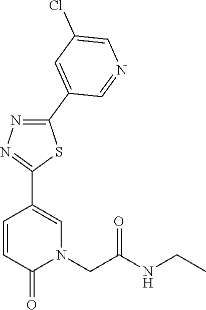 [SMILES: CCNC(=O)Cn1cc(-c2nnc(-c3cncc(Cl)c3)s2)ccc1=O] | 2-(5-(5-(5-chloropyridin-3-yl)- 1,3,4-thiadiazol-2-yl)-2- oxopyridin-1(2H)-yl)-N- ethylacetamide |
| 101 | 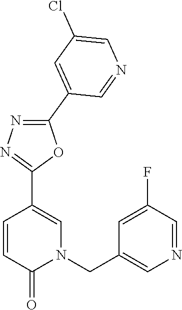 [SMILES: O=c1ccc(-c2nnc(-c3cncc(Cl)c3)o2)cn1Cc1cncc(F)c1] | 5-(5-(5-chloropyridin-3-yl)-1,3,4- oxadiazol-2-yl)-1-((5- fluoropyridin-3-yl)methyl)pyridin- 2(1H)-one |
| 102 | 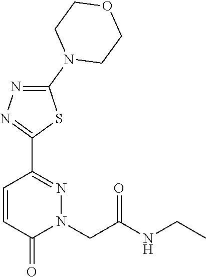 [SMILES: CCNC(=O)Cn1nc(-c2nnc(N3CCOCC3)s2)ccc1=O] | N-ethyl-2-(3-(5-morpholino-1,3,4- thiadiazol-2-yl)-6-oxopyridazin- 1(6H)-yl)acetamide |
| 103 | 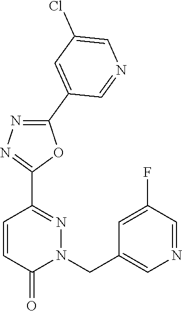 [SMILES: O=c1ccc(-c2nnc(-c3cncc(Cl)c3)o2)nn1Cc1cncc(F)c1] | 6-(5-(5-chloropyridin-3-yl)-1,3,4- oxadiazol-2-yl)-2-((5- fluoropyridin-3- yl)methyl)pyridazin-3(2H)-one |
| 104 | 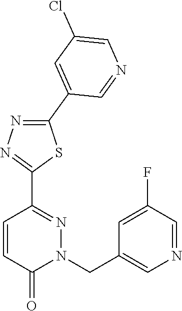 [SMILES: O=c1ccc(-c2nnc(-c3cncc(Cl)c3)s2)nn1Cc1cncc(F)c1] | 6-(5-(5-chloropyridin-3-yl)-1,3,4- thiadiazol-2-yl)-2-((5- fluoropyridin-3- yl)methyl)pyridazin-3(2H)-one |
| 105 | 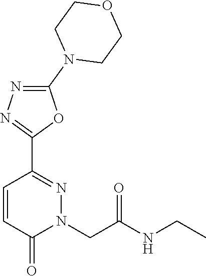 [SMILES: CCNC(=O)Cn1nc(-c2nnc(N3CCOCC3)o2)ccc1=O] | N-ethyl-2-(3-(5-morpholino-1,3,4- oxadiazol-2-yl)-6-oxopyridazin- 1(6H)-yl)acetamide |
| 106 | 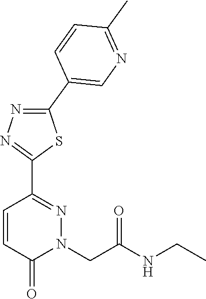 [SMILES: CCNC(=O)Cn1nc(-c2nnc(-c3ccc(C)nc3)s2)ccc1=O] | N-ethyl-2-(3-(5-(6-methylpyridin- 3-yl)-1,3,4-thiadiazol-2-yl)-6- oxopyridazin-1(6H)-yl)acetamide |
| 107 | 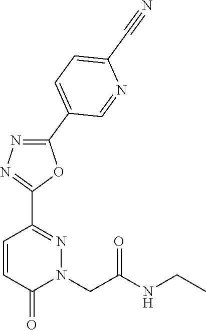 [SMILES: CCNC(=O)Cn1nc(-c2nnc(-c3ccc(C#N)nc3)o2)ccc1=O] | 2-(3-(5-(6-cyanopyridin-3-yl)- 1,3,4-oxadiazol-2-yl)-6- oxopyridazin-1(6H)-yl)-N- ethylacetamide |
| 108 | 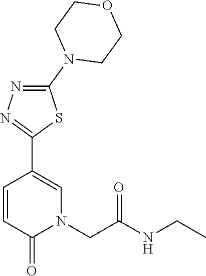 [SMILES: CCNC(=O)Cn1cc(-c2nnc(N3CCOCC3)s2)ccc1=O] | N-ethyl-2-(5-(5-morpholino-1,3,4- thiadiazol-2-yl)-2-oxopyridin- 1(2H)-yl)acetamide |
| 109 | 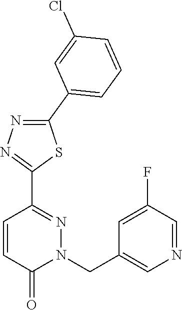 [SMILES: O=c1ccc(-c2nnc(-c3cccc(Cl)c3)s2)nn1Cc1cncc(F)c1] | 6-(5-(3-chlorophenyl)-1,3,4- thiadiazol-2-yl)-2-((5- fluoropyridin-3- yl)methyl)pyridazin-3(2H)-one |
| 110 | 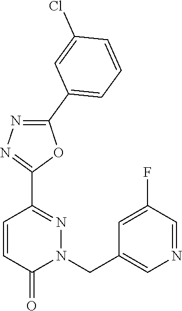 [SMILES: O=c1ccc(-c2nnc(-c3cccc(Cl)c3)o2)nn1Cc1cncc(F)c1] | 6-(5-(3-chlorophenyl)-1,3,4- oxadiazol-2-yl)-2-((5- fluoropyridin-3- yl)methyl)pyridazin-3(2H)-one |
| 111 | 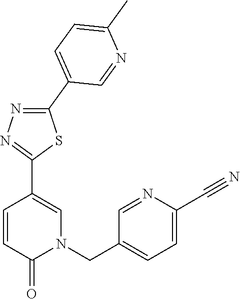 [SMILES: Cc1ccc(-c2nnc(-c3ccc(=O)n(Cc4ccc(C#N)nc4)c3)s2)cn1] | 5-((5-(5-(6-methylpyridin-3-yl)- 1,3,4-thiadiazol-2-yl)-2- oxopyridin-1(2H)- yl)methyl)picolinonitrile |
| 112 | 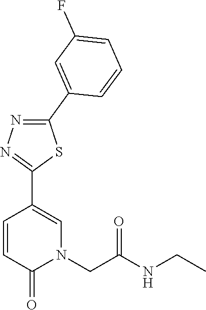 [SMILES: CCNC(=O)Cn1cc(-c2nnc(-c3cccc(F)c3)s2)ccc1=O] | N-ethyl-2-(5-(5-(3-fluorophenyl)- 1,3,4-thiadiazol-2-yl)-2- oxopyridin-1(2H)-yl)acetamide |
| 113 | 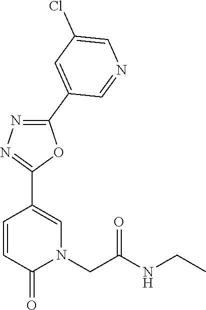 [SMILES: CCNC(=O)Cn1cc(-c2nnc(-c3cncc(Cl)c3)o2)ccc1=O] | 2-(5-(5-(5-chloropyridin-3-yl)- 1,3,4-oxadiazol-2-yl)-2- oxopyridin-1(2H)-yl)-N- ethylacetamide |
| 114 | 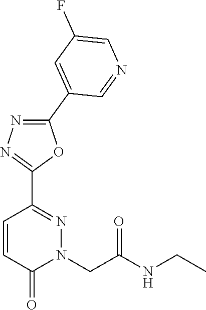 [SMILES: CCNC(=O)Cn1nc(-c2nnc(-c3cncc(F)c3)o2)ccc1=O] | N-ethyl-2-(3-(5-(5-fluoropyridin- 3-yl)-1,3,4-oxadiazol-2-yl)-6- oxopyridazin-1(6H)-yl)acetamide |
| 115 | 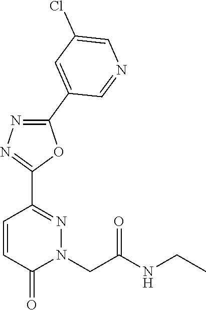 [SMILES: CCNC(=O)Cn1nc(-c2nnc(-c3cncc(Cl)c3)o2)ccc1=O] | 2-(3-(5-(5-chloropyridin-3-yl)- 1,3,4-oxadiazol-2-yl)-6- oxopyridazin-1(6H)-yl)-N- ethylacetamide |
| 116 | 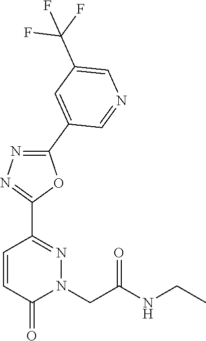 [SMILES: CCNC(=O)Cn1nc(-c2nnc(-c3cncc(C(F)(F)F)c3)o2)ccc1=O] | N-ethyl-2-(6-oxo-3-(5-(5- (trifluoromethyl)pyridin-3-yl)- 1,3,4-oxadiazol-2-yl)pyridazin- 1(6H)-yl)acetamide |
| 117 | 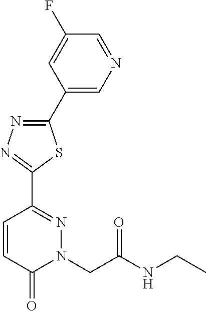 [SMILES: CCNC(=O)Cn1nc(-c2nnc(-c3cncc(F)c3)s2)ccc1=O] | N-ethyl-2-(3-(5-(5-fluoropyridin- 3-yl)-1,3,4-thiadiazol-2-yl)-6- oxopyridazin-1(6H)-yl)acetamide |
| 118 | 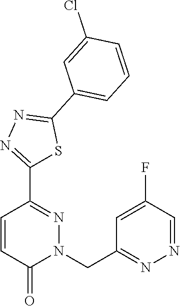 [SMILES: O=c1ccc(-c2nnc(-c3cccc(Cl)c3)s2)nn1Cc1cc(F)cnn1] | 6-(5-(3-chlorophenyl)-1,3,4- thiadiazol-2-yl)-2-((5- fluoropyridazin-3- yl)methyl)pyridazin-3(2H)-one |
| 119 | 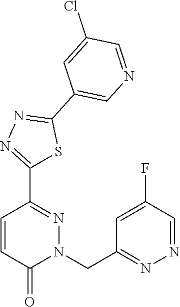 [SMILES: O=c1ccc(-c2nnc(-c3cncc(Cl)c3)s2)nn1Cc1cc(F)cnn1] | 6-(5-(5-chloropyridin-3-yl)-1,3,4- thiadiazol-2-yl)-2-((5- fluoropyridazin-3- yl)methyl)pyridazin-3(2H)-one |
| 120 | 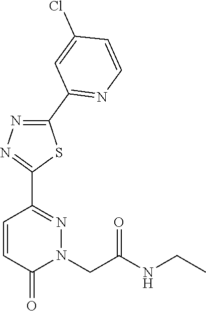 [SMILES: CCNC(=O)Cn1nc(-c2nnc(-c3cc(Cl)ccn3)s2)ccc1=O] | 2-(3-(5-(4-chloropyridin-2-yl)- 1,3,4-thiadiazol-2-yl)-6- oxopyridazin-1(6H)-yl)-N- ethylacetamide |
| 121 | 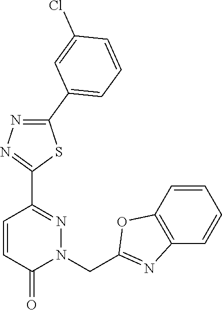 [SMILES: O=c1ccc(-c2nnc(-c3cccc(Cl)c3)s2)nn1Cc1nc2ccccc2o1] | 2-(benzo[d]oxazol-2-ylmethyl)-6- (5-(3-chlorophenyl)-1,3,4- thiadiazol-2-yl)pyridazin-3(2H)- one |
| 122 | 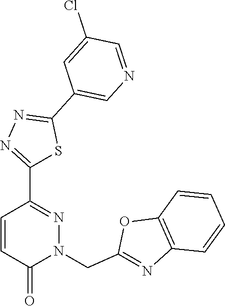 [SMILES: O=c1ccc(-c2nnc(-c3cncc(Cl)c3)s2)nn1Cc1nc2ccccc2o1] | 2-(benzo[d]oxazol-2-ylmethyl)-6- (5-(5-chloropyridin-3-yl)-1,3,4- thiadiazol-2-yl)pyridazin-3(2H)- one |
| 123 | 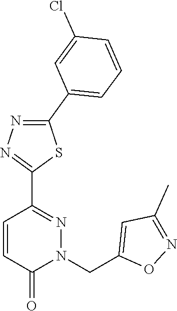 [SMILES: Cc1cc(Cn2nc(-c3nnc(-c4cccc(Cl)c4)s3)ccc2=O)on1] | 6-(5-(3-chlorophenyl)-1,3,4- thiadiazol-2-yl)-2-((3- methylisoxazol-5- yl)methyl)pyridazin-3(2H)-one |
| 124 | 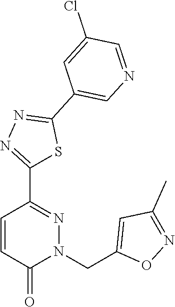 [SMILES: Cc1cc(Cn2nc(-c3nnc(-c4cncc(Cl)c4)s3)ccc2=O)on1] | 6-(5-(5-chloropyridin-3-yl)-1,3,4- thiadiazol-2-yl)-2-((3- methylisoxazol-5- yl)methyl)pyridazin-3(2H)-one |
| 125 | 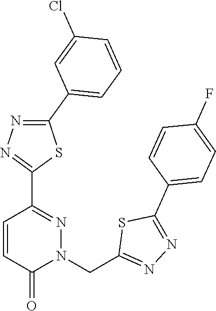 [SMILES: O=c1ccc(-c2nnc(-c3cccc(Cl)c3)s2)nn1Cc1nnc(-c2ccc(F)cc2)s1] | 6-(5-(3-chlorophenyl)-1,3,4- thiadiazol-2-yl)-2-((5-(4- fluorophenyl)-1,3,4-thiadiazol-2- yl)methyl)pyridazin-3(2H)-one |
| 126 | 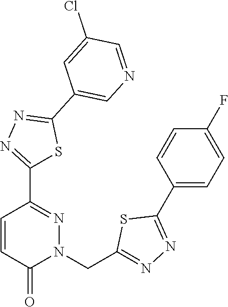 [SMILES: O=c1ccc(-c2nnc(-c3cncc(Cl)c3)s2)nn1Cc1nnc(-c2ccc(F)cc2)s1] | 6-(5-(5-chloropyridin-3-yl)-1,3,4- thiadiazol-2-yl)-2-((5-(4- fluorophenyl)-1,3,4-thiadiazol-2- yl)methyl)pyridazin-3(2H)-one |
| 127 | 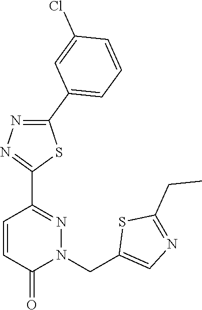 [SMILES: CCc1ncc(Cn2nc(-c3nnc(-c4cccc(Cl)c4)s3)ccc2=O)s1] | 6-(5-(3-chlorophenyl)-1,3,4- thiadiazol-2-yl)-2-((2-ethylthiazol- 5-yl)methyl)pyridazin-3(2H)-one |
| 128 | 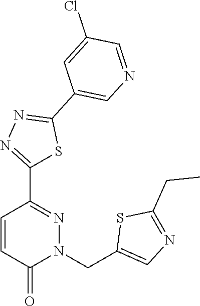 [SMILES: CCc1ncc(Cn2nc(-c3nnc(-c4cncc(Cl)c4)s3)ccc2=O)s1] | 6-(5-(5-chloropyridin-3-yl)-1,3,4- thiadiazol-2-yl)-2-((2-ethylthiazol- 5-yl)methyl)pyridazin-3(2H)-one |
| 129 | 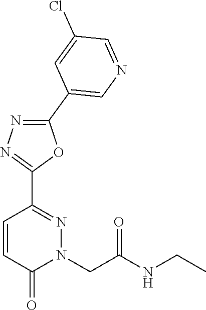 [SMILES: CCNC(=O)Cn1nc(-c2nnc(-c3cncc(Cl)c3)o2)ccc1=O] | 2-(3-(5-(5-chloropyridin-3-yl)- 1,3,4-oxadiazol-2-yl)-6- oxopyridazin-1(6H)-yl)-N- ethylacetamide |
| 130 | 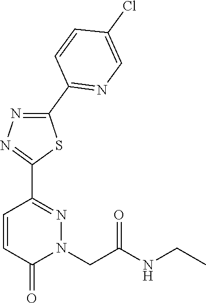 [SMILES: CCNC(=O)Cn1nc(-c2nnc(-c3ccc(Cl)cn3)s2)ccc1=O] | 2-(3-(5-(5-chloropyridin-2-yl)- 1,3,4-thiadiazol-2-yl)-6- oxopyridazin-1(6H)-yl)-N- ethylacetamide |
| 131 | 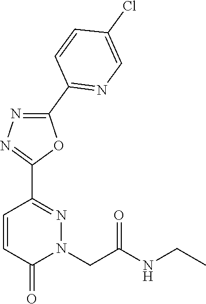 [SMILES: CCNC(=O)Cn1nc(-c2nnc(-c3ccc(Cl)cn3)o2)ccc1=O] | 2-(3-(5-(5-chloropyridin-2-yl)-1,3,4- oxadiazol-2-yl)-6-oxopyridazin-1(6H)- yl)-N-ethylacetamide |

In some embodiments, compounds of the disclosure of Formula (VI) are below in Table 6.

TABLE 6

| Compound | | |
|---|---|---|
| No. | Structure | Name |
| 150 | 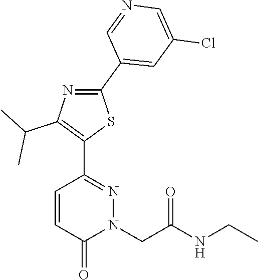 [SMILES: CCNC(=O)Cn1nc(-c2sc(-c3cncc(Cl)c3)nc2C(C)C)ccc1=O] | 2-(3-(2-(5-chloropyridin-3-yl)-4- isopropylthiazol-5-yl)-6- oxopyridazin-1(6H)-yl)-N- ethylacetamide |
| 151 | 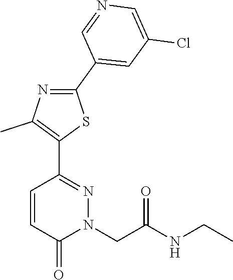 [SMILES: CCNC(=O)Cn1nc(-c2sc(-c3cncc(Cl)c3)nc2C)ccc1=O] | 2-(3-(2-(5-chloropyridin-3-yl)-4- methylthiazol-5-yl)-6- oxopyridazin-1(6H)-yl)-N- ethylacetamide |
| 152 | 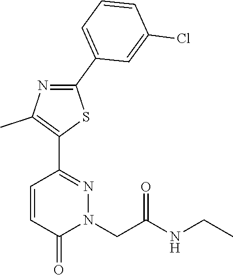 [SMILES: CCNC(=O)Cn1nc(-c2sc(-c3cccc(Cl)c3)nc2C)ccc1=O] | 2-(3-(2-(3-chlorophenyl)-4- methylthiazol-5-yl)-6- oxopyridazin-1(6H)-yl)-N- ethylacetamide |
| 210 | 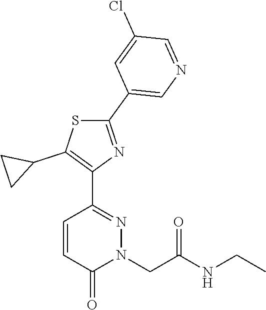 [SMILES: CCNC(=O)Cn1nc(-c2nc(-c3cncc(Cl)c3)sc2C2CC2)ccc1=O] | 2-(3-(2-(5-chloropyridin-3-yl)-5- cyclopropylthiazol-4-yl)-6- oxopyridazin-1(6H)-yl)-N- ethylacetamide |
| 211 | 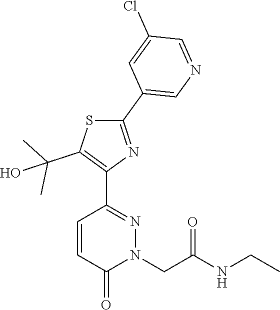 [SMILES: CCNC(=O)Cn1nc(-c2nc(-c3cncc(Cl)c3)sc2C(C)(C)O)ccc1=O] | 2-(3-(2-(5-chloropyridin-3-yl)-5- (2-hydroxypropan-2-yl)thiazol-4- yl)-6-oxopyridazin-1(6H)-yl)-N- ethylacetamide |
| 213 | 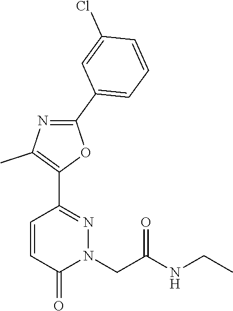 [SMILES: CCNC(=O)Cn1nc(-c2oc(-c3cccc(Cl)c3)nc2C)ccc1=O] | 2-(3-(2-(3-chlorophenyl)-4- methyloxazol-5-yl)-6-oxopyridazin- 1(6H)-yl)-N-ethylacetamide |
| 171 | 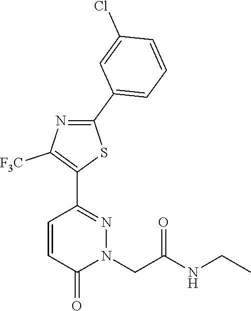 [SMILES: CCNC(=O)Cn1nc(-c2sc(-c3cccc(Cl)c3)nc2C(F)(F)F)ccc1=O] | 2-(3-(2-(3-chlorophenyl)-4- (trifluoromethyl)thiazol-5-yl)-6- oxopyridazin-1(6H)-yl)-N- ethylacetamide |
| 173 | 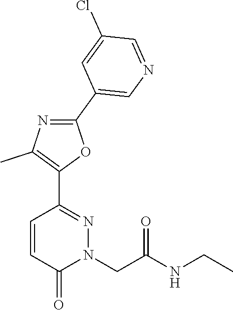 [SMILES: CCNC(=O)Cn1nc(-c2oc(-c3cncc(Cl)c3)nc2C)ccc1=O] | 2-(3-(2-(5-chloropyridin-3-yl)-4- methyloxazol-5-yl)-6-oxopyridazin- 1(6H)-yl)-N-ethylacetamide |

In some embodiments, compounds of the disclosure of Formula (VIII) are below in Table 7.

TABLE 7

| Compound No. | Structure | Name |
|---|---|---|
| 153 | 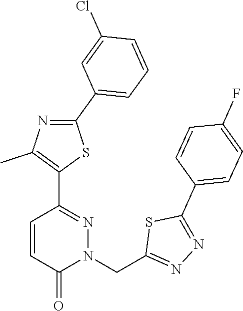 [SMILES: Cc1nc(-c2cccc(Cl)c2)sc1-c1ccc(=O)n(Cc2nnc(-c3ccc(F)cc3)s2)n1] | 6-(2-(3-chlorophenyl)-4- methylthiazol-5-yl)-2-((5- (4-fluorophenyl)-1,3,4- thiadiazol-2- yl)methyl)pyridazin- 3(2H)-one |
| 154 | 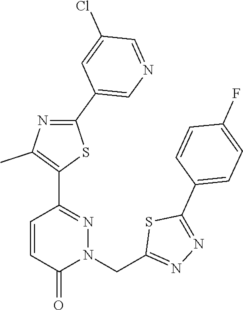 [SMILES: Cc1nc(-c2cncc(Cl)c2)sc1-c1ccc(=O)n(Cc2nnc(-c3ccc(F)cc3)s2)n1] | 6-(2-(5-chloropyridin-3- yl)-4-methylthiazol-5-yl)- 2-((5-(4-fluorophenyl)- 1,3,4-thiadiazol-2- yl)methyl)pyridazin- 3(2H)-one |
| 155 | 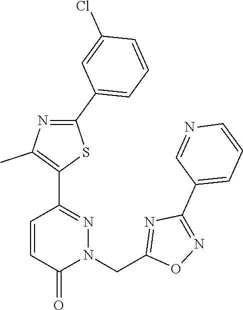 [SMILES: Cc1nc(-c2cccc(Cl)c2)sc1-c1ccc(=O)n(Cc2nc(-c3cccnc3)no2)n1] | 6-(2-(3-chlorophenyl)-4- methylthiazol-5-yl)-2-((3- (pyridin-3-yl)-1,2,4- oxadiazol-5- yl)methyl)pyridazin- 3(2H)-one |
| 156 | 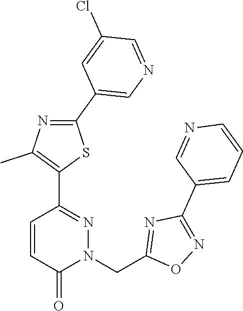 [SMILES: Cc1nc(-c2cncc(Cl)c2)sc1-c1ccc(=O)n(Cc2nc(-c3cccnc3)no2)n1] | 6-(2-(5-chloropyridin-3- yl)-4-methylthiazol-5-yl)- 2-((3-(pyridin-3-yl)-1,2,4- oxadiazol-5- yl)methyl)pyridazin- 3(2H)-one |
| 157 | 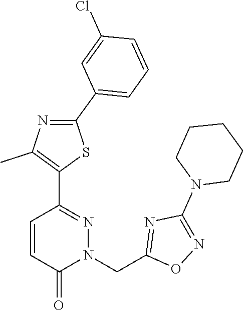 [SMILES: Cc1nc(-c2cccc(Cl)c2)sc1-c1ccc(=O)n(Cc2nc(N3CCCCC3)no2)n1] | 6-(2-(3-chlorophenyl)-4- methylthiazol-5-yl)-2-((3- (piperidin-1-yl)-1,2,4- oxadiazol-5- yl)methyl)pyridazin- 3(2H)-one |
| 158 | 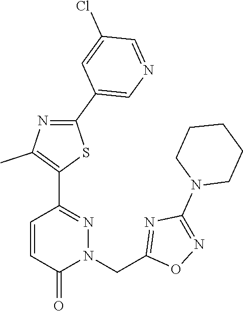 [SMILES: Cc1nc(-c2cncc(Cl)c2)sc1-c1ccc(=O)n(Cc2nc(N3CCCCC3)no2)n1] | 6-(2-(5-chloropyridin-3- yl)-4-methylthiazol-5-yl)- 2-((3-(piperidin-1-yl)- 1,2,4-oxadiazol-5- yl)methyl)pyridazin- 3(2H)-one |
| 159 | 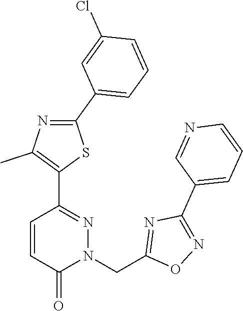 [SMILES: Cc1nc(-c2cccc(Cl)c2)sc1-c1ccc(=O)n(Cc2nc(-c3cccnc3)no2)n1] | 6-(2-(3-chlorophenyl)-4- methylthiazol-5-yl)-2-((3- (pyridin-3-yl)-1,2,4- oxadiazol-5- yl)methyl)pyridazin- 3(2H)-one |
| 160 | 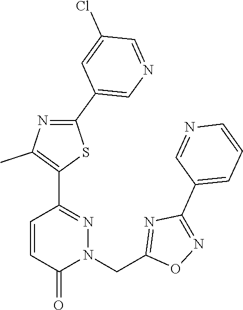 [SMILES: Cc1nc(-c2cncc(Cl)c2)sc1-c1ccc(=O)n(Cc2nc(-c3cccnc3)no2)n1] | 6-(2-(5-chloropyridin-3- yl)-4-methylthiazol-5-yl)- 2-((3-(pyridin-3-yl)-1,2,4- oxadiazol-5- yl)methyl)pyridazin- 3(2H)-one |
| 161 | 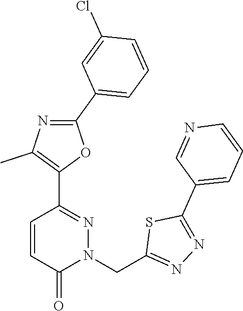 [SMILES: Cc1nc(-c2cccc(Cl)c2)oc1-c1ccc(=O)n(Cc2nnc(-c3cccnc3)s2)n1] | 6-(2-(3-chlorophenyl)-4- methyloxazol-5-yl)-2-((5- (pyridin-3-yl)-1,3,4- thiadiazol-2- yl)methyl)pyridazin- 3(2H)-one |
| 162 | 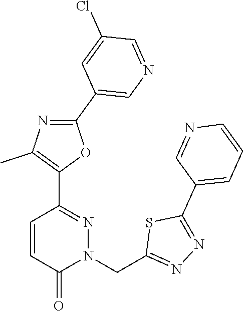 [SMILES: Cc1nc(-c2cncc(Cl)c2)oc1-c1ccc(=O)n(Cc2nnc(-c3cccnc3)s2)n1] | 6-(2-(5-chloropyridin-3- yl)-4-methyloxazol-5-yl)- 2-((5-(pyridin-3-yl)-1,3,4- thiadiazol-2- yl)methyl)pyridazin- 3(2H)-one |
| 163 | 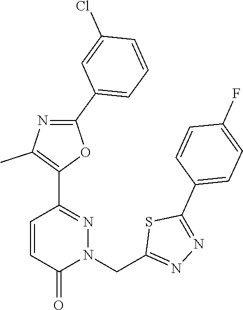 [SMILES: Cc1nc(-c2cccc(Cl)c2)oc1-c1ccc(=O)n(Cc2nnc(-c3ccc(F)cc3)s2)n1] | 6-(2-(3-chlorophenyl)-4- methyloxazol-5-yl)-2-((5- (4-fluorophenyl)-1,3,4- thiadiazol-2- yl)methyl)pyridazin- 3(2H)-one |
| 164 | 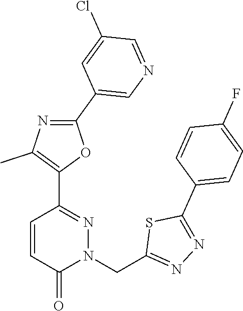 [SMILES: Cc1nc(-c2cncc(Cl)c2)oc1-c1ccc(=O)n(Cc2nnc(-c3ccc(F)cc3)s2)n1] | 6-(2-(5-chloropyridin-3- yl)-4-methyloxazol-5-yl)- 2-((5-(4-fluorophenyl)- 1,3,4-thiadiazol-2- yl)methyl)pyridazin- 3(2H)-one |
| 165 | 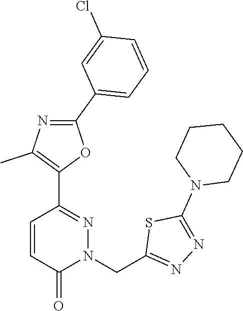 [SMILES: Cc1nc(-c2cccc(Cl)c2)oc1-c1ccc(=O)n(Cc2nnc(N3CCCCC3)s2)n1] | 6-(2-(3-chlorophenyl)-4- methyloxazol-5-yl)-2-((5- (piperidin-1-yl)-1,3,4- thiadiazol-2- yl)methyl)pyridazin- 3(2H)-one |
| 166 | 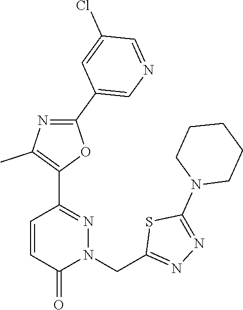 [SMILES: Cc1nc(-c2cncc(Cl)c2)oc1-c1ccc(=O)n(Cc2nnc(N3CCCCC3)s2)n1] | 6-(2-(5-chloropyridin-3- yl)-4-methyloxazol-5-yl)- 2-((5-(piperidin-1-yl)- 1,3,4-thiadiazol-2- yl)methyl)pyridazin- 3(2H)-one |
| 167 | 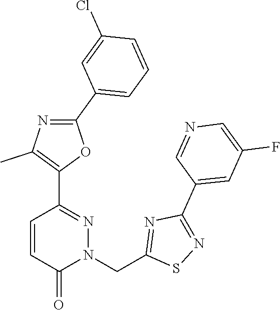 [SMILES: Cc1nc(-c2cccc(Cl)c2)oc1-c1ccc(=O)n(Cc2nc(-c3cncc(F)c3)ns2)n1] | 6-(2-(3-chlorophenyl)-4- methyloxazol-5-yl)-2-((3- (5-fluoropyridin-3-yl)- 1,2,4-thiadiazol-5- yl)methyl)pyridazin- 3(2H)-one |
| 168 | 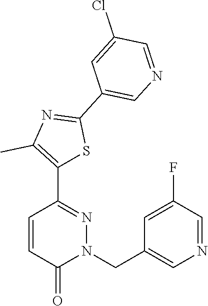 [SMILES: Cc1nc(-c2cncc(Cl)c2)sc1-c1ccc(=O)n(Cc2cncc(F)c2)n1] | 6-(2-(5-chloropyridin-3- yl)-4-methylthiazol-5-yl)- 2-((5-fluoropyridin-3- yl)methyl)pyridazin- 3(2H)-one |
| 169 | 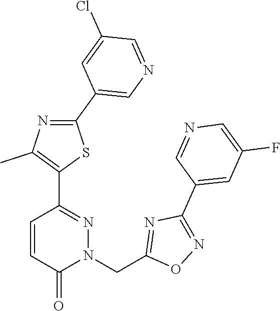 [SMILES: Cc1nc(-c2cncc(Cl)c2)sc1-c1ccc(=O)n(Cc2nc(-c3cncc(F)c3)no2)n1] | 6-(2-(5-chloropyridin-3- yl)-4-methylthiazol-5-yl)- 2-((3-(5-fluoropyridin-3- yl)-1,2,4-oxadiazol-5- yl)methyl)pyridazin- 3(2H)-one |
| 170 | 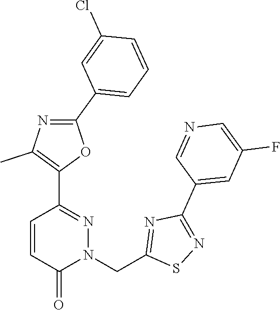 [SMILES: Cc1nc(-c2cccc(Cl)c2)oc1-c1ccc(=O)n(Cc2nc(-c3cncc(F)c3)ns2)n1] | 6-(2-(3-chlorophenyl)-4- methyloxazol-5-yl)-2-((3- (5-fluoropyridin-3-yl)- 1,2,4-thiadiazol-5- yl)methyl)pyridazin- 3(2H)-one |
| 172 | 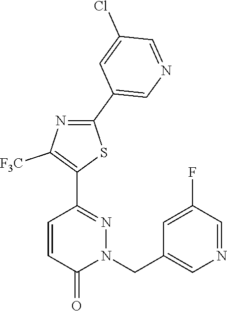 [SMILES: O=c1ccc(-c2sc(-c3cncc(Cl)c3)nc2C(F)(F)F)nn1Cc1cncc(F)c1] | 6-(2-(5-chloropyridin-3- yl)-4- (trifluoromethyl)thiazol-5- yl)-2-((5-fluoropyridin-3- yl)methyl)pyridazin- 3(2H)-one |
| 212 | 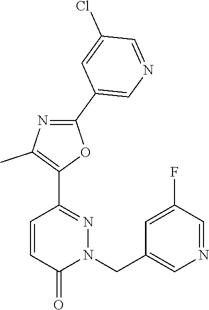 [SMILES: Cc1nc(-c2cncc(Cl)c2)oc1-c1ccc(=O)n(Cc2cncc(F)c2)n1] | 6-(2-(5-chloropyridin-3- yl)-4-methyloxazol-5-yl)- 2-((5-fluoropyridin-3- yl)methyl)pyridazin- 3(2H)-one |

In some embodiments, compounds of the disclosure of Formula (VII) are below in Table 8.

TABLE 8

| Compound No. | Structure | Name |
|---|---|---|
| 190 | 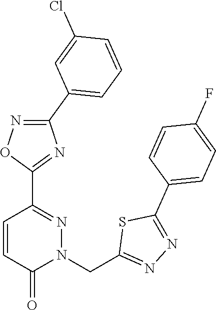 [SMILES: O=c1ccc(-c2nc(-c3cccc(Cl)c3)no2)nn1Cc1nnc(-c2ccc(F)cc2)s1] | 6-(3-(3- chlorophenyl)-1,2,4- oxadiazol-5-yl)-2- ((5-(4- fluorophenyl)-1,3,4- thiadiazol-2- yl)methyl)pyridazin- 3(2H)-one |
| 191 | 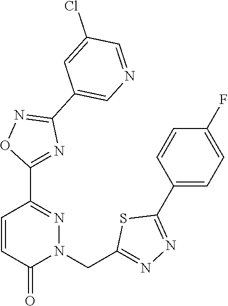 [SMILES: O=c1ccc(-c2nc(-c3cncc(Cl)c3)no2)nn1Cc1nnc(-c2ccc(F)cc2)s1] | 6-(3-(5- chloropyridin-3-yl)- 1,2,4-oxadiazol-5- yl)-2-((5-(4- fluorophenyl)-1,3,4- thiadiazol-2- yl)methyl)pyridazin- 3(2H)-one |
| 192 | 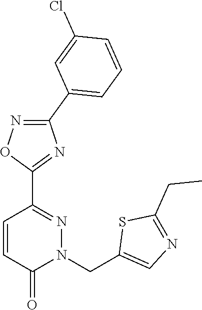 [SMILES: CCc1ncc(Cn2nc(-c3nc(-c4cccc(Cl)c4)no3)ccc2=O)s1] | 6-(3-(3- chlorophenyl)-1,2,4- oxadiazol-5-yl)-2- ((2-ethylthiazol-5- yl)methyl)pyridazin- 3(2H)-one |
| 193 | 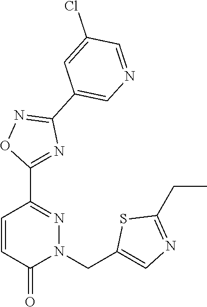 [SMILES: CCc1ncc(Cn2nc(-c3nc(-c4cncc(Cl)c4)no3)ccc2=O)s1] | 6-(3-(5- chloropyridin-3-yl)- 1,2,4-oxadiazol-5- yl)-2-((2- ethylthiazol-5- yl)methyl)pyridazin- 3(2H)-one |
| 194 | 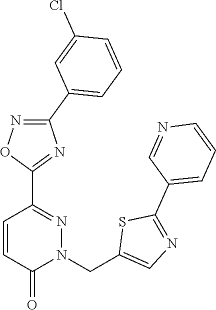 [SMILES: O=c1ccc(-c2nc(-c3cccc(Cl)c3)no2)nn1Cc1cnc(-c2cccnc2)s1] | 6-(3-(3- chlorophenyl)-1,2,4- oxadiazol-5-yl)-2- ((2-(pyridin-3- yl)thiazol-5- yl)methyl)pyridazin- 3(2H)-one |
| 195 | 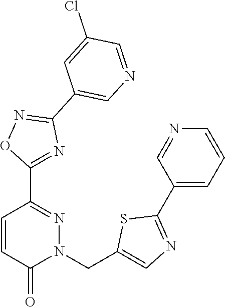 [SMILES: O=c1ccc(-c2nc(-c3cncc(Cl)c3)no2)nn1Cc1cnc(-c2cccnc2)s1] | 6-(3-(5- chloropyridin-3-yl)- 1,2,4-oxadiazol-5- yl)-2-((2-(pyridin-3- yl)thiazol-5- yl)methyl)pyridazin- 3(2H)-one |
| 196 | 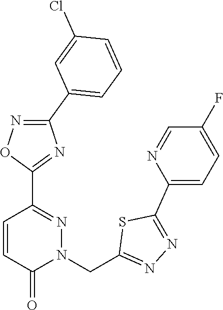 [SMILES: O=c1ccc(-c2nc(-c3cccc(Cl)c3)no2)nn1Cc1nnc(-c2ccc(F)cn2)s1] | 6-(3-(3- chlorophenyl)-1,2,4- oxadiazol-5-yl)-2- ((5-(5-fluoropyridin- 2-yl)-1,3,4- thiadiazol-2- yl)methyl)pyridazin- 3(2H)-one |
| 197 | 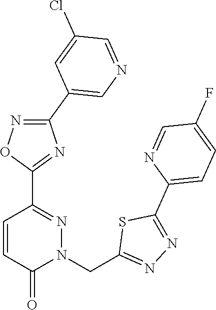 [SMILES: O=c1ccc(-c2nc(-c3cncc(Cl)c3)no2)nn1Cc1nnc(-c2ccc(F)cn2)s1] | 6-(3-(5- chloropyridin-3-yl)- 1,2,4-oxadiazol-5- yl)-2-((5-(5- fluoropyridin-2-yl)- 1,3,4-thiadiazol-2- yl)methyl)pyridazin- 3(2H)-one |
| 198 | 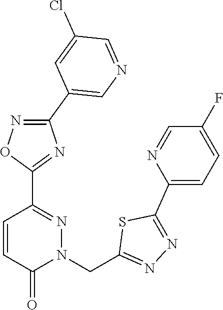 [SMILES: O=c1ccc(-c2nc(-c3cncc(Cl)c3)no2)nn1Cc1nnc(-c2ccc(F)cn2)s1] | 6-(3-(5- chloropyridin-3-yl)- 1,2,4-oxadiazol-5- yl)-2-((5-(5- fluoropyridin-2-yl)- 1,3,4-thiadiazol-2- yl)methyl)pyridazin- 3(2H)-one |
| 199 | 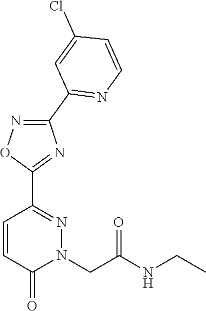 [SMILES: CCNC(=O)Cn1nc(-c2nc(-c3cc(Cl)ccn3)no2)ccc1=O] | 2-(3-(3-(4- chloropyridin-2-yl)- 1,2,4-oxadiazol-5- yl)-6-oxopyridazin- 1(6H)-yl)-N- ethylacetamide |

Skeletal IC₅₀ values of compounds of the disclosure appear in Table 10.

TABLE 10

| Compound No | IC₅₀ |
|---|---|
| 1 | A |
| 2 | A |
| 3 | C |
| 4 | B |
| 5 | A |
| 6 | B |
| 7 | C |
| 8 | A |
| 9 | C |
| 10 | A |
| 11 | B |
| 12 | B |
| 13 | C |
| 14 | C |
| 15 | C |
| 16 | B |
| 17 | C |
| 18 | C |
| 19 | C |
| 20 | C |
| 22 | C |
| 23 | C |
| 24 | C |
| 25 | C |
| 27 | A |
| 28 | C |
| 29 | A |
| 30 | A |
| 31 | B |
| 32 | C |
| 51 | B |
| 52 | A |
| 53 | A |
| 54 | A |
| 55 | C |
| 56 | C |
| 57 | B |
| 58 | B |
| 70 | A |
| 71 | A |
| 72 | C |
| 73 | A |
| 74 | A |
| 75 | A |
| 76 | A |
| 77 | C |
| 78 | A |
| 79 | A |
| 80 | A |
| 81 | C |
| 90 | A |
| 100 | C |
| 101 | C |
| 102 | C |
| 103 | A |
| 104 | A |
| 105 | C |
| 106 | B |
| 107 | C |
| 108 | C |
| 109 | A |
| 110 | A |
| 111 | C |
| 112 | C |
| 113 | C |
| 115 | B |
| 116 | B |
| 117 | B |
| 121 | A |
| 122 | A |
| 123 | A |
| 124 | A |
| 125 | A |
| 126 | A |
| 127 | A |
| 128 | A |
| 130 | A |
| 131 | B |
| 151 | A |
| 152 | A |
| 210 | C |
| 211 | C |
| 213 | C |
| 171 | C |
| 173 | C |
| 153 | A |
| 154 | A |
| 155 | A |
| 156 | A |
| 157 | A |
| 158 | A |
| 159 | A |
| 160 | A |
| 161 | C |
| 162 | C |
| 163 | C |
| 164 | C |
| 165 | A |
| 166 | A |
| 167 | A |
| 168 | A |
| 169 | A |
| 212 | A |
| 170 | C |
| 172 | C |
| 190 | C |
| 191 | C |
| 192 | A |
| 193 | A |
| 194 | C |
| 195 | A |
| 196 | C |
| 197 | A |
| 198 | A |
| 199 | A |

A = IC₅₀ is less than or equal to 10 μM; B = IC₅₀ is greater than 10 μM and less than 100 μM; and C = IC₅₀ is greater than or equal to 100 μM.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A compound represented by Formula (Ia) or (Ib): 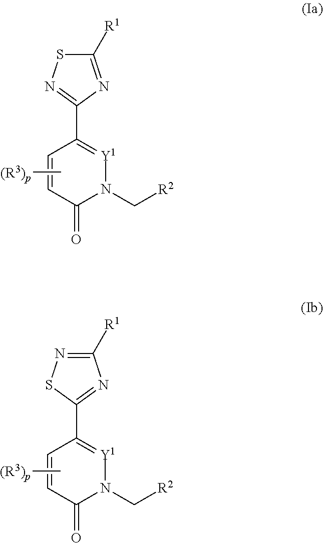
or a salt thereof, wherein:
Y¹ is N or CR⁴;
R¹ is selected from:
hydrogen;
halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, and —CN;
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —N(R⁶)C(O)N(R⁶)₂, —OC(O)N(R⁶)₂, —N(R⁶)C(O)OR⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵; and
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O) R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R² is selected from:
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
—C(O)NR⁷R⁸;
each R³ is independently selected from halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
R⁴ is selected from hydrogen, halogen, —OR⁶, —SR⁶, —N(R⁶)₂, —NO₂, —CN, and C₁₋₆ alkyl optionally substituted with one or more substituents independently selected from halogen, —OR⁶, —SR⁶, —N(R⁶)₂, —NO₂, and —CN;
each R⁵ is independently selected from:
halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN; and
C₁₋₃ alkyl, C₂₋₃ alkenyl, C₂₋₃ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), and —CN;
each R⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁷ is selected from hydrogen and C₁₋₆ alkyl optionally substituted with one or more substituents independently selected from halogen, —OR⁶, —SR⁶, —N(R⁶)₂, —NO₂, and —CN;
R⁸ is selected from:
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₃₋₁₀ carbocycle and 3- to 10-membered heterocycle, wherein the C₃₋₁₀ carbocycle and 3- to 10-membered heterocycle are each optionally substituted with one or more R⁵;
C₃₋₁₀ carbocycle and 3- to 10-membered heterocycle, each of which is optionally substituted with one or more substituents independently selected from: halogen, —OR⁶, —SR⁶, —N(R⁶)₂, —C(O)R⁶, —C(O)N(R⁶)₂, —N(R⁶)C(O)R⁶, —N(R⁶)C(O)N(R⁶)₂, —OC(O)N(R⁶)₂, —N(R⁶)C(O)OR⁶, —C(O)OR⁶, —OC(O)R⁶, —S(O)R⁶, —S(O)₂R⁶, —NO₂, ═O, ═S, ═N(R⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
p is 0, 1, or 2.

2. The compound or salt of claim 1, wherein the compound is represented by Formula (Ia).

3. The compound or salt of claim 1, wherein the compound is represented by Formula (Ib).

4. The compound or salt of claim 1, wherein Y¹ is N.

5. The compound or salt of claim 1, wherein Y¹ is CR⁴.

6. The compound or salt of claim 1, wherein R¹ is selected from:
halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, and —CN;
C₁₋₆ alkyl optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
C₃₋₁₀ carbocycle and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

7. The compound or salt of claim 1, wherein R¹ is selected from: phenyl, pyridinyl, and morpholinyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

8. The compound or salt of claim 1, wherein R¹ is selected from

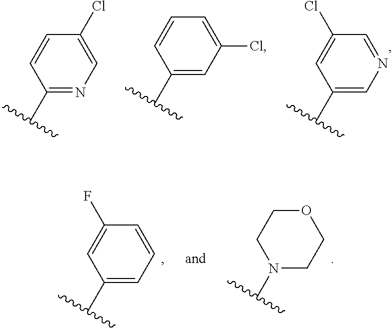 [SMILES: *c1ccc(Cl)cn1 ; *c1cccc(Cl)c1 ; *c1cncc(Cl)c1 ; *c1cccc(F)c1 ; *N1CCOCC1]

9. The compound or salt of claim 1, wherein R² is selected from:
C₁₋₆ alkyl, C₂₋₆ alkenyl, C₂₋₆ alkynyl, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁵;
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵; and
—C(O)NR⁷R⁸.

10. The compound or salt of claim 1, wherein R² is selected from:
C₃₋₁₀ carbocycle and 3- to 10-membered heterocycle, each of which is optionally substituted with one or more substituents independently selected from halogen, —OR⁶, —SR⁶, —N(R⁶)₂, —C(O)R⁶, —C(O)N(R⁶)₂, —N(R⁶)C(O)R⁶, —C(O)OR⁶, —OC(O)R⁶, —NO₂, —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵.

11. The compound or salt of claim 1, wherein R² is selected from:
pyridinyl optionally substituted with one or more substituents independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl.

12. The compound or salt of claim 1, wherein R² is selected from

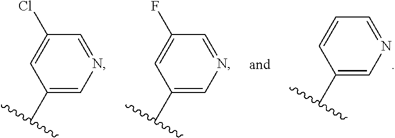 [SMILES: *c1cncc(Cl)c1 ; *c1cncc(F)c1 ; *c1cccnc1]

13. The compound or salt of claim 1, wherein R² is —C(O)NR⁷R⁸.

14. The compound or salt of claim 21, wherein R⁷ is selected from hydrogen and C₁₋₆ alkyl.

15. The compound or salt of claim 22, wherein R⁷ is selected from hydrogen.

16. The compound or salt of claim 1, wherein R⁸ is selected from:
C₁₋₆ alkyl, C₂₋₆ alkenyl, C₂₋₆ alkynyl, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle, and haloalkyl.

17. The compound or salt of claim 1, wherein R⁸ is selected from C₁₋₆ alkyl:

18. The compound or salt of claim 1, wherein R⁸ is selected from ethyl.

19. The compound or salt of claim 1, wherein each R³ is independently selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₆ alkyl optionally substituted with one or more substituents independently selected from halogen, —CN, —OH, —SH —NO₂, and —NH₂.

20. The compound or salt of claim 1, wherein each R³ is C₁₋₆ alkyl optionally substituted with one or more substituents independently selected from halogen, —CN, —OH, —SH —NO₂, and —NH₂.

21. The compound or salt of claim 1, wherein p is 1.

22. The compound or salt of claim 1, wherein p is 0.

23. The compound or salt of claim 1, wherein R⁴ is selected from hydrogen and C₁₋₆ alkyl optionally substituted with one or more substituents independently selected from halogen, —CN, —OH, —SH —NO₂, and —NH₂.

24. The compound or salt of claim 5, wherein R⁴ is hydrogen.

25. The compound or salt of claim 1, wherein each R⁵ is selected from halogen, —CN, —OH, —SH —NO₂, —NH₂, —O—C₁₋₆ alkyl, —S—C₁₋₆ alkyl, —N(C₁₋₆ alkyl)₂, —NH(C₁₋₆ alkyl) and C₁₋₃ alkyl optionally substituted with one or more substituents independently selected from halogen, —CN, —OH, —SH —NO₂, and —NH₂.

26. The compound or salt of claim 1, wherein each R⁶ is selected from hydrogen, halogen, and C₁₋₃ alkyl optionally substituted with one or more substituents independently selected from halogen, —CN, —OH, —SH —NO₂, and —NH₂.

27. The compound of claim 1, wherein the compound is selected from 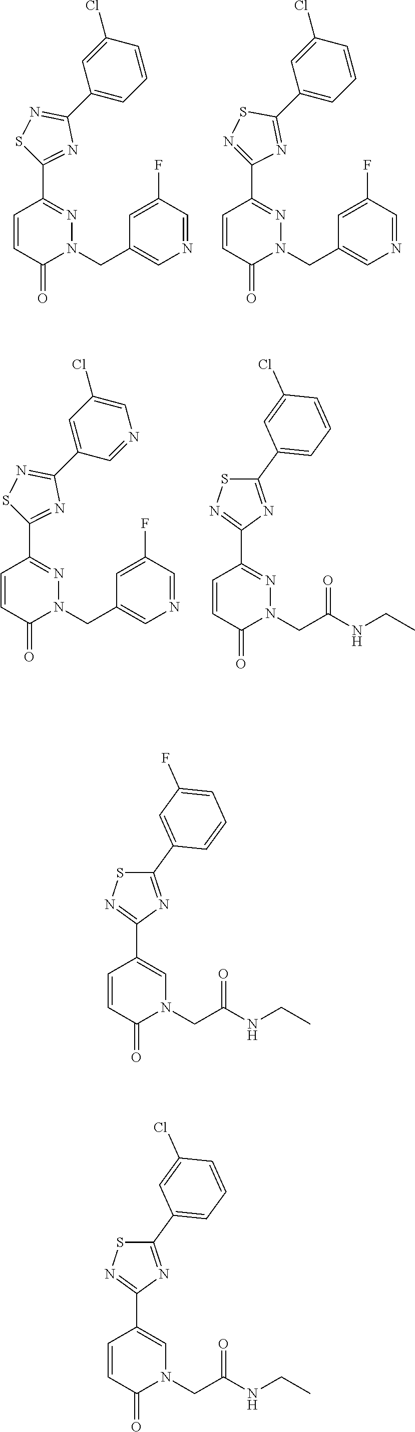 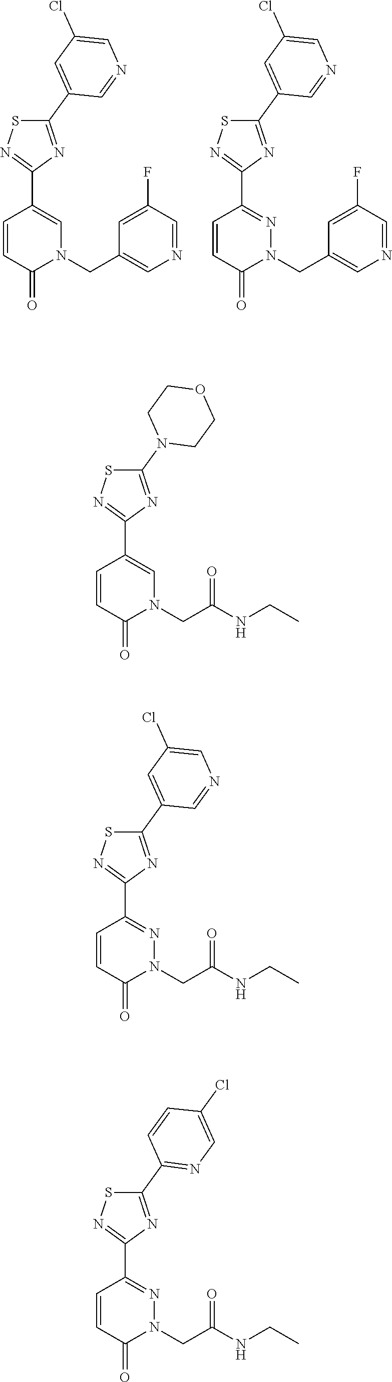
and a salt of ay one thereof.

28. The compound of claim 1, wherein the compound is selected from 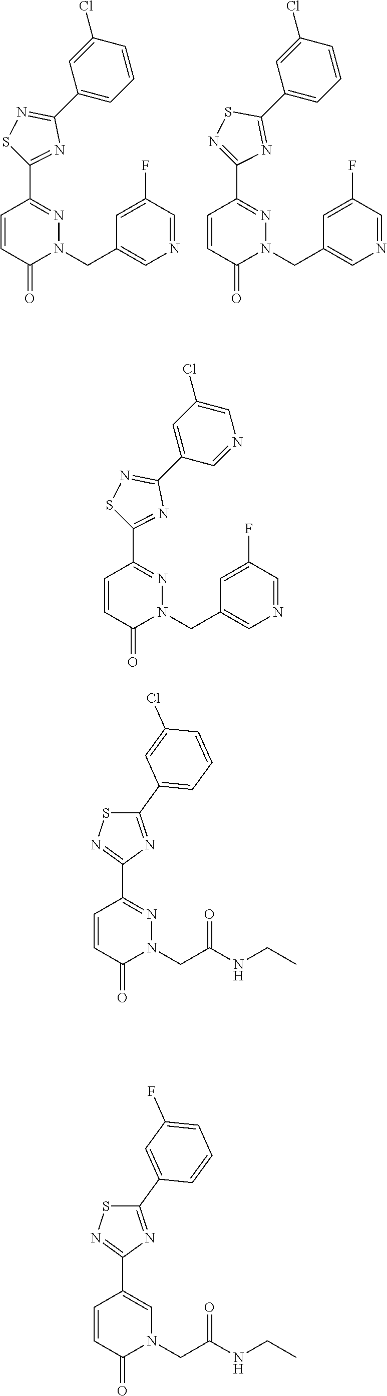 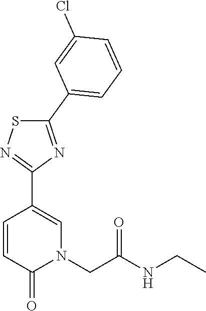 [SMILES: CCNC(=O)Cn1cc(-c2nsc(-c3cccc(Cl)c3)n2)ccc1=O]
and a salt of any one thereof.

29. A compound represented by Formula (II): 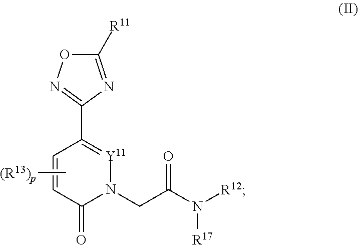
or a salt thereof, wherein:
Y¹¹ is N or CR¹⁴;
R¹¹ is selected from:
hydrogen;
halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, and —CN;
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —C(O)OR¹⁶, —OC(O)R¹⁶, —N(R¹⁶)C(O)N(R¹⁶)₂, —OC(O)N(R¹⁶)₂, —N(R¹⁶)C(O)OR¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
C₃₋₁₀ carbocycle and 3- to 10-membered heterocycle, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O) R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R¹⁵;
R¹² is selected from:
C₁₋₆ alkyl, C₂₋₆ alkenyl, C₂₋₆ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), —CN, C₃₋₁₀ carbocycle and 3- to 10-membered heterocycle, wherein the C₃₋₁₀ carbocycle and 3- to 10-membered heterocycle are each optionally substituted with one or more R¹⁵; and
C₃₋₁₀ carbocycle and 3- to 10-membered heterocycle, each of which is optionally substituted with one or more substituents independently selected from: halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R¹³ is independently selected from halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
R¹⁴ is independently selected from hydrogen, halogen, —OR¹⁶, —SR¹⁶, —N(R¹⁶)₂, —NO₂, —CN, and C₁₋₆ alkyl optionally substituted with one or more substituents independently selected from halogen, —OR¹⁶, —SR¹⁶, —N(R¹⁶)₂, —NO₂, and —CN;
each R¹⁵ is independently selected from:
halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN; and
C₁₋₃ alkyl, C₂₋₃ alkenyl, C₂₋₃ alkynyl, each of which is optionally substituted with one or more substituents independently selected from halogen, —OR¹⁶, —SR¹⁶, —N(R¹⁶)₂, —C(O)R¹⁶, —C(O)N(R¹⁶)₂, —N(R¹⁶)C(O)R¹⁶, —N(R¹⁶)C(O)N(R¹⁶)₂, —OC(O)N(R¹⁶)₂, —N(R¹⁶)C(O)OR¹⁶, —C(O)OR¹⁶, —OC(O)R¹⁶, —S(O)R¹⁶, —S(O)₂R¹⁶, —NO₂, ═O, ═S, ═N(R¹⁶), and —CN;
each R¹⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R¹⁷ is selected from hydrogen and C₁₋₆ alkyl optionally substituted with one or more substituents independently selected from halogen, —OR¹⁶, —SR¹⁶, —N(R¹⁶)₂, —NO₂, and —CN;
p is 0, 1, or 2.

30. A compound represented by Formula (III): 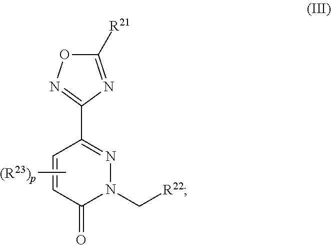
or a salt thereof, wherein:
R²¹ is selected from:
hydrogen;
halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, and —CN;
C₂₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —C(O)OR²⁶, —OC(O)R²⁶, —N(R²⁶)C(O)N(R²⁶)₂, —OC(O)N(R²⁶)₂, —N(R²⁶)C(O)OR²⁶, —S(O)R²⁶, —S(O)₂R²⁶, —NO₂, ═O, ═S, ═N(R²⁶), —CN, C₃₋₁₀ carbocycle and 3- to 10-membered heterocycle, wherein the C₃₋₁₀ carbocycle and 3- to 10-membered heterocycle are each optionally substituted with one or more R²⁵; and
C₃₋₁₀ carbocycle and 3- to 10-membered heterocycle, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O) R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R²⁵;
R²² is selected from:
C₁₋₆ alkyl, C₂₋₆ alkenyl, C₂₋₆ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R²⁵; and
C₃₋₁₀ carbocycle and 3- to 10-membered heterocycle, each of which is optionally substituted with one or more substituents independently selected from: halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R²⁵;
each R²³ is independently selected from halogen, —OR²⁶, —SR²⁶, —N(R²⁶)₂, —NO₂, —CN, and C₁₋₆ alkyl optionally substituted with one or more substituents independently selected from halogen, —OR²⁶, —SR²⁶, —N(R²⁶)₂, —NO₂, and —CN;
each R²⁵ is independently selected from:
halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN; and
C₁₋₃ alkyl, C₂₋₃ alkenyl, C₂₋₃ alkynyl, each of which is optionally substituted with one or more substituents independently selected from halogen, —OR²⁶, —SR²⁶, —N(R²⁶)₂, —C(O)R²⁶, —C(O)N(R²⁶)₂, —N(R²⁶)C(O)R²⁶, —N(R²⁶)C(O)N(R²⁶)₂, —OC(O)N(R²⁶)₂, —N(R²⁶)C(O)OR²⁶, —C(O)OR²⁶, —OC(O)R²⁶, —S(O)R²⁶, —S(O)₂R²⁶, —NO₂, ═O, ═S, ═N(R²⁶), and —CN;
each R²⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

31. A compound represented by Formula (IV): 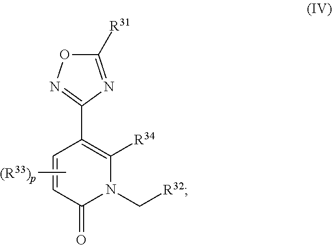
or a salt thereof, wherein:
R³¹ is selected from:
hydrogen;
halogen, —OR³⁶, —SR³⁶, —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂ and —CN;
C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —C(O)OR³⁶, —OC(O)R³⁶, —N(R³⁶)C(O)N(R³⁶)₂, —OC(O)N(R³⁶)₂, —N(R³⁶)C(O)OR³⁶, —S(O)R³⁶, —S(O)₂R³⁶, —NO₂, ═O, ═S, ═N(R³⁶), —CN, C₃₋₁₀ carbocycle and 3- to 10-membered heterocycle, wherein the C₃₋₁₀ carbocycle and 3- to 10-membered heterocycle are each optionally substituted with one or more R³⁵; and
C₃₋₁₀ carbocycle and 3- to 10-membered heterocycle, each of which is optionally substituted with one or more substituents independently selected from halogen, —OR³⁶, —SR³⁶, —N(R³⁶)₂, —C(O)R³⁶, —C(O)N(R³⁶)₂, —N(R³⁶)C(O) R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R³⁵;
R³² is selected from:
C₁₋₆ alkyl, C₂₋₆ alkenyl, C₂₋₆ alkynyl, each of which is optionally substituted with one or more substituents independently selected from halogen, —OR³⁶, —SR³⁶; —N(R³⁶)₂, —C(O)R³⁶, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R³⁵; and
3- to 10-membered heterocycle optionally substituted with one or more substituents independently selected from: halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R³⁵;
each R³³ is independently selected from halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
R³⁴ is independently selected from hydrogen, halogen, —OR³⁶, —SR³⁶, —N(R³⁶)₂, —NO₂, —CN, and C₁₋₆ alkyl optionally substituted with one or more substituents independently selected from halogen, —OR³⁶, —SR³⁶, —N(R³⁶)₂, —NO₂, and —CN;
each R³⁵ is independently selected from:
halogen, —OR³⁶, —SR³⁶, —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN; and
C₁₋₃ alkyl, C₂₋₃ alkenyl, C₂₋₃ alkynyl, each of which is optionally substituted with one or more substituents independently selected from halogen, —OR³⁶, —SR³⁶; —N(R³⁶)₂, —C(O)R³⁶, —C(O)N(R³⁶)₂, —N(R³⁶)C(O)R³⁶, —N(R³⁶)C(O)N(R³⁶)₂, —OC(O)N(R³⁶)₂, —N(R³⁶)C(O)OR³⁶, —C(O)OR³⁶, —OC(O)R³⁶, —S(O)R³⁶, —S(O)₂R³⁶, —NO₂, ═O, ═S, ═N(R³⁶), and —CN;
each R³⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl; and
p is 0, 1, or 2.

32. A compound represented by Formula (V): 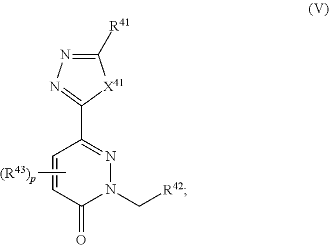
or a salt thereof, wherein:
X⁴¹ is O or S;
R⁴¹ is selected from:
hydrogen;
halogen, —OR⁴⁶, —SR⁴⁶, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂ and —CN;
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —C(O)OR⁴⁶, —OC(O)R⁴⁶, —N(R⁴⁶)C(O)N(R⁴⁶)₂, —OC(O)N(R⁴⁶)₂, —N(R⁴⁶)C(O)OR⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵; and
C₃₋₁₀ carbocycle and 3- to 10-membered heterocycle, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O) R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁴¹;
R⁴² is selected from:
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
—C(O)NR⁴⁷R⁴⁸;
each R⁴³ is independently selected from halogen, —OR⁴⁶, —SR⁴⁶, —N(R⁴⁶)₂, —NO₂, —CN, and C₁₋₆ alkyl optionally substituted with one or more substituents independently selected from halogen, —OR⁴⁶, —SR⁴⁶, —N(R⁴⁶)₂, —NO₂, and —CN;
each R⁴⁵ is independently selected from:
halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —NO₂, ═O, ═S, ═N(R⁴⁶), and —CN; and
C₁₋₃ alkyl, C₂₋₃ alkenyl, C₂₋₃ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), and —CN;
each R⁴⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁴⁷ is selected from hydrogen and C₁₋₆ alkyl optionally substituted with one or more substituents independently selected from halogen, —OR⁴⁶, —SR⁴⁶, —N(R⁴⁶)₂, —NO₂, and —CN;
R⁴⁸ is selected from:
C₁₋₆ alkyl, C₂₋₆ alkenyl, C₂₋₆ alkynyl, each of which is optionally substituted with one or more substituents independently selected from halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₃₋₁₀ carbocycle and 3- to 10-membered heterocycle, wherein the C₃₋₁₀ carbocycle and 3- to 10-membered heterocycle are each optionally substituted with one or more R⁴⁵;
C₃₋₁₀ carbocycle and 3- to 10-membered heterocycle, each of which is optionally substituted with one or more substituents independently selected from: halogen, —OR⁴⁶, —SR⁴⁶, —N(R⁴⁶)₂, —C(O)R⁴⁶, —C(O)N(R⁴⁶)₂, —N(R⁴⁶)C(O)R⁴⁶, —N(R⁴⁶)C(O)N(R⁴⁶)₂, —OC(O)N(R⁴⁶)₂, —N(R⁴⁶)C(O)OR⁴⁶, —C(O)OR⁴⁶, —OC(O)R⁴⁶, —S(O)R⁴⁶, —S(O)₂R⁴⁶, —NO₂, ═O, ═S, ═N(R⁴⁶), —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁴⁵; and
p is 0, 1, or 2.

33. A compound represented by Formula (VI): 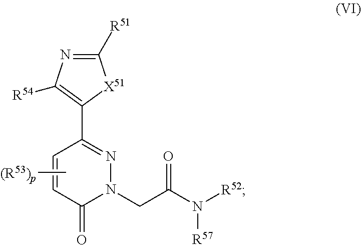
or a salt thereof, wherein:
X⁵¹ is selected from O and S;
R⁵¹ is selected from:
hydrogen;
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂ and —CN;
C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —C(O)OR⁵⁶, —OC(O)R⁵⁶, —N(R⁵⁶)C(O)N(R⁵⁶)₂, —OC(O)N(R⁵⁶)₂, —N(R⁵⁶)C(O)OR⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
C₃₋₁₀ carbocycle and 3- to 10-membered heterocycle, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O) R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R¹⁶, —NO₂, ═O, ═S, ═N(R⁵⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁵;
R⁵² is selected from:
C₁₋₆ alkyl, C₂₋₆ alkenyl, C₂₋₆ alkynyl, each of which is optionally substituted with one or more substituents independently selected from halogen, —OR⁵⁶, —SR⁵⁶, —N(R⁵⁶)₂, —C(O)R⁵⁶, —C(O)N(R¹⁶)₂, —N(R⁵⁶)C(O)R¹⁶, —N(R⁵⁶)C(O)N(R¹⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), —CN, C₃₋₁₀ carbocycle and 3- to 10-membered heterocycle, wherein the C₃₋₁₀ carbocycle and 3- to 10-membered heterocycle are each optionally substituted with one or more R⁵⁵; and
C₃₋₁₀ carbocycle and 3- to 10-membered heterocycle, each of which is optionally substituted with one or more substituents independently selected from: halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁵;
each R⁵³ is independently selected from halogen, —OR⁵⁶, —SR⁵⁶, —N(R⁵⁶)₂, —NO₂, —CN, C₁₋₆ alkyl optionally substituted with one or more substituents independently selected from halogen, —OR⁵⁶, —SR⁵⁶, —N(R⁵⁶)₂, —NO₂, and —CN;
R⁵⁴ is independently selected from hydrogen, halogen, —OR⁵⁶, —SR⁵⁶, —N(R⁵⁶)₂, —NO₂, —CN, C₁₋₆ alkyl, C₃₋₁₀ carbocycle and 3- to 10-membered heterocycle, wherein C₁₋₆ alkyl, C₃₋₁₀ carbocycle and 3- to 10-membered heterocycle are each optionally substituted with one or more substituents independently selected from halogen, —OR⁵⁶, —SR⁵⁶, —N(R⁵⁶)₂, —NO₂, and —CN;
each R⁵⁵ is independently selected from:
halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR⁵⁶, —C(O)OR⁵⁶, —OC(O)R⁵⁶, —S(O)R⁵⁶, —S(O)₂R⁵⁶, —NO₂, ═O, ═S, ═N(R⁵⁶), and —CN; and
C₁₋₃ alkyl, C₂₋₃ alkenyl, C₂₋₃ alkynyl, each of which is optionally substituted with one or more substituents independently selected from halogen, —OR⁵⁶, —SR⁵⁶, —N(R⁵⁶)₂, —C(O)R⁵⁶, —C(O)N(R⁵⁶)₂, —N(R⁵⁶)C(O)R⁵⁶, —N(R⁵⁶)C(O)N(R⁵⁶)₂, —OC(O)N(R⁵⁶)₂, —N(R⁵⁶)C(O)OR¹⁶, —C(O)OR¹⁶, —OC(O)R¹⁶, —S(O)R¹⁶, —S(O)₂R¹⁶, —NO₂, ═O, ═S, ═N(R⁵⁶), and —CN;
each R⁵⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
R⁵⁷ is selected from hydrogen and C₁₋₆ alkyl optionally substituted with one or more substituents independently selected from halogen, —OR⁵⁶, —SR⁵⁶, —N(R⁵⁶)₂, —NO₂, and —CN; and
p is 0, 1, or 2.

34. A compound represented by Formula (VII): 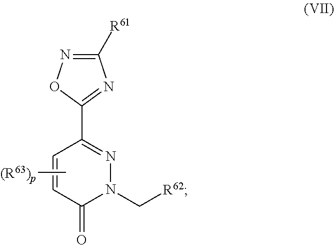
or a salt thereof, wherein:
R⁶¹ is selected from:
hydrogen;
halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, and —CN;
C₁₋₆ alkyl, C₂₋₆ alkenyl, C₂₋₆ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —C(O)OR⁶⁶, —OC(O)R⁶⁶, —N(R⁶⁶)C(O)N(R⁶⁶)₂, —OC(O)N(R⁶⁶)₂, —N(R⁶⁶)C(O)OR⁶⁶, —S(O)R⁶⁶, —S(O)₂R⁶⁶, —NO₂, ═O, ═S, ═N(R⁶⁶), —CN, C₃₋₁₀ carbocycle and 3- to 10-membered heterocycle, wherein the C₃₋₁₀ carbocycle and 3- to 10-membered heterocycle are each optionally substituted with one or more R⁶⁵; and
C₃₋₁₀ carbocycle and 3- to 10-membered heterocycle, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O) R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁶⁵;
R⁶² is selected from:
5-membered heterocycle optionally substituted with one or more substituents independently selected from: halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁶⁵;
each R⁶³ is independently selected from halogen, —OR⁶⁶, —SR⁶⁶, —N(R⁶⁶)₂, —NO₂, —CN, and C₁₋₆ alkyl optionally substituted with one or more substituents independently selected from halogen, —OR⁶⁶, —SR⁶⁶, —N(R⁶⁶)₂, —NO₂, and —CN;
each R⁶⁵ is independently selected from:
halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN; and
C₁₋₃ alkyl, C₂₋₃ alkenyl, C₂₋₃ alkynyl, each of which is optionally substituted with one or more substituents independently selected from halogen, —OR⁶⁶, —SR⁶⁶, —N(R⁶⁶)₂, —C(O)R⁶⁶, —C(O)N(R⁶⁶)₂, —N(R⁶⁶)C(O)R⁶⁶, —N(R⁶⁶)C(O)N(R⁶⁶)₂, —OC(O)N(R⁶⁶)₂, —N(R⁶⁶)C(O)OR⁶⁶, —C(O)OR⁶⁶, —OC(O)R⁶⁶, —S(O)R⁶⁶, —S(O)₂R⁶⁶, —NO₂, ═O, ═S, ═N(R⁶⁶), and —CN;
each R⁶⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

35. A compound represented by Formula (VIII): 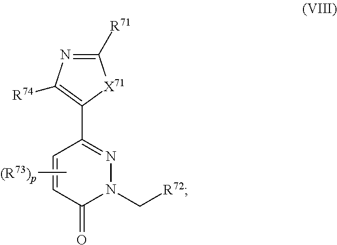
or a salt thereof, wherein:
X⁷¹ is selected from S and 0;
R⁷¹ is selected from:
hydrogen;
halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, and —CN;
C₁₋₆ alkyl, C₂₋₆ alkenyl, C₂₋₆ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —C(O)OR⁷⁶, —OC(O)R⁷⁶, —N(R⁷⁶)C(O)N(R⁷⁶)₂, —OC(O)N(R⁷⁶)₂, —N(R⁷⁶)C(O)OR⁷⁶, —S(O)R⁷⁶, —S(O)₂R⁷⁶, —NO₂, ═O, ═S, ═N(R⁷⁶), —CN, C₃₋₁₀ carbocycle and 3- to 10-membered heterocycle, wherein the C₃₋₁₀ carbocycle and 3- to 10-membered heterocycle are each optionally substituted with one or more R⁷⁵; and
C₃₋₁₀ carbocycle and 3- to 10-membered heterocycle, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O) R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), —CN, C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are each optionally substituted with one or more R⁷¹;
R⁷² is selected from:
3- to 10-membered heterocycle optionally substituted with one or more substituents independently selected from: halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are each optionally substituted with one or more R⁷¹;
each R⁷³ is independently selected from halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
R⁷⁴ is independently selected from hydrogen, halogen, —OR⁷⁶, —SR⁷⁶, —N(R⁷⁶)₂, —NO₂, —CN, and C₁₋₆ alkyl optionally substituted with one or more substituents independently selected from halogen, —OR⁷⁶, —SR⁷⁶, —N(R⁷⁶)₂, —NO₂, and —CN;
each R⁷⁵ is independently selected from:
halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN; and
C₁₋₃ alkyl, C₂₋₃ alkenyl, C₂₋₃ alkynyl, each of which is optionally substituted with one or more substituents independently selected from halogen, —OR⁷⁶, —SR⁷⁶, —N(R⁷⁶)₂, —C(O)R⁷⁶, —C(O)N(R⁷⁶)₂, —N(R⁷⁶)C(O)R⁷⁶, —N(R⁷⁶)C(O)N(R⁷⁶)₂, —OC(O)N(R⁷⁶)₂, —N(R⁷⁶)C(O)OR⁷⁶, —C(O)OR⁷⁶, —OC(O)R⁷⁶, —S(O)R⁷⁶, —S(O)₂R⁷⁶, —NO₂, ═O, ═S, ═N(R⁷⁶), and —CN;
each R⁷⁶ is independently selected from:
hydrogen;
C₁₋₆ alkyl, C₂₋₆ alkenyl, C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and haloalkyl;
p is 0, 1, or 2.

36. A pharmaceutical composition comprising a compound or salt of claim 1.

37. A method of treating activity-induced muscle damage, a metabolic myopathy, a neuromuscular condition, or a movement disorder, the method comprising administering to a subject in need thereof a compound or salt of claim 1.
